SEQUENCE LISTING (149 sequences)

SEQ ID NO: 1 - DNA, 1072 bp, Mattirolomyces terfezioides
  source 1..1072: mol_type = unassigned DNA
caagtctttaagtgttgatcgagcagtagaaatattaatcgcccaattaattgctcttactctaggaaatgtagtactcaagttggccctctaagtccctgattcagtgtcactttttctcctttgacagtaggctagattctagctcttgtgtccgtttcttaaaatcctgatatggcaccttagcgtcagagcagaatacctgttctcatacaccttggctatgatccgatatttaaatagagcaatcacccttctggatatcttccccatagctcccattgacctcaaacattttctaatcccctataccagccatattacttgtctaagcacttcacattcttcctcacaatacgaaacctcctaaaatgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacagtaaagatcgatgatgcctaatatgcctccataacactgacccgcgtgcacatggccgcatgaatgataagggggatatcgatgatgatgggattagttattggaattttcacaatggacgtcggcttggatttacaataatcgtttcatttgtattcaaatattcctatttccttgggtttttgtatttatctccttcatcacgccttctgaggccgtgggaagatgaatatgtaatcaaaagaagttaggatatgcatcatgtacagaaagtggaccgcaaccccttcagcgaaatgttataaagatgatatctaagacgccaaagcacattctcag

SEQ ID NO: 2 - DNA, 366 bp, Mattirolomyces terfezioides
  source 1..366: mol_type = unassigned DNA
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacagtaa

SEQ ID NO: 3 - protein, 121 aa, Mattirolomyces terfezioides [sequence omitted]

SEQ ID NO: 4 - protein, 20 aa, Mattirolomyces terfezioides
PDLSSFITIKNNSNHVFTRT

SEQ ID NO: 5 - DNA, 873 bp, Pisolithus tincturius
  source 1..873: mol_type = unassigned DNA
tcgtccgagcagataaacatcgacgggcaggatgtcatttctactgaatgtctggaacgctgtccaggcccgaggtacaatcttcctatcaacgacgcagacgaggtacaatactgctgtcaatgctacaaaatcatgccgggtcctgtgcatggccgtctgaggaggttcttctggtagaagtttaaaaggtctctgggcgacgggtgtaacttgccaatttcccctacatccgaacatggcagatgaacagaaggaactcgcaaccaacgtagccgaccatggtcttactggccaacttccctcatcaaacgaaaccacggagggactcggagtcacgcaagactgctcttgctacatcacgctccacaatgggaccgaccatgagcttgtgctcgtctacgctcaagagaaacacggtgaatggaagacccgccctgcggaaaccgtgagccagaagagcaatatcaagttttggctcaaagacttattccttggtcctggagcagagggtatggtgaagtatcgaatcggaagtaccgagcacaaggtgcagatgaacttcagctgtcctatgtcttctcccaactcggcgtcctggagtcaaggtgaacatgagattccaggcatctggttgccctgtccggaatacaataaatctgatgcgttgcatgccgtgtttgaagtacaacctgggaattaaggcgtcggggcggggagacgtactataccccattgtcgatagcctctggaagtgtcatcataatgactgtttgttttgtcattgaccggcgacttgtcattttgagttcgtttcccttggcctgaggcgcacttaccgggcatcgtcatagagctactgcttaccacaaa

SEQ ID NO: 6 - DNA, 464 bp, Pisolithus tincturius
  source 1..464: mol_type = unassigned DNA
atggcagatgaacagaaggaactcgcaaccaacgtagccgaccatggtcttactggccaacttccctcatcaaacgaaaccacggagggactcggagtcacgcaagactgctcttgctacatcacgctccacaatgggaccgaccatgagcttgtgctcgtctacgctcaagagaaacacggtgaatggaagacccgccctgcggaaaccgtgagccagaagagcaatatcaagttttggctcaaagacttattccttggtcctggagcagagggtatggtgaagtatcgaatcggaagtaccgagcacaaggtgcagatgaacttcagctgtcctatgtcttctcccaactcggcgtcctggagtcaaggtgaacatgagattccaggcatctggttgccctgtccggaatacaataaatctgatgcgttgcatgccgtgtttgaagtacaacctgggaatta

SEQ ID NO: 7 - protein, 154 aa, Pisolithus tincturius
MADEQKELATNVADHGLTGQLPSSNETTEGLGVTQDCSCYITLHNGTDHELVLVYAQEKHGEWKTRPAETVSQKSNIKFWLKDLFLGPGAEGMVKYRIGSTEHKVQMNFSCPMSSPNSASWSQGEHEIPGIWLPCPEYNKSDALHAVFEVQPGN

SEQ ID NO: 8 - protein, 121 aa, synthetic construct
  REGION 1..121: note = Mutated protein from Mattirolomyces terfezioides
MPDLSSFITIKHNSHHVFTRTAIYSKYAAVQWSQEPHLSISPGKWDLFILKDILSIRGTSGYVQYRVGAAQGWVRVTFSSLVGADEVAEWSSGDLPDGFVWHQQVTTGSRPLQATFEATKQ

SEQ ID NO: 9 - protein, 121 aa, synthetic construct
  REGION 1..121: note = Mutated protein from Mattirolomyces terfezioides
MPDLSSFITIKNNTDHVFTRTAIYSKYAAVQWSPQPQLSISHGKWDLFILKDILSIRGTSGYVQYRVGHGPDWVRVTFSSLVGADEVAEWSSGDLPDGFVWHEHVPMGSRPLQATFEATKQ

SEQ ID NO: 10 - protein, 121 aa, synthetic construct
  REGION 1..121: note = Mutated protein from Mattirolomyces terfezioides
MPDLSSFITIKNKPNHVFTRTAIYSKYAAVQWSSESQLSIPSGEWDLFILKDILSIRGTSGYVQYRVGVGSCWVRVTFSSLVGADEVAEWSSGDLPDGFVLQKSVQTGSRPLQATFEATKQ

SEQ ID NO: 11 - protein, 121 aa, synthetic construct
  REGION 1..121: note = Mutated protein from Mattirolomyces terfezioides
MPDLSSFITIKNHTNRVFTRTAIYSKYAAVQWTPESLMSISPGTWDLFILKDILSIRGTSGYVQYRVGDCPGWVRVTFSSLVGADEVAEWSSGDLPDGFVLQKPVQMGTRPLQATFEATKQ

SEQ ID NO: 12 - protein, 121 aa, synthetic construct
  REGION 1..121: note = Mutated protein from Mattirolomyces terfezioides
MPDLSSFITIKDNSNQVFTRTAIYSKYAAVQWCPELPRSISLGKWDLFILKDILSIRGTSGYVQYRVGDALAWVRVTFSSLVGADEVAEWSSGDLPDGFVLQKLVRNGSRPLQATFEATKQ

SEQ ID NO: 13 - protein, 121 aa, synthetic construct
  REGION 1..121: note = Mutated protein from Mattirolomyces terezoides
MPDLSSFITIKHNAHHVFTRTAIYSKYAAVQWSQEQQLSIAPGTWDLFILKDILSIRGTSGYVQYRVRDGPGWVRVTFSSLVGADEVAEWSSGDLPDGFVLQKQVRKRARPLQATFEATKQ

SEQ ID NO: 14 - protein, 121 aa, synthetic construct
  REGION 1..121: note = Mutated protein from Mattirolomyces terfezioides
MPDLSSFITIKNHSNNVFTRTAIYSKYAAVQWPRGPLLSISPRQWDLFILKDILSIRGTSGYVQYRVGDGRGWVRVTFSSLVGADEVAEWSSGDLPDGFVLQKRVGNGSRPLQATFEATKQ

SEQ ID NO: 15 - protein, 121 aa, synthetic construct
  REGION 1..121: note = Mutated protein from Mattirolomyces terfezioides
MPDLSSFITIKNNSNHVFTRTAIYSKYAAVQWSHQPRPSIWPDMWDLFILKDILSIRGTSGYVQYRVGDDPGWVRVTFSSLVGADEVAEWSSGDLPDGFVLRMPDPTGSRPLQATFEATKQ

SEQ ID NO: 16 - protein, 121 aa, synthetic construct
  REGION 1..121: note = Mutated protein from Mattirolomyces terfezioides
MPDLSSFITIKDDPNQVFTRTAIYSKYAAVQWSPEHQFSISPGKWDLFILKDILSIRGTSGYVQYRVCDCPGWVRVTFSSLVGADEVAEWSSGDLPDGFVFQTPVTTGPRPLQATFEATKQ

SEQ ID NO: 17 - protein, 121 aa, synthetic construct
  REGION 1..121: note = Mutated protein from Mattirolomyces terfezioides
MPDLSSFITIKNKTNHVFTRTAIYSKYAAVQWTQQPQISITQGKWDLFILKDILSIRGTSGYVQYRVGDDPGWVRVTFSSLVGADEVAEWSSGDLPDGFVLRKPLRMGSRPLQATFEATKQ

SEQ ID NO: 18 - DNA, 58 bp, synthetic construct
  misc_feature 1..58: note = Synthetic Adapter Sequence
  source 1..58: mol_type = other DNA
aatgatacggcgaccaccgagatctacactctttccctacacgacgctcttccgatct

SEQ ID NO: 19 - DNA, 58 bp, synthetic construct
  misc_feature 1..58: note = Synthetic Adapter Sequence
  misc_feature 34: note = insertion of i7 barcode
  source 1..58: mol_type = other DNA
gatcggaagagcacacgtctgaactccagtcacnatctcgtatgccgtcttctgcttg

SEQ ID NO: 20 - DNA, 384 bp, synthetic construct
  misc_feature 1..384: note = Modified coding sequence for E. coli expression for protein from Mattirolomyces terfezioides with His-Tag
  source 1..384: mol_type = other DNA
  CDS 1..384
atgccggatttgagctcgttcattactattaagaataactctaaccatgtttttacgcgtaccgcaatctattccaaatatgccgcagtccagtggagcccggaaccgcagctgagcattagcccaggtaaatgggacctgttcatcctgaaagacatcctgagcatccgcggtacgtccggctacgtgcagtaccgtgtcggcgacggtccgggctgggtgcgtgttacctttagcagcctggtgggtgcggacgaagttgctgagtggagcagcggtgatctgccggatggctttgttctgcaaaagcctgtccgcaccggtagccgtccgctgcaagcgacgttcgaggcgaccaagcaacatcaccatcaccaccactaa

SEQ ID NO: 21 - protein, 127 aa, synthetic construct
  REGION 1..127: note = Synthetic Construct
MPDLSSFITIKNNSNHVFTRTAIYSKYAAVQWSPEPQLSISPGKWDLFILKDILSIRGTSGYVQYRVGDGPGWVRVTFSSLVGADEVAEWSSGDLPDGFVLQKPVRTGSRPLQATFEATKQHHHHHH

SEQ ID NO: 22 - DNA, 366 bp, synthetic construct
  misc_feature 1..366: note = Modified coding sequence for Saccharomyces cerevisiae expression for protein from Mattirolomyces terfezioides with His-Tag
  source 1..366: mol_type = other DNA
atgcctgatttatcaagttttatcaccattaaaaataactctaaccatgtttttactagaacagccatctactcaaagtacgcagccgtccaatggtccccagaacctcagttgtctatatctccaggtaaatgggatcttttcatcttaaaagatattctaagtattagaggcacttctggttacgtacagtatcgtgttggtgatggacctggttgggttagagtaacattcagctcattggttggggctgacgaagtggctgagtggtcctcaggtgacctcccagatggcttcgtgctgcaaaagccagtcagaactggatctagaccattgcaagcgacattcgaagcaacaaagcaatga

SEQ ID NO: 23 - DNA, 384 bp, synthetic construct
  misc_feature 1..384: note = Coding Sequence for modfied protein from Mattirolomyces terfezioides including His-Tag
  source 1..384: mol_type = other DNA
atgccggaattgagctcgttcattactattaagaataactctaaccatgtttttacgcgtaccgcaatctattccaaatatgccgcagtccagtggagcccggaaccgcagctgagcattagcccaggtaaatgggacctgttcatcctgaaagacatcctgagcatccgcggtacgtccggctacgtgcagtaccgtgtcggcgacggtccgggctgggtgcgtgttacctttagcagcctggtgggtgcggacgaagttgctgagtggagcagcggtgatctgccggatggctttgttctgcaaaagcctgtccgcaccggtagccgtccgctgcaagcgacgttcgaggcgaccaagcaacatcaccaccaccaccactaa

SEQ ID NO: 24 - protein, 127 aa, synthetic construct
  REGION 1..127: note = Modified protein from Mattirolomyces terfezioides with optional His-Tag
  REGION 122..127: note = Absent or Histidine
MPELSSFITIKNNSNHVFTRTAIYSKYAAVQWSPEPQLSISPGKWDLFILKDILSIRGTSGYVQYRVGDGPGWVRVTFSSLVGADEVAEWSSGDLPDGFVLQKPVRTGSRPLQATFEATKQXXXXXX

SEQ ID NO: 25 - DNA, 384 bp, synthetic construct
  misc_feature 1..384: note = Coding Sequence of modified protein from Mattirolomyces terfezioides with His-Tag
  source 1..384: mol_type = other DNA
atgccggatttgagctcgttcattactattagaaataactctaaccatgtttttacgcgtaccgcaatctattccaaatatgccgcagtccagtggagcccggaaccgcagctgagcattagcccaggtaaatgggacctgttcatcctgaaagacatcctgagcatccgcggtacgtccggctacgtgcagtaccgtgtcggcgacggtccgggctgggtgcgtgttacctttagcagcctggtgggtgcggacgaagttgctgagtggagcagcggtgatctgccggatggctttgttctgcaaaagcctgtccgcaccggtagccgtccgctgcaagcgacgttcgaggcgaccaagcaacatcaccaccaccaccactaa

SEQ ID NO: 26 - protein, 127 aa, synthetic construct
  REGION 1..127: note = Modified protein from Mattirolomyces terfezioides with optional His-Tag
  REGION 122..127: note = Absent or Histidine
MPDLSSFITIRNNSNHVFTRTAIYSKYAAVQWSPEPQLSISPGKWDLFILKDILSIRGTSGYVQYRVGDGPGWVRVTFSSLVGADEVAEWSSGDLPDGFVLQKPVRTGSRPLQATFEATKQXXXXXX

SEQ ID NO: 27 - DNA, 384 bp, synthetic construct
  misc_feature 1..384: note = Coding Sequence of modified protein from Mattirolomyces terfezioides with His-Tag
  source 1..384: mol_type = other DNA
atgccggatttgagctcgttcattactattaagaataactctaaccatgtttttacgaaaaccgcaatctattccaaatatgccgcagtccagtggagcccggaaccgcagctgagcattagcccaggtaaatgggacctgttcatcctgaaagacatcctgagcatccgcggtacgtccggctacgtgcagtaccgtgtcggcgacggtccgggctgggtgcgtgttacctttagcagcctggtgggtgcggacgaagttgctgagtggagcagcggtgatctgccggatggctttgttctgcaaaagcctgtccgcaccggtagccgtccgctgcaagcgacgttcgaggcgaccaagcaacatcaccaccaccaccactaa

SEQ ID NO: 28 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTKTAIYSKYAAVQWSPEPQLSISPGKWDLFILKDILSIRGTSGYVQYRVGDGPGWVRVTFSSLVGADEVAEWSSGDLPDGFVLQKPVRTGSRPLQATFEATKQXXXXXX

SEQ ID NO: 29 - DNA, 384 bp, synthetic construct
  misc_feature 1..384: note = Coding Sequence of modified protein from Mattirolomyces terfezioides with His-Tag
  source 1..384: mol_type = other DNA
atgccggatttgagctcgttcattactattaagaataactctaaccatgtttttacgcgtaccgcaatctattcccgctatgccgcagtccagtggagcccggaaccgcagctgagcattagcccaggtaaatgggacctgttcatcctgaaagacatcctgagcatccgcggtacgtccggctacgtgcagtaccgtgtcggcgacggtccgggctgggtgcgtgttacctttagcagcctggtgggtgcggacgaagttgctgagtggagcagcggtgatctgccggatggctttgttctgcaaaagcctgtccgcaccggtagccgtccgctgcaagcgacgttcgaggcgaccaagcaacatcaccaccaccaccactaa

SEQ ID NO: 30 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSRYAAVQWSPEPQLSISPGKWDLFILKDILSIRGTSGYVQYRVGDGPGWVRVTFSSLVGADEVAEWSSGDLPDGFVLQKPVRTGSRPLQATFEATKQXXXXXX

SEQ ID NO: 31 - DNA, 384 bp, synthetic construct
  misc_feature 1..384: note = Coding Sequence of modified protein from Mattirolomyces terfezioides with His-Tag
  source 1..384: mol_type = other DNA
atgccggatttgagctcgttcattactattaagaataactctaaccatgtttttacgcgtaccgcaatctattccaaatatgccgcagtccagtggagcccggatccgcagctgagcattagcccaggtaaatgggacctgttcatcctgaaagacatcctgagcatccgcggtacgtccggctacgtgcagtaccgtgtcggcgacggtccgggctgggtgcgtgttacctttagcagcctggtgggtgcggacgaagttgctgagtggagcagcggtgatctgccggatggctttgttctgcaaaagcctgtccgcaccggtagccgtccgctgcaagcgacgttcgaggcgaccaagcaacatcaccaccaccaccactaa

SEQ ID NO: 32 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPDPQLSISPGKWDLFILKDILSIRGTSGYVQYRVGDGPGWVRVTFSSLVGADEVAEWSSGDLPDGFVLQKPVRTGSRPLQATFEATKQXXXXXX

SEQ ID NO: 33 - DNA, 384 bp, synthetic construct
  misc_feature 1..384: note = Coding Sequence of modified protein from Mattirolomyces terfezioides with His-Tag
  source 1..384: mol_type = other DNA
atgccggatttgagctcgttcattactattaagaataactctaaccatgtttttacgcgtaccgcaatctattccaaatatgccgcagtccagtggagcccggaaccgcagctgagcattagcccaggtcgctgggacctgttcatcctgaaagacatcctgagcatccgcggtacgtccggctacgtgcagtaccgtgtcggcgacggtccgggctgggtgcgtgttacctttagcagcctggtgggtgcggacgaagttgctgagtggagcagcggtgatctgccggatggctttgttctgcaaaagcctgtccgcaccggtagccgtccgctgcaagcgacgttcgaggcgaccaagcaacatcaccaccaccaccactaa

SEQ ID NO: 34 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPQLSISPGRWDLFILKDILSIRGTSGYVQYRVGDGPGWVRVTFSSLVGADEVAEWSSGDLPDGFVLQKPVRTGSRPLQATFEATKQXXXXXX

SEQ ID NO: 35 - DNA, 384 bp, synthetic construct
  misc_feature 1..384: note = Coding Sequence of modified protein from Mattirolomyces terfezioides with His-Tag
  source 1..384: mol_type = other DNA
atgccggatttgagctcgttcattactattaagaataactctaaccatgtttttacgcgtaccgcaatctattccaaatatgccgcagtccagtggagcccggaaccgcagctgagcattagcccaggtaaatgggaactgttcatcctgaaagacatcctgagcatccgcggtacgtccggctacgtgcagtaccgtgtcggcgacggtccgggctgggtgcgtgttacctttagcagcctggtgggtgcggacgaagttgctgagtggagcagcggtgatctgccggatggctttgttctgcaaaagcctgtccgcaccggtagccgtccgctgcaagcgacgttcgaggcgaccaagcaacatcaccaccaccaccactaa

SEQ ID NO: 36 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPQLSISPGKWELFILKDILSIRGTSGYVQYRVGDGPGWVRVTFSSLVGADEVAEWSSGDLPDGFVLQKPVRTGSRPLQATFEATKQXXXXXX

SEQ ID NO: 37 - DNA, 384 bp, synthetic construct
  misc_feature 1..384: note = Coding Sequence of modified protein from Mattirolomyces terfezioides with His-Tag
  source 1..384: mol_type = other DNA
atgccggatttgagctcgttcattactattaagaataactctaaccatgtttttacgcgtaccgcaatctattccaaatatgccgcagtccagtggagcccggaaccgcagctgagcattagcccaggtaaatgggacctgttcatcctgcgcgacatcctgagcatccgcggtacgtccggctacgtgcagtaccgtgtcggcgacggtccgggctgggtgcgtgttacctttagcagcctggtgggtgcggacgaagttgctgagtggagcagcggtgatctgccggatggctttgttctgcaaaagcctgtccgcaccggtagccgtccgctgcaagcgacgttcgaggcgaccaagcaacatcaccaccaccaccactaa

SEQ ID NO: 38 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPQLSISPGKWDLFILRDILSIRGTSGYVQYRVGDGPGWVRVTFSSLVGADEVAEWSSGDLPDGFVLQKPVRTGSRPLQATFEATKQXXXXXX

SEQ ID NO: 39 - DNA, 384 bp, synthetic construct
  misc_feature 1..384: note = Coding Sequence of modified protein from Mattirolomyces terfezioides with His-Tag
  source 1..384: mol_type = other DNA
atgccggatttgagctcgttcattactattaagaataactctaaccatgtttttacgcgtaccgcaatctattccaaatatgccgcagtccagtggagcccggaaccgcagctgagcattagcccaggtaaatgggacctgttcatcctgaaagaaatcctgagcatccgcggtacgtccggctacgtgcagtaccgtgtcggcgacggtccgggctgggtgcgtgttacctttagcagcctggtgggtgcggacgaagttgctgagtggagcagcggtgatctgccggatggctttgttctgcaaaagcctgtccgcaccggtagccgtccgctgcaagcgacgttcgaggcgaccaagcaacatcaccaccaccaccactaa

SEQ ID NO: 40 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPQLSISPGKWDLFILKEILSIRGTSGYVQYRVGDGPGWVRVTFSSLVGADEVAEWSSGDLPDGFVLQKPVRTGSRPLQATFEATKQXXXXXX

SEQ ID NO: 41 - DNA, 384 bp, synthetic construct
  misc_feature 1..384: note = Coding Sequence of modified protein from Mattirolomyces terfezioides with His-Tag
  source 1..384: mol_type = other DNA
atgccggatttgagctcgttcattactattaagaataactctaaccatgtttttacgcgtaccgcaatctattccaaatatgccgcagtccagtggagcccggaaccgcagctgagcattagcccaggtaaatgggacctgttcatcctgaaagacatcctgagcatcaaaggtacgtccggctacgtgcagtaccgtgtcggcgacggtccgggctgggtgcgtgttacctttagcagcctggtgggtgcggacgaagttgctgagtggagcagcggtgatctgccggatggctttgttctgcaaaagcctgtccgcaccggtagccgtccgctgcaagcgacgttcgaggcgaccaagcaacatcaccaccaccaccactaa

SEQ ID NO: 42 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPQLSISPGKWDLFILKDILSIKGTSGYVQYRVGDGPGWVRVTFSSLVGADEVAEWSSGDLPDGFVLQKPVRTGSRPLQATFEATKQXXXXXX

SEQ ID NO: 43 - DNA, 384 bp, synthetic construct
  misc_feature 1..384: note = Coding Sequence of modified protein from Mattirolomyces terfezioides with His-Tag
  source 1..384: mol_type = other DNA
atgccggatttgagctcgttcattactattaagaataactctaaccatgtttttacgcgtaccgcaatctattccaaatatgccgcagtccagtggagcccggaaccgcagctgagcattagcccaggtaaatgggacctgttcatcctgaaagacatcctgagcatccgcggtacgtccggctacgtgcagtacaaagtcggcgacggtccgggctgggtgcgtgttacctttagcagcctggtgggtgcggacgaagttgctgagtggagcagcggtgatctgccggatggctttgttctgcaaaagcctgtccgcaccggtagccgtccgctgcaagcgacgttcgaggcgaccaagcaacatcaccaccaccaccactaa

SEQ ID NO: 44 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPQLSISPGKWDLFILKDILSIRGTSGYVQYKVGDGPGWVRVTFSSLVGADEVAEWSSGDLPDGFVLQKPVRTGSRPLQATFEATKQXXXXXX

SEQ ID NO: 45 - DNA, 384 bp, synthetic construct
  misc_feature 1..384: note = Coding Sequence of modified protein from Mattirolomyces terfezioides with His-Tag
  source 1..384: mol_type = other DNA
atgccggatttgagctcgttcattactattaagaataactctaaccatgtttttacgcgtaccgcaatctattccaaatatgccgcagtccagtggagcccggaaccgcagctgagcattagcccaggtaaatgggacctgttcatcctgaaagacatcctgagcatccgcggtacgtccggctacgtgcagtaccgtgtcggcgagggtccgggctgggtgcgtgttacctttagcagcctggtgggtgcggacgaagttgctgagtggagcagcggtgatctgccggatggctttgttctgcaaaagcctgtccgcaccggtagccgtccgctgcaagcgacgttcgaggcgaccaagcaacatcaccaccaccaccactaa

SEQ ID NO: 46 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPQLSISPGKWDLFILKDILSIRGTSGYVQYRVGEGPGWVRVTFSSLVGADEVAEWSSGDLPDGFVLQKPVRTGSRPLQATFEATKQXXXXXX

SEQ ID NO: 47 - DNA, 384 bp, synthetic construct
  misc_feature 1..384: note = Coding Sequence of modified protein from Mattirolomyces terfezioides with His-Tag
  source 1..384: mol_type = other DNA
atgccggatttgagctcgttcattactattaagaataactctaaccatgtttttacgcgtaccgcaatctattccaaatatgccgcagtccagtggagcccggaaccgcagctgagcattagcccaggtaaatgggacctgttcatcctgaaagacatcctgagcatccgcggtacgtccggctacgtgcagtaccgtgtcggcgacggtccgggctgggtgaaagttacctttagcagcctggtgggtgcggacgaagttgctgagtggagcagcggtgatctgccggatggctttgttctgcaaaagcctgtccgcaccggtagccgtccgctgcaagcgacgttcgaggcgaccaagcaacatcaccaccaccaccactaa

SEQ ID NO: 48 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPQLSISPGKWDLFILKDILSIRGTSGYVQYRVGDGPGWVKVTFSSLVGADEVAEWSSGDLPDGFVLQKPVRTGSRPLQATFEATKQXXXXXX

SEQ ID NO: 49 - DNA, 384 bp, synthetic construct
  misc_feature 1..384: note = Coding Sequence of modified protein from Mattirolomyces terfezioides with His-Tag
  source 1..384: mol_type = other DNA
atgccggatttgagctcgttcattactattaagaataactctaaccatgtttttacgcgtaccgcaatctattccaaatatgccgcagtccagtggagcccggaaccgcagctgagcattagcccaggtaaatgggacctgttcatcctgaaagacatcctgagcatccgcggtacgtccggctacgtgcagtaccgtgtcggcgacggtccgggctgggtgcgtgttacctttagcagcctggtgggtgcggaagaagttgctgagtggagcagcggtgatctgccggatggctttgttctgcaaaagcctgtccgcaccggtagccgtccgctgcaagcgacgttcgaggcgaccaagcaacatcaccaccaccaccactaa

SEQ ID NO: 50 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPQLSISPGKWDLFILKDILSIRGTSGYVQYRVGDGPGWVRVTFSSLVGAEEVAEWSSGDLPDGFVLQKPVRTGSRPLQATFEATKQXXXXXX

SEQ ID NO: 51 - DNA, 384 bp, synthetic construct
  misc_feature 1..384: note = Coding Sequence of modified protein from Mattirolomyces terfezioides with His-Tag
  source 1..384: mol_type = other DNA
atgccggatttgagctcgttcattactattaagaataactctaaccatgtttttacgcgtaccgcaatctattccaaatatgccgcagtccagtggagcccggaaccgcagctgagcattagcccaggtaaatgggacctgttcatcctgaaagacatcctgagcatccgcggtacgtccggctacgtgcagtaccgtgtcggcgacggtccgggctgggtgcgtgttacctttagcagcctggtgggtgcggacgatgttgctgagtggagcagcggtgatctgccggatggctttgttctgcaaaagcctgtccgcaccggtagccgtccgctgcaagcgacgttcgaggcgaccaagcaacatcaccaccaccaccactaa

SEQ ID NO: 52 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPQLSISPGKWDLFILKDILSIRGTSGYVQYRVGDGPGWVRVTFSSLVGADDVAEWSSGDLPDGFVLQKPVRTGSRPLQATFEATKQXXXXXX

SEQ ID NO: 53 - DNA, 384 bp, synthetic construct
  misc_feature 1..384: note = Coding Sequence of modified protein from Mattirolomyces terfezioides with His-Tag
  source 1..384: mol_type = other DNA
atgccggatttgagctcgttcattactattaagaataactctaaccatgtttttacgcgtaccgcaatctattccaaatatgccgcagtccagtggagcccggaaccgcagctgagcattagcccaggtaaatgggacctgttcatcctgaaagacatcctgagcatccgcggtacgtccggctacgtgcagtaccgtgtcggcgacggtccgggctgggtgcgtgttacctttagcagcctggtgggtgcggacgaagttgctgattggagcagcggtgatctgccggatggctttgttctgcaaaagcctgtccgcaccggtagccgtccgctgcaagcgacgttcgaggcgaccaagcaacatcaccaccaccaccactaa

SEQ ID NO: 54 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPQLSISPGKWDLFILKDILSIRGTSGYVQYRVGDGPGWVRVTFSSLVGADEVADWSSGDLPDGFVLQKPVRTGSRPLQATFEATKQXXXXXX

SEQ ID NO: 55 - DNA, 384 bp, synthetic construct
  misc_feature 1..384: note = Coding Sequence of modified protein from Mattirolomyces terfezioides with His-Tag
  source 1..384: mol_type = other DNA
atgccggatttgagctcgttcattactattaagaataactctaaccatgtttttacgcgtaccgcaatctattccaaatatgccgcagtccagtggagcccggaaccgcagctgagcattagcccaggtaaatgggacctgttcatcctgaaagacatcctgagcatccgcggtacgtccggctacgtgcagtaccgtgtcggcgacggtccgggctgggtgcgtgttacctttagcagcctggtgggtgcggacgaagttgctgagtggagcagcggtgagctgccggatggctttgttctgcaaaagcctgtccgcaccggtagccgtccgctgcaagcgacgttcgaggcgaccaagcaacatcaccaccaccaccactaa

SEQ ID NO: 56 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPQLSISPGKWDLFILKDILSIRGTSGYVQYRVGDGPGWVRVTFSSLVGADEVAEWSSGELPDGFVLQKPVRTGSRPLQATFEATKQXXXXXX

SEQ ID NO: 57 - DNA, 384 bp, synthetic construct
  misc_feature 1..384: note = Coding Sequence of modified protein from Mattirolomyces terfezioides with His-Tag
  source 1..384: mol_type = other DNA
atgccggatttgagctcgttcattactattaagaataactctaaccatgtttttacgcgtaccgcaatctattccaaatatgccgcagtccagtggagcccggaaccgcagctgagcattagcccaggtaaatgggacctgttcatcctgaaagacatcctgagcatccgcggtacgtccggctacgtgcagtaccgtgtcggcgacggtccgggctgggtgcgtgttacctttagcagcctggtgggtgcggacgaagttgctgagtggagcagcggtgatctgccggaaggctttgttctgcaaaagcctgtccgcaccggtagccgtccgctgcaagcgacgttcgaggcgaccaagcaacatcaccaccaccaccactaa

SEQ ID NO: 58 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPQLSISPGKWDLFILKDILSIRGTSGYVQYRVGDGPGWVRVTFSSLVGADEVAEWSSGDLPEGFVLQKPVRTGSRPLQATFEATKQXXXXXX

SEQ ID NO: 59 - DNA, 384 bp, synthetic construct
  misc_feature 1..384: note = Coding Sequence of modified protein from Mattirolomyces terfezioides with His-Tag
  source 1..384: mol_type = other DNA
atgccggatttgagctcgttcattactattaagaataactctaaccatgtttttacgcgtaccgcaatctattccaaatatgccgcagtccagtggagcccggaaccgcagctgagcattagcccaggtaaatgggacctgttcatcctgaaagacatcctgagcatccgcggtacgtccggctacgtgcagtaccgtgtcggcgacggtccgggctgggtgcgtgttacctttagcagcctggtgggtgcggacgaagttgctgagtggagcagcggtgatctgccggatggctttgttctgcaacgccctgtccgcaccggtagccgtccgctgcaagcgacgttcgaggcgaccaagcaacatcaccaccaccaccactaa

SEQ ID NO: 60 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPQLSISPGKWDLFILKDILSIRGTSGYVQYRVGDGPGWVRVTFSSLVGADEVAEWSSGDLPDGFVLQRPVRTGSRPLQATFEATKQXXXXXX

SEQ ID NO: 61 - DNA, 384 bp, synthetic construct
  misc_feature 1..384: note = Coding Sequence of modified protein from Mattirolomyces terfezioides with His-Tag
  source 1..384: mol_type = other DNA
atgccggatttgagctcgttcattactattaagaataactctaaccatgtttttacgcgtaccgcaatctattccaaatatgccgcagtccagtggagcccggaaccgcagctgagcattagcccaggtaaatgggacctgttcatcctgaaagacatcctgagcatccgcggtacgtccggctacgtgcagtaccgtgtcggcgacggtccgggctgggtgcgtgttacctttagcagcctggtgggtgcggacgaagttgctgagtggagcagcggtgatctgccggatggctttgttctgcaaaagcctgtcaaaaccggtagccgtccgctgcaagcgacgttcgaggcgaccaagcaacatcaccaccaccaccactaa

SEQ ID NO: 62 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPQLSISPGKWDLFILKDILSIRGTSGYVQYRVGDGPGWVRVTFSSLVGADEVAEWSSGDLPDGFVLQKPVKTGSRPLQATFEATKQXXXXXX

SEQ ID NO: 63 - DNA, 384 bp, synthetic construct
  misc_feature 1..384: note = Coding Sequence of modified protein from Mattirolomyces terfezioides with His-Tag
  source 1..384: mol_type = other DNA
atgccggatttgagctcgttcattactattaagaataactctaaccatgtttttacgcgtaccgcaatctattccaaatatgccgcagtccagtggagcccggaaccgcagctgagcattagcccaggtaaatgggacctgttcatcctgaaagacatcctgagcatccgcggtacgtccggctacgtgcagtaccgtgtcggcgacggtccgggctgggtgcgtgttacctttagcagcctggtgggtgcggacgaagttgctgagtggagcagcggtgatctgccggatggctttgttctgcaaaagcctgtccgcaccggtagcaaaccgctgcaagcgacgttcgaggcgaccaagcaacatcaccaccaccaccactaa

SEQ ID NO: 64 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPQLSISPGKWDLFILKDILSIRGTSGYVQYRVGDGPGWVRVTFSSLVGADEVAEWSSGDLPDGFVLQKPVRTGSKPLQATFEATKQXXXXXX

SEQ ID NO: 65 - DNA, 384 bp, synthetic construct
  misc_feature 1..384: note = Coding Sequence of modified protein from Mattirolomyces terfezioides with His-Tag
  source 1..384: mol_type = other DNA
atgccggatttgagctcgttcattactattaagaataactctaaccatgtttttacgcgtaccgcaatctattccaaatatgccgcagtccagtggagcccggaaccgcagctgagcattagcccaggtaaatgggacctgttcatcctgaaagacatcctgagcatccgcggtacgtccggctacgtgcagtaccgtgtcggcgacggtccgggctgggtgcgtgttacctttagcagcctggtgggtgcggacgaagttgctgagtggagcagcggtgatctgccggatggctttgttctgcaaaagcctgtccgcaccggtagccgtccgctgcaagcgacgttcgatgcgaccaagcaacatcaccaccaccaccactaa

SEQ ID NO: 66 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPQLSISPGKWDLFILKDILSIRGTSGYVQYRVGDGPGWVRVTFSSLVGADEVAEWSSGDLPDGFVLQKPVRTGSRPLQATFDATKQXXXXXX

SEQ ID NO: 67 - DNA, 384 bp, synthetic construct
  misc_feature 1..384: note = Coding Sequence of modified protein from Mattirolomyces terfezioides with His-Tag
  source 1..384: mol_type = other DNA
atgccggatttgagctcgttcattactattaagaataactctaaccatgtttttacgcgtaccgcaatctattccaaatatgccgcagtccagtggagcccggaaccgcagctgagcattagcccaggtaaatgggacctgttcatcctgaaagacatcctgagcatccgcggtacgtccggctacgtgcagtaccgtgtcggcgacggtccgggctgggtgcgtgttacctttagcagcctggtgggtgcggacgaagttgctgagtggagcagcggtgatctgccggatggctttgttctgcaaaagcctgtccgcaccggtagccgtccgctgcaagcgacgttcgaggcgacccgccaacatcaccaccaccaccactaa

SEQ ID NO: 68 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPQLSISPGKWDLFILKDILSIRGTSGYVQYRVGDGPGWVRVTFSSLVGADEVAEWSSGDLPDGFVLQKPVRTGSRPLQATFEATRQXXXXXX

SEQ ID NO: 69 - protein, 5 aa, synthetic construct
  REGION 1..5: note = Amino Acid Linker
  REPEAT 1..5: note = May be repeated n times, where n is any integer
GGGGS

SEQ ID NO: 70 - protein, 5 aa, synthetic construct
  REGION 1..5: note = Amino Acid Linker
  REPEAT 1..5: note = May be repeated n times, where n is any integer
EAAAK

SEQ ID NO: 71 - protein, 121 aa, synthetic construct
  REGION 1..121: note = Consensus Sequence 1
  VARIANT 3: note = X is any amino acid
  VARIANT 11..16: note = X is any amino acid
  VARIANT 26: note = X is any amino acid
  VARIANT 33..34: note = X is any amino acid
  VARIANT 51: note = X is any amino acid
  VARIANT 57: note = X is any amino acid
  VARIANT 66: note = X is any amino acid
  VARIANT 89: note = X is any amino acid
  VARIANT 120: note = X is any amino acid
  VARIANT 41..43: note = X is any amino acid
  VARIANT 68..72: note = X is any amino acid
  VARIANT 101..110: note = X is any amino acid
  VARIANT 36..38: note = X is any amino acid
  VARIANT 117: note = X is any amino acid
  VARIANT 85..86: note = X is any amino acid
  VARIANT 97: note = X is any naturally occurring amino acid
MPXLSSFITIXXXXXXVFTRTAIYSXYAAVQWXXEXXXSIXXXKWDLFILXDILSIXGTSGYVQYXVXXXXXWVRVTFSSLVGAXXVAXWSSGDLPXGFVXXXXXXXXXXPLQATFXATXQ

SEQ ID NO: 72 - protein, 121 aa, synthetic construct
  REGION 1..121: note = Consensus sequence 2
  VARIANT 3: note = X is any amino acid
  VARIANT 11..16: note = X is any amino acid
  VARIANT 26: note = X is any amino acid
  VARIANT 33: note = X is any amino acid
  VARIANT 37..38: note = X is any amino acid
  VARIANT 43: note = X is any amino acid
  VARIANT 51: note = X is any amino acid
  VARIANT 57: note = X is any amino acid
  VARIANT 68..70: note = X is any amino acid
  VARIANT 72: note = X is any amino acid
  VARIANT 85..86: note = X is any amino acid
  VARIANT 89: note = X is any amino acid
  VARIANT 101..103: note = X is any amino acid
  VARIANT 105..110: note = X is any amino acid
  VARIANT 41: note = X is any amino acid
  VARIANT 117: note = X is any amino acid
  VARIANT 120: note = X is any amino acid
  VARIANT 66: note = X is any amino acid
  VARIANT 97: note = X is any amino acid
MPXLSSFITIXXXXXXVFTRTAIYSXYAAVQWXPEPXXSIXPXKWDLFILXDILSIXGTSGYVQYXVXXXPXWVRVTFSSLVGAXXVAXWSSGDLPXGFVXXXPXXXXXXPLQATFXATXQ

SEQ ID NO: 73 - protein, 121 aa, synthetic construct; identical to SEQ ID NO: 72
  REGION 1..121: note = Consensus Sequence 3
  VARIANT 3: note = X is Asp, Glu, Asn or Gln
  VARIANT 11: note = X is Lys, Arg or His
  VARIANT 12..13: note = X is Asp, Glu, Asn or Gln
  VARIANT 14: note = X is Ser, Cys, Thr or Met
  VARIANT 15: note = X is Asp, Glu, Asn or Gln
  VARIANT 16: note = X is Val, Gly, Ala, Leu or Ile
  VARIANT 26: note = X is Lys, Arg or His
  VARIANT 33: note = X is Ser, Cys, Thr or Met
  VARIANT 37: note = X is Asp, Glu, Asn or Gln
  VARIANT 38: note = X is Val, Gly, Ala, Leu or Ile
  VARIANT 41: note = X is Ser, Cys, Thr or Met
  VARIANT 43: note = X is Val, Gly, Ala, Leu or Ile
  VARIANT 51: note = X is Lys, Arg or His
  VARIANT 66: note = X is Lys, Arg or His
  VARIANT 68: note = X is Val, Gly, Ala, Leu or Ile
  VARIANT 69: note = X is Asp, Glu, Asn or Gln
  VARIANT 70: note = X is Val, Gly, Ala, Leu or Ile
  VARIANT 85..86: note = X is Asp, Glu, Asn or Gln
  VARIANT 89: note = X is Asp, Glu, Asn or Gln
  VARIANT 97: note = X is Asp, Glu, Asn or Gln
  VARIANT 101: note = X is Val, Gly, Ala, Leu or Ile
  VARIANT 103: note = X is Lys, Arg or His
  VARIANT 105: note = X is Val, Gly, Ala, Leu or Ile
  VARIANT 107: note = X is Ser, Cys, Thr or Met
  VARIANT 108: note = X is Val, Gly, Ala, Leu or Ile
  VARIANT 120: note = X is Lys, Arg or His
  VARIANT 57: note = X is Lys, Arg or His
  VARIANT 117: note = X is Asp, Glu, Asn or Gln
  VARIANT 72: note = X is Val, Gly, Ala, Leu or Ile
  VARIANT 106: note = X is Lys, Arg or His
  VARIANT 102: note = X is Asp, Glu, Asn or Gln
  VARIANT 110: note = X is Lys, Arg or His
  VARIANT 109: note = X is Ser, Cys, Thr or Met

SEQ ID NO: 74 - protein, 121 aa, synthetic construct; identical to SEQ ID NO: 72
  REGION 1..121: note = Consensus Sequence 4
  VARIANT 3: note = X is Asp or Glu
  VARIANT 11: note = X is Lys or Arg
  VARIANT 12..13: note = X is Asn or Gln
  VARIANT 14: note = X is Ser or Thr
  VARIANT 15: note = X is Asn or Gln
  VARIANT 16: note = X is Leu, Val or Ile
  VARIANT 26: note = X is Lys or Arg
  VARIANT 37: note = X is Asn or Gln
  VARIANT 38: note = X is Leu, Val, or Ile
  VARIANT 41: note = X is Ser or Thr
  VARIANT 43: note = X is Gly or Ala
  VARIANT 51: note = X is Lys or Arg
  VARIANT 57: note = X is Lys or Arg
  VARIANT 66: note = X is Lys or Arg
  VARIANT 68: note = X is Gly or Ala
  VARIANT 70: note = X is Gly or Ala
  VARIANT 72: note = X is Gly or Ala
  VARIANT 85..86: note = X is Asp or Glu
  VARIANT 89: note = X is Asp or Glu
  VARIANT 97: note = X is Asp or Glu
  VARIANT 102: note = X is Asn or Gln
  VARIANT 103: note = X is Lys or Arg
  VARIANT 105: note = X is Leu, Val or Ile
  VARIANT 107: note = X is Ser or Thr
  VARIANT 110: note = X is Lys or Arg
  VARIANT 120: note = X is Lys or Arg
  VARIANT 69: note = X is Asp or Glu
  VARIANT 108: note = X is Gly or Ala
  VARIANT 109: note = X is Ser or Thr
  VARIANT 33: note = X is Ser or Thr
  VARIANT 106: note = X is Lys or Arg
  VARIANT 117: note = X is Asp or Glu
  VARIANT 101: note = X is Leu, Val or Ile

SEQ ID NO: 75 - protein, 121 aa, synthetic construct
  REGION 1..121: note = Consensus Sequence 5
  VARIANT 3: note = X is Asp or Glu
  VARIANT 11: note = X is Lys or Arg
  VARIANT 26: note = X is Lys or Arg
  VARIANT 51: note = X is Lys or Arg
  VARIANT 57: note = X is Lys or Arg
  VARIANT 66: note = X is Lys or Arg
  VARIANT 69: note = X is Asp or Glu
  VARIANT 85: note = X is Asp or Glu
  VARIANT 86: note = X is Asp or Glu
  VARIANT 89: note = X is Asp or Glu
  VARIANT 103: note = X is Lys or Arg
  VARIANT 106: note = X is Lys or Arg
  VARIANT 110: note = X is Lys or Arg
  VARIANT 117: note = X is Asp or Glu
  VARIANT 97: note = X is Asp or Glu
  VARIANT 120: note = X is Lys or Arg
MPXLSSFITIXNNSNHVFTRTAIYSXYAAVQWSPEPQLSISPGKWDLFILXDILSIXGTSGYVQYXVGXGPGWVRVTFSSLVGAXXVAXWSSGDLPXGFVLQXPVXTGSXPLQATFXATXQ

SEQ ID NO: 76 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctattccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 77 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctactccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 78 - protein, 127 aa, synthetic construct
  REGION 1..127: note = Modified protein from Mattirolomyces terfezioides with optional His-Tag
  REGION 122..127: note = Absent or Histidine
MPDLYSFITIKNNSNHVFTRTAIYSKYAAVQWSPEPQLSISPGKWDLFILKDILSIRGTSGYVQYRVGDGPGWVRVTFSSLVGADEVAEWSSGDLPDGFVLQKPVRTGSRPLQATFEATKQXXXXXX

SEQ ID NO: 79 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctatttcattacgattaagaacaactctaaccacgtcttcactcggacagccatctactcaaagtacgcagccgtccaatggtccccagaacctcagttgtctatatctccaggtaaatgggatcttttcatcttaaaagatattctaagtattagaggcacttctggttacgtacagtatcgtgttggtgatggacctggttgggttagagtaacattcagctcattggttggggctgacgaagtggctgagtggtcctcaggtgacctcccagatggcttcgtgctgcaaaagccagtcagaactggatctagaccattgcaagcgacattcgaagcaacaaagcaacatcaccaccaccaccactaa

SEQ ID NO: 80 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctacttcattacgattaagaacaactctaaccacgtcttcactcggacagccatctactcaaagtacgcagccgtccaatggtccccagaacctcagttgtctatatctccaggtaaatgggatcttttcatcttaaaagatattctaagtattagaggcacttctggttacgtacagtatcgtgttggtgatggacctggttgggttagagtaacattcagctcattggttggggctgacgaagtggctgagtggtcctcaggtgacctcccagatggcttcgtgctgcaaaagccagtcagaactggatctagaccattgcaagcgacattcgaagcaacaaagcaacatcaccaccaccaccactaa

SEQ ID NO: 81 - protein, 127 aa, synthetic construct
  REGION 1..127: note = Modified protein from Mattirolomyces terfezioides with optional His-Tag
  REGION 122..127: note = Absent or Histidine
MPDLSYFITIKNNSNHVFTRTAIYSKYAAVQWSPEPQLSISPGKWDLFILKDILSIRGTSGYVQYRVGDGPGWVRVTFSSLVGADEVAEWSSGDLPDGFVLQKPVRTGSRPLQATFEATKQXXXXXX

SEQ ID NO: 82 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtctatactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 83 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtctacactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 84 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVYTRTAIYSKYAAVQWSPEPQLSISPGKWDLFILKDILSIRGTSGYVQYRVGDGPGWVRVTFSSLVGADEVAEWSSGDLPDGFVLQKPVRTGSRPLQATFEATKQXXXXXX

SEQ ID NO: 85 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattataagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 86 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattacaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 87 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYYKYAAVQWSPEPQLSISPGKWDLFILKDILSIRGTSGYVQYRVGDGPGWVRVTFSSLVGADEVAEWSSGDLPDGFVLQKPVRTGSRPLQATFEATKQXXXXXX

SEQ ID NO: 88 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgctcagtggagtcccgagccgcaactctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 89 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgcccagtggagtcccgagccgcaactctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 90 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgcacagtggagtcccgagccgcaactctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 91 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgcgcagtggagtcccgagccgcaactctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 92 - protein, 143 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAAQWSPEPQLSISPGKWDLFILKDILSIRGTSGYVQYRVGDGPGWVRVTFSSLVGADEVAEWSSGDLPDGFVLQKPVRTGSRPLDGFVLQKPVRTGSRPLQATFEATKQXXXXXX

SEQ ID NO: 93 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgaaactctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 94 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgaagctctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 95 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPKLSISPGKWDLFILKDILSIRGTSGYVQYRVGDGPGWVRVTFSSLVGADEVAEWSSGDLPDGFVLQKPVRTGSRPLQATFEATKQXXXXXX

SEQ ID NO: 96 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccggaactctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 97 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccggagctctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 98 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPELSISPGKWDLFILKDILSIRGTSGYVQYRVGDGPGWVRVTFSSLVGADEVAEWSSGDLPDGFVLQKPVRTGSRPLQATFEATKQXXXXXX

SEQ ID NO: 99 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgaatctctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 100 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgaacctctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 101 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPNLSISPGKWDLFILKDILSIRGTSGYVQYRVGDGPGWVRVTFSSLVGADEVAEWSSGDLPDGFVLQKPVRTGSRPLQATFEATKQXXXXXX

SEQ ID NO: 102 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggctttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 103 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggccttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 104 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggcattgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 105 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggcgttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 106 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPQLSISPGKWALFILKDILSIRGTSGYVQYRVGDGPGWVRVTFSSLVGADEVAEWSSGDLPDGFVLQKPVRTGSRPLQATFEATKQXXXXXX

SEQ ID NO: 107 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtatattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 108 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtacattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 109 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPQLSISPGKWDLYILKDILSIRGTSGYVQYRVGDGPGWVRVTFSSLVGADEVAEWSSGDLPDGFVLQKPVRTGSRPLQATFEATKQXXXXXX

SEQ ID NO: 110 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgctcaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 111 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgcccaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 112 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgcacaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 113 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgcgcaatatcgggttggggatggtcctggatgggttagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 114 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPQLSISPGKWDLFILKDILSIRGTSGYAQYRVGDGPGWVRVTFSSLVGADEVAEWSSGDLPDGFVLQKPVRTGSRPLQATFEATKQXXXXXX

SEQ ID NO: 115 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggctagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 116 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggccagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 117 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggcaagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 118 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggcgagggtcaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 119 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPQLSISPGKWDLFILKDILSIRGTSGYVQYRVGDGPGWARVTFSSLVGADEVAEWSSGDLPDGFVLQKPVRTGSRPLQATFEATKQXXXXXX

SEQ ID NO: 120 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggttagggctaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 121 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggttagggccaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 122 - DNA, 384 bp, synthetic construct
  source 1..384: mol_type = other DNA
  misc_feature 1..122: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggttagggcaaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 123 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggttagggcgaccttttcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 124 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPQLSISPGKWDLFILKDILSIRGTSGYVQYRVGDGPGWVRATFSSLVGADEVAEWSSGDLPDGFVLQKPVRTGSRPLQATFEATKQXXXXXX

SEQ ID NO: 125 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggttagggtcacctattcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 126 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggttagggtcacctactcttctctagtcggggccgatgaagtggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 127 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPQLSISPGKWDLFILKDILSIRGTSGYVQYRVGDGPGWVRVTYSSLVGADEVAEWSSGDLPDGFVLQKPVRTGSRPLQATFEATKQXXXXXX

SEQ ID NO: 128 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagctgcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 129 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagccgcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 130 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagcagcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 131 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagcggcagagtggagctcaggtgacctacctgatggctttgttctccaaaaaccagttcgcactgggtccaggcctttgcaggcaacgttcgaggctacaaaacaacatcaccaccaccaccactaa

SEQ ID NO: 132 - protein, 127 aa, synthetic construct
  REGION 1..127: note = Modified protein from Mattirolomyces terfezioides with optional His-Tag
  REGION 122..127: note = Absent or Histidine
MPDLSSFITIKNNSNHVFTRTAIYSKYAAVQWSPEPQLSISPGKWDLFILKDILSIRGTSGYVQYRVGDGPGWVRVTFSSLVGADEAAEWSSGDLPDGFVLQKPVRTGSRPLQATFEATKQXXXXXX

SEQ ID NO: 133 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgctctccaaaaaccagttcgcactgggtccaggcctttgcaggcaacgttcgaggctacaaaacaacatcaccaccaccaccactaa

SEQ ID NO: 134 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgccctccaaaaaccagttcgcactgggtccaggcctttgcaggcaacgttcgaggctacaaaacaacatcaccaccaccaccactaa

SEQ ID NO: 135 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgcactccaaaaaccagttcgcactgggtccaggcctttgcaggcaacgttcgaggctacaaaacaacatcaccaccaccaccactaa

SEQ ID NO: 136 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgcgctccaaaaaccagttcgcactgggtccaggcctttgcaggcaacgttcgaggctacaaaacaacatcaccaccaccaccactaa

SEQ ID NO: 137 - protein, 127 aa, synthetic construct
  REGION 1..127: note = modified protein from M. terfezioides with optional His-tag
MPDLSSFITIKNNSNHVFTRTAIYSKYAAVQWSPEPQLSISPGKWDLFILKDILSIRGTSGYVQYRVGDGPGWVRVTFSSLVGADEVAEWSSGDLPDGFALQKPVRTGSRPLQATFEATKQXXXXXX

SEQ ID NO: 138 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctcaaaaaaccagttcgcactgggtccaggcctttgcaggcaacgttcgaggctacaaaacaacatcaccaccaccaccactaa

SEQ ID NO: 139 - DNA, 384 bp, synthetic construct
  source 1..384: mol_type = other DNA
  misc_feature 1..384: note = Coding Sequence of modified protein from Mattirolomyces terfezioides with His-Tag
atgcctgatctctcctccttcattacgattaagaacaactctaaccacgtcttcactcggacggcaatttattctaagtatgccgctgtgcagtggagtcccgagccgcaactctcaatctctcccggcaaatgggatttgtttattttaaaggacatcttgtctatccgtgggacttcgggatatgtacaatatcgggttggggatggtcctggatgggttagggtcaccttttcttctctagtcggggccgatgaagtggcagagtggagctcaggtgacctacctgatggctttgttctcaagaaaccagttcgcactgggtccaggcctttgcaggcaacgttcgaggctacaaaacaacatcaccaccaccaccactaa

SEQ ID NO: 140 - protein, 127 aa, synthetic construct; identical to SEQ ID NO: 124
  REGION 1..127: note = Modified protein from Mattirolomyces terfezioides with optional His-Tag
  VARIANT 122..127: note = X is Histidine or Absent

SEQ ID NO: 141 - protein, 120 aa, Mattirolomyces terfezioides
PDLSSFITIKNNSNHVFTRTAIYSKYAAVQWSPEPQLSISPGKWDLFILKDILSIRGTSGYVQYRVGDGPGWVRVTFSSLVGADEVAEWSSGDLPDGFVLQKPVRTGSRPLQATFEATKQ

SEQ ID NO: 142 - protein, 126 aa, synthetic construct
  source 1..126: note = with His-tag
PDLSSFITIKNNSNHVFTRTAIYSKYAAVQWSPEPQLSISPGKWDLFILKDILSIRGTSGYVQYRVGDGPGWVRVTFSSLVGADEVAEWSSGDLPDGFVLQKPVRTGSRPLQATFEATKQHHHHHH

SEQ ID NO: 143 - protein, 127 aa, synthetic construct
  source 1..127: note = optional His-tag
  VARIANT 122..127: note = X is H or is absent
MPDLSSFITIKNNSNHVFTRTAIYSKYAAVQWCPEPQLSISPGKWDLFCLKDILSIRGTSGYVQYRVGDGPGWVRVTFSSLVGADEVAEWSSGDLPDGFVLQKPVRTGSRPLQATFEATKQXXXXXX

SEQ ID NO: 144 - DNA, 363 bp, synthetic construct
  source 1..363: mol_type = other DNA
atgccggatctgagcagctttattaccattaaaaacaacagcaaccatgtgtttacccgcaccgcgatttatagcaaatatgcggcggtgcagtggtgcccggaaccgcagctgagcattagcccgggcaaatgggatctgttttgcctgaaagatattctgagcattcgcggcaccagcggctatgtgcagtatcgcgtgggcgatggcccgggctgggtgcgcgtgacctttagcagcctggtgggcgcggatgaagtggcggaatggagcagcggcgatctgccggatggctttgtgctgcagaaaccggtgcgcaccggcagccgcccgctgcaggcgacctttgaagcgaccaaacag

SEQ ID NO: 145 - protein, 121 aa, synthetic construct
MPDLSSFITIKNNSNHVFTRTAICSKYAAVQWSPEPQLSISPGKWDLFILKDILSIRGTSGCVQYRVGDGPGWVRVTFSSLVGADEVAEWSSGDLPDGFVLQKPVRTGSRPLQATFEATKQ

SEQ ID NO: 146 - protein, 120 aa, synthetic construct
PDLSSFITIKNNSNHVFTRTAIYSKYAAVQWCPEPQLSISPGKWDLFCLKDILSIRGTSGYVQYRVGDGPGWVRVTFSSLVGADEVAEWSSGDLPDGFVLQKPVRTGSRPLQATFEATKQ

SEQ ID NO: 147 - protein, 120 aa, synthetic construct
PDLSSFITIKNNSNHVFTRTAICSKYAAVQWSPEPQLSISPGKWDLFILKDILSIRGTSGCVQYRVGDGPGWVRVTFSSLVGADEVAEWSSGDLPDGFVLQKPVRTGSRPLQATFEATKQ

SEQ ID NO: 148 - DNA, 363 bp, synthetic construct
  source 1..363: mol_type = other DNA; note = no taa and no His-tag
atgccggacctgtcttcctttatcactatcaaaaataactctaaccatgttttcacccgcacggcgatctacagcaaatacgccgcggtgcagtggtgtccggaaccgcagctgtctatcagcccgggcaaatgggacctgttctgtctgaaagatatcctgagcatccgtggtacctctggttatgttcagtaccgtgtcggcgatggtccgggttgggttcgcgttaccttcagctctctggttggtgcggacgaagttgcggaatggtcctctggtgacctgccagatggtttcgtgctgcagaaaccggttcgtactggctctcgtccgctccaggccactttcgaagctaccaaacaa

SEQ ID NO: 149 - DNA, 21 bp, synthetic construct
  source 1..21: mol_type = other DNA
caacatcaccatcaccaccac

(19) United States
(12) Patent Application Publication
(10) Pub. No.: US 2025/0136649 A1
(43) Pub. Date: May 1, 2025

(54) HONEY TRUFFLE SWEETENER (HTS) VARIANTS

(71) Applicant: MYCOTECHNOLOGY, INC., Aurora, CO (US)

(72) Inventors: Marina NADAL, Aurora, CO (US); Anthony J. CLARK, Aurora, CO (US); Zheyuan GUO, Aurora, CO (US); Stephen A. GRAVINA, Aurora, CO (US); Anthony WESTGATE, Aurora, CO (US); Bassam ALKOTAINI, Aurora, CO (US); Brendan SHARKEY, Aurora, CO (US); Joseph MEILEN, Aurora, CO (US); James Patrick LANGAN, Aurora, CO (US); Miles INGWERS, Aurora, CO (US); Daniel Edward CONNORS, Aurora, CO (US); Michael G. EDWARDS, Aurora, CO (US); Ranjan PATNAIK, Aurora, CO (US); Karli R. VAN SIMAEYS, Aurora, CO (US); Benjamin JAACKS, Aurora, CO (US)

(73) Assignee: MYCOTECHNOLOGY, INC, Aurora, CO (US)

(21) Appl. No.: 19/003,764

(22) Filed: Dec. 27, 2024

Related U.S. Application Data
(63) Continuation of application No. PCT/US2024/015023, filed on Feb. 8, 2024.
(60) Provisional application No. 63/444,185, filed on Feb. 8, 2023.
(60) Provisional application No. 63/524,794, filed on Jul. 3, 2023.
(60) Provisional application No. 63/541,591, filed on Sep. 29, 2023.
(60) Provisional application No. 63/618,616, filed on Jan. 8, 2024.

(51) Int. Cl. C07K 14/37; A23L 2/60; A23L 27/30; C07K 1/16; C07K 1/20

(52) U.S. Cl. CPC C07K 14/37; A23L 2/60; A23L 27/31; C07K 1/165; C07K 1/20; C07K 2319/21

(57) ABSTRACT

Fungal sweet-taste modifying protein variants (designated Honey Truffle Sweetener (HTS) protein variants), and polynucleotides encoding the protein variants are described. Specifically, Myd (HTS) protein variants with sweet taste modulation activity, and the cDNA encoding the same, are described, along with methods for isolating such cDNA and for isolating and expressing such proteins. Also disclosed are sweetening compositions which include the proteins of the invention, and methods to provide improved flavor to a product for oral administration.

### CROSS-REFERENCE TO RELATED APPLICATIONS

This application is a continuation application of International Application no. PCT/US2024/015023, filed Feb. 8, 2024, which application claims the benefit of U.S. provisional application No. 63/444,185, filed Feb. 8, 2023, U.S. provisional application No. 63/524,794, filed Jul. 3, 2023, U.S. provisional application No. 63/541,591, filed Sep. 29, 2023, and U.S. provisional application No. 63/618,616, filed Jan. 8, 2024, each of which is incorporated by reference herein in its entirety.

### INCORPORATION-BY-REFERENCE OF MATERIAL SUBMITTED ELECTRONICALLY

Incorporated by reference in its entirety herein is a computer-readable nucleotide/amino acid sequence listing identified as follows: One 282 KB XML file named “0640-47_US.xml” created on Dec. 27, 2024.

### FIELD OF THE INVENTION

This invention includes embodiments of sweet-tasting (Honey Truffle Sweetener (HTS)) proteins, genes, and cDNA encoding said proteins, and to methods of using such proteins, genes, and cDNA in the modulation of the taste of foods. More particularly, the invention includes embodiments of HTS variant sweet proteins, and to the genes and cDNA encoding such proteins.

### BACKGROUND OF THE INVENTION

Excess intake of nutritive sweeteners has long been associated with diet-related health issues, such as obesity, heart disease, metabolic disorders and dental problems. Consequently, consumers are increasingly looking for ways to decrease the amount of nutritive sweeteners in their diets; and manufacturers are trying to respond to this demand by attempting to replace nutritive sweeteners with substitutes to mimic the desirable taste and functional properties of the nutritive sweeteners.

Zero or low-calorie sweeteners derived from, preferably, natural sources are desired to limit the negative effects of high sugar consumption (e.g., diabetes and obesity, among others). But, commonly-known zero or low-calorie sweetener substitutes such as aspartame, acesulfame potassium, luo han guo (monk) fruit extract, neotame, saccharin, stevia and sucralose have undesirable taste defects such as bitterness.

Zero or low-calorie sweeteners derived from natural sources may be preferred to limit the negative effects of high sugar consumption. So far there are only seven known sweet and taste-modifying proteins, namely monellin, thaumatin, brazzein, curculin, mabinlin, miraculin and pentadin. The key residues on the protein surface responsible for biological activity have not yet been identified with certainty for any of these proteins. Monellin was found to be 100,000 times sweeter than sucrose on a molar basis, followed by thaumatin and brazzein which are 3000 times and 500 times sweeter than sucrose, respectively, on a gram basis. Most of them share no sequence homology or structural similarity; and thaumatin shares extensive similarity at the protein sequence level with certain non-sweet proteins found in other plants.

International patent application PCT/US2020/012955, filed Jan. 9, 2020, published as WO2020/146650 on Jul. 16, 2020 relates to a sweetening composition comprising (i) mycelia of an ascomycete or an aqueous extract thereof or (ii) an aqueous extract of a fruiting body of an ascomycete and use of such composition to provide improved flavor to a product for oral administration. The application also relates to compositions comprising combinations of sweetening compositions and a product for oral administration.

International patent application PCT/US2021/039176, filed Jun. 25, 2021, published as WO2021/263158 on Dec. 30, 2021, relates to newly identified fungal sweet-taste modifying proteins and the cDNA encoding the proteins. Sweet-taste modifying proteins were identified in the truffle, Mattirolomyces terfeziodes. The application further relates to Myd proteins as sweeteners and in sweet taste activation/modulation and the cDNA encoding the same and methods for isolating such cDNA and for isolating and expressing such proteins. The application also relates to a sweetening composition which includes the proteins and methods to provide improved flavor to a product for oral administration.

International patent application PCT/US2022/82443, filed Dec. 27, 2022, and published as WO2023/129938, on Jul. 6, 2023, relate to sweet protein variants from truffle, e.g., Mattirolomyces terfeziodes, including proteins, genes, cDNA encoding said proteins, and compositions thereof, and are hereby incorporated by reference in their entirety.

A need still remains for new low or zero calorie sweeteners with improved taste from natural sources, particularly from ascomycetes fungal species. A need also remains for producing fungally-derived low or zero calorie sweeteners with improved taste and methods of using such ingredients in final food products without compromising on taste and with superior taste profiles.

### SUMMARY OF THE DISCLOSURE

The invention meets these and other needs by providing fungally-derived sweet-tasting proteins identified herein as the Myd family of proteins, as well as the genes and cDNA encoding such proteins, and methods of using such proteins, genes, and cDNA in the modulation of the taste of foods. The invention provides variants of MYD1 (also referred to as mycodulcein, and Honey Truffle Sweetener protein (HTS protein)). Polypeptides of the invention exhibit flavor-modifying properties and, in particular, modulate sweet taste and perception, either alone, or in combination with food, beverages, dietary supplements, or pharmaceuticals. In embodiments, Myd1 variants of the invention can provide sweet-taste to materials, such as food, beverages, dietary supplements, or pharmaceuticals that are ingested. In embodiments, Myd1 variants of the invention can reduce the sourness, bitterness or astringency of foods and drinks. In embodiments, Myd1 variants of the invention can exhibit enhancement of the taste of foods and drinks, namely a taste-modifying activity. In embodiments, Myd1 variants, can exhibit in addition to sweet-taste modulation activity, enhanced thermal stability compared to native forms of HTS protein. In embodiments, Myd1 variants can exhibit flavor-modifying properties. In embodiments, Myd1 variants, can exhibit in addition to flavor modifying properties, enhanced thermal stability compared to native forms of HTS protein. In an embodiment, the invention provides naturally-occurring Myd proteins. In an embodiment, the invention provides variant Myd proteins other than naturally-occurring proteins.

Naturally-occurring Myd protein exists as two isoforms; HTS-1 (SEQ ID NO:3) and HTS-2 (SEQ ID NO:141), as isolated from Mattirolomyces terfeziodes. The relative amounts of HTS-1 and HTS-2 isolated from Mattirolomyces terfeziodes range from about 40% to 60% by weight HTS-2 to 60% to 40% by weight HTS-1. In embodiments, the invention provides non-naturally occurring sweet proteins which include non-naturally occurring mixtures of the two isoforms HTS-1 and HTS-2. In embodiments, the invention provides sweet protein generated by recombinant expression of the coding sequence of SEQ ID NO:2 or a codon-optimized version of the coding sequence of SEQ ID NO:2 in a non-native host (e.g., a host other than Mattirolomyces terfezioides truffle), as well as methods of generating sweet protein by recombinant expression.

In an embodiment the encoded polypeptide is the polypeptide having the amino acid sequence of SEQ ID NO:3 (also designated as the HTS-1 isoform). In an embodiment the encoded polypeptide is the polypeptide having the amino acid sequence of SEQ ID NO:3, except that the methionine at position 1 in SEQ ID NO:3 is absent, designated hereinafter as the polypeptide having the amino acid sequence of SEQ ID NO:141 (also designated as the HTS-2 isoform). In embodiments, the encoded polypeptide is the polypeptide having amino acid sequence SEQ ID NO:5 which is the HTS-1 isoform with a (His-tag) 6. In embodiments, the encoded polypeptide is the polypeptide having amino acid sequence SEQ ID NO: 142 which is the HTS-2 isoform with a (His-tag) 6.

Accordingly, one aspect of the invention provides a polynucleotide (e.g., isolated polynucleotide) encoding a polypeptide, wherein the encoded polypeptide has a sweet-taste modulation activity and which is different in amino acid sequence from the polypeptide of SEQ ID NO: 3 or optionally different from the polypeptide of SEQ ID NO:141. In embodiments, the encoded polypeptide is selected from the group consisting of: (a) a polypeptide sequence selected from the group consisting of the amino acid sequences of a variant listed in Table 8; Table 9 or Table 10 (b) a polypeptide having at least 80% sequence identity to a polypeptide sequence selected from the group consisting of the amino acid sequences of a variant listed in Table 8, Table 9 or Table 10; and (c) a polypeptide sequence modified from the polypeptide sequence selected from the group consisting of the amino acid sequences of a variant listed in Table 8, Table 9 or Table 10 by deletion, insertion, substitution, or addition of no more than 24 amino acids, and wherein the polypeptide sequence is different from that of SEQ ID NO:3 and optionally different from that of SEQ ID NO:141. In an embodiment, the polynucleotide encoding the polypeptide is other than the polynucleotide of SEQ ID NO:2. In an embodiment, the polynucleotide encoding the polypeptide is the polynucleotide of SEQ ID NO:2 which is codon optimized for expression in a bacterium, yeast or fungus. In an embodiment, the polynucleotide encoding the polypeptide is the polynucleotide of SEQ ID NO:6 with optional His-tag which is codon optimized for expression in E. coli. In an embodiment, the polynucleotide encoding the polypeptide is the polynucleotide of SEQ ID NO:7 with optional His-tag which is codon optimized for expression in Saccharomyces cerevisiae.

In an embodiment, the encoded polypeptide is a polypeptide having the amino acid sequence of SEQ ID NO:3, except that the methionine at position 1 in SEQ ID NO:3 is absent, presented herein as the polypeptide having the amino acid sequence of SEQ ID NO:141. In an embodiment, the encoded polypeptide is a polypeptide other than the polypeptide having the amino acid sequence of SEQ ID NO:141. In embodiments, the encoded polypeptide exhibits sweet taste. In embodiments, the encoded polypeptide exhibits sweet taste, when present in a composition at a concentration of 30 ppm or higher. In embodiments, the encoded polypeptide exhibits sweet taste, when present in a composition at a concentration of 40 ppm or higher.

In a related aspect, the invention provides a polynucleotide (e.g., isolated polynucleotide) encoding a polypeptide, wherein the encoded polypeptide has flavor modifying properties, and which is different in amino acid sequence from polypeptide SEQ ID NO:3 and optionally from SEQ ID NO:141. In embodiments, the encoded polypeptide is selected from the group consisting of: (a) a polypeptide sequence selected from the group consisting of the amino acid sequences of a variant listed in Table 8; (b) a polypeptide having at least 80% sequence identity to a polypeptide sequence selected from the group consisting of the amino acid sequences of a variant listed in Table 8; and (c) a polypeptide sequence modified from the polypeptide sequence selected from the group consisting of the amino acid sequences of a variant listed in Table 8, by deletion, insertion, substitution, or addition of no more than 24 amino acids, and wherein the polypeptide sequence is different from that of SEQ ID NO:3 and optionally from SEQ ID NO: 141. In embodiments, the encoded polypeptide is a polypeptide selected from the group consisting of the amino acid sequences of a variant listed in Table 8, that is other than a variant listed in Table 9 or Table 10. In an embodiment, the polynucleotide encoding the polypeptide is a polynucleotide other than the polynucleotide of SEQ ID NO:2. In an embodiment, the encoded polypeptide is a polypeptide having the amino acid sequence of SEQ ID NO:141. In an embodiment, the encoded polypeptide is a polypeptide other than the polypeptide having the amino acid sequence of SEQ ID NO:141. In embodiments, the encoded polypeptide does not exhibit sweet taste. In embodiments, the encoded polypeptide does not exhibit sweet taste when present in a composition at a concentration of 30 ppm or higher. In embodiments, the encoded polypeptide does not exhibit sweet taste when present in a composition at a concentration of 40 ppm or higher. In embodiments, the encoded polypeptide only exhibits sweet taste when present in a composition at a concentration of 30 ppm or higher.

In another aspect of the invention, amino acid mutations of HTS polypeptide and protein variants are summarized in Table 8. The mutations of Table 8 are identified relative to the amino acid sequence of SEQ ID NO:3. Thus, a polypeptide having an amino acid sequence of a variant listed in Table 8 will have the amino acid sequence of SEQ ID NO:3, but with one or more site mutations identified in Table 8. Mutants of Table 8 also include those in which the methionine at position 1 of the protein is absent. In a related aspect, the invention provides HTS polypeptide and protein variants, wherein the variants have the single site mutations of Table 8 relative to the amino acid sequence of SEQ ID NO:3 and in addition wherein the methionine at position 1 in SEQ ID NO:3 is absent.

In another aspect of the invention, amino acid mutations of HTS polypeptide and protein variants are summarized in Table 9. The mutations of Table 9 are identified relative to the amino acid sequence of SEQ ID NO:3. Thus, a polypeptide having an amino acid sequence of a variant listed in Table 9 will have the amino acid sequence of SEQ ID NO:3, but with one or more site mutations identified in Table 9. Mutants of Table 9 also include those in which the methionine at position 1 of the protein is absent. In a related aspect, the invention provides HTS polypeptide and protein variants, wherein the variants have the single site mutations of Table 9 relative to the amino acid sequence of SEQ ID NO:3 and in addition wherein the methionine at position 1 in SEQ ID NO:3 is absent.

In another aspect of the invention, amino acid mutations of HTS polypeptide and protein variants are summarized in Table 10. The mutations of Table 10 are identified relative to the amino acid sequence of SEQ ID NO:3. Thus, a polypeptide having an amino acid sequence of a variant listed in Table 10 will have the amino acid sequence of SEQ ID NO:3, but with one or more site mutations identified in Table 10. Mutants of Table 10 also include those in which the methionine at position 1 of the protein is absent. In a related aspect, the invention provides HTS polypeptide and protein variants, wherein the variants have the single site mutations of Table 8 relative to the amino acid sequence of SEQ ID NO:3 and in addition wherein the methionine at position 1 in SEQ ID NO:3 is absent.

In another aspect of the invention, the HTS polypeptide and protein variants are those having the single-site mutations listed in Table 7, which are identified relative to the amino acid sequence of SEQ ID NO:3. In a related aspect of the invention, the HTS polypeptide and protein variants are those having the single-site mutations listed in Table 7 relative to the amino acid sequence of SEQ ID NO:3, and in addition wherein the methionine at position 1 in SEQ ID NO:3 is absent.

In another aspect of the invention, the HTS polypeptide and protein variants are other than those having the single-site mutations listed in Table 7 which are identified relative to the amino acid sequence of SEQ ID NO:3. In a related aspect, the present invention excludes polynucleotides encoding the HTS polypeptide and protein variants having the single-site mutations listed in Table 7. In a related aspect of the invention, the HTS polypeptide and protein variants are other than those having the single-site mutations listed in Table 7 and wherein the methionine at position 1 in SEQ ID NO:3 is absent. In a related aspect, the present invention excludes polynucleotides encoding the HTS polypeptide and protein variants having the single-site mutations listed in Table 7 and wherein the methionine of SEQ ID NO:3 is absent.

In another aspect of the invention, the HTS polypeptide or protein variants do not include a mutation of Table 7. In a related aspect, the present invention excludes HTS polypeptide or protein variants comprising a mutation listed in Table 7. In a related aspect, the present invention excludes polynucleotides encoding a HTS polypeptide or protein variant comprising a mutation listed in Table 7.

In another aspect of the invention, the HTS polypeptide or protein variants do not include a mutation of Table 3 or Table 6. In a related aspect, the present invention excludes HTS polypeptide or protein variants comprising a mutation listed in Table 3 or Table 6. In a related aspect, the present invention excludes polynucleotides encoding a HTS polypeptide or protein variant comprising a mutation listed in Table 3 or Table 6.

In yet another aspect of the invention, the HTS polypeptide or protein variant does not include a mutation of Table 8, Table 9 or Table 10, but does include 1 or more conservative mutations of the amino acid sequence of SEQ ID NO:3 or optionally of SEQ ID NO:141, wherein, in the variant, the methionine at position 1 is present or absent. In a related aspect, the present invention includes polynucleotides encoding a HTS polypeptide or protein variant that does not include a mutation of Table 8, Table 9 or Table 10, but does include 1 or more conservative mutations of the amino acid sequence of SEQ ID NO:3, wherein, in the variant, the methionine at position 1 is present or absent.

In other aspects, the polynucleotide encoding the polypeptide having flavor-modulation or sweet-taste modulation activity is optionally operably linked to a heterologous regulatory element. Additionally or alternatively, the polynucleotide sequence further encodes a protein/peptide tag or label. The protein/peptide tag is optionally an affinity tag. The protein tag is optionally a histidine tag (His tag). Optionally the protein tag is (His) 6. In an embodiment, the polynucleotide encoding the polypeptide having sweet-taste modulation activity is other than the nucleotide of SEQ ID NO:2 further encoding a protein/peptide tag or label.

An aspect of the invention provides a polynucleotide (e.g., isolated polynucleotide) encoding a polypeptide, where the encoded polypeptide is selected from the group consisting of: (a) a polypeptide sequence selected from the group consisting of the amino acid sequences of a variant listed in Table 8, Table 9 or Table 10, where the polypeptide further consists of a protein/peptide tag, optionally an affinity tag, or optionally a histidine tag; (b) a polypeptide having at least 80% sequence identity to a polypeptide sequence selected from the group consisting of the amino acid sequences of a variant listed in Table 8, Table 9 or Table 10, where the polypeptide further consists of a protein/peptide tag, optionally an affinity tag, or optionally a histidine tag; and (c) a polypeptide sequence modified from the polypeptide sequence selected from the group consisting of the amino acid sequences of a variant listed in Table 8, Table 9 or Table 10 by deletion, insertion, substitution, or addition of no more than 24 amino acids and wherein the polypeptide further consists of a protein/peptide tag, optionally an affinity tag or optionally a histidine tag and where the polypeptide sequence is different from that of SEQ ID NO: 3 containing the histidine tag. In a related embodiment, the encoded polypeptide is different from that of SEQ ID NO: 142 containing a histidine tag.

In specific aspects, the polynucleotide encoding the polypeptide having sweet-taste modulation activity is other than SEQ ID NO:76, SEQ ID NO:77, SEQ ID NO:79, SEQ ID NO: 80, SEQ ID NO:82, SEQ ID NO:83, SEQ ID NO:85, SEQ ID NO:86, SEQ ID NO:88, SEQ ID NO: 89, SEQ ID NO:90, SEQ ID NO:91, SEQ ID NO:93, SEQ ID NO:94, SEQ ID NO:96, SEQ ID NO:97, SEQ ID NO:99, SEQ ID NO:100, SEQ ID NO: 102, SEQ ID NO: 103, SEQ ID NO: 104, SEQ ID NO: 105, SEQ ID NO: 107, SEQ ID NO: 108, SEQ ID NO: 110, SEQ ID NO: 111, SEQ ID NO:112, SEQ ID NO:113, SEQ ID NO:115, SEQ ID NO:116, SEQ ID NO: 117, SEQ ID NO:118, SEQ ID NO:120, SEQ ID NO:121, SEQ ID NO: 122, SEQ ID NO: 123, SEQ ID NO:125, SEQ ID NO:126, SEQ ID NO: 128, SEQ ID NO: 129, SEQ ID NO: 130, SEQ ID NO:131, SEQ ID NO:133, SEQ ID NO:134, SEQ ID NO:135, SEQ ID NO: 136, SEQ ID NO:138, SEQ ID NO:139 or SEQ ID NO:140 as identified in international patent application PCT/US2022/82443 and U.S. patent application Ser. No. 18/146,958.

Another aspect of the invention provides a polynucleotide other than the polynucleotide of SEQ ID NO:4, which corresponds to the coding sequence optimized for His tagged HTS in E. coli (wherein residues 364-381 correspond to an optional His tag sequence). Another aspect of the invention provides a polynucleotide other than the polynucleotide of SEQ ID NO:6, which corresponds to the coding sequence of HTS optimized for expression in S. cerevisiae (to which a coding sequence for a His-tag, e.g., (His) 6 is added).

In a specific embodiment, the invention provides Myd variants which exhibit enhanced thermostability with respect to native isoforms of Myd proteins (HTS-1 and HTS-2). In specific embodiment, the invention provides a

[symbol]

In additional aspects, the invention provides an expression cassette comprising a polynucleotide herein and a vector comprising the polynucleotide, as well as a host cell transformed with the vector. Additionally, a method of producing a protein having sweet-taste modulation activity, comprising culturing the transformed host cell in a medium under conditions that result in producing the protein having sweet-taste modulation activity also is provided.

In additional aspects, the invention provides a host cell which expresses a polypeptide as described herein. In embodiments, the host cell is E. coli. In embodiments, the host cell is a plant cell.

Another aspect of the invention provides a polypeptide (e.g., isolated polypeptide) having sweet-taste modulation activity and comprising (i) a polypeptide sequence selected from those amino acid sequences of a variant listed in Table 8, Table 9 or Table 10; wherein the polypeptide sequence is different from SEQ ID NO:3 and optionally different from SEQ ID NO:141 or (ii) a polypeptide having at least 80% sequence identity to a polypeptide sequence selected from the group consisting of those amino acid sequences of a variant listed in Table 8, Table 9 or Table 10, wherein the polypeptide sequence is different from SEQ ID NO:3 and optionally different from SEQ ID NO:141. In additional aspects, the polypeptide (a) contains at least one modification relative to a polypeptide sequence selected from the group consisting of an amino acid sequence of a variant listed in Table 8, Table 9 or Table 10 by deletion, insertion, substitution, or addition of no more than 24 amino acids, wherein the polypeptide is different from polypeptide SEQ ID NO:3 and optionally different from SEQ ID NO:141, and/or (b) further comprises a protein/peptide tag, optionally an affinity tag, and particularly a histidine tag, and wherein the polypeptide has sweet-taste modulation activity.

In another aspect, the invention provides a polypeptide having sweet-taste modulation activity (e.g., isolated polypeptide) comprising the amino acid sequence of SEQ ID NO:3 which is modified by at least one mutation selected from those listed in Table 8, Table 9 or Table 10. In a related aspect, the invention provides a polypeptide having sweet-taste modulation activity (e.g., isolated polypeptide) comprising the amino acid sequence of SEQ ID NO:3 which is modified by two mutations at different positions selected from those listed in Table 8, Table 9 or Table 10. In a related aspect, the invention provides a polypeptide having sweet-taste modulation activity (e.g., isolated polypeptide) comprising the amino acid sequence of SEQ ID NO:3 which is modified by three mutations at different positions selected from those listed in Table 8, Table 9 or Table 10. In a related aspect, the invention provides a polypeptide having sweet-taste modulation activity (e.g., isolated polypeptide) comprising the amino acid sequence of SEQ ID NO: 3 which is modified by four mutations at different positions selected from those listed in Table 8, Table 9 or Table 10. In a related aspect, the invention provides a polypeptide having sweet-taste modulation activity (e.g., isolated polypeptide) comprising the amino acid sequence of SEQ ID NO:3 which is modified by five mutations at different positions selected from those listed in Table 8, Table 9 or Table 10. In a related aspect, the invention provides a polypeptide having sweet-taste modulation activity (e.g., isolated polypeptide) comprising the amino acid sequence of SEQ ID NO:3 which is modified by six mutations at different positions selected from those listed in Table 8, Table 9 or Table 10. In further embodiments of the preceding embodiments, the 1-6 modifications are other than removal of the methionine at position 1. In embodiments, in the polypeptides having 1-6 amino acid modifications, the methionine at position 1 is present or absent. The invention also provides polynucleotides encoding the foregoing mutant (variant) polypeptides of SEQ ID NO:3. The invention further provides the foregoing mutant polypeptides which further comprise a protein/peptide tag, optionally an affinity tag and more specifically a histidine tag. The invention also provides polynucleotides encoding the foregoing mutant polypeptides of SEQ ID NO:3 which mutants further comprise a protein/peptide tag, optionally an affinity tag and more specifically a histidine tag. In an embodiment, the polypeptide excludes polypeptides comprising a mutation listed in Table 7.

In a related aspect, the invention provides a polypeptide having sweet-taste modulation activity (e.g., isolated polypeptide) comprising the amino acid sequence of SEQ ID NO:3 which is modified by seven mutations at different positions selected from those listed in Table 8, Table 9 or Table 10. In a related aspect, the invention provides a polypeptide having sweet-taste modulation activity (e.g., isolated polypeptide) comprising the amino acid sequence of SEQ ID NO: 3 which is modified by eight mutations at different positions selected from those listed in Table 8, Table 9 or Table 10. In a related aspect, the invention provides a polypeptide having sweet-taste modulation activity (e.g., isolated polypeptide) comprising the amino acid sequence of SEQ ID NO:3 which is modified by nine mutations at different positions selected from those listed in Table 8, Table 9 or Table 10. In a related aspect, the invention provides a polypeptide having sweet-taste modulation activity (e.g., isolated polypeptide) comprising the amino acid sequence of SEQ ID NO:3 which is modified by ten mutations at different positions selected from those listed in Table 8, Table 9 or Table 10. In a related aspect, the invention provides a polypeptide having sweet-taste modulation activity (e.g., isolated polypeptide) comprising the amino acid sequence of SEQ ID NO:3 which is modified by eleven mutations at different positions selected from those listed in Table 8, Table 9 or Table 10. In a related aspect, the invention provides a polypeptide having sweet-taste modulation activity (e.g., isolated polypeptide) comprising the amino acid sequence of SEQ ID NO:3 which is modified by twelve mutations at different positions selected from those listed in Table 8, Table 9 or Table 10. In further embodiments of the preceding embodiments, the 1-12 modifications are other than removal of the methionine at position 1. In embodiments, in the polypeptides having 1-12 amino acid modification, the methionine at position 1 is present or absent. The invention also provides polynucleotides encoding the foregoing mutant polypeptides of SEQ ID NO:3. The invention further provides the foregoing mutant polypeptides which further comprise a protein/peptide tag, optionally an affinity tag and more specifically a histidine tag. The invention also provides polynucleotides encoding the foregoing mutant polypeptides of SEQ ID NO:3 which further comprise a protein/peptide tag, optionally an affinity tag and more specifically a histidine tag. In an aspect, the polypeptide excludes polypeptides comprising a mutation listed in Table 7.

In a related aspect, the invention provides a polypeptide having sweet-taste modulation activity (e.g., isolated polypeptide) comprising the amino acid sequence of SEQ ID NO:3 which is modified by thirteen mutations at different positions selected from those listed in Table 8, Table 9 or Table 10. In a related aspect, the invention provides a polypeptide having sweet-taste modulation activity (e.g., isolated polypeptide) comprising the amino acid sequence of SEQ ID NO: 3 which is modified by fourteen mutations at different positions selected from those listed in Table 8, Table 9 or Table 10. In a related aspect, the invention provides a polypeptide having sweet-taste modulation activity (e.g., isolated polypeptide) comprising the amino acid sequence of SEQ ID NO:3 which is modified by fifteen mutations at different positions selected from those listed in Table 8, Table 9 or Table 10. In a related aspect, the invention provides a polypeptide having sweet-taste modulation activity (e.g., isolated polypeptide) comprising the amino acid sequence of SEQ ID NO:3 which is modified by sixteen mutations at different positions selected from those listed in Table 8, Table 9 or Table 10. In a related aspect, the invention provides a polypeptide having sweet-taste modulation activity (e.g., isolated polypeptide) comprising the amino acid sequence of SEQ ID NO:3 which is modified by seventeen mutations at different positions selected from those listed in Table 8, Table 9 or Table 10. In a related aspect, the invention provides a polypeptide having sweet-taste modulation activity (e.g., isolated polypeptide) comprising the amino acid sequence of SEQ ID NO:3 which is modified by eighteen mutations at different positions selected from those listed in Table 8, Table 9 or Table 10. In further embodiments of the preceding embodiments, the 1-18 modifications are other than removal of the methionine at position 1. In embodiments, in the polypeptides having 1-18 amino acid modification, the methionine at position 1 is present or absent. The invention also provides polynucleotides encoding the foregoing mutant polypeptides of SEQ ID NO:3. The invention further provides the foregoing mutant polypeptides which further comprise a protein/peptide tag, optionally an affinity tag and more specifically a histidine tag. The invention also provides polynucleotides encoding the foregoing mutant polypeptides of SEQ ID NO:3 which further comprise a protein/peptide tag, optionally an affinity tag and more specifically a histidine tag. In an aspect, the polypeptide excludes polypeptides comprising a mutation listed in Table 7.

In a related aspect, the invention provides a polypeptide having sweet-taste modulation activity (e.g., isolated polypeptide) comprising the amino acid sequence of SEQ ID NO:3 which is modified by nineteen mutations at different positions selected from those listed in Table 8, Table 9 or Table 10. In a related aspect, the invention provides a polypeptide having sweet-taste modulation activity (e.g., isolated polypeptide) comprising the amino acid sequence of SEQ ID NO: 3 which is modified by twenty mutations at different positions selected from those listed in Table 8, Table 9 or Table 10. In a related aspect, the invention provides a polypeptide having sweet-taste modulation activity (e.g., isolated polypeptide) comprising the amino acid sequence of SEQ ID NO:3 which is modified by twenty-one mutations at different positions selected from those listed in Table 8, Table 9 or Table 10. In a related aspect, the invention provides a polypeptide having sweet-taste modulation activity (e.g., isolated polypeptide) comprising the amino acid sequence of SEQ ID NO:3 which is modified by twenty-two mutations at different positions selected from those listed in Table 8, Table 9 or Table 10. In a related aspect, the invention provides a polypeptide having sweet-taste modulation activity (e.g., isolated polypeptide) comprising the amino acid sequence of SEQ ID NO:3 which is modified by twenty-three mutations at different positions selected from those listed in Table 8, Table 9 or Table 10. In a related aspect, the invention provides a polypeptide having sweet-taste modulation activity (e.g., isolated polypeptide) comprising the amino acid sequence of SEQ ID NO:3 which is modified by twenty-four mutations at different positions selected from those listed in Table 8, Table 9 or Table 10. In further embodiments of the preceding embodiments, the 1-24 modifications are other than removal of the methionine at position 1. In embodiments, in the polypeptides having 1-24 amino acid modification, the methionine at position 1 is present or absent. The invention also provides polynucleotides encoding the foregoing mutant polypeptides of SEQ ID NO:3. The invention further provides the foregoing mutant polypeptides which further comprise a protein/peptide tag, optionally an affinity tag, and more specifically a histidine tag. The invention also provides polynucleotides encoding the foregoing mutant polypeptides of SEQ ID NO:3 which further comprise a protein/peptide tag, optionally an affinity tag and more specifically a histidine tag. In an aspect, the polypeptide excludes polypeptides comprising a mutation listed in Table 7.

Another aspect of the invention provides a polypeptide having sweet-taste modulation activity (e.g., isolated polypeptide) comprising the amino acid sequence of SEQ ID NO:3 which is modified by at least one mutation, where the at least one mutation is selected from the mutations listed in Table 8, Table 9 or Table 10 and is not a mutation listed in Table 7, and where the polypeptide is also modified by one or more additional mutations, where the one or more additional mutations are selected from the mutations listed in Table 7. In a further aspect, the polypeptide is modified by two or more mutations selected from the mutations listed in Table 8, Table 9 or Table 10. In an aspect, the polypeptide is also modified by two or more additional mutations, where the two or more additional mutations are selected from the mutations listed in Table 7. In an aspect, the polypeptide has at least 80% sequence identity to a polypeptide sequence selected from the group consisting of those amino acid sequences of variants listed in Table 8, Table 9 or Table 10. The forgoing polypeptides optionally further comprise a protein/peptide tag, optionally an affinity tag or optionally a histidine tag.

In another aspect, the polypeptides of the invention are optionally isolated and/or optionally purified. In another aspect, the polynucleotides of the invention are optionally isolated and/or purified. In another aspect, the polypeptides of the invention optionally comprise a protein/peptide tag, optionally an affinity tag or optionally a histidine tag. In another aspect, the polypeptides of the invention optionally comprise an affinity tag. In another aspect, the polypeptides of the invention optionally comprise a histidine tag. In another aspect, the polypeptides of the invention optionally have a methionine at position 1. In another aspect, the polypeptides of the invention are optionally derivatized as described herein. In another aspect, the polypeptides of the invention are acylated, and optionally acetylated, at the N-terminus. In another aspect, the polypeptides of the invention are acylated at the N-terminus. In another aspect, the polypeptides of the invention have a methionine at position 1 and the methionine is derivatized and optionally the S of the methionine is oxidized. In another aspect, the polypeptides of the invention have a methionine at position 1 and the methionine is derivatized wherein the S of the methionine is oxidized. In another aspect, the polypeptides of the invention are pegylated at the N-terminus. In another aspect, the polypeptides of the invention are derivatized at the C-terminus as described herein. In another aspect, the polypeptides of the invention are esterified at the C-terminus.

In other aspects, certain polypeptides of the invention exhibit sweet-taste modulation activity that is enhanced compared to the polypeptide of SEQ ID NO:3. In other aspects certain polypeptides of the invention exhibit thermal stability that is enhanced compared to the polypeptide of SEQ ID NO:3.

Another aspect of the invention provides a composition which comprises one or more polypeptides of this invention which exhibit flavor modulation and/or sweet taste modulation activity and/or exhibit sweet taste. The composition includes a combination of (a) a product for oral administration other than Mattirolomyces terfezioides truffle, and (b) a sweetening composition comprising the polypeptide, wherein the combination has enhanced sweet taste and/or change or enhancement in flavor compared to the product for oral administration. The polypeptide includes one or more polypeptide sequences, either individually or in a combination of two or more thereof having flavor modulation and/or sweet-taste modulation activity and comprising (i) a polypeptide sequence selected from an amino acid sequence of a variant of Table 8, Table 9 or Table 10, wherein the polypeptide sequence is different from SEQ ID NO:3 and optionally different from SEQ ID NO:141; or (ii) a polypeptide having at least 80% sequence identity to a polypeptide sequence selected from the group consisting of an amino acid sequence of a variant of Table 8, Table 9 or Table 10, wherein the polypeptide sequence is different from SEQ ID NO:3 and optionally different from SEQ ID NO:141; or (iii) wherein the polypeptide of a variant of Table 8, Table 9 or Table 10 further comprises a protein/peptide tag, or affinity tag, wherein the protein/peptide tag is optionally a histidine tag.

Another aspect of the invention is a sweetening composition which comprises one or more polypeptides of the invention (e.g., HTS variants) exhibiting sweet taste. A related aspect of the invention is a flavor modulation composition which comprises one or more polypeptides of the invention (e.g., HTS variants) exhibiting flavor modulation activity. In embodiments, sweetening compositions contain 30 ppm or more of total HTS variant polypeptide. In embodiments, flavor modulation compositions contain 30 ppm or less of total HTS variant polypeptide.

Another more specific aspect of the invention provides a composition including a combination of (a) a product for oral administration other than Mattirolomyces terfezioides truffle, and (b) a sweetening composition comprising the polypeptide, wherein the combination has enhanced sweet taste compared to the product for oral administration. In embodiments, the polypeptide includes (a) one or more amino acid sequences selected from the group consisting of the amino acid sequences of a variant of Table 8, Table 9 or Table 10. In embodiments, the polypeptide is not a variant listed in Table 7, Table 3 or Table 6.

Another aspect of the invention provides a method for modulating the taste of a product for oral administration. The method comprises combining the product for oral administration with an effective amount of a flavor modulation composition or a sweetening composition comprising the polypeptide (e.g., a HTS variant), wherein the product for oral administration differs from Mattirolomyces terfezioides truffle, and wherein the combination has enhanced flavor or sweet taste, respectively, compared to the product for oral administration. In embodiments, the polypeptide includes one or more sequences as listed herein, particularly those having one or more mutations of Table 8, Table 9 or Table 10, either individually or in a combination of two or more thereof. In embodiments, the polypeptide is not a variant listed in Table 7, Table 3 or Table 6.

Another aspect of the invention provides a method of purifying a polypeptide having flavor modulation activity or sweet-taste modulation activity. The method includes: (a) conducting hydrophobic interaction chromatography (HIC) on a composition comprising a polypeptide, and/or (b) conducting size exclusion chromatography (SEC) on the composition comprising the polypeptide. In embodiments, the method includes (a) conducting hydrophobic interaction chromatography (HIC) on a composition comprising a polypeptide, or (b) conducting size exclusion chromatography (SEC) on the composition comprising the polypeptide. In embodiments, the method includes (a) conducting hydrophobic interaction chromatography (HIC) on a composition comprising a polypeptide to generate a purified composition, and thereafter (b) conducting size exclusion chromatography (SEC) on the purified composition comprising the polypeptide. In embodiments, the polypeptide includes one or more sequences, either individually or in a combination of two or more thereof having sweet-taste modulation activity and comprising (i) a polypeptide sequence selected from that of a variant of Table 8, Table 9 or Table 10, wherein the polypeptide sequence is different from SEQ ID NO:3 and optionally different from SEQ ID NO:141; or (ii) a polypeptide having at least 80% sequence identity to a polypeptide sequence selected from the group consisting of that of a variant of Table 8, Table 9 or Table 10, wherein the polypeptide sequence is different from SEQ ID NO:3 and optionally different from SEQ ID NO:141; (iii) wherein the polypeptide sequence of the variants of Table 8, Table 9 or Table 10 further comprises a protein/peptide tag, wherein the protein tag is optionally an affinity tag or more specifically a histidine tag; (iv) wherein the polypeptide contains 1-24 mutations selected from those listed in Table 8, Table 9 or Table 10 and optionally further comprises a protein/peptide tag, wherein the protein tag is optionally an affinity tag and more specifically a histidine tag. In a related aspect, the invention provided polypeptides that are purified by the method described herein.

Another aspect of the invention provides sweetener compositions. In some embodiments, the sweetener compositions include: (a) one or more polypeptide, wherein the one or more polypeptide comprises: (i) a polypeptide sequence selected from amino acid sequences of any variant listed in Table 8, Table 9 or Table 10; (ii) a polypeptide sequence having at least 80% sequence identity to a polypeptide sequence selected from the group consisting of amino acid sequences of any variant listed in Table 8; Table 9 or Table 10; (iii) a polypeptide sequence containing at least one modification by deletion, insertion, substitution, or addition of no more than 24 amino acids, relative to a polypeptide sequence selected from the group consisting of an amino acid sequence of a variant listed in Table 8, Table 9 or Table 10; or (iv) a polypeptide of any of (i)-(iii), wherein the methionine at position 1 is absent; and/or the polypeptide further comprises a protein/peptide tag, optionally an affinity tag or optionally a histidine tag or optionally a (His) 6 tag; and (b) at least one additional sweetener other than a Myd sweet protein.

The additional sweetener is optionally selected from a steviol glycoside sweetener, a mogroside sweetener, sucrose, allulose, sucralose, a polyol, and high fructose corn syrup (HFCS). In other embodiments, the sweetener compositions include: (a) one or more polypeptide, wherein the one or more polypeptide comprises: (i) a polypeptide sequence selected from amino acid sequences of any variant listed in Table 8, Table 9 or Table 10; (ii) a polypeptide sequence having at least 80% sequence identity to a polypeptide sequence selected from the group consisting of amino acid sequences of any variant listed in Table 8; Table 9 or Table 10; (iii) a polypeptide sequence containing at least one modification by deletion, insertion, substitution, or addition of no more than 24 amino acids, relative to a polypeptide sequence selected from the group consisting of an amino acid sequence of a variant listed in Table 8, Table 9 or Table 10; or (iv) a polypeptide of any of (i)-(iii), wherein the methionine at position 1 is absent; and/or the polypeptide further comprises a protein/peptide tag, optionally an affinity tag or optionally a histidine tag or optionally a (His) 6 tag; and (b) at least one additional component selected from monosaccharides, disaccharides (e.g., sucrose), sugar alcohols (e.g., mannitol), amino acids (e.g., leucine), organic acids (e.g., citric acid), hypoxanthine, theophylline, vitamins and combinations thereof.

In embodiments, sweetener composition herein comprise at least one additional component selected from sucrose, mannitol, citric acid, hypoxanthine, theophylline, leucine, and combinations thereof.

In specific embodiments, the polypeptide of the sweetener compositions has the amino acid sequence of SEQ ID NO:3. In specific embodiments, the polypeptide of the sweetener composition has the amino acid sequence of SEQ ID NO:3 where the methionine at position 1 is missing. In embodiments, the polypeptide of the sweetener is a mixture of a polypeptide having amino acid sequence of SEQ ID NO:3 and a polypeptide having amino acid sequence of SEQ ID NO: 3 where the methionine at position 1 is missing.

In embodiments, the polypeptide of the sweetener compositions is present in an amount between about 1 ppm and about 50 ppm. In some embodiments, the polypeptide is present in an amount between about 1 ppm and about 40 ppm. In further embodiments, the polypeptide is present in an amount selected from an amount between about 1 ppm and about 30 ppm, about 1 ppm and about 25 ppm, about 1 ppm and about 20 ppm or about 1 ppm and about 15 ppm. In some embodiments, the polypeptide is present in an amount between about 5 ppm and about 50 ppm. In some embodiments, the polypeptide is present in an amount between about 10 ppm and about 50 ppm. In some embodiments, the polypeptide is present in an amount between about 15 ppm and about 50 ppm. In some embodiments, the polypeptide is present in an amount between about 20 ppm and about 50 ppm. In some embodiments, the polypeptide is present in an amount between about 25 ppm and about 50 ppm. In some embodiments, the polypeptide is present in an amount between about 30 ppm and about 50 ppm. In some embodiments, the polypeptide is present in an amount between about 35 ppm and about 50 ppm. In some embodiments, the polypeptide is present in an amount between about 40 ppm and about 50 ppm. In some embodiments, the polypeptide is present in an amount between about 45 ppm and about 50 ppm. In some embodiments, the polypeptide is present in an amount between about 10 ppm and about 40 ppm. In some embodiments, the polypeptide is present in an amount between about 20 ppm and about 30 ppm. In some embodiments, the steviol glycoside sweetener is selected from rebaudioside M (“Reb M”), RebM80, rebaudioside D (“Reb D”), Reb A95 and rebaudioside A (“Reb A”). In embodiments, the mogroside sweetener is selected from siamenoside I and mogroside V.

In another aspect, the invention provides a combination of a product for oral administration, wherein the product is not Mattirolomyces terfezioides truffle, and a sweetening composition described herein. In some embodiments, the product for oral administration has at least one improved organoleptic property compared to a product for oral administration that does not contain the sweetener composition, wherein the organoleptic property is selected from the group consisting of aroma, flavor, basic taste (sweetness, sour, saltiness, bitterness or umami), aftertaste or linger, temporal profile, mouthfeel or a combination thereof. In embodiments, the product for oral administration is a solid food product or a beverage product. In embodiments, the product for oral administration is a yogurt product. In embodiments, the product for oral administration is a chewing gum product.

In another aspect, the invention provides a beverage or beverage product comprising the sweetener composition described herein. In some embodiments, the beverage or beverage product has at least one improved organoleptic property compared to a beverage or beverage product that does not contain the sweetener composition, wherein the organoleptic property is selected from the group consisting of aroma, flavor, basic taste (sweetness, sour, saltiness, bitterness or umami), aftertaste or linger, temporal profile, mouthfeel or a combination thereof. In some embodiments, the beverage or beverage product is selected from a low-calorie or no-calorie beverage or beverage product. In embodiments, the beverage is selected from cola, ginger-ale, soft drinks, root beer, fruit juice, fruit-flavored juice, vegetable juice, vegetable-flavored juice, sports drinks, energy drinks, plant protein beverage, near water drinks (e.g., water with natural or synthetic flavorants), tea type (e.g. black tea, green tea, red tea, oolong tea), coffee, cocoa drink, beverage containing milk components (e.g. milk beverages, coffee containing milk components, café au lait, milk tea, fruit milk beverages). In embodiments, the beverage or beverage product includes at least one organic acid additive salt that is a sodium, calcium, potassium, or magnesium salt of an organic acid. In embodiments, the organic acid is selected from citric acid, malic acid, tartaric acid, fumaric acid, lactic acid, alginic acid, ascorbic acid, benzoic acid and adipic acid.

In another aspect, the invention provides methods of improving at least one organoleptic property of the product for oral administration described herein. In embodiments the product for oral administration is a solid food product or a liquid beverage product. In an embodiment, the invention provides methods of improving at least one organoleptic property of the product for oral administration described herein. The methods include adding a sweetener composition as described herein to a solid or liquid matrix, thereby providing the product having at least one improved organoleptic property. In embodiments, the improved organoleptic property is selected from the group consisting of aroma, flavor, basic taste (sweetness, sour, saltiness, bitterness or umami), aftertaste or linger, temporal profile, mouthfeel or a combination thereof. In embodiments, the amount of polypeptide present in the product improved by the methods is an amount between about 1 ppm and about 50 ppm, about 1 ppm and about 40 ppm, about 1 ppm and about 30 ppm, about 1 ppm and about 25 ppm, about 1 ppm and about 20 ppm or about 1 ppm and about 15 ppm. In embodiments, the product is a solid food product and the sweetener composition is added to, combined with or mixed into the solid food product.

In embodiments, the invention provides methods of improving at least one organoleptic property of the beverage or beverage product described herein. The methods include adding a sweetener composition as described herein to a liquid matrix, thereby providing a beverage or beverage product having at least one improved organoleptic property. In embodiments, the improved organoleptic property is selected from the group consisting of aroma, flavor, basic taste (sweetness, sour, saltiness, bitterness or umami), aftertaste or linger, temporal profile, mouthfeel or a combination thereof. In embodiments, the amount of polypeptide present in the beverage or beverage product improved by the methods is an amount between about 1 ppm and about 50 ppm, about 1 ppm and about 40 ppm, about 1 ppm and about 30 ppm, about 1 ppm and about 25 ppm, about 1 ppm and about 20 ppm or about 1 ppm and about 15 ppm.

In another aspect, the invention provides methods of changing/modifying at least one organoleptic property of a product for oral administration as described herein. The methods include adding a sweetener composition as described herein to the product, thereby providing a product having at least one changed/modified organoleptic property. In embodiments, the changed/modified organoleptic property is selected from the group consisting of aroma, flavor, basic taste (sweetness, sour, saltiness, bitterness or umami), aftertaste or linger, temporal profile, mouthfeel or a combination thereof. In embodiments, the amount of polypeptide present in the product changed/modified by the methods is an amount between about 1 ppm and about 50 ppm, about 1 ppm and about 40 ppm, about 1 ppm and about 30 ppm, about 1 ppm and about 25 ppm, about 1 ppm and about 20 ppm or about 1 ppm and about 15 ppm. In embodiments, the product for oral administration is a food product or a beverage product. In embodiments, the product is a solid food product and the sweetener composition is added to, combined with or mixed into the solid food product.

In another aspect, the invention provides methods of changing/modifying at least one organoleptic property of the beverage or beverage product described herein. The methods include adding a sweetener composition as described herein to a liquid matrix, thereby providing a beverage or beverage product having at least one changed/modified organoleptic property. In embodiments, the changed organoleptic property is selected from the group consisting of aroma, flavor, basic taste (sweetness, sour, saltiness, bitterness or umami), aftertaste or linger, temporal profile, mouthfeel or a combination thereof. In embodiments, the amount of polypeptide present in the beverage or beverage product changed/modified by the methods is an amount between about 1 ppm and about 50 ppm, about 1 ppm and about 40 ppm, about 1 ppm and about 30 ppm, about 1 ppm and about 25 ppm, about 1 ppm and about 20 ppm or about 1 ppm and about 15 ppm.

Other aspects and embodiments of the invention will be apparent on review of the figures, detailed description and non-limiting examples herein.

### BRIEF DESCRIPTION OF THE FIGURES

FIG. 1 shows a Coomassie-stained SDS-PAGE gel of proteins obtained from fractions of partially purified M. terfezioides gleba.

FIG. 2 shows a SDS-PAGE gel, Coomassie stained, of purification steps of His-tagged sweet polypeptide expressed in E. coli from the coding region of SEQ ID NO:4 lane 1: molecular weight standards; lane 3, crude lysate; lane 4, flow through fraction from HisPur™ Ni-NTA; lane 5, wash 1; lane 6, wash 2; lane 7, wash 3; lane 8, elution fraction. The Hist-tagged sweet protein expressed in E. coli is the His-tagged version of HTS-2 in which methionine is absent (SEQ ID NO:142)

FIG. 3 shows the concentration-response functions for sweet taste for mycodulcein (Honey Truffle Sweetener (HTS)), aspartame, thaumatin, rebaudioside A. Data are plotted as the proportion (p) of responses occurring on the 200 mM sucrose-associated (“sweet”) target. Each data point in the curves for mycodulcein, aspartame, thaumatin, rebaudioside A was calculated as the average across 32 replicates and averaged across 16 replicates for the sucrose curve; error bars are SEM. Points for water and sucrose controls were similarly calculated as the average across 128 and 64 replicates, respectively. Curves were fit by non-linear regression.

FIG. 4A shows SDS-PAGE analysis, Coomassie stain, of the eluted fraction from Capto MMC a multimodal weak cation exchange resin. M: protein marker; lane 1: eluted fraction, showing low purity after cation exchange. Arrow indicates mycodulcein (native HTS) band.

FIG. 4B shows SDS-PAGE analysis, Coomassie stain, of two eluted fractions collected during the gradient elution from the HiScreen Capto Butyl (Cytiva Sweden AB, Upsala, Sweden) column analyzed on SDS-PAGE. Lane 1 shows eluted fraction 1 not containing mycodulcein and Lane 2 shows eluted mycodulcein. The purity of the eluted fraction was determined by GelAnalyzer to be ˜86%. Arrow indicates mycodulcein (native HTS) band.

FIG. 4C shows SDS-PAGE analysis, Coomassie stain, the eluted protein from the HIC column after chromatographing on HiPrep® 26/60 Sephacryl® S-200 (Cytiva Sweden AB, Upsala, Sweden). Lane 1 shows purified his-tag mycodulcein and Lane 2 shows purified native mycodulcein. The purity of the eluted fraction was determined by GelAnalyzer to be ˜98%. Arrow indicates mycodulcein (native HTS) band.

FIG. 5 is a map of the pD44-CH vector which is isopropyl beta-D-1-thiogalactopyranoside (IPTG) inducible.

FIGS. 6A and 6B include graphs of exemplary melting temperature results obtained from GloMelt™ thermostability assays comparing native HTS (1) and HTS variant S33C_I49C (double mutant, 2). FIG. 6A compares melt curves for native HTS (1) and the double mutant (2).

FIG. 6B compares melt peak curves for 1 and 2. Relative fluorescence (RFU) is measured as a function of temperature. An increase in average melting temperature of 7.5° C. is observed with the double mutant compared to native HTS protein.

FIG. 7 shows melting temperature results obtained from GloMelt™ thermostability assays comparing native HTS protein with HTS variant double mutation S33C_I49C. In this assay, the sample was brought to a low pasteurization temperature (63° C.) and held for an hour.

### DETAILED DESCRIPTION OF THE DISCLOSURE

The invention provides embodiments of isolated nucleotides encoding Myd1 polypeptide variants, the encoded Myd1 polypeptide variants (also called Honey Truffle Sweetener (HTS) proteins and variants thereof) that are capable of flavor modulation and/or modulating sweet taste and perception, and methods of making and using such. The invention provides embodiments of MYD1 variants and Myd1 polypeptide variants either alone, or in combination with food, beverage, dietary supplement, or pharmaceuticals; and methods of modifying the taste of such foods, beverages, dietary supplements, or pharmaceutical compositions with the isolated polynucleotides and polypeptides of the invention. The invention provides naturally occurring Myd1 polypeptides and nucleic acids encoding these polypeptides. The invention provides non-naturally occurring Myd1 polypeptide variants and nucleic acids encoding these polypeptides.

An embodiment of the invention provides Myd polypeptide variants (also referred to as HTS variants). The term “Myd polypeptide variants” is used herein to identify non-naturally occurring HTS polypeptides and includes any of the polypeptides according to the present invention which e.g., have at least 80% sequence identity to a variant of Table 8, Table 9 or Table 10 and also have flavor modulation (or modifying) and/or sweet-taste modifying activity.

A sweet tasting partially purified extract of terfezioides gleba was subjected to de novo amino acid sequencing to identify a 20-mer N-terminal sequence. The Myd1 coding sequence (putatively derived from the MYD1 gene) was identified after the whole transcriptome of the Mattirolomyces. terfeziodes gleba was de novo assembled using RNAseq reads. Screening the M. terfeziodes whole transcriptome using the 20-mer N-terminus sequences identified a transcript predicted to encode a protein with 100% identity at the N-terminus. The identified transcript is predicted to encode a 121 amino acid protein. This method identified the polynucleotide of SEQ ID NO: 1. Start and stop codons were identified in the transcript to identify putative coding sequence SEQ ID NO:2. SEQ ID NO:3 is the putative encoded protein, a 121 amino acid protein. The naturally-occurring protein isolated from M. terfeziodes gleba was subsequently found to be a mixture of two isoforms the protein of SEQ ID NO:3 and a mature protein of SEQ ID NO: 3 with the methionine (Met) residue at amino acid position 1 deleted (designated SEQ ID NO:141 hereinafter). Identity between the predicted protein SEQ ID NO:3, and other protein sequences in GENBANK were 31% or less.

Native HTS appears to be a mixture of two isoforms (HTS-1 and HTS-2). HTS-1 is the polypeptide of SEQ ID NO:3. HTS-2 is the polypeptide of SEQ ID NO:141. Native HTS isolated from M. terfeziodes, as described herein, is a mixture of about 40-60% by weight HTS-2 and 60 to 40% by weight HTS-1. The relative amount of HTS-1 and HTS-2 isolated from M. terfeziodes varies at least by truffle source. As isolated, about 80% of HTS-1 is acetylated at the N-terminal amine group. No significant difference in sweet taste has as yet been observed for the two sequence isoforms of HTS. Acetylation of the N-terminus of the HTS polypeptide HTS-1 also does not have a significant effect on sweet taste.

The polynucleotide encoding the polypeptide of SEQ ID NO:3 has been successfully expressed in multiple hosts. In some cases, the polynucleotide sequence encoding the polypeptide of SEQ ID NO:3 has been codon-optimized, as known in the art, for expression in a given host. For example, intracellular expression in E. coli using optimized codons to express the polynucleotide encoding SEQ ID NO:3 using nucleotide coding sequence of SEQ ID NO:4 results in a sweet tasting protein. For example, intracellular expression in E. coli using optimized codons to express the polynucleotide encoding SEQ ID NO:3 and a (His) 6 tag results in a sweet tasting protein. The sweet protein with His-tag expressed from SEQ ID NO:4 in E. coli has subsequently been determined by proteome analysis to be SEQ ID NO:141 (SEQ ID NO:3 without methionine in position 1) with a His-tag (SEQ ID NO: 148) expression of the HTS-1 isoform of the protein is not observed. The sweet protein of SEQ ID NO:141 (without His-tag) is the protein expressed on expression of the optimized E. coli coding region without the His-tag (SEQ ID NO:148).

For example, intracellular expression in Saccharomyces cerevisiae using optimized codons (SEQ ID NO:6) to express the polynucleotide encoding SEQ ID NO:3 with an additional His-tag coding sequence results in sweet tasting protein. The protein expressed is a mixture of HTS-1 and HTS-2 isoforms each with His-tags. The relative amounts of HTS-2 to HTS-1 is about 60% to 40% by weight. Extracellular expression in Saccharomyces cerevisiae using optimized codons and an appropriate signal peptide at the N-terminal to facilitate secretion into the fermentation media resulted in an expression product that was not sweet.

For example, intracellular expression in Yarrowia lipolytica using optimized codons to express the polynucleotide encoding SEQ ID NO:3 results in sweet tasting protein. The protein expressed is a mixture of HTS-1 and HTS-2 isoforms. The relative amounts of HTS-2 to HTS-1 is about 20% to 80% by weight.

For example, intracellular expression in Pichia pastoris using optimized codons to express the polynucleotide encoding SEQ ID NO:3 results in sweet tasting protein. The protein expressed is a mixture of HTS-1 and HTS-2 isoforms. The relative amounts of HTS-2 to HTS-1 is about 90% to 10% by weight.

The coding sequences for native mycodulcein (HTS), which have been codon-optimized for expression in E. coli and Saccharomyces cerevisiae correspond to the nucleic acid sequences of SEQ ID NO:4 and SEQ ID NO:6, respectively (which both encode the amino acid sequence of SEQ ID NO:3 with an optional 6 residue histidine tag, i.e., the amino acid sequence of SEQ ID NO: 5).

In embodiments, the invention provides non-naturally occurring sweet proteins which include non-naturally occurring mixtures of the two isoforms HTS-1 and HTS-2. In embodiments, the invention provides HTS-2 (SEQ ID NO:141) substantially free of the other isoform HTS-1, where substantially free means less than 10% by weight of HTS-1 (SEQ ID NO: 3). In embodiments, the invention provides HTS-2 (SEQ ID NO:141) free of the other isoform HTS-1, where free means less than 1% by weight of HTS-1 (SEQ ID NO:3). In embodiments, the invention provides HTS-1 (SEQ ID NO:3) substantially free of the other isoform HTS-2, where substantially free means less than 10% by weight of HTS-1 (SEQ ID NO: 141). In embodiments, the invention provides HTS-1 (SEQ ID NO:3) free of the other isoform HTS-2, where free means less than 1% by weight of HTS-2 (SEQ ID NO:141). In embodiments, the invention provides non-naturally occurring mixtures of HTS-1 and HTS-2, specifically those enriched in HTS-2 where the amount of HTS-2 is greater than 60% by weight and for example is 65% or more by weight, 70% or more by weight, 75% or more by weight, 80% or more by weight, 85% or more by weight, 90% or more by weight, or 95% or more by weight. In embodiments, the invention provides non-naturally occurring mixtures of HTS-1 and HTS-2, specifically those enriched in HTS-1 where the amount of HTS-1 is greater than 60% by weight and for example is 65% or more by weight, 70% or more by weight, 75% or more by weight, 80% or more by weight, 85% or more by weight, 90% or more by weight, or 95% or more by weight.

In embodiments, the invention provides sweet protein generated by recombinant expression of the coding sequence of SEQ ID NO:2 or a codon-optimized version of the coding sequence of SEQ ID NO:2 in a non-native host (e.g., a host other than Mattirolomyces terfezioides truffle. In specific embodiment, the sweet protein is generated by recombinant expression in a bacterium, yeast or fungus. In specific embodiments, the non-native host is Escherichia coli. In specific embodiments, the non-native host is Saccharomyces cerevisiae. In specific embodiments, the non-native host is Yarrowia lipolytica. In specific embodiments, the non-native host is Pichia pastoris. In embodiments, recombinant expression in a non-native host results in non-naturally occurring mixtures of the HTS-1 and HTS-2 naturally-occurring isoforms of mycodulcein (Myd).

### Polynucleotides

An embodiment of the invention comprises a polynucleotide (e.g., isolated polynucleotide) encoding a polypeptide variant having sweet-taste modulation activity. Examples of polynucleotides encoding a polypeptide having sweet-taste modulation activity include polynucleotides able to encode polypeptides such as, but not limited to, a variant of Table 8, Table 9 or Table 10, or a polypeptide thereof further having a histidine tag, or an nucleic acid sequence with at least 80% (e.g., 81%, 82%, 83%, 84%, 85%, 86%, 87%, 88%, 89%, 90%, 91%, 92%, 93%, 94%, 95%, 96%, 97%, 98%, or 99%) sequence identity to a polynucleotides encoding a variant of Table 8, Table 9, or Table 10 or a polypeptide thereof further having a protein/peptide tag, optionally an affinity tag or optionally a histidine tag. It will be understood that the polynucleotides, including or excluding the sequence encoding the protein/peptide tag, affinity tag or histidine tag, are both provided and useful in this invention. It will be further understood that for any specific polynucleotide indicated to encode a given histidine tag (e.g., (His) 6), the sequence encoding the histidine tag can be substituted with a sequence encoding a different His tag or a sequence encoding a different protein/peptide tag or affinity tag. In embodiments, the protein/peptide, affinity or histidine tag is one that is encoded by 3-30 or 3-18 nucleotides. In embodiments, the variants recited herein above include a methionine at position 1 or the methionine at position 1 is absent. In embodiments, variant HTS polypeptides are other than the polypeptides of SEQ ID NO:3 or SEQ ID NO:141.

In an embodiment, the polynucleotide comprises, consists essentially of, or consists of a polynucleotide selected from the group consisting of polynucleotide encoding a variant of Table 8, Table 9 or Table 10, or a nucleic acid sequence with at least 80% (e.g., 81%, 82%, 83%, 84%, 85%, 86%, 87%, 88%, 89%, 90%, 91%, 92%, 93%, 94%, 95%, 96%, 97%, 98%, or 99%) sequence identity to a polynucleotide able to encode a variant of Table 8, Table 9 or Table 10. In embodiments, the encoded variant polypeptide is other than the polypeptides of SEQ ID NO:3 or SEQ ID NO:141.

Unless expressly stated otherwise, when a polynucleotide sequence has a plurality of nucleotide modifications, each modification may independently be modified with a modification of choice such as deletion, insertion, substitution, or addition, regardless of what the other modifications are. Additionally, the plurality of modifications in the polynucleotide sequence may be the same or differ from each other; and the modification options for each modification may independently vary such deletion, insertion, substitution, or addition, etc. unless expressly stated otherwise. When there are a plurality of modifications, each modification may independently be a substitution, addition, insertion, or deletion regardless of what the other modification is or are. In an embodiment, the polynucleotide sequence has at least one substitute modification. In a particular embodiment, the polynucleotide sequence has a plurality of substitution modifications; and each substitution may independently be substituted with a substitution of choice regardless of what the other substitutions are. Additionally, the plurality of substitutions in the polynucleotide sequence may be the same or differ from each other; and the options for nucleotide substitutions for each substitution may independently vary unless expressly stated otherwise. When the polynucleotide sequence has a plurality of substitutions, each substitution may independently be chosen regardless of what the other substitutions is or are. In embodiments, polynucleotides herein are optionally isolated and/or optionally purified.

In embodiments, at least 80% sequence identity with respect to a polynucleotide includes, without limitation, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% and at least 99% sequence identity. In embodiments, at least 80% sequence identity with respect to a polynucleotide also includes, without limitation, 81%, 82%, 83%, 84%, 85%, 86%, 87%, 88%, 89%, 90%, 91%, 92%, 93%, 94%, 95%, 96%, 97%, 98% and 99% sequence identity.

In an embodiment, the polynucleotide encodes a polypeptide sequence comprising, consisting essentially of, or consisting of a polypeptide selected from the group consisting of: (a) the amino acid sequence of a variant of Table 8, Table 9 or Table 10, or a polypeptide thereof further having a protein/peptide tag, an affinity tag or a histidine tag; (b) an amino acid sequence having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity to the amino acid sequence of a variant of Table 8, Table 9 or Table 10, or a polypeptide thereof further having a protein/peptide tag, an affinity tag or a histidine tag; and (c) an amino acid sequence modified from an amino acid sequence of a variant of Table 8, Table 9 or Table 10 by deletion, insertion, substitution, or addition of no more than 24 amino acids (e.g., modification of 1, 2, 3, 4, 5, 6, 7, 8, 9, 10, 11, 12, 13, 14, 15, 16, 17, 18, 19, 20, 21, 22, 23, or 24 amino acids), or a polypeptide thereof further having a histidine tag. In the above listed embodiments, the polypeptide encoded by the polynucleotide is optionally a polypeptide other than that of SEQ ID NO:3 and optionally is other than that of SEQ ID NO:141. When the amino acid sequence has a plurality of modifications, the number of modifications the amino acid has may range from at least one and up to 24 modifications, such as 1, 2, 3, 4, 5, 6, 7, 8, 9, 10, 11, 12, 13, 14, 15, 16, 17, 18, 19, 20, 21, 22, 23, 24 modifications, as desired. Furthermore, unless expressly stated otherwise, when an amino acid sequence has a plurality of modifications, each modification may independently be modified with a modification of choice such as deletion, insertion, substitution, or addition, regardless of what the other modifications are. Additionally, the plurality of modifications in the amino acid sequence may be the same or differ from each other; and the modification options for each modification may independently vary such deletion, insertion, substitution, or addition, etc. unless expressly stated otherwise. When the amino acid sequence has a plurality of modifications, each modification may independently be a substitution, addition, insertion, or deletion regardless of what the other modification is or are. In an embodiment, the amino acid sequence has at least one substitute modification. In a particular embodiment, the amino acid sequence has a plurality of substitution modifications; and each substitution modification may independently be substituted with a substitution of choice regardless of what the other substitutions are. In an embodiment, the amino acid sequence has one substitution modification (a single mutant) or two substitution modifications (a double mutant) compared to SEQ ID NO:3 or SEQ ID NO:141. Additionally, the plurality of substitutions in the amino acid sequence may be the same or differ from each other; and the options for substitutions for each amino acid may independently vary unless expressly stated otherwise. When the amino acid sequence has a plurality of substitutions, each substitution may independently be chosen regardless of what the other substitutions is or are. In embodiments, the polypeptide encoded may include 1-6, 1-12, 1-18 or 1-24 substitutions compared to the polypeptide of SEQ ID NO:3, wherein the substitutions are those listed in Table 8. In embodiments, the polypeptide encoded may include 1-6, 1-12, 1-18 or 1-24 substitutions compared to the polypeptide of SEQ ID NO:3, wherein the substitutions are those listed in Table 9. In embodiments, the polypeptide encoded may include 1-6, 1-12, 1-18 or 1-24 substitutions compared to the polypeptide of SEQ ID NO:3, wherein the substitutions are those listed in Table 10. In embodiments, the polypeptide encoded may include 1-6, 1-12, 1-18 or 1-24 substitutions compared to the polypeptide of SEQ ID NO:3, wherein the substitutions are any one or more of those listed in Table 3, Table 6, Table 7, Table 8, Table 9 or Table 10. In embodiments, the polypeptide encoded may include 1-6, 1-12, 1-18 or 1-24 substitutions compared to the polypeptide of SEQ ID NO:3, wherein the substitutions are other than those listed in Table 7. In embodiments, the polypeptide encoded may include 1-6, 1-12, 1-18 or 1-24 substitutions compared to the polypeptide of SEQ ID NO:3, wherein the substitutions are other than those listed in Table 3 or Table 6. In embodiments, the polypeptide encoded may include 1-6, 1-12, 1-18 or 1-24 substitutions compared to the polypeptide of SEQ ID NO:3, wherein the substitutions are other than those listed in Table 3 or Table 6. In embodiments, the polypeptide encoded may include 1-6, 1-12, 1-18 or 1-24 substitutions compared to the polypeptide of SEQ ID NO:3, wherein the substitutions are those listed in Tables 9 and 10 and are other than those listed in Table 3 or Table 6 or Table 7.

In certain embodiments of polynucleotides herein, the polynucleotide encodes a polypeptide herein and also encodes a protein/peptide tag, an affinity tag or a histidine tag. In all such cases in which a specific polynucleotide encodes such a tag, the invention also provides the polynucleotide excluding the sequence encoding the tag. In additional embodiments, in those polynucleotides herein encoding a histidine tag, the sequence encoding the histidine tag can be deleted or excluded, or can be replaced with a coding sequence for a different protein/peptide tag or a different affinity tag, including a different histidine tag.

The invention provides isolated and/or purified embodiments of the respective polynucleotides, when applicable. The invention provides embodiments of each of the polynucleotides able to encode the variants of Table 8, Table 9 or Table 10 herein with or without being isolated and with or without being purified, when applicable. The invention provides embodiments of each of the polynucleotides able to encode the variants of Table 8, Table 9 or Table 10 herein with one or more mutations, when applicable.

The invention also provides an expression cassette comprising one or more polynucleotides encoding a Myd polypeptide variant (HTS variant) and a host cell transformed with the vector.

The polynucleotide encoding the HTS variant of the present invention can be in the form of a single-stranded or double-stranded DNA, RNA or an artificial nucleic acid, or can be a cDNA or a chemically synthesized DNA which does not comprise any intron. The term “MYD family” can refer to polymorphic variants, including natural alleles, mutants, alleles, and interspecies homologs that encode polypeptides that: (1) have at least about 35 to 50% amino acid sequence identity, optionally about 60, 75, 80, 85, 90, 95, 96, 97, 98, or 99% amino acid sequence identity to SEQ ID NO:3 over a window of about 25 amino acids, optionally 50-100 amino acids. In one aspect, the term “isolated” encompasses products that have been removed from a biological environment (e.g., a cell, tissue, culture medium, body fluid, etc.), or otherwise increased in purity to any degree (e.g., isolated from a synthesis medium). Isolated products, thus, can be synthetic or naturally produced. In an embodiment, the term “isolated” encompasses products that have been isolated from other proteins that are present in the natural environment. In embodiments, the term “isolated” encompasses products that have been isolated from carbohydrates that are present in the natural environment.

The term “nucleic acid” or “nucleic acid sequence” refers to a deoxy-ribonucleotide or ribonucleotide oligonucleotide in either single- or double-stranded form. The term encompasses nucleic acids, i.e., oligonucleotides, containing known analogs of natural nucleotides. The term also encompasses nucleic-acid-like structures with synthetic backbones (see e.g., Oligonucleotides and Analogues, a Practical Approach, ed. F. Eckstein, Oxford Univ. Press (1991); Antisense Strategies, Annals of the N.Y. Academy of Sciences, Vol. 600, Eds. Baserga et al. (NYAS 1992); Milligan J. Med. Chem. 36:1923-1937 (1993); Antisense Research and Applications (1993, CRC Press), WO 97/03211; WO 96/39154; Mata, Toxicol. Appl. Pharmacol. 144:189-197 (1997); Strauss-Soukup, Biochemistry 36:8692-8698 (1997); Samstag, Antisense Nucleic Acid Drug Dev, 6:153-156 (1996)).

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are optionally directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

The polynucleotide or polypeptide can be naturally occurring or non-naturally occurring (e.g., synthetic, recombinant, modified, and/or variant products). In one aspect, the naturally occurring or non-naturally occurring products are isolated or purified. In another aspect, the naturally occurring or non-naturally occurring products are not isolated or are not purified. In embodiments herein, the polynucleotides and polypeptides are non-naturally occurring. In embodiments herein, the polynucleotides and polypeptides are non-naturally occurring variants of the naturally-occurring mycoduclein (HTS) polypeptide and the naturally-occurring polynucleotide sequence that encodes the naturally-occurring mycoduclein (HTS).

As used herein, “recombinant” refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide. “Recombinant means” also encompass the ligation of nucleic acids having various coding regions or domains or promoter sequences from different sources into an expression cassette or vector for expression of, e.g., inducible or constitutive expression of a fusion protein comprising a translocation domain of the invention and a nucleic acid sequence amplified using a primer of the invention.

As used herein, the terms “amplifying” and “amplification” refer to the use of any suitable amplification methodology for generating or detecting recombinant or naturally expressed nucleic acid, as described in detail, below. For example, the invention provides methods and reagents (e.g., specific degenerate oligonucleotide primer pairs) for amplifying (e.g., by polymerase chain reaction, PCR) naturally expressed (e.g., genomic or mRNA) or recombinant (e.g., cDNA) nucleic acids of the invention (e.g., taste stimulus-binding sequences of the invention) in vivo or in vitro.

As used herein, the term “isolated,” when referring to a nucleic acid or polypeptide refers to a state of purification or concentration different than that which occurs naturally. Any degree of purification or concentration greater than that which occurs naturally, including (1) the purification from other naturally occurring associated structures or compounds (e.g., other proteins, carbohydrates), or (2) the association with structures or compounds to which it is not normally associated in the body are within the meaning of “isolated” as used herein. The nucleic acids or polypeptides described herein may be isolated or otherwise associated with structures or compounds to which they are not normally associated in nature, according to a variety of methods and processes known to those of skill in the art. In one embodiment, the polypeptides described herein contain at most 5% (e.g., at most 4%, at most 3%, at most 2% at most 1%) by weight of other fungal proteins, e.g., fungal proteins other than Myd proteins.

“Modified” or “variant” products refer to products (e.g., polynucleotides or polypeptides) that have been altered from the original (e.g., naturally occurring) structure. As described herein, variants encompass polynucleotides or polypeptides with one or more changes to the nucleic acid or amino acid sequences, respectively. Changes include modifications to the nucleic acid or amino acid sequence, including additions, deletions, insertions, and substitutions. Modified or variant products also can encompass those modified to include disulfide bond formation, as well as those that are derivatized by glycosylation using post-translation modification methods, lipidation, acylation, acetylation, phosphorylation, or any other manipulation, such as conjugation with a labeling component, relative to the original structure. As is known in the art, derivatization can be accomplished by chemical or enzymatic methods after translation of a polypeptide. Derivatization can also be accomplished with post-translation modification during expression of the polypeptide in a selected host.

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating, e.g., sequences in which the third position of one or more selected codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). The term nucleic acid is used interchangeably with gene, cDNA, mRNA, oligonucleotide, and polynucleotide.

Herein where a specific polynucleotide is indicated to encode a histidine tag, it will be understood that the polynucleotide excluding the sequence encoding the histidine tag is also provided. It will be further understood that for any specific polynucleotide indicated to encode a given histidine tag (e.g., (His) 6), the sequence encoding the histidine tag can be substituted with a sequence encoding a different His tag or a sequence encoding a different protein/peptide tag or affinity tag.

### Polypeptides

It should be appreciated that embodiments of the invention also encompass Myd polypeptide variants (HTS variants) encoded by one or more polynucleotides. The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymers.

An embodiment of the invention includes a polypeptide comprising, consisting essentially of, or consisting of a polypeptide sequence having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity to a sequence selected from the group consisting of a variant of Table 8 or a variant of Table 8 further having a histidine tag. In embodiments, the polypeptide is not the polypeptide of SEQ ID NO:3. In embodiments, the polypeptide is not the polypeptide of SEQ ID NO:141. In embodiments, the polypeptide is the polypeptide of SEQ ID NO:3 which is substantially free (less than 95% or more specifically less than 99% by weight being present) of the polypeptide of SEQ ID NO:141. In embodiments, the polypeptide is the polypeptide of SEQ ID NO:141 which is substantially free (less than 95% or more specifically less than 99% by weight being present) of the polypeptide of SEQ ID NO:3. Optionally, the amino acid sequence has at least one and up to 24 modifications. When the amino acid sequence has a plurality of modifications, the number of modifications the amino acid has may range from at least one and up to 24 modifications such as 1, 2, 3, 4, 5, 6, 7, 8, 9, 10, 11, 12, 13, 14, 15, 16, 17, 18, 19, 20, 21, 22, 23, 24 modifications, as desired. Furthermore, unless expressly stated otherwise, when an amino acid sequence has a plurality of modifications, each modification may independently be modified with a modification of choice such as deletion, insertion, substitution, or addition, regardless of what the other modifications are. Additionally, the plurality of modifications in the amino acid sequence may be the same or differ from each other; and the modification options for each modification may independently vary such deletion, insertion, substitution, or addition, etc. unless expressly stated otherwise. When the amino acid sequence has a plurality of modifications, each modification may independently be a substitution, addition, insertion, or deletion regardless of what the other modification is or are. In an embodiment, the amino acid sequence has at least one substitute modification. In a particular embodiment, the amino acid sequence has a plurality of substitution modifications; and each substitution modification may independently be substituted with a substitution of choice regardless of what the other substitutions are. Additionally, the plurality of substitutions in the amino acid sequence may be the same or differ from each other; and the options for substitutions for each amino acid may independently vary unless expressly stated otherwise. When the amino acid sequence has a plurality of substitutions, each substitution may independently be chosen regardless of what the other substitutions is or are. The term “consisting essentially of” allows for the inclusion of components that are not essential to the function or activity of the product and do not materially affect the function or activity, such as an anti-caking agent, filler, stabilizer (e.g., thermal stabilizer), and bulking agent (e.g., maltodextrose, gum acacia and the like).

Another embodiment of the invention includes a recombinant polypeptide having sweet modulation activity comprising, consisting essentially of, or consisting of a sequence having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity to a variant of Table 8, Table 9 or Table 10, or a variant of these Tables further having a histidine tag, or being fused to a heterologous signal peptide or transit peptide. The term “consisting essentially of” allows for the inclusion of components that are not essential to the function or activity of the product and do not materially affect the function or activity, such an anti-caking agent, filler, stabilizer (e.g., thermal stabilizer), and bulking agent (e.g., maltodextrose, gum acacia and the like).

In an embodiment, the polypeptide having sweet-taste modulation activity comprises, consists essentially of or consists of a modified SEQ ID NO:3 wherein the peptide has at least 1 and up to 24 amino acid modifications as shown in Table 8, Table 9 or Table 10. In another embodiment, the polypeptide has at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity to a modified polypeptide of SEQ ID NO: 3 with at least 1 and up to 24 amino acid modifications as shown in Table 8, Table 9 or Table 10; and the polypeptide differs from the polypeptide of amino acid sequence SEQ ID NO:3 and optionally differs from the polypeptide of amino acid sequence SEQ ID NO:141.

In an embodiment, the polypeptide having sweet-taste modulation activity comprises, consists essentially of or consists of a modified SEQ ID NO:3 wherein the peptide has at least 1 and up to 24 amino acid modifications as shown in Table 8, and further has 1-24 amino acid modifications as shown in Table 7, wherein the total number of modifications is 1-24. In another embodiment, the polypeptide has at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity to a modified polypeptide of SEQ ID NO: 3 with at least 1 and up to 24 amino acid modifications as shown in Table 8 and 1-24 amino acid modifications as shown in Table 7.

In an embodiment, the polypeptide having sweet-taste modulation activity comprises, consists essentially of or consists of a modified SEQ ID NO:3 wherein the peptide has at least 1 and up to 12 amino acid modifications as shown in Table 8, and further has 1-12 amino acid modifications as shown in Table 7. In another embodiment, the polypeptide has at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity to a modified polypeptide of SEQ ID NO: 3 with at least 1 and up to 12 amino acid modifications as shown in Table 8 and 1-12 amino acid modifications as shown in Table 7.

In an embodiment, the polypeptide having sweet-taste modulation activity comprises, consists essentially of or consists of a modified SEQ ID NO:3 wherein the peptide has at least 1 and up to 6 amino acid modifications as shown in Table 8, and further has 1-6 amino acid modifications as shown in Table 7. In another embodiment, the polypeptide has at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity to a modified polypeptide of SEQ ID NO: 3 with at least 1 and up to 6 amino acid modifications as shown in Table 8 and 1-6 amino acid modifications as shown in Table 7.

In an embodiment, the polypeptide having sweet-taste modulation activity comprises, consists essentially of or consists of a modified SEQ ID NO:3 wherein the peptide has at least 1 and up to 24 amino acid modifications as shown in Table 8, Table 9, Table 10, Table 7, Table 6 or Table 3. In another embodiment, the polypeptide has at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity to a modified polypeptide of SEQ ID NO: 3 with at least 1 and up to 24 amino acid modifications as shown in Table 8, Table 9, Table 10, Table 7, Table 6 or Table 3.

In an embodiment, the polypeptide having sweet-taste modulation activity comprises, consists essentially of or consists of a modified SEQ ID NO:3 wherein the peptide has at least 1 and up to 24 amino acid modifications as shown in Table 8, Table 9 or Table 10 and no modifications as shown in Table 7, Table 3 or Table 6. In another embodiment, the polypeptide has at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity to a modified polypeptide of SEQ ID NO: 3 with at least 1 and up to 24 amino acid modifications as shown in Table 8, Table 9, Table 10 and no modifications as shown in Table 7, Table 3 or Table 6.

In an embodiment, the polypeptide having sweet-taste modulation activity comprises, consists essentially of or consists of a modified SEQ ID NO:3 wherein the peptide has at least 1 and up to 6 amino acid modifications as shown in Table 8, Table 9 or Table 10. In another embodiment, the polypeptide has at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity to a modified polypeptide of SEQ ID NO:3 with at least 1 and up to 6 amino acid modifications as shown in Table 8, Table 9 or Table 10.

In certain embodiments herein, a specific polypeptide is described as having a histidine tag or as optionally having a histidine tag (noted as XXXXXX, where X is His, in the sequence listing herein). The invention provides all specific polypeptides including or optionally including a histidine tag as well as the corresponding polypeptides excluding the histidine tag. The invention also provides all specific polypeptides having a histidine tag or an optional histidine tag as well as the polypeptides excluding the histidine tag or wherein the histidine tag is replaced with a different protein tag including a different histidine tag.

In an embodiment, the Myd peptide (HTS) variants as described herein are capable of sweet-taste modulation activity. HTS polypeptide variants of the invention may have e.g., functional, physical and chemical effects at taste receptors, such as sweet taste receptors. “Sweet-taste modulation activity” may refer to inhibitory, activating, e.g., agonist or antagonist properties of a polypeptide of the invention, identified using in vitro and in vivo assays for taste transduction. Proteins with inhibitory activity may bind to, partially or totally block stimulation, decrease, prevent, delay activation, inactivate, desensitize, or down regulate taste transduction, e.g., antagonists. Activating polypeptides may bind to, stimulate, increase, open, activate, facilitate, enhance activation, sensitize, or up regulate taste transduction, e.g., agonists. Activating polypeptides are preferred.

Sweet taste modulation also refers to enhancing the taste, such as a sweet taste, of a particular product for oral administration when administered as a combination. In addition to sweet taste, HTS variants, may also exhibit differences in intensity of sweetness and duration of sweetness. These attributes of sweetness can be assessed in taste tests as described herein.

Sweet taste modulation also refers to polypeptide variants herein which exhibit sweet taste or convey sweet taste to compositions or formulations that do not themselves exhibit sweet taste when the polypeptide variant is added to the compositions or formulations.

In some embodiments, the Myd (HTS) polypeptide variants of the invention include polypeptides that are at least as sweet as sucrose (on a w/w basis) (e.g., 1×), or alternatively, are 2×, 5×, 10×, 50×, 100×, 200×, 400×, 600×, 800×, 1000×, 1500×, 2000×, 3000×, 5000×, 10,000×, 20,000× or sweeter than sucrose, as measured by any of the methods described above or known in the art. In other embodiments, the Myd polypeptide variants are at least 1% (at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%) as sweet as sucrose.

In embodiments, the polypeptides having sweet-taste modulation activity include modified SEQ ID NO:3 with 1 to up to 24 different amino acid modifications as shown in Table 8, Table 9 or Table 10. In embodiments, the polypeptides having sweet-taste modulation activity include modified SEQ ID NO:141, with 1 to up to 24 different amino acid modifications as shown in Table 8, Table 9 or Table 10.

In an embodiment, the Myd (HTS) peptide variants as described herein have flavor modifying properties (FMP) or activity. A compound having FMP is a compound, including a protein or polypeptide that causes a change in any attributes of perceived flavor of a formulation relative to a formulation without the compound, but does not directly provide a flavor attribute. Compounds may have flavor modifying properties below a perceptible sweetness threshold concentration and provide a direct flavor sensation above that threshold. HTS proteins/polypeptide or variants can exhibit a FMP threshold for the wild-type protein in a formulation, where below the threshold, FMP are observed, and above the threshold the HTS proteins or variants directly causes sweetness perception. The exact threshold for this perception change is HTS protein and application specific.

In an embodiment, a composition or formulation containing HTS proteins or variants at about 1 to about 50 ppm exhibits flavor modification, but does not provide additive sweetness to the composition or formulation. In an embodiment, a composition or formulation containing HTS proteins or variants at about 1 to about 40 ppm exhibits flavor modification, but does not provide additive sweetness to the composition or formulation. In an embodiment, a composition or formulation containing HTS proteins or variants at about 1 to about 30 ppm exhibits flavor modification, but does not provide additive sweetness to the composition or formulation. In an embodiment, a composition or formulation containing HTS proteins or variants at about 1 to about 25 ppm exhibits flavor modification, but does not provide additive sweetness to the composition or formulation. In an embodiment, a composition or formulation containing HTS proteins or variants at about 1 to about 20 ppm exhibits flavor modification, but does not provide additive sweetness to the composition or formulation. In an embodiment, a composition or formulation containing HTS proteins or variants at about 1 to about 15 ppm exhibits flavor modification, but does not provide additive sweetness to the composition or formulation. In an embodiment, a composition or formulation containing HTS proteins or variants at about 5 to about 50 ppm exhibits flavor modification, but does not provide additive sweetness to the composition or formulation. In an embodiment, a composition or formulation containing HTS proteins or variants at about 10 to about 50 ppm exhibits flavor modification, but does not provide additive sweetness to the composition or formulation. In an embodiment, a composition or formulation containing HTS proteins or variants at about 15 to about 50 ppm exhibits flavor modification, but does not provide additive sweetness to the composition or formulation. In an embodiment, a composition or formulation containing HTS proteins or variants at about 20 to about 50 ppm exhibits flavor modification, but does not provide additive sweetness to the composition or formulation. In an embodiment, a composition or formulation containing HTS proteins or variants at about 25 to about 50 ppm exhibits flavor modification, but does not provide additive sweetness to the composition or formulation. In an embodiment, a composition or formulation containing HTS proteins or variants at about 30 to about 50 ppm exhibits flavor modification, but does not provide additive sweetness to the composition or formulation. In an embodiment, a composition or formulation containing HTS proteins or variants at about 35 to about 50 ppm exhibits flavor modification, but does not provide additive sweetness to the composition or formulation. In an embodiment, a composition or formulation containing HTS proteins or variants at about 40 to about 50 ppm exhibits flavor modification, but does not provide additive sweetness to the composition or formulation. In an embodiment, a composition or formulation containing HTS proteins or variants at about 45 to about 50 ppm exhibits flavor modification, but does not provide additive sweetness to the composition or formulation. In an embodiment, a composition or formulation containing HTS proteins or variants at about 10 to about 40 ppm exhibits flavor modification, but does not provide additive sweetness to the composition or formulation. In an embodiment, a composition or formulation containing HTS proteins or variants at about 20 to about 30 ppm exhibits flavor modification, but does not provide additive sweetness to the composition or formulation.

In embodiments, a composition or formulation containing HTS proteins or variants at concentrations above 30 ppm, and particularly at concentrations above 40 ppm, provides sweetness to the composition or formulation. In embodiments, compositions or formulations can contain from 1-100 ppm of HTS proteins or variants. In embodiments, compositions or formulations can contain from 1-5, 6, 7, 8, 9, 10, 11, 12, 13, 14, 15, 16, 17 18, 19, 20, 21, 22, 23, 24, 25, 26, 27, 28, 29 or 30 ppm of total HTS protein or variant. In embodiments, compositions or formulations can contain from 1-5, 5-10, 1-10, 1-30, or 1-30 ppm of total HTS protein or variant. In embodiments, compositions or formulations can contain from 30-100, 30-40, 40-50, 50-60, 70-80, 80-90, 90-100, 40-100 or 50-100 ppm of total HTS protein or variant.

In embodiments, at least 80% sequence identity with respect to a polypeptide includes, without limitation, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% and at least 99% sequence identity. In further embodiments, at least 80% sequence identity with respect to a polypeptide also includes, without limitation, 81%, 82%, 83%, 84%, 85%, 86%, 87%, 88%, 89%, 90%, 91%, 92%, 93%, 94%, 95%, 96%, 97%, 98% and 99% sequence identity.

### Sweetness and Thermal Stability Comparison of Modified Peptide to Unmodified Polypeptide

The Myd (HTS) protein variants described herein also include “analogs,” or “conservative variants” and “mimetics” (“peptidomimetics”) with structures and activity that substantially correspond to the exemplary sequences. Thus, the terms “conservative variant” or “analog” or “mimetic” refer to a polypeptide which has a modified amino acid sequence, such that the change(s) do not substantially alter the polypeptide's (the conservative variant's) structure and/or activity, as defined herein. These include conservatively modified variations of an amino acid sequence, i.e., amino acid substitutions, additions or deletions of those residues that are not critical for protein activity, or substitution of amino acids with residues having similar properties (e.g., acidic, basic, positively or negatively charged, polar or non-polar, etc.) such that the substitutions of even critical amino acids does not substantially alter structure and/or activity.

More particularly, “conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein.

For instance, the codons GCA, GCC, GCG and GCU all encode the amino acid alanine. Thus, at every position where an alanine is specified by a codon, the codon can be altered to any of the corresponding codons described without altering the encoded polypeptide.

Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

When it is desired to express a coding sequence in a heterologous host (i.e., a non-naturally occurring host), codon optimization can be employed as is known in the art to enhance expression levels in a selected heterologous host. Codon optimization involves replacing codons in a given naturally-occurring coding sequence with codons that have a higher usage level in the selected heterologous host. In general, codon optimization is performed by comparing the codon frequency in the naturally-occurring coding sequence with the codon frequency in the selected heterologous host and where a given codon is not one typically employed in the selected heterologous host replacing that codon with one that is used by the selected heterologous host more frequently. One to all codons in a given naturally-occurring coding sequence can be optimized. Dependent upon the codon frequencies in the naturally-occurring coding sequence and the heterologous host and the specific coding sequence, at least 50%, at least 75%, at least 85%, or at least 95% of the codons can be replaced. A number of codon optimization tools are known and readily available in the art from various sources. For example, OPTIMIZER is an on-line application for codon optimization (P. Puigbo et al. (2007) “OPTIMIZER; a web server for optimizing the codon usage of DNA sequences” Nucleic Acids Res. 35: W126-W131). A recent review of codon optimization methods is provided in H. Fu et al. “Codon optimization with deep learning to enhance protein expression” Nature Research Scientific Reports (2020) 10:17617. Additional references regarding methods of codon optimization include, among others: N. M. Marlatt et al. (2010) “Codon optimization for enhanced Escherichia coli expression of human S100A11 and S100A1 proteins” Protein Expr. Purif. 73 (1): 58-64; A. Mellitzer et al. (2012) “Expression of lignocellulolytic enzymes in Pichia pastoris” Microb. Cell Fact. 11 (1) 61; and E. Angov et al. (2008) “Heterologous protein expression is enhanced by harmonizing the codon usage frequencies of the target gene with those of the expression host” PLOS ONE 3 (5): e21899).

Conservative substitution tables providing functionally similar amino acids are well known in the art. For example, one exemplary guideline to select conservative substitutions includes (original residue followed by exemplary substitution): ala/gly or ser; arg/lys; asn/gln or his; asp/glu; cys/ser; gln/asn; gly/asp; gly/ala or pro; his/asn or gln; ile/leu or val; leu/ile or val; lys/arg or gln or glu; met/leu or tyr or ile; phe/met or leu or tyr; ser/thr; thr/ser; trp/tyr; tyr/trp or phe; val/ile or leu. An alternative exemplary guideline uses the following six groups, each containing amino acids that are conservative substitutions for one another: 1) Alanine (A), Serine(S), Threonine (T); 2) Aspartic acid (D), Glutamic acid (E); 3) Asparagine (N), Glutamine (Q); 4) Arginine (R), Lysine (I); 5) Isoleucine (I), Leucine (L), Methionine (M), Valine (V); and 6) Phenylalanine (F), Tyrosine (Y), Tryptophan (W); (see also, e.g., Creighton, Proteins, W.H. Freeman and Company (1984); Schultz and Schimer, Principles of Protein Structure, Springer-Vrlag (1979)). Another alternative exemplary guidelines uses the following six groups, where proline is unique: 1) Gly (G), Ala (A), Val (V), Leu (L), Ile (I); 2) Ser(S), Cys (C), Thr (T), Met (M); 3) Pro (P); 4) Phe (F), Tyr (Y), Try (W); 5) His (H), Lys (K), Arg (R); and 6) Asp (D), Glu (E), Gln (N). One of skill in the art will appreciate that the above-identified substitutions are not the only possible conservative substitutions. For example, for some purposes, one may regard all charged amino acids as conservative substitutions for each other whether they are positive or negative. In addition, individual substitutions, deletions or additions that alter, add or delete a single amino acid or a small percentage of amino acids in an encoded sequence can also be considered “conservatively modified variations.” One of skill in the art would be familiar with codon selection in a given host that is expressing the protein of interest.

Nucleotide and amino acid sequence information for MYD family members may also be used to construct models of sweet modulating polypeptides in a computer system and how they interact with sweet receptors and computer system models of same. Sweet taste receptors are composed of a heterodimer of taste 1 receptor member 2 (T1R2) and taste 1 receptor member 3 (T1R3). These models can be subsequently used to identify variants and mutations of Myd that can increase activation of sweet receptors and identify more active versions of Myd.

Various conservative mutations as well as the various less conservative mutations as listed in Table 8, Table 9, Table 10, Table 7, Table 6 and Table 3, and substitutions are envisioned to be within the scope of the invention. Mutations of Table 9, Table 10, Table 7, Table 3 and Table 6, which exhibit sweet taste, are currently preferred mutants. For instance, it is within the level of skill in the art to perform amino acid substitutions using known protocols of recombinant gene technology including PCR, gene cloning, site-directed mutagenesis of cDNA, transfection of host cells, and in-vitro transcription. The variants are then be screened for sweet-taste modulation activity and particularly for sweet taste as well as for organoleptic properties or changes in such activities or properties. For example, variants generated are screened employing sensory tested as described herein and as understood in the art. For example, variants generated are screened for taste receptor agonist functional activity as is known in the art.

In embodiments, HTS variant polypeptides herein exhibit enhanced sweetness compared to the unmodified polypeptide SEQ ID NO:3. In some embodiments, the sweetness of respective HTS variants is enhanced at least 10% (or an enhancement ranging from 10% to 100%, or an enhancement ranging from 10% to 200%, or at least 20%, at least 30%, at least 40%, at least 50%, at least 60%, at least 70%, at least 80%, at least 90% or 100% or more) compared to the unmodified polypeptide of SEQ ID NO:3. In other embodiments, HTS variants exhibit comparable sweetness compared to the unmodified polypeptide of SEQ ID NO:3. Comparable sweetness of a modified polypeptide, as described above, compared to the unmodified polypeptide SEQ ID NO:3 is at least 10% (or at least 20%, at least 30%, at least 40%, at least 50%, at least 60%, at least 70%, at least 80%, at least 90% or 100%) as sweet as the unmodified polypeptide of SEQ ID NO:3. Sweetness is measured by any method known in the art for comparison of sweetness, and more particularly by a method for assessing sweetness as described herein.

Flavor modification activity and sweet-taste modulation activity may be detected by methods known in the art, e.g., in vitro methods, or in vivo by animal or human sensory testing. While not wishing to be bound to any particular theory, Myd (HTS) is involved in sweet taste activation e.g., is an agonist of taste 1 receptor member 2 (Tas1R2) and/or taste 1 receptor member 3 (Tas1R3). However, Myd (HTS) agonizes other taste receptors, such as bitter, umami, sour and salty. Such functional effects can be measured by any means known to those skilled in the art, e.g., measurement of binding to taste receptors Tas1Rs via changes in spectroscopic characteristics (e.g., fluorescence, absorbance, refractive index), hydrodynamic (e.g., shape), chromatographic, or solubility properties, patch clamping, voltage-sensitive dyes, whole cell currents, radioisotope efflux, inducible markers, transcriptional activation of Tas1R genes; ligand-binding assays; voltage, membrane potential and conductance changes; ion flux assays; changes in intracellular second messengers such as cAMP, cGMP, and inositol triphosphate (IP3); changes in intracellular calcium levels; neurotransmitter release, and the like.

Sensory testing (human or animal) may also be employed to determine whether a Myd (HTS) candidate polypeptide has sweet-taste modulation activity. Sensory evaluation is a scientific discipline that analyses and measures human responses to the composition of food and drink, e.g., appearance, touch, odor, texture, temperature and taste. Measurements using people as the instruments are sometimes necessary. Selection of an appropriate method to determine sweetening can be determined by one of skill in the art, and includes, e.g., discrimination tests or difference tests, designed to measure the likelihood that two products are perceptibly different. Responses from the evaluators are tallied for correctness, and statistically analyzed to see if there are more correct than would be expected due to chance alone. The food industry had the first need to develop this measurement tool as the sensory characteristics of flavor and texture were obvious attributes that cannot be measured easily by instruments. For sweetness perception, for example, samples of, for example, one or more of 5% sucrose, 6% sucrose, 7% sucrose, 8% sucrose, 9% sucrose, 10% sucrose, and a test sample can be ranked by trained panelists in order of sweet taste intensity from low sweet to high sweet. In the instant invention, it should be understood that there are any number of ways one of skill in the art could measure the organoleptic properties (e.g., sensory differences). For example, organoleptic response can be measured using trained panelist who rate their responses on an accepted scale or rating system, such as a hedonic rating system. Alternatively or in addition, instrumental methods are available or can be readily adapted when a subject's (panelist's) response to an organoleptic property can be linked to a property measured by the instrumental method. See, for example, Ray, S. (2221) “Sensory Properties of Foods and Their Measurement Methods.” In: Khan, M. S., Shafiur Rahman, M. (eds) Techniques to Measure Food Safety and Quality. Springer, Cham. https://doi.org/10.1007/978-3-030-68636-9_15.

Brix measurement (or Brix scale) is a well-known application in the food and beverage industry that determines pure sucrose content in water: 1 degree Brix (° Bx)=1 g of sucrose/100 g of solution and represents the strength of the solution as percentage by mass. 8° Bx is equivalent to approximately an 8% sucrose solution. As described in the Examples, purified polypeptide corresponding to SEQ ID NO:5 was tasted at 0.03 mg/ml by a trained sensory scientist (0.2 mL aliquot) and found to have a sweetness equivalent to 8° Bx (approximately 8% sucrose solution) (see Examples 4, 5, 9, and 10).

### Thermal Stability

Thermal stability of sweet-taste modifying polypeptides can impact the potential applications of the polypeptide, for example, for food applications at elevated temperature. In embodiments, HTS variants herein exhibit comparable thermal stability compared to the unmodified polypeptide of SEQ ID NO:3. Comparable thermal stability of a modified polypeptide, as described above, compared to the unmodified polypeptide of SEQ ID NO:3 is substantially the same which herein means less than or equal to a change in thermal stability of 4.5% (including less than or equal to 1%, less than or equal to 2%, less than or equal to 3%, less than or equal to 4%) compared to the thermal stability of the unmodified polypeptide of SEQ ID NO: 3.

In some embodiments, HTS variants herein exhibit enhanced thermal stability compared to the unmodified SEQ ID NO:3. In other embodiments, enhanced thermal stability of a modified polypeptide, as described above, compared to the unmodified polypeptide of SEQ ID NO:3 is enhanced greater than 4.5% (including, 4.5 to 15% enhanced, greater than 5% enhanced, greater than 6% enhanced, greater than 7% enhanced, greater than 8% enhanced, greater than 9% enhanced, greater than 10% enhanced, greater than 11% enhanced greater than 12% enhanced, greater than 13% enhanced, greater than 14% enhanced, greater than 5% up to 15% enhanced, greater than 6% up to 15% enhanced, greater than 7% up to 15% enhanced, greater than 8% up to 15% enhanced, greater than 9% up to 15% enhanced, greater than 10% up to 15% enhanced, greater than 11% up to 15% enhanced, greater than 12% up to 15% enhanced, greater than 13% up to 15% enhanced, greater than 14% up to 15% enhanced, or up to 15% enhanced) compared to the thermal stability of the unmodified polypeptide of SEQ ID NO:3. Thermal stability is measured by any method known in the art for assessing thermal stability, and more particularly by a method for assessing thermal stability as described herein. In embodiments, sweet-taste modifying polypeptides herein exhibit comparable sweetness and comparable thermal stability compared to that of the unmodified polypeptide of SEQ ID NO:3. In embodiments, sweet-taste modifying polypeptides herein exhibit comparable sweetness and enhanced thermal stability compared to that of the unmodified polypeptide of SEQ ID NO:3. In embodiments, sweet-taste modifying polypeptides herein exhibit enhanced sweetness and comparable thermal stability compared to that of the unmodified polypeptide of SEQ ID NO:3. In embodiments, sweet-taste modifying polypeptides herein exhibit enhanced sweetness and enhanced thermal stability compared to that of the unmodified polypeptide of SEQ ID NO:3.

In some embodiments, the HTS variant herein exhibits comparable sweetness and comparable thermal stability compared to that of the unmodified polypeptide of SEQ ID NO:3. In some embodiments, the HTS variant herein exhibits enhanced sweetness and comparable thermal stability compared to that of the unmodified polypeptide of SEQ ID NO:3. In some embodiments, the HTS variant herein exhibits comparable sweetness and enhanced thermal stability compared to that of the unmodified polypeptide of SEQ ID NO:3. In some embodiments, the HTS variant herein exhibits enhanced sweetness and enhanced thermal stability compared to that of the unmodified polypeptide of SEQ ID NO:3.

Comparable thermal stability of a modified polypeptide, as described above, compared to the unmodified polypeptide of SEQ ID NO:3 is substantially the same, which herein means less than or equal to a change in thermal stability of 4.5% (including less than or equal to 1%, less than or equal to 2%, less than or equal to 3%, less than or equal to 4%) compared to the thermal stability of the unmodified polypeptide of SEQ ID NO:3.

Enhanced thermal stability of a modified polypeptide, as described above, compared to the unmodified polypeptide of SEQ ID NO:3 is enhanced greater than 4.5% (including, 4.5 to 15% enhanced, greater than 5% enhanced, greater than 6% enhanced, greater than 7% enhanced, greater than 8% enhanced, greater than 9% enhanced, greater than 10% enhanced, greater than 11% enhanced, greater than 12% enhanced, greater than 13% enhanced, greater than 15% enhanced, greater than 5% up to 15% enhance, greater than 6% up to 15% enhanced, greater than 7% up to 15% enhanced, greater than 8% up to 15% enhanced, greater than 9% up to 15% enhanced, greater than 10% up to 15% enhanced, greater than 11% up to 15% enhanced, greater than 12% up to 15% enhanced, greater than 13% up to 15% enhanced, greater than 14% up to 15% enhanced, or up to 15% enhanced) compared to the thermal stability of the unmodified polypeptide of SEQ ID NO:3.

In embodiments, the polypeptide variant herein exhibits sweet-taste modulation activity. Non-limiting examples of sweet-taste modulation activity include providing a sweet taste.

In another aspect, the invention provides a polypeptide having sweet-taste modulation activity (e.g., isolated polypeptide) comprising the amino acid sequence of SEQ ID NO:3 which is modified by at least one mutation selected from those listed in Tables 8 and optionally one deletion such as the deleition of Met at position 1. In another aspect, the invention provides a polypeptide having sweet-taste modulation activity (e.g., isolated polypeptide) comprising the amino acid sequence of SEQ ID NO:3 which is modified by at least one mutation up to 24 mutations at different amino acid positions selected from those listed in Tables 8.

In another aspect, the invention provides a polypeptide having sweet-taste modulation activity (e.g., isolated polypeptide) comprising the amino acid sequence of SEQ ID NO:3 which is modified by at least one mutation selected from those listed in Table 8 and at least one mutation selected from Table 7 and optionally one deletion such as the deleition of Met at position 1. In another aspect, the invention provides a polypeptide having sweet-taste modulation activity (e.g., isolated polypeptide) comprising the amino acid sequence of SEQ ID NO:3 which is modified by at least one mutation up to 24 mutations at different amino acid positions selected from those listed in Table 8 and from one to 24 mutations selected from Table 7.

In another aspect, the invention provides a polypeptide having sweet-taste modulation activity (e.g., isolated polypeptide) comprising the amino acid sequence of SEQ ID NO:3 which is modified by at least one mutation up to 24 selected from those listed in Table 8, Table 9 or Table 10 and optionally one deletion such as the deletion of Met at position 1. In another aspect, the invention provides a polypeptide having sweet-taste modulation activity (e.g., isolated polypeptide) comprising the amino acid sequence of SEQ ID NO:3 which is modified by at least one mutation up to 24 mutations at different amino acid positions selected from those listed in Table 8, Table 9 or Table 10.

In a related aspect, the invention provides a polypeptide having sweet-taste modulation activity (e.g., isolated polypeptide) comprising the amino acid sequence of SEQ ID NO:3 which is modified by two mutations at different positions selected from those listed in Table 8, Table 9 or Table 10 and optionally one deletion such as the deletion of Met at position 1. In a related aspect, the invention provides a polypeptide having sweet-taste modulation activity (e.g., isolated polypeptide) comprising the amino acid sequence of SEQ ID NO:3 which is modified by three mutations at different positions selected from those listed in Table 8, Table 9 or Table 10 and optionally one deletion such as the deletion of Met at position 1. In a related aspect, the invention provides a polypeptide having sweet-taste modulation activity (e.g., isolated polypeptide) comprising the amino acid sequence of SEQ ID NO:3 which is modified by four mutations at different positions selected from those listed in Table 8, Table 9 or Table 10 and optionally one deletion such as the deletion of Met at position 1. In a related aspect, the invention provides a polypeptide having sweet-taste modulation activity (e.g., isolated polypeptide) comprising the amino acid sequence of SEQ ID NO:3 which is modified by five mutations at different positions selected from those listed in Table 8, Table 9 or Table 10 and optionally one deletion such as the deletion of Met at position 1. In a related aspect, the invention provides a polypeptide having sweet-taste modulation activity (e.g., isolated polypeptide) comprising the amino acid sequence of SEQ ID NO:3 which is modified by six mutations at different positions selected from those listed in Table 8, Table 9 or Table 10 and optionally one deletion such as the deletion of Met at position 1.

In a related aspect, the invention provides a polypeptide having sweet-taste modulation activity (e.g., isolated polypeptide) comprising the amino acid sequence of SEQ ID NO:3 which is modified by seven mutations at different positions selected from those listed in Table 8, Table 9 or Table 10 and optionally one deletion such as the deletion of Met at position 1. In a related aspect, the invention provides a polypeptide having sweet-taste modulation activity (e.g., isolated polypeptide) comprising the amino acid sequence of SEQ ID NO:3 which is modified by eight mutations at different positions selected from those listed in Table 8, Table 9 or Table 10 and optionally one deletion such as the deletion of Met at position 1. In a related aspect, the invention provides a polypeptide having sweet-taste modulation activity (e.g., isolated polypeptide) comprising the amino acid sequence of SEQ ID NO:3 which is modified by nine mutations at different positions selected from those listed in Table 8, Table 9 or Table 10 and optionally one deletion such as the deletion of Met at position 1. In a related aspect, the invention provides a polypeptide having sweet-taste modulation activity (e.g., isolated polypeptide) comprising the amino acid sequence of SEQ ID NO:3 which is modified by ten mutations at different positions selected from those listed in Table 8, Table 9 or Table 10 and optionally one deletion such as the deletion of Met at position 1. In a related aspect, the invention provides a polypeptide having sweet-taste modulation activity (e.g., isolated polypeptide) comprising the amino acid sequence of SEQ ID NO:3 which is modified by eleven mutations at different positions selected from those listed in Table 8, Table 9 or Table 10 and optionally one deletion such as the deletion of Met at position 1. In a related aspect, the invention provides a polypeptide having sweet-taste modulation activity (e.g., isolated polypeptide) comprising the amino acid sequence of SEQ ID NO: 3 which is modified by twelve mutations at different positions selected from those listed in Table 8, Table 9 or Table 10 and optionally one deletion such as the deletion of Met at position 1.

In a related aspect, the invention provides a polypeptide having sweet-taste modulation activity (e.g., isolated polypeptide) comprising the amino acid sequence of SEQ ID NO:3 which is modified by thirteen mutations at different positions selected from those listed in Table 8, Table 9 or Table 10 and optionally one deletion such as the deletion of Met at position 1. In a related aspect, the invention provides a polypeptide having sweet-taste modulation activity (e.g., isolated polypeptide) comprising the amino acid sequence of SEQ ID NO:3 which is modified by fourteen mutations at different positions selected from those listed in Table 8, Table 9 or Table 10 and optionally one deletion such as the deletion of Met at position 1. In a related aspect, the invention provides a polypeptide having sweet-taste modulation activity (e.g., isolated polypeptide) comprising the amino acid sequence of SEQ ID NO:3 which is modified by fifteen mutations at different positions selected from those listed in Table 8, Table 9 or Table 10 and optionally one deletion such as the deletion of Met at position 1. In a related aspect, the invention provides a polypeptide having sweet-taste modulation activity (e.g., isolated polypeptide) comprising the amino acid sequence of SEQ ID NO:3 which is modified by sixteen mutations at different positions selected from those listed in Table 8, Table 9 or Table 10 and optionally one deletion such as the deletion of Met at position 1. In a related aspect, the invention provides a polypeptide having sweet-taste modulation activity (e.g., isolated polypeptide) comprising the amino acid sequence of SEQ ID NO:3 which is modified by seventeen mutations at different positions selected from those listed in Table 8, Table 9 or Table 10 and optionally one deletion such as the deletion of Met at position 1. In a related aspect, the invention provides a polypeptide having sweet-taste modulation activity (e.g., isolated polypeptide) comprising the amino acid sequence of SEQ ID NO:3 which is modified by eighteen mutations at different positions selected from those listed in Table 8, Table 9 or Table 10 and optionally one deletion such as the deletion of Met at position 1.

In a related aspect, the invention provides a polypeptide having sweet-taste modulation activity (e.g., isolated polypeptide) comprising the amino acid sequence of SEQ ID NO:3 which is modified by nineteen mutations at different positions selected from those listed in Table 8, Table 9 or Table 10 and optionally one deletion such as the deletion of Met at position 1. In a related aspect, the invention provides a polypeptide having sweet-taste modulation activity (e.g., isolated polypeptide) comprising the amino acid sequence of SEQ ID NO:3 which is modified by twenty mutations at different positions selected from those listed in Table 8, Table 9 or Table 10 and optionally one deletion such as the deletion of Met at position 1. In a related aspect, the invention provides a polypeptide having sweet-taste modulation activity (e.g., isolated polypeptide) comprising the amino acid sequence of SEQ ID NO:3 which is modified by twenty-one mutations at different positions selected from those listed in Table 8, Table 9 or Table 10 and optionally one deletion such as the deletion of Met at position 1. In a related aspect, the invention provides a polypeptide having sweet-taste modulation activity (e.g., isolated polypeptide) comprising the amino acid sequence of SEQ ID NO:3 which is modified by twenty-two mutations at different positions selected from those listed in Table 8, Table 9 or Table 10 and optionally one deletion such as the deletion of Met at position 1. In a related aspect, the invention provides a polypeptide having sweet-taste modulation activity (e.g., isolated polypeptide) comprising the amino acid sequence of SEQ ID NO:3 which is modified by twenty-three mutations at different positions selected from those listed in Table 8, Table 9 or Table 10 and optionally one deletion such as the deletion of Met at position 1. In a related aspect, the invention provides a polypeptide having sweet-taste modulation activity (e.g., isolated polypeptide) comprising the amino acid sequence of SEQ ID NO:3 which is modified by twenty-four mutations at different positions selected from those listed in Table 8, Table 9 or Table 10 and optionally one deletion such as the deletion of Met at position 1. The invention also provides polynucleotides encoding the foregoing mutant polypeptides of SEQ ID NO:3 or SEQ ID NO: 141. The invention further provides the foregoing mutant polypeptides which further comprise a protein tag and more specifically a histidine tag. The invention also provides polynucleotides encoding the foregoing mutant polypeptides of SEQ ID NO:3 or SEQ ID NO: 141 which further comprise a protein tag and more specifically a histidine tag. In embodiments, HTS variants do not include any one or more of the mutations listed in Table 3, Table 6 or Table 7.

In some embodiments, the polypeptide having sweet-taste modulation activity also has enhanced thermal stability compared to the polypeptide of SEQ ID NO:3. In an embodiment, the polypeptide having sweet-taste modulation activity and enhanced thermal stability comprises a modified SEQ ID NO:3, wherein the polypeptide has at least 1 and up to 6 amino acid modifications as shown in Table 8, Table 9 or Table 10 and optionally one deletion such as the deletion of Met at position 1. In an embodiment, the polypeptide having sweet-taste modulation activity and enhanced thermal stability comprises a modified SEQ ID NO:3, wherein the polypeptide has at least 1 and up to 6 amino acid modifications as shown in Table 7 and optionally one deletion such as the deletion of Met at position 1. In an embodiment, the polypeptide having sweet-taste modulation activity and enhanced thermal stability comprises a modified SEQ ID NO:3, wherein the polypeptide has at least 1 and up to 6 amino acid modifications as shown in Table 8, Table 9 or Table 10 and further has 1-6 amino acid modifications as shown in Table 7 and optionally one deletion such as the deletion of Met at position 1. In further embodiments of the preceding embodiments, the polypeptide having sweet-taste modulation activity (including sweet taste) and enhanced thermal stability compared to the polypeptide of SEQ ID NO:141.

In another embodiment, the polypeptide having sweet-taste modulation activity (and particularly sweet taste) and enhanced thermal stability compared to the polypeptide of SEQ ID NO: 3 has at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity to a modified polypeptide of SEQ ID NO:3 with at least 1 and up to 6 amino acid modifications as shown in Table 8, Table 9 or Table 10 and 1-6 amino acid modifications as shown in Table 7 and optionally one deletion such as the deletion of Met at position 1. In further embodiments of the preceding embodiments, the polypeptide has sweet-taste modulation activity (including sweet taste) and enhanced thermal stability compared to the polypeptide of SEQ ID NO: 141.

In another embodiment, the polypeptide having sweet-taste modulation activity (and particularly sweet taste) and enhanced thermal stability compared to the polypeptide of SEQ ID NO: 3 has both mutations I49C and S33C (compared to SEQ ID NO:3) and optionally 1, 2, 3, 4, 5 or 6 other mutations as shown in Table 8, Table 9, Table 10, Table 3, Table 6 or Table 7 and in particular optionally one deletion such as the deletion of Met at position 1. In a related embodiment, the polypeptide having sweet-taste modulation activity (and particularly sweet taste) and enhanced thermal stability compared to the polypeptide of SEQ ID NO:3 has both mutations I49C and S33C (compared to SEQ ID NO:3) and has at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity to a modified polypeptide of SEQ ID NO:3. In further embodiments of the preceding embodiments, the polypeptide has sweet-taste modulation activity (including sweet taste) and enhanced thermal stability compared to the polypeptide of SEQ ID NO:141.

In another embodiment, the polypeptide having sweet-taste modulation activity (and particularly sweet taste) and enhanced thermal stability compared to the polypeptide of SEQ ID NO: 3 has both mutations Y23C and Y61C (compared to SEQ ID NO:3) and optionally 1, 2, 3, 4, 5 or 6 other mutations as shown in Table 8, Table 9, Table 10, Table 3, Table 6 or Table 7 and optionally one deletion such as the deletion of Met at position 1. In a related embodiment, the polypeptide having sweet-taste modulation activity (and particularly sweet taste) and enhanced thermal stability compared to the polypeptide of SEQ ID NO:3 has both mutations I49C and S33C (compared to SEQ ID NO:3) and has at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity to a modified polypeptide of SEQ ID NO: 3. In further embodiments of the preceding embodiments, the polypeptide has sweet-taste modulation activity (including sweet taste) and enhanced thermal stability compared to the polypeptide of SEQ ID NO:141.

In an embodiment, the polypeptide having sweet-taste modulation activity and enhanced thermal stability comprises a modified SEQ ID NO:3 wherein the polypeptide has two modifications as shown in Table 7 and/or Table 8, Table 9 or Table 10 and optionally one deletion such as the deletion of Met at position 1. In an embodiment, the polypeptide having sweet-taste modulation activity and enhanced thermal stability comprises a modified SEQ ID NO: 3 wherein the polypeptide has two modifications as shown in Table 7 and/or Table 8, Table 9 or Table 10 that result in disulfide bond formation between the two modified amino acids and optionally one deletion such as the deletion of Met at position 1. In one embodiment, the polypeptide having sweet-taste modulation activity and enhanced thermal stability comprises a modified SEQ ID NO:3, wherein the polypeptide has at least two modifications as shown in Table 7 and/or Table 8, Table 9 or Table 10 that result in disulfide bond formation between the at least two modified amino acids and optionally one deletion such as the deletion of Met at position 1. In further embodiments of the preceding embodiments, the polypeptide has sweet-taste modulation activity (including sweet taste) and enhanced thermal stability compared to the polypeptide of SEQ ID NO:141.

In an embodiment, the polypeptide having sweet-taste modulation activity (and particularly sweet taste) and enhanced thermal stability compared to the polypeptide of SEQ ID NO: 3 comprises the polypeptide of SEQ ID NO: 143 (double mutant S33C_I49C) or SEQ ID NO: 145 (double mutant Y24C_Y62C). In a related embodiment, the polypeptide having sweet-taste modulation activity (and particularly sweet taste) and enhanced thermal stability compared to the polypeptide of SEQ ID NO:3 comprises the polypeptide of SEQ ID NO:142 or SEQ ID NO: 145 and in addition the methionine at position 1 is absent SEQ ID NO:146 and SEQ ID NO: 147. In an embodiment, the polypeptide having sweet-taste modulation activity (and particularly sweet taste) and enhanced thermal stability compared to the polypeptide of SEQ ID NO: 3 is the polypeptide of SEQ ID NO: 143. In an embodiment, the polypeptide having sweet-taste modulation activity (and particularly sweet taste) and enhanced thermal stability compared to the polypeptide of SEQ ID NO:3 is the polypeptide of SEQ ID NO: 145. In an embodiment, the polypeptide having sweet-taste modulation activity (and particularly sweet taste) and enhanced thermal stability compared to the polypeptide of SEQ ID NO: 3 is the polypeptide of SEQ ID NO:146. In an embodiment, the polypeptide having sweet-taste modulation activity (and particularly sweet taste) and enhanced thermal stability compared to the polypeptide of SEQ ID NO: 3 is the polypeptide of SEQ ID NO: 147.

In some embodiments, polypeptides of the invention exhibit stability to low pH (<pH 7) that is enhanced compared to the polypeptide of SEQ ID NO:3. In some embodiments, polypeptides of the invention exhibit stability to high pH (>pH 7) that is enhanced compared to the polypeptide of SEQ ID NO:3. In one embodiment, polypeptides of the invention exhibit greater stability at pH of about 2 as compared to the polypeptide of SEQ ID NO:3. In another embodiment, polypeptides of the invention exhibit greater stability at pH of about 10 as compared to the polypeptide of SEQ ID NO:3. In one embodiment, polypeptides of the invention having methionine present at position 1 exhibit greater stability at pH of about 2 as compared to the polypeptide of SEQ ID NO:3. In another embodiment, polypeptides of the invention having methionine present at position 1 exhibit greater stability at pH of about 10 as compared to the polypeptide of SEQ ID NO:3.

The term “expression vector” or “expression cassette” refers to any recombinant expression system for the purpose of expressing a nucleic acid sequence of the invention in vitro or in vivo, constitutively or inducibly, in any cell, including prokaryotic, yeast, fungal, plant, insect or mammalian cell. The term includes linear or circular expression systems. The term includes expression systems that remain episomal or integrate into the host cell genome. The expression systems can have the ability to self-replicate or not, i.e., drive only transient expression in a cell. The term includes recombinant expression cassettes which contain only the minimum elements needed for transcription of the recombinant nucleic acid.

A recent review of methods for expression of recombinant proteins is found in Tripathi & Shrivastava (2019) “Recent Developments in Bioprocessing of Recombinant Proteins; Expression Hosts and Process Development,” Frontiers in Bioeng. Biotech. 7:420, doi: 10.3389/fbio.2019.00420. This reference is incorporated by reference herein in its entirety for details of host expression systems and methods for expression of recombinant proteins.

By “host cell” is meant a cell that contains an expression vector and supports the replication or expression of the expression vector. In one embodiment, the host cell is a prokaryotic cell. In one embodiment, the host cell is a eukaryotic cell. Host cells may be prokaryotic cells such as E. coli, or eukaryotic cells such as yeast, insect, amphibian, or mammalian cells such as CHO, HeLa, HEK-293, and the like, e.g., cultured cells, explants, and cells in vivo.

In an embodiment, the host cell is selected from the group consisting of Escherichia coli, Klebsiella oxytoca, Anaerobiospirillum succiniciproducens, Actinobacillus succinogenes, Mannheimia succiniciproducens, Agrobacterium tumefaciens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, Pseudomonas putida, Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, Rhizopus oryzae, Yarrowia lipolytica, Candida albicans, Issatchenkia orientalis, Scheffersomyces stipitis, Yarrowia lipolytica, Ogataea polymorpha, Phaffia rhodozyma, Candida utilis, Arxula adeninivorans, Debaryomyces hansenii, Debaryomyces polymorphus, and Schwanniomyces occidentalis.

In an embodiment, the host cell is selected from the group consisting of Qualified Presumption of Safety (QPS) recommended biological agents. A list of such hosts is available on the website: efsa.europa.eu/efsajournal EFSA Journal 2021; 19 (7): 6689. In an embodiment, the host organism is selected from the group consisting of Bacillus megaterium, Trichoderma reesei, Bifidobacterium adolescentis, Bifidobacterium animalis, Bifidobacterium bifidum, Bifidobacterium breve, Bifidobacterium Longum, Carnobacterium divergens, Lactobacillus acidophilus, Lactobacillus amylolyticus, Lactobacillus amylovorus, Lactobacillus animalis, Lactobacillus alimentarius, Lactobacillus aviaries, Lactobacillus brevis, Lactobacillus buchneri, Lactobacillus casei, Lactobacillus cellobiosus, Lactobacillus collinoides, Lactobacillus coryniformis, Lactobacillus crispatus, Lactobacillus curvatus, Lactobacillus delbrueckii, Lactobacillus dextrinicus, Lactobacillus diolivorans, Lactobacillus farciminis, Lactobacillus fermentum, Lactobacillus gallinarum, Lactobacillus gasseri, Lactobacillus helveticus, Lactobacillus hilgardii, Lactobacillus johnsonii, Lactobacillus kefiranofaciens, Lactobacillus kefiri, Lactobacillus mucosae, Lactobacillus panis, Lactobacillus paracasei, Lactobacillus parafarraginis, Lactobacillus paraplantarum, Lactobacillus pentosus, Lactobacillus plantarum, Lactobacillus pontis, Lactobacillus reuteri, Lactobacillus rhamnosus, Lactobacillus sakei, Lactobacillus salivarius, Lactobacillus Sanfranciscensis, Leuconostoc citreum, Leuconostoc lactis, Leuconostoc mesenteroides, Leuconostoc Pseudomesenteroides, Microbacterium imperial, Oenococcus oeni, Pasteuria nishizawae, Pediococcus acidilactici, Pediococcus parvulus, Pediococcus pentosaceus, Propionibacterium acidipropionic, Propionibacterium freudenreichii, Streptococcus thermophilus, Bacillus amyloliquefaciens, Bacillus atrophaeus, Bacillus circulans, Bacillus clausii, Bacillus coagulans, Bacillus flexus, Bacillus fusiformis, Bacillus lentus, Bacillus licheniformis, Bacillus megaterium, Bacillus mojavensis, Bacillus paralicheniformis, Bacillus pumilus, Bacillus smithii, Bacillus subtilis, Bacillus vallismortis, Bacillus velezensis, Geobacillus stearothermophilus, Paenibacillus illinoisensis, Parageobacillus thermoglucosidasius, Gluconobacter oxydans, Komagataeibacter sucrofermentans, Xanthomonas campestris, Candida cylindracea, Cyberlindnera jadinii, Debaryomyces hansenii, Hanseniaspora uvarum, Kluyveromyces lactis, Kluyveromyces marxianus, Komagataella pastoris, Komagataella phaffi, Lindnera jadinii, Ogataea angusta, Saccharomyces bayanus, Schizosaccharomyces pombe, Wickerhamomyces anomalus, Xanthophyllomyces dendrorhous, or Zygosaccharomyces rouxii.

In another aspect, the host cell is selected from the group consisting of gram-positive non-spore forming bacteria, gram-positive spore forming bacteria, gram negative bacteria, yeast, and protists/algae. In other aspects, the host cell is selected from plant cells. In other aspects, the host cell is selected from insect cells. With respect to insect cells, Baculo virus insect expression systems are useful. Insect cells useful as hosts for production of recombinant protein include, among others, Spodoptera frugiperda cells (e.g., Sf9, Sf21), Drosophila cells (e.g., S2), Trichoplusia ni cells (e.g., Tn-368, High-Five™ (Thermo Fisher Scientific, Waltham, MA)). Various host cells are known in the art and available from commercial sources, among others.

Non-limiting examples of gram-positive non-spore forming bacteria include Bifidobacterium adolescentis, Bifidobacterium animalis, Bifidobacterium bifidum, Bifidobacterium breve, Bifidobacterium longum, Carnobacterium divergens, Corynebacterium ammoniagenes, Corynebacterium glutamicum, Lactobacillus acidophilus, Lactobacillus amylolyticus, Lactobacillus amylovorus, Lactobacillus animalis, Lactobacillus alimentarius, Lactobacillus aviaries, Lactobacillus brevis, Lactobacillus buchneri, Lactobacillus casei, Lactobacillus cellobiosus, Lactobacillus collinoides, Lactobacillus coryniformis, Lactobacillus crispatus, Lactobacillus curvatus, Lactobacillus delbrueckii, Lactobacillus dextrinicus, Lactobacillus diolivorans, Lactobacillus farciminis, Lactobacillus fermentum, Lactobacillus gallinarum, Lactobacillus gasseri, Lactobacillus helveticus, Lactobacillus hilgardii, Lactobacillus johnsonii, Lactobacillus kefiranofaciens, Lactobacillus kefiri, Lactobacillus mucosae, Lactobacillus panis, Lactobacillus paracasei, Lactobacillus parafarraginis, Lactobacillus paraplantarum, Lactobacillus pentosus, Lactobacillus plantarum, Lactobacillus pontis, Lactobacillus reuteri, Lactobacillus rhamnosus, Lactobacillus sakei, Lactobacillus salivarius, Lactobacillus sanfranciscensis, Lactococcus lactis, Leuconostoc citreum, Leuconostoc lactis, Leuconostoc mesenteroides, Leuconostoc pseudomesenteroides, Microbacterium imperial, Oenococcus oeni, Pasteuria nishizawae, Pediococcus acidilactic, Pediococcus parvulus, Pediococcus pentosaceus, Propionibacterium acidipropioni, Propionibacterium freudenreichii, and Streptococcus thermophiles.

Non-limiting examples of gram-positive spore forming bacteria include Bacillus amyloliquefaciens, Bacillus atrophaeus, Bacillus circulans, Bacillus clausii, Bacillus coagulans, Bacillus flexus, Bacillus fusiformis, Bacillus lentus, Bacillus licheniformis, Bacillus megaterium, Bacillus mojavensis, Bacillus pumilus, Bacillus smithii, Bacillus subtilis, Bacillus vallismortis, Bacillus velezensis, Geobacillus stearothermophilus, Paenibacillus illinoisensis, and Parageobacillus thermoglucosidasius. Non-limiting examples of gram-negative bacteria include Cupriavidus necator, Gluconobacter oxydans, Komagataeibacter sucrofermentans, and Xanthomonas campestris.

Non-limiting examples of yeast include Candida cylindracea, Debaryomyces hansenii, Hanseniaspora uvarum, Kluyveromyces lactis, Kluyveromyces marxianus, Komagataella pastoris, Komagataella phaffi, Lindnera jadinii, Ogataea angusta, Saccharomyces bayanus, Saccharomyces cerevisiae, Saccharomyces pastorianus, Schizosaccharomyces pombe, Wickerhamomyces anomalus, Xanthophyllomyces dendrorhous, Yarrowia lipolytica, and Zygosaccharomyces rouxii.

Non-limiting examples of protists/algae include Aurantiochytrium limacinum, Euglena gracilis, and Tetraselmis chuii.

In certain embodiments, recombinant HTS is produced by a transgenic mammal, i.e., in milk.

The expression of HTS (or a variant thereof) may be stable or transient. In a stable expression system, the exogenous DNA is integrated into the chromosomes, or as an episome (a separate piece of nuclear DNA) and is passed on to future generations of the host cell.

The terms “mimetic” and “peptidomimetic” refer to a synthetic chemical compound that has substantially the same structural and/or functional characteristics of the polypeptides, e.g., translocation domains, ligand-binding domains, or chimeric receptors of the invention. The mimetic can be either entirely composed of synthetic, non-natural analogs of amino acids, or may be a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity.

As with polypeptides of the invention which are conservative variants, routine experimentation will determine whether a mimetic is within the scope of the invention, i.e., that its structure and/or function is not substantially altered. Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(O)—CH₂— for —C(O)—NH—), aminomethylene (CH₂—NH), ethylene, olefin (CH═CH), ether (CH₂—O), thioether (CH—S), tetrazole (CN4), thiazole, retroamide, thioamide, or ester (see, e.g., Spatola, Chemistry and Biochemistry of Amino Acids, Peptides and Proteins, Vol. 7, pp 267-357, “Peptide Backbone Modifications,” Marcell Dekker, NY (1983)). A polypeptide can also be characterized as a mimetic by containing all or some non-natural residues in place of naturally occurring amino acid residues; non-natural residues are well described in the scientific and patent literature. Phyre2 is a suite of tools available on the web to predict and analyze protein structure, function and mutations.

Examples of conservatively modified variations of Myd1 protein structure may be derived using homology-modelling algorithms: SWISS-MODEL, PHYRE2.0, and Jpred to identify a sequence-based consensus loop regions, as known in the art, see, e.g., Pechmann, S. & Frydman, J. Interplay between Chaperones and Protein Disorder Promotes the Evolution of Protein Networks. PLOS Computational Biology 10, e1003674 (2014).

Specific regions of the MYD/Myd nucleotide and amino acid sequences may be used to identify polymorphic variants, interspecies homologs, and alleles of Myd family members. This identification can be made in vitro, e.g., under stringent hybridization conditions or PCR (e.g., using primers encoding the Myd sequences identified herein), or by using the sequence information in a computer system for comparison with other nucleotide sequences. Different alleles of MYD genes within a single species population will also be useful in determining whether differences in allelic sequences correlate to differences in taste perception between members of the population. Classical PCR-type amplification and cloning techniques are useful for isolating orthologs, for example, where degenerate primers are sufficient for detecting related genes across species.

For instance, primers designed using the sequences disclosed herein can be used can be used to amplify and clone MYD-related genes from different fungal genomes. In contrast, genes within a single species that are related to MYD are best identified using sequence pattern recognition software to look for related sequences. Typically, identification of polymorphic variants and alleles of MYD family members can be made by comparing an amino acid sequence of about 25 amino acids or more, e.g., 50-100 amino acids. Amino acid identity of approximately at least 35 to 50%, and optionally 60%, 70%, 75%, 80%, 85%, 90%, 95-99%, or above typically demonstrates that a protein is a polymorphic variant, interspecies homolog, or allele of a MYD family member. Sequence comparison can be performed using any of the sequence comparison algorithms discussed below. Antibodies that bind specifically to Myd polypeptides or a conserved region thereof can also be used to identify alleles, interspecies homologs, and polymorphic variants.

In an embodiment, hybrid protein-coding sequences comprising nucleic acids encoding Myd variant fusion proteins may be constructed. These nucleic acid sequences can be operably linked to transcriptional or translational control elements, e.g., transcription and translation initiation sequences, promoters and enhancers, transcription and translation terminators, polyadenylation sequences, and other sequences useful for transcribing DNA into RNA. Fusion proteins may include C-terminal or N-terminal translocation sequences. Further, fusion proteins can comprise additional elements, e.g., for protein detection, purification, or other applications. Detection and purification facilitating domains include, e.g., metal chelating peptides such as polyhistidine tracts, histidine-tryptophan modules, or other domains that allow purification on immobilized metals; maltose binding protein; protein A domains that allow purification on immobilized immunoglobulin; or the domain utilized in the FLAGS extension/affinity purification system (Immunex Corp, Seattle Wash.).

In an embodiment, the fusion protein comprises a peptide or protein tag (e.g., for protein purification or detection). Protein/peptide tags are peptide sequences genetically grafted into a recombinant (e.g., fusion) protein. Peptide/protein tags are known in the art, such as those described in Johnson, “Protein/Peptide Tags,” DOI//dx.doi.org/10.13070/mm.en.2.116 including but not limited to green fluorescent protein (GFP), FLAG, Myc epitope, polyhistidine, glutathione-S-transferase (GST), HA, V5, ABDz1-tag, Adenylate kinase (AK-tag), BC2-tag, Calmodulin-binding peptide, CusF, Fc, Fh8, Halo tag, Heparin binding peptide (HB-tag), Ketosteroid isomerase (KSI), maltose-binding protein (MBP), thioredoxin, PA (NZ-1), Poly-Arg, Poly-Lys, S-tag, SBP/Streptavidin-Binding Peptide, SNAP, Strep-II (Twin-Strep), and SUMO/SUMO2.

Affinity tags are a type of protein tag that is appended to proteins so that they can be purified from their crude biological source using an affinity technique. Affinity tags are known in the art, such as those described in Kimple et al. Curr Protoc Protein Sci.; 73: Unit-9.9, doi: 10.1002/0471140864.ps0909s73. These include, among others, polyhistidine, GST, MBP, Calmodulin-binding peptide, intein-chitin binding domain, Streptavidin/Biotin-based tags, and His-Patch ThioFusion (thioredoxin). Affinity tags include small (e.g., 20 or less amino acid residues) or large affinity tags. Examples of small affinity tags include His, FLAG, Strep II, and S-peptide, and examples of large affinity tags include MBP, GST, cellulose binding domains, calmodulin binding peptide, and His-patch thioredoxin.

Protein/peptide tags include epitope tags and reporter tags. Reporter tags serve as reporters of protein expression and protein-protein interaction. Reporter tags include, but are not limited to, enzymes such as β-galactosidase (β-gal), alkaline phosphatase (AP), chloramphenicol acetyl transferase (CAT), and horseradish peroxidase (HRP).

Epitope tags include FLAG, hemagglutinin (HA), c-myc, T7, and Glu-Glu, which are used for the detection of fusion proteins in vitro and in cell culture. Their short, linear recognition motifs rarely affect the properties of the protein of interest and are usually very specific for their respective primary antibodies. If the anti-myc antibody is used, specificity can be increased by using an enzyme-linked secondary antibody to detect a conjugated anti-myc primary antibody instead of using an HRP- or AP-anti-myc conjugate alone.

Protein/peptide tags also include solubilization tags which are used to assist in the proper folding of proteins and keep them from aggregating in inclusion bodies. In embodiments, solubilization tags are employed for proteins expressed in E. coli. Solubilization tags include thioredoxin and poly (NANP), among others. Some affinity tags can also assist in solubilization, such as MBP and GST.

Protein/peptide tags can be at either end of the target protein. Some tags, such as FLAG, are often used in tandem to increase their desired features, or in combination with another tag, such as in the construct of His-Myc and His-V5.

Tandem affinity purification (TAP) is a dual-affinity purification method based on the fusion of two affinity tags to a protein of interest, which allows purification of a tagged protein and isolation of protein complexes interacting with the protein of interest. The use of TAP is encompassed within the present invention.

In one embodiment, the fusion protein comprises a histidine tag that comprises 2-10 (e.g., 2, 3, 4, 5, 6, 7, 8, 9, or 10) histidine residues. For example, the histidine tag can comprise 6 histidine residues.

The inclusion of a cleavable linker sequences such as Factor Xa (see, e.g., Ottavi, Biochimie 80:289-293 (1998)), subtilisin protease recognition motif (see, e.g., Polyak, Protein Eng. 10:615-619 (1997)); enterokinase (Invitrogen, San Diego, Calif.), and the like, between the translocation domain (for efficient plasma membrane expression) and the rest of the newly translated polypeptide may be useful to facilitate purification. For example, one construct can include a polypeptide encoding a nucleic acid sequence linked to six histidine residues followed by a thioredoxin, an enterokinase cleavage site (see, e.g., Williams, Biochemistry 34:1787-1797 (1995)), and a C-terminal translocation domain. The histidine residues facilitate detection and purification while the enterokinase cleavage site provides a means for purifying the desired protein(s) from the remainder of the fusion protein. Technology pertaining to vectors encoding fusion proteins and application of fusion proteins are well described in the scientific and patent literature, see, e.g., Kroll, DNA Cell. Biol. 12:441-53 (1993).

The fusion protein can contain one or more linkers (e.g., flexible linkers, rigid linkers, and in vivo cleavable linkers). Besides the basic role in linking the functional domains together (as in flexible and rigid linkers) or releasing free functional domain in vivo (as in in vivo cleavable linkers), linkers offer many other advantages for the production of fusion proteins, such as improving biological activity, increasing expression yield, and achieving desirable pharmacokinetic profiles. Linkers are known in the art (see, e.g., Chen et al., Adv Drug Deliv Rev. 65 (10): 1357-1369 (2013)).

Flexible linkers are used when the joined domains require a certain degree of movement or interaction. They are generally composed of small, non-polar (e.g. Gly) or polar (e.g. Ser or Thr) amino acids. The small size of these amino acids provides flexibility and allows for mobility of the connecting functional domains. The incorporation of Ser or Thr can maintain the stability of the linker in aqueous solutions by forming hydrogen bonds with the water molecules, and therefore reduces the unfavorable interaction between the linker and the protein moieties.

The most commonly used flexible linkers have sequences consisting primarily of stretches of Gly and Ser residues (“GS” linker). An example of the most widely used flexible linker has the sequence of (Gly-Gly-Gly-Gly-Ser), (SEQ ID NO:7). By adjusting the copy number “n”, the length of this GS linker can be optimized to achieve appropriate separation of the functional domains, or to maintain necessary inter-domain interactions. Besides the GS linkers, many other flexible linkers have been designed for recombinant fusion proteins. These flexible linkers are also rich in small or polar amino acids such as Gly and Ser but can contain additional amino acids such as Thr and Ala to maintain flexibility, as well as polar amino acids such as Lys and Glu to improve solubility.

Rigid linkers keep a fixed distance between the domains and to maintain their independent functions. Examples of rigid linkers include alpha helix-forming linkers with the sequence of (EAAAK), (SEQ ID NO:8) and linkers with a Pro-rich sequence, (XP) n, with X designating any amino acid, preferably Ala, Lys, or Glu.

The polypeptide of the present invention also can contain a signal peptide (i.e., a signal sequence, targeting signal, localization signal, localization sequence, transit peptide, leader sequence, or leader peptide), which is a short peptide present at the N-terminus or occasionally C-terminus of most newly synthesized proteins that are destined toward the secretory pathway. These proteins include those that reside either inside certain organelles (the endoplasmic reticulum, Golgi or endosomes), secreted from the cell, or inserted into most cellular membranes. Exemplary signal peptides are known in the art and a person of ordinary sill in the art would recognize how to select a particular signal peptide for use in the invention.

### Protein Derivatization

HTS proteins/polypeptide and sequence variants thereof can be further derivatized or modified other than by substitution of one or more amino acids. HTS wild-type (native) protein/polypeptide and HTS protein/polypeptide variants of this disclosure can be derivatized at the N-terminus, the C-terminus or at an amino acid side chain without loss of taste modulation activity. Derivatization, in general includes, acylation, esterification, glycosylation, oxidation, methylation, reductive alkylation, phosphorylation (as phospho-amino acids), sulfurylation, sulfonylation, or oxidation or reduction of side chain heteroatoms. Derivatization can be accomplished by chemical methods using reagents and methods that are well-known in the art. Alternatively, derivatization can be accomplished by biological methods, such as treatment with one or more enzymes, or by post-translational methods (post-translational modification). One of ordinary skill in the art can selected one or more enzymes to effect desired derivatization of HTS proteins/polypeptides or variants thereof. The HTS protein or polypeptide, preferably isolated, is treated with the selected enzyme to achieve the desired derivatization. Post-translational modifications (PTMs) are covalent modification to a protein/polypeptide by proteolytic cleavage (e.g., removal of N-terminal methionine) and/or addition of a modifying group, such as acetyl (more broadly acyl), a phosphoryl, a glycosyl and/or a methyl, to one or more amino acids. Other chemical modification of an amino acid of a protein/polypeptide (e.g., oxidation of the sulfur of a methionine group) can be achieved by both chemical and biological means to one skilled in the art. Post-translational modification can occur during native expression of a protein/polypeptide or post-translational modification can be controlled during protein expression in a non-native host using recombinant methods. Such recombinant methods rely on the use of specially constructed expression vectors including, for example, coding sequences for expression of one or more enzyme to actuate a selected post-translational modification. One of ordinary skill in the art, is aware of chemical or recombinant techniques to derivative proteins at one or more position on a given protein. In an embodiment, preferred protein/polypeptide derivatizations are those that do not significantly affect the sweet taste-modulation activity of the HTS protein/polypeptide variants of this disclosure. In an embodiment, preferred protein/polypeptide derivatizations are those that do not significantly detrimentally affect the sweet taste of the HTS protein/polypeptide variants of this disclosure (e.g., do not significantly decrease the sweet taste of the HTS variants herein). In embodiments, derivatization of the HTS variant can enhance flavor modification, sweet taste modification or sweet taste activity of HTS variants.

Derivatization of an HTS protein/polypeptide or variant, by any known method, can occur at the N-terminus, the C-terminus, or at one or more amino acid side group (e.g., a side-chain sulfur, a side chain amine or a side-chain carboxylic acid). In embodiments, a HTS protein/polypeptide or HTS variant of this disclosure can have 1, 2, 3, 4, 5 or 6 different derivations. Preferably, the HTS protein or HTS variant has a single derivatization. For example, the N-terminus of the HTS protein/polypeptide or variant can be derivatized by acylation and more specifically by acetylation. In more specific embodiments, the N-terminus of the protein/polypeptide or variant is a methionine that is N-acetylated. In embodiments, the derivatization is of the N-terminal amino acid or of an amine side chain of an amino acid of the HTS protein/polypeptide. The N-terminal amine acid or an amine side chain (e.g., a lysine side-chain) can be derivatized by acylation; glycosylation; methylation; reductive amination to form —NH—CH2-R, where R is an alkyl group (e.g., an alkyl group having 1-19 carbon atoms); phosphorylation of amino acid side chains such as serine or threonine (—OH) with kinases to form a phospho-polypeptides; reaction of sulfur in amino acid side chains with monooxygenases to produce sulfoxide derivatives. The N-terminal amine or an amine side-chain can be derivatized by chemical methods or by biological processes after the translation of the polypeptide, resulting in a post-translational modification of the nitrogen. Reactive side chains of other amino acids may be derivatized by chemical methods or biological processes after the translation of the polypeptide, resulting in post-translational modification of those side chains.

In specific embodiments, the derivatization is an addition, subtraction, or substitution of one or more of the following chemical moieties to an amine nitrogen: acyl (R—CO—, where R is a straight-chain or branched alkyl group having 1-20 carbon atoms); acetyl (CH₃CO), formyl (HCO), glycosyl (e.g., C₆H₁₁O₆—), hydroxyl (HO—), methyl (CH₃—) or other alkyl group, a phosphatidyl (PO₄), a phosphonyl (PO₂), a sulfhydryl (SH—), or a sulfonyl (HSO₂—).

In specific embodiments, wherein the first amino acid of the HTS protein/polypeptide is a methionine, the delta-position sulfur of the first amino acid is modified, by chemical methods, by enzymatic methods (in vitro or in vivo) or by biological processes after the translation of the polypeptide, resulting in a post-translational modification of the sulfur. In embodiments, the modification of the delta-position sulfur of the first amino acid is an addition, subtraction, or substitution of any of the following chemical moieties to the delta sulfur: acyl (R—CO—, where R is a straight-chain or branched alkyl group having 1-20 carbon atoms); acetyl (CH3CO), formyl (HCO), glycosyl (e.g., C6H11O6-), hydroxyl (HO—), methyl (CH3-) or other alkyl group, a phosphatidyl (PO4), a phosphonyl (PO2), a sulfhydryl (SH—), or a sulfonyl (HSO2-). In embodiments, the modification of the delta-position sulfur of the first amino acid is the oxidation of the sulfur, resulting in a sulfoxide derivative.

As used herein, “at least 80% identity” with reference to an amino acid sequence or a nucleotide sequence refers to 80%, 81%, 82%, 83%, 84%, 85%, 86%, 87%, 88%, 89%, 90%, 91%, 92%, 93%, 94%, 95%, 96%, 97%, 98%, or 99% or more identity.

As used herein, examples of “an amino acid sequence modified by deletion, insertion, substitution, or addition of one or more amino acids” include an amino acid sequence modified by deletion, insertion, substitution, or addition of 1 or more to 30 or less, preferably 20 or less, more preferably 10 or less, and further preferably 5 or less amino acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or any ranges thereof). As used herein, examples of “a nucleotide sequence modified by deletion, insertion, substitution, or addition of one or more nucleotides” include a nucleotide sequence modified by deletion, insertion, substitution, or addition of 1 or more to 90 or less, preferably 60 or less, more preferably 30 or less, further preferably 15 or less, and further more preferably 10 or less nucleotides (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, or any ranges thereof).

For example, in sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, as described below for the BLASTN and BLASTP programs, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J Mol. Biol. 48:443 (1970), by the search for similarity method of Pearson & Lipman, Proc. Natl. Acad Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of an algorithm that is suitable for determining percent sequence identity and sequence similarity are the BLAST and BLAST 2.0 algorithms, which are described in Altschul at al., Nuc. Acids Res. 25:3389-3402 (1977) and Altschul et al., J Mol. Biol. 215:403-410 (1990), respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., Altschul et al., Nuc. Acids Res. 25:3389-3402 (1977) and Altschul et al., J Mol. Biol. 215:403-410 (1990)). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad Sci. USA 89:10915 (1989)) alignments (B) of 50, expectation (E) of 10, M=5, N=−4, and a comparison of both strands.

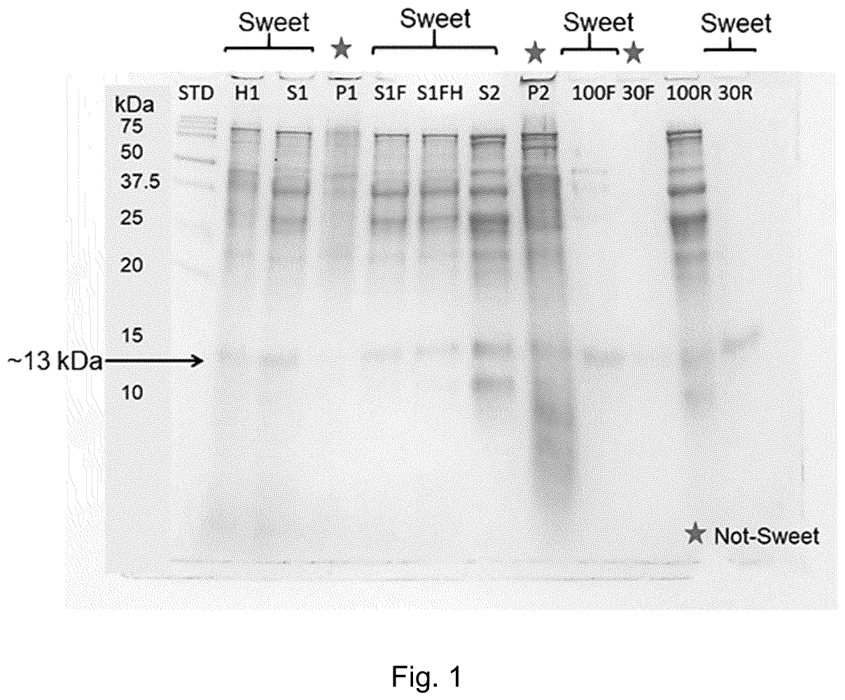

Another example of a useful algorithm is PILEUP. PILEUP creates a multiple sequence alignment from a group of related sequences using progressive, pairwise alignments to show relationship and percent sequence identity. It also plots a so-called “tree” or “dendogram” showing the clustering relationships used to create the alignment (see, e.g., FIG. 1). PILEUP uses a simplification of the progressive alignment method of Feng & Doolittle, J Mol. Evol. 35:351-360 (1987). The method used is similar to the method described by Higgins & Sharp, CABIOS 5:151-153 (1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395 (1984) encoded by the genes were derived by conceptual translation of the corresponding open reading frames.

The polynucleotides encoding the polypeptides of the present invention can be synthesized chemically or by genetic engineering based on the amino acid sequence of a Myd. For example, the polynucleotide can be synthesized chemically based on the amino acid sequence of the polypeptides of the present invention or preprotein thereof. A contract synthesis service of nucleic acid (provided from, for example, Medical & Biological Laboratories Co., Ltd., Genscript etc.) can be used for the chemical synthesis of the polynucleotide. Further, the synthesized polynucleotide can be amplified by PCR and cloning etc.

The polypeptides of the present invention can be produced, for example, by expressing a gene encoding a Myd polypeptide variant of the present invention. Preferably, a Myd polypeptide variant of the present invention can be produced from a transformant in which the polynucleotide encoding a Myd polypeptide variant of the present invention is introduced. For example, a Myd polypeptide variant of the present invention is produced from a polynucleotide encoding a Myd polypeptide variant of the present invention introduced in a transformant after a polynucleotide encoding a Myd polypeptide variant of the present invention or a vector comprising it is introduced into a host to obtain a transformant and the transformant is cultured in an appropriate medium. The proteins of the present invention can be obtained by isolating or purifying the produced Myd polypeptide variant from the culture.

Therefore, the present invention further provides a polynucleotide encoding a Myd polypeptide variant of the present invention and a vector comprising it. The present invention further provides a method of manufacturing a transformant, comprising introducing a polynucleotide encoding a Myd polypeptide variant of the present invention or a vector comprising it into a host. The present invention further provides a transformant comprising a polynucleotide encoding a Myd polypeptide variant of the present invention or a vector comprising it introduced from the outside of a cell. The present invention further provides a method of manufacturing a Myd polypeptide variant of the present invention, comprising culturing the transformant.

The present invention also includes the polynucleotides of the invention, operably linked to a heterologous regulatory element. The invention may include an expression cassette or vector comprising the polynucleotides of the present invention, and a host cell transformed with a vector of the invention.

Alternatively, the polynucleotide encoding a Myd polypeptide variant of the present invention can be produced by introducing one or more mutation into the polynucleotide synthesized according to the procedure with known mutagenesis methods such as the ultraviolet irradiation and site-directed mutagenesis. For example, the polynucleotide encoding the polypeptides of the present invention can be obtained by introducing one or more mutation into the polynucleotide of SEQ ID NO:1 or SEQ ID NO:2 with a known method, expressing the obtained polynucleotide, investigating the expressed protein's sweet-modification activity, and selecting a polynucleotide encoding the protein having desired sweet modification activity.

Site-directed mutagenesis of a polynucleotide can be performed with any methods such as, for example, inverse PCR and annealing (Muramatsu et al. edit., “Revised 4th edition New genetic engineering handbook”, YODOSHA, p. 82-88). A variety of commercially available kits for site-directed mutagenesis such as QuickChange II Site-Directed Mutagenesis Kit from Stratagene and QuickChange Multi Site-Directed Mutagenesis Kit can be used as needed.

Examples of the type of a vector comprising the polynucleotide encoding the polypeptides of the present invention include, without limitation, a vector usually used for gene cloning, for example, a plasmid, a cosmid, a phage, a virus, a YAC and a BAC. Examples of vectors include plasmids (e.g., DNA plasmids), yeast (e.g., Saccharomyces), and viral vectors, such as poxvirus, retrovirus, adenovirus, adeno-associated virus, herpes virus, polio virus, alphavirus, baculovirus, Sindbis virus, plant viruses (e.g., Alphaflexiviridae or Potyviridae), and insect viruses (e.g., Baculoviridae).

Among these, a plasmid vector is preferred and for example a commercially available plasmid vector for protein expression, for example, pUC19, pUC118, pUC119, pBR322 etc. (all of which are from TAKARA BIO INC.) can be used.

The vector can comprise a DNA region comprising a replication initiation region or a replication origin of DNA. Alternatively, a regulatory sequence such as a promoter region for initiating transcription of the gene, a terminator region or a secretory signal region for secreting an expressed protein to the outside of a cell can be operably liked to the upstream of the polynucleotide encoding the proteins of the present invention (i.e. the MYD gene of the present invention) in the vector. As used herein, a gene and a regulatory sequence being “operably liked” refers to a condition in which the gene and the regulatory region are positioned so that the gene can be expressed under the regulation by the regulatory region.

The type of the regulatory sequence of a promoter region, a terminator, and a secretory signal region etc. is not specifically limited, and a promoter and a secretory signal sequence usually used can be selected to use as appropriate depending on the host into which the sequence is introduced. For example, preferred examples of the regulatory sequence which can be incorporated to the vector of the present invention include the cbh1 promoter sequence derived from Trichoderma reesei (Curr, Genet, 1995, 28 (1): 71-79).

Alternatively, a marker gene to select a host into which the vector is appropriately introduced (for example, a resistance gene to an agent such as ampicillin, neomycin, kanamycin and chloramphenicol) can be further incorporated into the vector of the present invention. Alternatively, a gene encoding a synthase of a required nutrient can be incorporated into the vector as a marker gene, when an auxotrophic strain is used as a host. Alternatively, a related gene of the metabolism can be incorporated into the vector as a marker gene, when a selective medium requiring specific metabolism for growth is used. Examples of such a metabolism related gene include an acetamidase gene for using acetamide as a nitrogen source.

Ligation between the polynucleotide encoding a Myd polypeptide variant of the present invention and a regulatory sequence and a marker gene can be performed by a known method in the art such as SOE (splicing by overlap extension)-PCR (Gene, 1989, 77:61-68). The procedure for introducing a ligated fragment into a vector is known in the art.

Examples of a host of a transformant into which the vector is introduced include a microorganism such as a bacterium or filamentous fungus. Examples of the bacterium include Escherichia coli and a bacterium belonging to Staphylococcus, Enterococcus, Listeria and Bacillus, of which Escherichia coli and Bacillus bacteria (for example, Bacillus subtilis or a mutant thereof) are preferred. Examples of the Bacillus subtilis mutant can include protease 9 double deficient strain KA8AX described in J. Biosci. Bioeng., 2007, 104 (2): 135-143 and a DBPA strain, a mutant from protease 8 double deficient strain described in Biotechnol. Lett., 2011, 33 (9): 1847-1852, of which protein folding efficiency is improved. Examples of the filamentous fungus include Trichoderma, Aspergillus and Rhizopus. Also, for example, Pichia pastoris, Saccharomyces cerevisiae, Hansenula polymorpha, Yarrowia lipolytica, Schizosaccharomyces pombe, Kluyveromyces lactis are appropriate expression hosts. In embodiments, the host cell is a cell of a fungus other than Mattirolomyces terfezioides. In embodiments, the HTS protein variant can be expressed in mycelium.

In embodiments, the HTS protein variant can be expressed in a plant cell, a plant organ, a leaf, a root or in a whole plant.

In yet another aspect, the invention includes a host cell comprising one or more of the expression cassettes described herein operably linked to control elements compatible with expression in the cell. The cell can be, for example, a mammalian cell (e.g., BHK, VERO, HT1080, 293, RD, COS-7, or CHO cells), an insect cell (e.g., Trichoplusia ni (Tn5) or Sf9), a bacterial cell, a plant cell, or a yeast cell.

In a particular embodiment, the HTS (or a variant thereof) is produced in a yeast expression system (i.e., yeast-derived HTS or variant thereof), for example, in the genera Kluyveromyces (e.g., K. lactis), Lactococcus (e.g., L. lactis), Lactobacillus, Saccharomyces (e.g., S. cerevisiae) Pichia (e.g., P. pastoris), Hansenula (e.g., H. polymorpha) or Yarrowia (e.g., Y. lipolytica).

In another particular embodiment, the HTS (or a variant thereof) is produced in a bacterial expression system (i.e., bacteria-derived HTS or variant thereof), for example, in Escherichia coli or Bacillus subtilis. In one embodiment, the HTS (or a variant thereof) is not produced in E. coli.

In a further particular embodiment, the HTS (or a variant thereof) is produced in an insect expression system (i.e., insect-derived HTS or variant thereof), for example, in baculovirus infected or non-lytic insect cells (e.g., sf9, Sf21).

In another embodiment, the HTS (or a variant thereof) is produced in a fungal expression system (i.e., fungi-derived HTS or variant thereof), for example in the genera Chrysosporium, Thielavia, Talaromyces, Trichoderma, Thermomyces or Thermoascus.

In yet another embodiment, HTS (or a variant thereof) is produced in a mammalian expression system (i.e., mammalian-derived HTS or variant thereof), for example in Chinese hamster ovary (CHO) cells, human embryonic kidney (HEK), COS and baby hamster kidney (BHK) cells. Alternatively, the HTS (or a variant thereof) may be produced in vitro using a cell-free expression system, such as an E. coli S30 extract.

### Purification

The recombinantly expressed polypeptides from Myd-encoding expression cassettes are typically isolated from lysed cells or culture media. Purification can be carried out by methods known in the art including salt fractionation, ion exchange chromatography, gel filtration, size-exclusion chromatography, size-fractionation, affinity chromatography, filtration, electrophoresis, hydrophobic interaction chromatography, gel filtration chromatography, reverse phase chromatography, concanavalin A chromatography, chromatofocusing and differential precipitation or solubilization. Immunoaffinity chromatography can be employed using antibodies generated based on, for example, Gag antigens.

The invention provides a method of purifying a polypeptide having sweet-taste modulation activity comprising (a) obtaining a composition comprising the polypeptide, and (b) purifying the composition via hydrophobic interaction chromatography (HIC) followed by size exclusion chromatography (SEC).

One of skill in the art is familiar with the purification techniques of hydrophobic interaction chromatography (HIC) and size exclusion chromatography (SEC), including the selection of appropriate columns, buffers, and eluting solutions. Exemplary HIC and SEC purification techniques are described herein in Example 11. In an exemplary aspect, the purity of the polypeptide following purification by HIC and SEC is at least 90%, at least 91%, at least 92%, at least 93%, at least 94%, at least 95%, at least 96%, at least 97%, at least 98%, at least 99%, or any ranges of values thereof.

In a cell-based system, the first step in the protein purification process extracts the protein from the cells by lysing or breaking them open. Any suitable cell lysis method may be used, for example, mechanical disruption, chemical breakdown, freeze-thaw cycles or enzymatic digestion. The protein can then by purified by any suitable protein purification method for example, affinity chromatography, ion exchange chromatography, filtration, electrophoresis, hydrophobic interaction chromatography, gel filtration chromatography, reverse phase chromatography, concanavalin A chromatography, chromatofocusing and differential precipitation or solubilization.

The yield of in vivo production of HTS (or a variant thereof) may vary. In a particular embodiment, HTS represents at least about 1% of total cellular proteins. In a particular embodiment, HTS represents between about 1% and about 5% of total cellular proteins. In another embodiment, HTS represents between about 5% and about 10% or total cellular proteins. In a further embodiment, HTS represents between 10% and about 20% of total cellular proteins. In certain embodiments, HTS represents more than 20% of total cellular proteins.

In another particular embodiment, HTS is purified to provide a yield between about 1 mg/mL and about 200 mg/mL, more particularly, between about 5 mg/mL and about 195 mg/mL, between about 10 mg/mL and about 190 mg/mL, between about 15 mg/mL and about 185 mg/mL, between about 20 mg/mL and about 180 mg/mL, between about 25 mg/mL and about 175 mg/mL, between about 30 mg/mL and about 170 mg/mL or about 35 mg/mL and about 165 mg/mL. In one embodiment, the HTS is purified to provide a yield of about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 75 mg/mL, about 100 mg/mL, about 125 mg/mL, about 150 mg/mL, about 175 mg/mL, or about 200 mg/L or more. Optionally, the HTS is produced as a fusion protein further comprising a tag and the yields described above reflect both purification and removal of the tag.

In a particular embodiment, the HTS (or a variant thereof) is substantially pure. In one embodiment, HTS (or a variant thereof) is at least about 80% pure, at least about 85% pure, at least about 90% pure, at least about 95% pure or at least about 99% pure. In another embodiment, HTS (or a variant thereof) is about 90%, about 91%, about 92%, about 93%, about 94%, about 95%, about 96%, about 97%, about 98% or about 99% pure.

### Plants

The invention also contemplates a transgenic plant comprising a heterologous polynucleotide and/or heterologous polypeptide of the invention as described herein. The plant has an altered phenotype due to the expression of the heterologous nucleic acid sequence. The altered phenotype may include a phenotype with increased sweetness in any plant part, including fruits. The transgenic plant may contain an expression cassette as defined herein as a part of the plant, the cassette having been introduced by transformation of a plant with a vector of this invention. Such expression cassettes include regulatory sequences for expression of heterologous coding sequences in plants, including plant-expressible promoters and terminators. A transgenic plant can be any type of plant which can express the heterologous nucleic acid sequences described herein. The term “plant” includes whole plants, plant organs (e.g., leaves, stems, roots, etc.), seeds and plant cells and progeny of same. The class of plants which can be used in the method of the invention is generally as broad as the class of higher plants amenable to transformation techniques, including both monocotyledonous (monocots) and dicotyledonous (dicots) plants. It includes plants of a variety of ploidy levels, including polyploid, diploid and haploid. For example, the transgenic plant can be an apple or strawberry. An HTS protein/polypeptide or variant of this disclosure can be produced in a plant or a plant culture.

Techniques for transforming a wide variety of plant species are well known in the art and described in the technical and scientific literature. See, for example, Weising et al. (1988) Ann. Rev. Genet., 22:421-477 and Joung et al. (2015) “Plant Transformation Methods and Applications,” in Current Technology in Plant Molecular Breeding, (Koh et al., eds) Springer Dordrecht Heidelberg New York London, Chapter 9, pages 297-344. Any method known in the art for transformation of plant cells, including plant protoplasts, or plant tissue can be employed for plant transformation. Specific methods for plant transformation include among others, bolistic methods (gene guns), electroporation, microinjection, protoplast fusion and Agrobacterium-mediated transformation. Agrobacterium-mediated transformation can, for example, employ binary vectors that replicate in Escherichia coli and Agrobacterium tumefaciens or other Agrobacterium strains. A variety of such binary vectors are known in the art and can be employed to introduce heterologous polynucleotides into plant cells and plant tissue. Plant expression vectors which include regulatory sequences for expression of heterologous coding sequences, including plant-expressible promoter sequences and other plant regulatory sequences, in plant cells and plant tissue are known in the art and can be employed to transform plants to express polypeptides as described herein.

A variety of plant-expressible promoters are known in the art and are available for use in heterologous constructs, vectors and transformed plant materials herein which contain polynucleotides encoding protein having sweet-taste modulation activity. Plant-expressible promoters can derive from natural plant sources, plant virus sources and from bacteria, such as Agrobacterium strains, having promoters that are plant-expressible. Plant-expressible promoters include, among others, Cauliflower Mosaic Virus promoter (CaMV 35S), octopine and nopaline synthase promoters (e.g., nos promoter), plant ubiquitin promoter (Ubi), rice actin promoter (Act-1), and maize alcohol dehydrogenase (Adh-1). Plant-expressible promoters include constitutive promoters, inducible promoters, tissue-specific promoters, developmental stage-specific promoters and examples of each type of promoter are known in the art. Tissue-specific promoters include, among others, those that direct expression in plant roots, plant leaves, fruit, flowers, pollen or cells engaged in active photosynthesis (e.g., phosphoenolpyruvate promoters (PEP)). Development stage-specific promoters include those that direct expression during fruit ripening, flowering or seed set. Synthetic plant promoters are also known in the art and are useful in heterologous constructs, vectors and transformed plant materials (see, e.g., Ali S. & Kim W-C (2019) Frontiers in Plant Science, 10, article 1433).

Techniques for regeneration of plants from transformed protoplasts, plant cells, callus, or other plant tissue are well known in the art and can be employed to regenerated whole plants and plant parts from such transformed plant material. Regeneration methods include organogenesis and embryogenesis. See: Handbook of plant cell culture. Volume 1: Techniques for propagation and breeding (1983) Edited by D. A. Evans et al., Macmillan (New York); R. H. Smith, Plant Tissue Culture: Techniques and Experiments, 3rd Edition (2012) Academic Press (New York); M. R. Davey & P. Anthony, Plant Cell Culture: Essential Methods (2010) John Wiley & Sons (New York), particularly Chapters 3 and 9.

In embodiments, HTS (or a variant thereof) is produced in an algal expression system (i.e., algae-derived HTS or variant thereof).

In embodiments, HTS (or a variant thereof) is produced in a plant expression system (i.e., plant-derived HTS or variant thereof), for example, in maize, corn, tobacco, melon (e.g., watermelon), potatoes, strawberries, duckweed or sugarcane. In one embodiment, the plant expression system is a plant cell culture expression system.

In a particular embodiment, HTS (or a variant thereof) is produced in maize, and more particularly, the seeds of maize. In another particular embodiment, HTS (or a variant thereof) is produced in corn, and more particularly, the seeds of corn. According to these embodiments, HTS (or a variant thereof) may be utilized as HTS-containing germ flour.

### Methods of Producing a Protein Having Sweet-Taste Modulation Activity (in a Host or Cell-Free Expression System)

A method usually used in the field such as protoplast method and electroporation can be used as a method of introducing a vector into a host. A transformant of interest can be obtained by selecting a strain in which a vector is appropriately introduced using an index such as the expression of a marker gene and/or auxotrophy.

Alternatively, a fragment in which the polynucleotide encoding a Myd polypeptide variant of the present invention, a regulatory sequence and a marker gene are ligated can be directly introduced into the genome of a host. For example, the polynucleotide encoding a Myd polypeptide variant of the present invention is introduced into the genome of a host by constructing a DNA fragment added with a sequence complementary to the genome of the host at both ends of the ligated fragment, introducing the fragment into the host and inducing homologous recombination between the host genome and the DNA fragment by SOE-PCR.

Culturing the thus obtained transformant, in which the polynucleotide encoding a Myd polypeptide variant of the present invention or a vector comprising is introduced, in an appropriate medium, results in the expression of the MYD cDNA on the vector, and then the production of a Myd polypeptide variant of the present invention. The medium used for the culture of such transformant can be selected depending on the type of microorganism of such transformant by those skilled in the art as appropriate.

Alternatively, a Myd polypeptide variant of the present invention can be expressed from the polynucleotide encoding a Myd polypeptide variant of the present invention or a transcription product thereof using a cell-free translation system. “Cell-free translation system” refers to an in vitro transcription-translation system or an in vitro translation system constructed by adding reagents such as amino acids required for the translation of a protein into a suspension obtained by mechanically destructing cells to be a host.

Cell-free systems that can be used to produce HTS for use in the compositions described herein include, but are not limited to, protein expression components from eukaryotic, prokaryotic, and/or viral sources. For example, cell-free systems as used herein can include mammalian and/or bacterial protein expression systems derived from mammalian and/or bacterial lysates.

A Myd polypeptide variant of the present invention produced in the culture or cell-free translation system can be isolated or purified, if desired, by using a general method used for the purification of a protein, for example, centrifugation, ammonium sulfate precipitation, gel chromatography, ion-exchange chromatography and affinity chromatography etc. alone or in combination as appropriate. Here, when the gene encoding a Myd polypeptide variant of the present invention and the secretory signal sequence are operably liked on the vector within the transformant, the produced Myd polypeptide variant can be collected more easily from the culture because the Myd polypeptide variant is secreted to the outside of a cell. The Myd polypeptide variant collected from the culture can be further purified by any known means.

In embodiments, the Myd protein is solubilized in a liquid solution, such as e.g., a buffered solution or any solution that readily dissolves the Myd protein into solution. In embodiments, the Myd protein contained in a liquid solution is lyophilized to form a powder. In embodiments, the Myd protein contained in a liquid solution is dried to form a powder, such as e.g., by using a spray dryer. In embodiments, spray drying includes the use of a carrier as is known in the art. In embodiments, the carrier used for spray drying is maltodextrin, gum Arabic or whey protein concentrate.

The present invention also includes a method for producing a protein having sweet-taste modulation activity, comprising culturing the host cells of the invention in a medium under conditions that result in producing the protein having sweet-taste modulation activity, similar to a known sweet flavoring agent or compound.

### Sweet Compositions-Foods, Beverages, Supplements, Medicinal Products

Disclosed herein are sweetener compositions and flavor modifying compositions, in each case, containing Myd (HTS) or variants thereof. In certain embodiments, the sweetener compositions and flavor modifying compositions change (e.g., improve) one or more sensory experiences of a subject who consumes the same. In a particular embodiment, the sweetener composition and flavor modifying compositions disclosed herein comprise a HTS variant. In one embodiment, the HTS variant differs from wild-type HTS at least one amino acid position and more particularly, at one, two, three or more amino acid positions.

As used herein, a “sweet flavoring agent,” “sweet compound” or “sweet receptor activating compound” refers to a composition that elicits a detectable sweet flavor in a subject, e.g., sucrose, fructose, glucose, and other known natural saccharide-based sweeteners, or known artificial sweeteners such as saccharine, cyclamate, aspartame, and the like as is further discussed herein, or a material that activates a T1R2/T1R3 receptor in vitro. The subject may be a human or an animal.

A sweet flavoring agent or sweetening composition may be used in an effective amount, which refers to an amount of a sweetening composition of the invention that is sufficient to induce sweet taste in a subject when present in a product for oral administration.

An embodiment of the present invention includes a composition. In an embodiment, the composition comprises, consists essentially of, or consists of a combination of a product for oral administration and one or more sweetening composition comprising an isolated Myd polypeptide variant according to the invention, as described herein. In an embodiment, the combination has enhanced sweet taste compared to a product for oral administration lacking the Myd polypeptide variant (control). In one embodiment, the product for oral administration is not Mattirolomyces terfezioides truffle. The term “consisting essentially of” allows for the inclusion of components that are not essential to the function or activity of the product and do not materially affect the function or activity, such an anti-caking agent, filler, stabilizer (e.g., thermal stabilizer), and bulking agent (e.g., maltodextrose, gum acacia and the like). In an embodiment, the composition includes a plurality of isolated Myd polypeptides. In a particular embodiment, the composition includes a plurality of isolated Myd polypeptides which differ from each other to enhance taste. It should be appreciated that compositions comprising one or more Myd polypeptides of the invention are not limited by the form, shape, and means of administration, and encompass solids, liquids, powder, and other forms, either individually or in combinations of two or more thereof. Furthermore, the compositions may be administered or consumed orally, by injection, etc.

In another embodiment, compositions comprising an isolated Myd protein of the invention include formulations that provide enhanced functionality to the isolated Myd protein. For example, a composition may include a formulation that stabilizes the Myd protein against thermal, osmotic, pH, or other types of degradation. In one embodiment, the formulation stabilizes the Myd protein against thermal degradation. Exemplary compounds for stabilizing the Myd protein includes, for example, L-arginine glycine, L-proline, L-histidine, β-alanine, L-serine, L-arginine ethyl ester dihydrochloride, L-argininamide dihydrochloride, 6-aminohexanoic acid, gly-gly, gly-gly-gly, tryptone, betaine monohydrate, D-(+)-trehalose dihydrate, xylitol, D-sorbitol, sucrose, hydroxyectoine, trimethylamine n-oxide dihydrate, methyl-α-d-glucopyranoside, triethylene glycol, spermine tetrahydrochloride, spermidine, 5-aminovaleric acid, glutaric acid, adipic acid, ethylenediamine dihydrochloride, guanidine hydrochloride, urea, N-methylurea, N-ethylurea, N-methylformamide, hypotaurine, TCEP hydrochloride, GSH (1-glutathione reduced), benzamidine hydrochloride, ethylenediaminetetraacetic acid disodium salt dihydrate, magnesium chloride hexahydrate, cadmium chloride hydrate, non-detergent sulfobetaine 195 (ndsb-195), non-detergent sulfobetaine 201 (NDSB-201), non-detergent sulfobetaine 211 (NDSB-211), non-detergent sulfobetaine 221 (NDSB-221), non-detergent sulfobetaine 256 (NDSB-256), taurine, acetamide, oxalic acid dihydrate, sodium malonate pH 7.0, succinic acid pH 7.0, tacsimate pH 7.0, tetraethylammonium bromide, choline acetate, 1-ethyl-3-methylimidazolium acetate, 1-butyl-3-methylimidazolium chloride, ethylammonium nitrate, ammonium sulfate, ammonium chloride, magnesium sulfate hydrate, potassium thiocyanate, gadolinium (III) chloride hexahydrate, cesium chloride, 4-aminobutyric acid (GABA), lithium nitrate, DL-malic acid pH 7.0, lithium citrate tribasic tetrahydrate, ammonium acetate, sodium benzenesulfonate, sodium p-toluenesulfonate, sodium chloride, potassium chloride, sodium phosphate monobasic monohydrate, sodium sulfate decahydrate, lithium chloride, sodium bromide, glycerol, ethylene glycol, polyethylene glycol 200, polyethylene glycol monomethyl ether 550, polyethylene glycol monomethyl ether 750, formamide, polyethylene glycol 400, pentaerythritol ethoxylate (15/4 EO/OH), 1,2-propanediol, polyethylene glycol monomethyl ether 1,900, polyethylene glycol 3,350, polyethylene glycol 8,000, polyvinylpyrrolidone k15, polyethylene glycol 20,000, (2-hydroxypropyl)-β-cyclodextrin, α-cyclodextrin, β-cyclodextrin, methyl-β-cyclodextrin.

In embodiments, the sweetening composition includes one or more Myd polypeptides described above. In an embodiment, the sweetening composition includes a plurality of Myd polypeptides described above. In a particular embodiment, the plurality of Myd polypeptides differ from each other.

The present invention also includes a method for modulating the taste of a product for oral administration, comprising combining the product for oral administration with an effective amount of an isolated Myd polypeptide variant, as described herein. In one aspect, the combination has enhanced sweet taste compared to a product for oral administration lacking the Myd polypeptide variant (control). In one embodiment, the product for oral administration is not Mattirolomyces terfezioides truffle.

The product for oral administration may be a food, a beverage, a dietary supplement composition, or a pharmaceutical composition.

The term “product for oral administration” may refer to a comestible product (consumable) such as a food product, a beverage product, a medicinal (pharmaceutical) product, or a dietary supplement product such as an herbal supplement. As used herein, the term “consumable” may be used interchangeably with the term “product(s) for oral administration”. As used herein, the term “medicinal product” includes both solids and liquid compositions which are ingestible non-toxic materials which have medicinal value or comprise medicinally active agents such as cough syrups, cough drops, aspirin and chewable medicinal tablets. An oral hygiene product is also a product for oral administration and includes solids and liquids such as toothpaste or mouthwash. In general terms, the present invention contemplates that food or beverage products may include an isolated sweet protein of the invention in an effective amount, e.g., in an amount of up to about 99% by weight relative to the total weight of the food or beverage product, for example in an amount from about 0.01% by weight to about 99% by weight. All intermediate weights (i.e., 0.01%, 0.05%, 0.1%, 0.5%, 1%, 2%, 3%, 4%, . . . 90%, 95%, 99%) by weight relative to the total weight of the food or beverage products are contemplated, as are all intermediate ranges based on these amounts. The compositions of the invention may include a “comestibly, biologically or medicinally acceptable carrier or excipient” which can include a solid or liquid medium and/or composition that is used to prepare a desired dosage form of a Myd polypeptide variant, in order to administer a Myd polypeptide variant in a dispersed/diluted form, so that the biological effectiveness of a Myd polypeptide variant is maximized. A comestibly, biologically or medicinally acceptable carrier includes many common food ingredients, such as water at neutral, acidic, or basic pH, fruit or vegetable juices, vinegar, marinades, beer, wine, natural water/fat emulsions such as milk or condensed milk, edible oils and shortenings, fatty acids, low molecular weight oligomers of propylene glycol, glyceryl esters of fatty acids, and dispersions or emulsions of such hydrophobic substances in aqueous media, salts such as sodium chloride, wheat flours, solvents such as ethanol, solid edible diluents such as vegetable powders or flours, or other liquid vehicles, dispersion or suspension aids, surface active agents, isotonic agents; thickening or emulsifying agents, preservatives, solid binders, lubricants and the like.

A medicinally acceptable carrier or excipient can include excipients which allow for microencapsulation of the Myd polypeptide variant to enhance functionality, such as protecting and extending the sweetness sensation. In fact, microencapsulation is known in the art to be a technology that can facilitate regular use in addition to creating many possible new uses for sweeteners. See, e.g., Favaro-Trindade, Carmen & Rocha-Selmi, Glaucia & dos Santos, Milla. (2015). Microencapsulation of Sweeteners. 10.1016/B978-0-12-800350-3.00022-4. In one embodiment, microencapsulation methods known in the art for stabilizing and/or modifying (e.g., extending) the sweetness release of Myd. For example, sugar-free chewing gums and chewable confections usually have encapsulated sweeteners in their formulations to prolong their sweet taste during chewing. It should be appreciated that food or beverage products and compositions comprising one or more Myd polypeptides as described are not limited by the form and shape and encompass solids, liquids, powder, and other forms, either individually or in combinations of two or more thereof. Examples of food or beverage products of the present invention include such as, but not limited to, baked goods; sweet bakery products, (including, but not limited to, rolls, cakes, pies, pastries, and cookies); pre-made sweet bakery mixes for preparing sweet bakery products; pie fillings and other sweet fillings (including, but not limited to, fruit pie fillings and nut pie fillings such as pecan pie filling, as well as fillings for cookies, cakes, pastries, confectionary products and the like, such as fat-based cream fillings); desserts, gelatins and puddings; frozen desserts (including, but not limited to, frozen dairy desserts such as ice cream—including regular ice cream, soft serve ice cream and all other types of ice cream—and frozen non-dairy desserts such as non-dairy ice cream, sorbet and the like); carbonated beverages (including, but not limited to, soft carbonated beverages); non-carbonated beverages (including, but not limited to, soft non-carbonated beverages such as flavored waters and sweet tea or coffee based beverages); beverage concentrates (including, but not limited to, liquid concentrates and syrups as well as non-liquid concentrates, such as freeze-dried and/or powder preparations); yogurts (including, but not limited to, full fat, reduced fat and fat-free dairy yogurts, as well non-dairy and lactose-free yogurts and frozen equivalents of all of these); snack bars (including, but not limited to, cereal, nut, seed and/or fruit bars); bread products (including, but not limited to, leavened and unleavened breads, yeasted and un-yeasted breads such as soda breads, breads comprising any type of wheat flour, breads comprising any type of non-wheat flour (such as potato, rice and rye flours), gluten-free breads); pre-made bread mixes for preparing bread products; sauces, syrups and dressings; sweet spreads (including, but not limited to, jellies, jams, butters, nut spreads and other spreadable preserves, conserves and the like); confectionary products (including, but not limited to, jelly candies, soft candies, hard candies, chocolates and gums); sweetened breakfast cereals (including, but not limited to, extruded (KIX type) breakfast cereals, flaked breakfast cereals and puffed breakfast cereals); and cereal coating compositions for use in preparing sweetened breakfast cereals. Other types of food and beverage products not mentioned here but which conventionally include one or more nutritive sweetener may also be contemplated in the context of the present invention.

In embodiments, the product for oral administration is a food product which is warmed or heated prior to eating or which is served for consumption warm or hot. In embodiments, polypeptide variants herein which exhibits enhanced thermal stability compared to the polypeptide of SEQ ID NO:3 are preferred for application in compositions for oral administration (e.g., food products) which are to be cooked, warmed or heated prior to administration or consumption or which are to be administered or consumed warm or hot.

As a consequence of the complete or partial replacement of nutritive sweeteners in the food or beverage products of the present invention, the food or beverage products of the present invention may be useful as low calorie or dietetic products, medical foods/products (including pills and tablets), and sports nutrition products, and may be particularly suitable for food or beverage products requiring a lower sweetness at a given soluble solids level.

In some embodiments, the sweetening composition of the invention can be supplemented with other nutritional or non-nutritional sweeteners to form a sweetener system. The sweetener system may comprise the sweetening composition of the invention, a bulking agent such as maltodextrose, gum acacia and the like, and at least one high intensity sweetener. The composition may be provided as liquid composition or a dried blend.

As used herein the term “high-intensity sweetener,” refers to any synthetic or semi-synthetic sweetener or sweetener found in nature. High-intensity sweeteners are compounds or mixtures of compounds which are sweeter than sucrose. High-intensity sweeteners are typically many times (e.g., 20 times and more, 30 times and more, 50 times and more or 100 times or more) sweeter than sucrose).

In an embodiment, the present invention includes a process for enhancing the sweet taste of a product for oral administration, comprising the addition of a Myd polypeptide variant of the invention.

In another embodiment, the methods of the invention include a method for improving the sweet flavor of a product for oral administration, comprising adding to the product for oral administration a sweetening composition made by the methods of the invention. Amounts to add can be determined by methods known in the art, e.g., using sensory testing as a guide.

In another embodiment, the methods of the invention include methods for modifying the flavor of a product for oral administration, comprising adding to the product for oral administration a flavor modifying composition made by the methods of the invention. Amounts to add can be determined by methods known in the art, e.g., using sensory testing as a guide. A flavor modifying composition may modify (e.g., enhance, inhibit or change) a taste, aroma and/or texture of a given composition, e.g., a consumable. In a particular embodiment, the flavor modifying composition modifies (e.g., enhances, inhibits or changes) a particular taste(s), In another embodiment, the flavor modifying composition modifies (e.g., enhances, inhibits of changes) a given texture. In certain embodiments, the flavor modifying composition modifies (e.g., enhances, inhibits or changes) both a given taste(s) and texture.

A flavor modifying composition may be sweetened or unsweetened. Therefore, in some embodiments, the addition of a flavor modifying composition may serve both to add flavor modifiers and may further provide sweetness to a composition selected for taste adjustment. The addition of a sweetened flavor modifying composition may be used in addition to or alternatively to addition of another sweetening composition.

The sweetener compositions and flavor modifying compositions disclosed herein contain Myd (HTS) and variants thereof. In certain embodiments, HTS (or variant thereof) is the only sweet tasting component in the sweetener composition or flavor modifying composition. In certain embodiments, the sweetener composition or flavor modifying composition further comprises one or more additional sweet tasting components (i.e., additional sweetener or high-intensity sweetener), In embodiments, the additional sweetener is a polypeptide or protein sweetener other than HTS or a variant thereof. In embodiments, the sweetener is a carbohydrate sweetener. In embodiments, the additional sweetener is a synthetic sweetener. In a particular embodiment, the one or more sweet tasting components include steviol glycosides (e.g., Reb M, Reb A) and high fructose corn syrup (HFCS).

High-fructose corn syrup (HFCS), also known as glucose-fructose, isoglucose and glucose-fructose syrup, is a sweetener made from corn starch. As in the production of conventional corn syrup, the starch is broken down into glucose by enzymes.

Steviol glycosides are compounds responsible for the sweet taste of leaves of the plant Stevia rebaudiana and several related plants. Certain steviol glycosides are the ingredients in or are precursors to ingredients in stevia sweeteners. Steviol glycoside may be a single compound or a mixture of compounds. Steviol glycosides include, among others, stevioside, dulcoside A, rebaudioside A (Reb A), rebaudioside M (Reb M), rebaudioside B (Reb B), rebaudioside C (Reb C), rebaudioside D (Reb D), rebaudioside E, rebaudioside F, rubusoside, steviolbioside and combinations thereof.

Mogrosides are glycosides of cucurbitane derivatives, including mogrol, associated with the sweet taste of extracts of Siraitia grosvenorii (monkfruit or luo han guo). Certain mogrosides are the ingredients in monkfruit sweeteners. Mogrosides include, among others, mogroside II A₁, mogroside II B, 7-oxomogroside II E. 11-oxomogroside A₁, mogroside III A₂, 11-deoxymogroside III, 11-oxomogroside IV A, mogroside V, 7-oxomogroside V, 11-oxo-mogroside V, mogroside VI, siamenoside I and combinations thereof. Preferred mogrosides are mogroside V, mogroside VI and siamenoside.

In one embodiment, the one or more additional sweetener may be a carbohydrate sweetener. Non-limiting examples of suitable carbohydrate sweeteners include sucrose, fructose, glucose, erythritol, maltitol, lactitol, sorbitol, mannitol, xylitol, D-tagatose, trehalose, galactose, rhamnose, cyclodextrin (e.g., α-cyclodextrin, β-cyclodextrin, and γ-cyclodextrin), ribulose, threose, arabinose, xylose, lyxose, allose, altrose, mannose, idose, lactose, maltose, invert sugar, isotrehalose, neotrehalose, palatinose or isomaltulose, erythrose, deoxyribose, gulose, idose, talose, erythrulose, xylulose, psicose, turanose, cellobiose, glucosamine, mannosamine, fucose, fuculose, glucuronic acid, gluconic acid, glucono-lactone, abequose, galactosamine, xylo-oligosaccharides (xylotriose, xylobiose and the like), gentio-oligosaccharides (gentiobiose, gentiotriose, gentiotetraose and the like), galacto-oligosaccharides, sorbose, ketotriose (dehydroxyacetone), aldotriose (glyceraldehyde), nigero-oligosaccharides, fructooligosaccharides (kestose, nystose and the like), maltotetraose, maltotriol, tetrasaccharides, mannan-oligosaccharides, malto-oligosaccharides (maltotriose, maltotetraose, maltopentaose, maltohexaose, maltoheptaose and the like), dextrins, lactulose, melibiose, raffinose, rhamnose, ribose, isomerized liquid sugars such as high fructose com/starch syrup (HFCS/HFSS) (e.g., HFCS55, HFCS42, or HFCS90), coupling sugars, soybean oligosaccharides, glucose syrup and combinations thereof.

In other embodiments, the at least one additional sweetener is a synthetic sweetener. As used herein, the phrase “synthetic sweetener” refers to any composition which is not found naturally in nature and characteristically has a sweetness potency greater than sucrose, fructose, or glucose, yet has less calories. Non-limiting examples of synthetic high-potency sweeteners suitable for embodiments of this disclosure include sucralose, potassium acesulfame, aspartame, alitame, saccharin, neohesperidin dihydrochalcone, cyclamate, neotame, advantame, glucosylated steviol glycosides (GSGs) and combinations thereof.

In other embodiments, the at least one additional sweetener is a protein sweetener (i.e., a polypeptide or protein that tastes sweet). Typically, such protein sweeteners are extracted from plants. Non-limiting examples of protein sweeteners are monellin, thaumatin, brazzein, curculin, pentadin, and mabinlin.

In a particular embodiment, the sweetener composition and flavor modifying compositions disclosed herein comprise a Myd (HTS) variant. In one embodiment, the HTS variant differs from wild-type HTS (with or without methionine at position 1) at least one amino acid position and more particularly, at one, two, three or more amino acid positions.

In one embodiment, the sweetener composition and flavor modifying composition disclosed herein comprises a HTS variant having a sweetness equal to or greater than wild-type HTS having an amino acid sequence of SEQ ID NO:3.

In one embodiment, the sweetener composition and flavor modifying composition disclosed herein comprise a HTS variant having a stability equal to or greater than wild-type HTS having an amino acid sequence of SEQ ID NO:3.

In one embodiment, the sweetener composition and flavor modifying composition disclosed herein comprises a HTS variant having a sweetness equal to or greater than wild-type HTS having an amino acid sequence of SEQ ID NO:3 without methionine at position 1.

In one embodiment, the sweetener composition and flavor modifying composition disclosed herein comprise a HTS variant having a stability equal to or greater than wild-type HTS having an amino acid sequence of SEQ ID NO:3 without methionine at position 1.

HTS (or variants thereof) suitable for use in compositions disclosed herein (e.g., sweetener compositions, flavor and/or taste modifying compositions, consumables) may be produced in any suitable manner as previously described herein. Representative methods of production include extraction, chemical synthesis (i.e., solid state synthesis) or recombinant production (i.e., in vivo production or in vitro production).

In one embodiment, HTS used in the compositions disclosed herein is isolated from the edible (i) mycelia truffle of family Terfeziaceae or an aqueous extract thereof or (ii) an aqueous extract of a fruiting body of truffle of family Terfeziaceae. In one embodiment, the HTS is isolated from the mycelia or fruiting body of a Mattirolomyces terfezioides truffle.

In another embodiment, HTS (or variant thereof) used in compositions disclosed herein is produced in vivo. In one embodiment, the nucleic acid coding sequence for HTS isolated from Mattirolomyces terfezioides truffle, optionally optimized, is introduced into a suitable vector, which is then cloned into a host cell in an appropriate growth system/environment-resulting in expression of the protein in recombinant fashion. Suitable host cells and expression systems are previously described herein.

The amount of HTS (or a variant thereof) in the sweetener composition and flavor modifying compositions disclosed herein may vary. In one embodiment, the HTS (or a variant thereof) is present in the sweetener composition above its sweetness threshold concentration.

In one embodiment, HTS (or a variant thereof) is present in the sweetener composition or flavor modifying composition in any amount to impart the desired sweetness when the sweetener composition or flavor modifying composition is added to a consumable (e.g., beverage), either alone or in combination with one or more additional sweet tasting components (e.g., steviol glycosides, HFCS) present in the sweetener composition or flavor modifying composition, i.e., before such compositions are added to the consumable.

In a particular embodiment, the desired sweetness of the consumable is isosweet to a sucrose-sweetened consumable having a sweetness from at least about 8 degrees Brix, such as, for example, about 9 degrees Brix, about 10 degrees Brix, about 11 degrees Brix, about 12 degrees Brix, about 13 degrees Brix, about 14 degrees Brix or about 15 degrees Brix.

In another embodiment, the desired sweetness of the consumable is isosweet to a sucrose-sweetened consumable having a sweetness from about 10 degrees Brix to about 15 degrees Brix, such as, for example, from about 10 degrees Brix to about 14 degrees Brix, from about 10 degrees Brix to about 13 degrees Brix, from about 10 degrees Brix to about 12 degrees Brix, from about 10 degrees Brix to about 11 degrees Brix, from about 11 degrees Brix to about 15 degrees Brix, from about 11 degrees Brix to about 14 degrees Brix, from about 11 degrees Brix to about 13 degrees Brix, from about 11 degrees Brix to about 12 degrees Brix, from about 12 degrees Brix to about 15 degrees Brix, from about 12 degrees Brix to about 14 degrees Brix, from about 12 degrees Brix to about 13 degrees Brix, from about 13 degrees Brix to about 15 degrees Brix, from about 13 degrees Brix to about 14 degrees Brix and from about 14 degrees Brix to about 15 degrees Brix.

In one embodiment, the Myd polypeptide (or variant thereof) is present in the sweetener composition or flavor modifying composition in an amount that enhances the sweetness of the consumable to which it is added by about 1.0% (w/v) sucrose equivalence (SE) or greater, either alone or in combination with one or more additional sweet tasting component (e.g. steviol glycosides, HFCS) present in the sweetener composition or flavor modifying composition, i.e., before such compositions are added to the consumable.

In a particular embodiment, the Myd polypeptide (or variant thereof) is present in the sweetener composition in an amount that enhances the sweetness of the consumable to which it is added from about 1.0% to about 3.0% (w/v) sucrose equivalence (SE), such as, for example, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9% or about 3.0% sucrose equivalence, either alone or in combination with one or more additional sweet tasting components (e.g., steviol glycosides, HFCS) present in the sweetener composition or flavor modifying composition, i.e., before such compositions are added to the consumable.

In another particular embodiment, the Myd polypeptide (or variant thereof) is present in the sweetener composition or flavor modifying composition in an amount that enhances the sweetness of the consumable to which it is added by about 3.0% to about 5% (w/v) sucrose equivalence (SE), for example, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9% or about 5.0%, cither alone or in combination with one or more additional sweet tasting components (e.g., steviol glycosides, HFCS) present in the sweetener composition or flavor modifying composition, i.e., before such compositions are added to the consumable.

The sweetness of a given composition is typically measured with reference to a solution of sucrose. See generally “A Systematic Study of Concentration-Response Relationships of Sweeteners,” G. E. DuBois, D. E. Walters, S. S. Schiffman, Z. S. Warwick, B. J. Booth, S. D. Pecore, K, Gibes, B, T, Carr, and L. M. Brands, in Sweeteners: Discovery, Molecular Design and Chemoreception, D. B. Walters, F. T. Orthoefer, and G. E. DuBois, Eds., American Chemical Society, Washington, D.C. (1991), pp 261-276.

The amount of sucrose in a reference solution may be described in degrees Brix (Bx). One degrees Brix is 1 gram of sucrose in 100 grams of solution and represents the strength of the solution as percentage by weight (% w/w) (strictly speaking, by mass).

In one embodiment, a sweetener composition is provided that contains Myd polypeptide (or variant thereof) in an amount effective to provide sweetness equivalent from about 1 to about 12 degrees Brix of sugar when added to a consumable, such as, for example, from about 2 to about 9 degrees Brix, from about 3 to about 8 degrees Brix, from about 4 to about 7 degrees Brix, or about 5 degrees Brix, either alone or together with one or more sweet tasting components (e.g., steviol glycosides, HFCS) present in the sweetener composition or flavor modifying composition or consumable.

In another embodiment, Myd polypeptide (or variant thereof) is present in an amount effective to provide sweetness equivalent to about 10 degrees Brix when added to a sweetenable composition, either alone or in combination with one or more sweet tasting component (e.g., steviol glycosides, HFCS) present in the sweetener composition, flavor modifying composition or the consumable to which it is added.

The sweetness of a non-sucrose sweetener can also be measured against a sucrose reference by determining the non-sucrose sweetener's sucrose equivalence. Typically, taste panelists are trained to detect sweetness of reference sucrose solutions containing between 1-15% sucrose (w/v), Other non-sucrose sweeteners are then tasted at a series of dilutions to determine the concentration of the non-sucrose sweetener that is as sweet as a given percent sucrose reference. For example, if a 1% solution of a sweetener is as sweet as a 10% sucrose solution, then the sweetener is said to be 10 times as potent as sucrose.

In one embodiment, the amount of Myd polypeptide (or variant thereof) present in the sweetener composition or flavor modifying composition disclosed herein is any amount that contributes to one or more improved organoleptic properties of the consumable (e.g., beverage) to which the sweetener composition or taste modifying composition is added. In a particular embodiment, the improved organoleptic property is associated with basic taste. In one embodiment, improving one or more organoleptic property results in the improvement of the taste profile. The overall taste profile of a composition is an interplay of several different tastes, such as sweetness, sourness, saltiness, bitterness, umami and the like.

As used herein, “organoleptic properties” are the aspects of food, water or other substances that create an individual experience via the senses—including taste, sight, smell, and touch. Organoleptic properties include, e.g., appearance, texture, color, odor, size, shape and flavor. It is the qualitative evaluation based on the study of morphological and sensory profiles of the food, water, or other substance (such as, e.g., sweetener compositions).

Examples of improved organoleptic properties can include, for example, a reduction in bitterness, a reduction in astringent and liquorice notes, slower onset of sweetness, a reduction in lingering sweetness, a reduction in lingering bitterness, a reduction in bitter aftertaste, a reduction in metallic aftertaste, a reduction in chemical and synthetic aftertaste, and a combination thereof. In a particular embodiment, the term “improved organoleptic properties” means that the sweetened or taste modified composition (e.g., a beverage) will have one or more improved organoleptic properties for the majority of users. The improvement may be expressed qualitatively or quantitatively, e.g. as a percentage improvement.

The improved organoleptic property may be measured by or using technical means such as a taste sensing system (TSS), a term referring to analytical sensory array units (e.g., electrochemical, gravimetrical, optic or biosensors) which can detect specific substances. Sliwi'nska, M et al. J. Agric. Food Chem. (2014), 62, 1423-1448.

In a particular embodiment, the Myd polypeptide (or variant thereof) is present in the sweetener composition or flavor modifying composition in any amount that reduces, suppresses or masks the bitterness of a consumable (e.g., a beverage) to which the sweetener or flavor modifying composition is added, either alone or together with one or more sweet tasting components (e.g., steviol glycosides, HFCS) in the sweetener composition or flavor modifying composition, i.e., before it is added to the consumable. The comparison is made to a consumable to which the sweetener or flavor modifying composition has not been added.

In a particular embodiment, the Myd polypeptide (or variant thereof) is present in the sweetener composition or flavor modifying composition in an amount that reduces the bitterness of consumable (e.g., beverage) to which it is added by at least about 5%, at least about 10%, at least about 15%, at least about 20% or at least about 25% or more, either alone or in combination with one or more sweet taste components (e.g., steviol glycosides, HFCS) in the sweetener composition or flavor modifying composition, i.e., before it is added to the consumable. In one embodiment, the reduction in bitterness is experienced by a majority of subjects. The comparison is made to a consumable to which the sweetener or flavor modifying composition has not been added.

In a particular embodiment, the Myd polypeptide (or variant thereof) is present in the sweetener composition or flavor modifying composition in any amount that reduces the bitter aftertaste of a consumable (e.g., a beverage) to which the sweetener or flavor modifying composition is added, either alone or in combination with one or more sweet taste components (e.g., steviol glycosides, HFCS) in the sweetener composition or flavor modifying composition, i.e., before it is added to the consumable. In a particular embodiment, the Myd polypeptide (or variant thereof) is present in the sweetener composition or flavor modifying composition in an amount that reduces bitter aftertaste of consumable (e.g., beverage) to which it is added by at least about 5%, at least about 10%, at least about 15%, at least about 20% or at least about 25% or more. In one embodiment, the reduction in bitter aftertaste is experienced by a majority of subjects. The comparison is made to a consumable to which the sweetener or flavor modifying composition has not been added.

In another embodiment, the Myd polypeptide (or variant thereof) is present in the sweetener composition or taste modifying composition in any amount reduces the sweetness linger of a consumable (e.g., a beverage) to which the sweetener or taste modifying composition is added, Sucrose exhibits a sweet taste in which the maximal response is perceived quickly and where perceived sweetness disappears relatively quickly on swallowing a food or beverage. In contrast, the sweet tastes of essentially all high-potency sweeteners reach their maximal responses somewhat more slowly and they then decline in intensity more slowly than is the case for sucrose. This decline in sweetness is often referred to as “sweetness linger” and is a major limitation for high-potency sweeteners including NHPSs, Slow onset of sweetness also can be a problem. In general, however, sweetness linger is a more significant problem, And so, preferred embodiments of this invention exhibit significant reductions in sweetness linger.

In a particular embodiment, the Myd polypeptide (or variant thereof) is present in the sweetener composition or flavor modifying compositions in an amount that reduces the sweetness linger of the consumable (e.g., beverage) to which it is added by at least about 5%, at least about 10%, at least about 15%, at least about 20% or at least about 25% or more, either alone or in combination with one or more sweet tasting components (e.g., steviol glycosides, HFCS) in the sweetener composition or flavor modifying composition before it is added to the consumable. In one embodiment, the majority of subjects perceive the reduction in sweetness linger. In a particular embodiment, the comparison is made to a consumable to which the sweetener composition or flavor composition has not been added.

In a particular embodiment, the Myd polypeptide (or variant thereof) is present in the sweetener composition or flavor modifying compositions in an amount that results in at least one change/modification in organoleptic property in a consumable (e.g., beverage) compared to a consumable that does not contain the sweetener composition, wherein the organoleptic property is selected from the group consisting of aroma, flavor, basic taste (sweetness, sour, saltiness, bitterness or umami), aftertaste or linger, temporal profile, mouthfeel or a combination thereof. In this embodiment, a change or modification can be any perceived difference in the organoleptic property or properties that may or may not be considered an improvement. For example, if the Myd polypeptide being present in a consumable results in a change of a flavor from chocolate to caramel, this would be considered a “change” but not necessarily an “improvement”.

In certain embodiments, the sweetener composition or flavor and/or taste modifying compositions contain one or more additional sweeteners. In one embodiment, the additional sweetener is present above its sweetness threshold concentration. In certain embodiments, the sweetener composition containing Myd and the one or more additional sweeteners synergistically enhance the sweetness of the consumable to which the sweetener composition is added. In one embodiment, the sweetness of the consumable is enhanced in a manner that would be unexpected to one of skill in the art.

The additional sweetener can be any type of sweetener, for example, a natural, non-natural, or synthetic sweetener.

In at least one embodiment, the at least one additional sweetener is chosen from natural sweeteners other than Stevia sweeteners. In another embodiment, the at least one additional sweetener is chosen from synthetic high potency sweeteners (SHPS).

In a particular embodiment, the one or more additional sweetener may be a natural high potency sweetener (NHPS). Suitable natural high potency sweeteners include, but are not limited to, rebaudioside A, rebaudioside B, rebaudioside C, rebaudioside D, rebaudioside E, rebaudioside F, dulcoside A, dulcoside B, rubusoside, Stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, steviolbioside and cyclocarioside I. The natural high potency sweetener can be provided as a pure compound or, alternatively, as part of an extract. For example, rebaudioside A can be provided as a sole compound or as part of a Stevia extract.

In one embodiment, the one or more additional sweeteners is selected from the group consisting of rebaudioside M, rebaudioside A, siamenoside I and mogroside V.

In a particular embodiment, the sweetener composition and/or flavor modifying composition of the present invention comprises Myd polypeptide (or variant thereof) and siamenoside I.

In another particular embodiment, the sweetener composition and/or flavor modifying composition of the present invention comprises Myd polypeptide (or variant thereof) and mogroside V.

In another embodiment, the one or more additional sweeteners is selected from the group consisting of rebaudioside D, rebaudioside N, rebaudioside O, rebaudioside E, steviolmonoside, steviolbioside, rubusoside, dulcoside B, dulcoside A, rebaudioside B, rebaudioside G, stevioside, rebaudioside C, rebaudioside F, rebaudioside I, rebaudioside H, rebaudioside L, rebaudioside K, rebaudioside J, rebaudioside M2, rebaudioside D2, rebaudioside S, rebaudioside T, rebaudioside U, rebaudioside V, rebaudioside W, rebaudioside Z1, rebaudioside Z2, rebaudioside IX, enzymatically glucosylated steviol glycosides and combinations thereof.

In a further embodiment, the one or more additional sweeteners is selected from the group consisting of mogroside IA, mogroside IE, 11-oxomogroside IA, mogroside II, mogroside II A, mogroside II B, mogroside II E, 7-oxomogroside II E, mogroside III, Mogroside Ille, 11-deoxymogroside III, mogroside IV, 11-oxomogroside IV, 11-oxomogroside IV A, 11-deoxymogroside V, 7-oxomogroside V, 11-oxomogroside V, isomogroside V, mogroside VI, mogrol, 11-oxomogrol, the 1,6-α isomer of siamenoside I, monk fruit extract, and combinations thereof.

In a particular embodiment, the one or more additional sweeteners is rebaudioside M (13-[2-O-β-D-glucopyranosyl-3-O-β-D-glucopyranosyl-β-D-glucopyranosyl)oxy] Ent Kaur-16-end-19-oil acid-[2-O-β-D-glucopyranosyl-3-O-β-D-glycopyranosyl) ester having the formula of Formula (I):

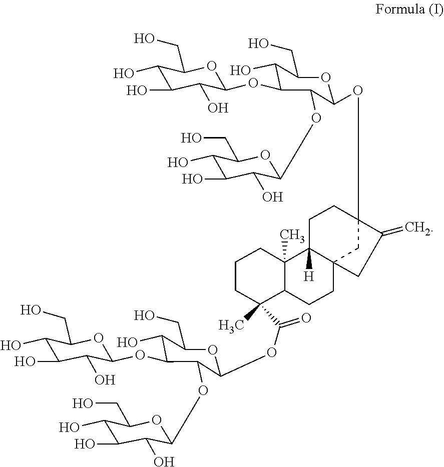

Reb M may be provided in a purified or unpurified form, i.e., as part of a naturally occurring mixture that contains Reb M. In one embodiment, Reb M can be obtained from a Stevia extract by any suitable purification method. Suitable purification methods are known in the art, including, but not limited to, column chromatography, recrystallization, phase separation, extraction, high performance liquid chromatography and combinations thereof.

In another embodiment, the additional sweetener is a steviol glycoside composition. An exemplary steviol glycoside composition is A95, which contains primarily reb D and reb M with minor amounts of one or more of the following: Reb E, Reb O, Reb N, Reb A, Stevioside, Reb C and Reb B. Methods of obtaining A95 are provided in WO 2017/059414, incorporated herein by reference.

The amount of Reb M in the sweeter composition or taste modifying composition may vary. In one embodiment, Reb M is present in a sweetener composition in any amount to impart the desired sweetness when the sweetener composition is added to a consumable (e.g., a beverage), In a particular embodiment, the desired sweetness of the consumable is greater than about 10 degrees Brix.

In one embodiment, the sweetener composition contains Reb M in an amount effective to provide sweetness equivalent from about 1 to 12 degrees Brix when added to a consumable (e.g., a beverage), such as, for example, from about 2 to about 9 degrees Brix, from about 3 to about 8 degrees Brix, from about 4 to about 7 degrees Brix, or about 5 degrees Brix.

In a particular embodiment, Reb M is present in an effective amount to provide a sucrose equivalence (SE) of about 8 or less, such as for example, about 7, about 6.5, about 6, about 5.5, or about 5 SE.

In another particular embodiment, Reb M is present in an effective amount to provide a sucrose equivalence of about 8 or greater, such as, for example, about 9, about 9.5, about 10, about 10.5, about 11, about 11.5, about 12, about 12.5, about 13, about 13.5, about 14, about 14.5 or about 15.

In one embodiment, Reb M is present in the flavor modifying composition in any amount to impart the desired flavor when the flavor modifying composition is added to a flavor modifiable composition (e.g., a beverage). In a particular embodiment, the desired flavor is a more sugar-like temporal or taste profile.

In one embodiment, the Reb M and the Myd polypeptide (or variant thereof) produce a synergistic effect, e.g., synergistic sweetness, i.e., the sweetness of combination is greater than the sum of the individual sweeteners. In a particular embodiment, the Reb M and the Myd polypeptide (or variant thereof) produce an effect that would be unexpected by one of skill in the art.

Reb M may be provided in a purified form or as a component of a mixture containing Reb M and one or more additional components. In one embodiment, Reb X is provided as a component of a mixture. In a particular embodiment, the mixture is a Stevia extract. The Stevia extract may contain Reb M in an amount that ranges from about 5% to about 100% by weight on a dry basis, such as, for example, from about 10% to about 100%, from about 20% to about 100%, from about 30% to about 100%, from about 40% to about 100%, from about 50% to about 100%, from about 60% to about 100%, from about 70% to about 100%, from about 80% to about 100% and from about 90% to about 100%. In still further embodiments, the Stevia extract contains Reb M in an amount greater than about 90% by weight on a dry basis, for example, greater than about 91%, greater than about 92%, greater than about 93%, greater than about 94%, greater than about 95%, greater than about 96%, greater than about 97%, greater than about 98% and greater than about 99%.

In one embodiment, Reb M is provided as a component of a steviol glycoside mixture, i.e., a mixture of steviol glycosides wherein the remainder of the non-Reb M portion of the mixture is comprised entirely of steviol glycosides. The identities of steviol glycosides are known in the art and include, but are not limited to, steviol, steviol monoside, rubososide, steviolbiocide, stevioside, rebaudioside A, rebaudioside B, rebaudioside C, rebaudioside D, rebaudioside E, rebaudioside F and dulcoside A. The steviol glycoside mixture may contain from about 5% to about 100% Reb M by weight on a dry basis. For example, a steviol glycoside mixture may contain from about 10% to about 100%, from about 20% to about 100%, from about 30% to about 100%, from about 40% to about 100%, from about 50% to about 100%, from about 60% to about 100%, from about 70% to about 100%, from about 80% to about 100% and from about 90% to about 100% Reb M by weight on a dry basis. In still further embodiments, the steviol glycoside mixture may contain greater than about 90%, for example, greater than about 91%, greater than about 92%, greater than about 93%, greater than about 94%, greater than about 95%, greater than about 96%, greater than about 97%, greater than about 98% and greater than about 99% Reb M by weight on a dry basis.

RebM80 refers to a Stevia extract or steviol glycoside composition having about 80% Reb M by weight.

In a particular embodiment, the one or more additional sweeteners is rebaudioside A.

Reb A may be provided in a purified or unpurified form, i.e., as part of a naturally occurring mixture that contains Reb a. In one embodiment, Reb A can be obtained from a Stevia extract by any suitable purification method Suitable purification methods are known in the art, including, but not limited to, column chromatography, recrystallization, phase separation, extraction, high performance liquid chromatography and combinations thereof.

Reb A may be provided in a purified form or as a component of a mixture containing Reb A and one or more additional components. In one embodiment, Reb A is provided as a component of a mixture. In a particular embodiment, the mixture is a Stevia extract. The Stevia extract may contain Reb A in an amount that ranges from about 5% to about 100% by weight on a dry basis, such as, for example, from about 10% to about 100%, from about 20% to about 100%, from about 30% to about 100%, from about 40% to about 100%, from about 50% to about 100%, from about 60% to about 100%, from about 70% to about 100%, from about 80% to about 100% and from about 90% to about 100%. In still further embodiments, the Stevia extract contains Reb A in an amount greater than about 90% by weight on a dry basis, for example, greater than about 91%, greater than about 92%, greater than about 93%, greater than about 94%, greater than about 95%, greater than about 96%, greater than about 97%, greater than about 98% and greater than about 99%.

In one embodiment, Reb A is provided as a component of a steviol glycoside mixture, i.e., a mixture of steviol glycosides wherein the remainder of the non-Reb A portion of the mixture is comprised entirely of steviol glycosides. The steviol glycoside mixture may contain from about 5% to about 100% Reb A weight on a dry basis. For example, a steviol glycoside mixture may contain from about 10% to about 100%, from about 20% to about 100%, from about 30% to about 100%, from about 40% to about 100%, from about 50% to about 100%, from about 60% to about 100%, from about 70% to about 100%, from about 80% to about 100% and from about 90% to about 100% Reb A by weight on a dry basis. In still further embodiments, the steviol glycoside mixture may contain greater than about 90%, for example, greater than about 91%, greater than about 92%, greater than about 93%, greater than about 94%, greater than about 95%, greater than about 96%, greater than about 97%, greater than about 98% and greater than about 99% Reb A by weight on a dry basis.

The amount of Reb A in the sweetener composition or taste modifying composition may vary. In one embodiment, Reb A is present in the sweetener composition in any amount to impart the desired sweetness when the sweetener composition is added to a sweetenable composition. In a particular embodiment, the desired sweetness of the sweetened composition is greater than about 10 degrees Brix.

In one embodiment, the Reb A and the Myd polypeptide (or variant thereof) produce a synergistic effect, e.g., synergistic sweetness, i.e., the sweetness of combination is greater than the sum of the individual sweeteners. In certain embodiments, the Reb A and the Myd polypeptide (or variant thereof) produce an effect that one of skill in the art would not have expected.

In a particular embodiment, Reb A is present in an effective amount to provide a sucrose equivalence (SE) of about 8 or less, such as for example, about 7, about 6.5, about 6, about 5.5, or about 5 SE.

In another particular embodiment, Reb A is present in an effective amount to provide a sucrose equivalence (SE) of about 8 or greater, such as, for example, about 9, about 9.5, about 10, about 10.5, about 11, about 11.5, about 12, about 12.5, about 13, about 13.5, about 14, about 14.5 or about 15.

In another particular embodiment, Reb A is present in an effective amount to provide a sucrose equivalence of greater than about 8 SE, e.g., about 9 SE, about 9.5 SE, about 10 SE.

In one embodiment, Reb A is present in the flavor modifying composition in any amount to impart the desired taste when the taste modifying composition is added to a taste modifiable composition (e.g., a beverage). In a particular embodiment, the desired taste is a sugar-like taste.

In a particular embodiment, the Myd polypeptide (or variant thereof) and Reb A produce a synergistic effect. In one embodiment, the Myd polypeptide (or variant thereof) and Reb A produce an effect that would been unexpected to one of skill in the art.

The sweetener compositions can be customized to obtain a desired calorie content. For example, sweetener compositions can be “high-calorie”, such that they impart the desired sweetness when added to a sweetenable composition (such as, for example, as beverage) and have about 120 calories per 8 oz serving.

The sweetener compositions can be customized to obtain a desired calorie content. For example, sweetener compositions can be “mid-calorie”, such that they impart the desired sweetness when added to a sweetenable composition (such as, for example, as beverage) and have about 80 calories per 8 oz serving.

For example, sweetener compositions can be “low-calorie”, such that they impart the desired sweetness when added to a sweetenable composition (such as, for example, as beverage) and have less than 40 calories per 8 oz serving.

In other embodiments, the sweetener compositions can be “zero-calorie”, such that they impart the desired sweetness when added to a sweetenable composition (such as, for example, a beverage) and have less than 5 calories per 8 oz. serving,

### Additives

In addition to Myd (HTS) polypeptides (or variants thereof) and, optionally, one or more additional sweeteners (e.g., one or more steviol glycosides), the sweetener compositions or flavor modifying compositions disclosed herein can optionally include additional additives, detailed herein below. In some embodiments, the sweetener composition contains additives including, but not limited to, carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, flavorants and flavoring ingredients, astringent compounds, proteins or protein hydrolysates, surfactants, emulsifiers, flavonoids, alcohols, polymers and combinations thereof. In some embodiments, the additives act to improve the temporal and flavor profile of the sweetener to provide a sweetener composition with a taste similar to sucrose.

In one embodiment, the sweetener compositions or flavor modifying compositions contain one or more polyols. The term “polyol”, as used herein, refers to a molecule that contains more than one hydroxyl group. A polyol may be a diol, triol, or a tetraol which contains 2, 3, and 4 hydroxyl groups respectively. A polyol also may contain more than 4 hydroxyl groups, such as a pentaol, hexaol, heptaol, or the like, which contain 5, 6, or 7 hydroxyl groups, respectively. Additionally, a polyol also may be a sugar alcohol, polyhydric alcohol, or polyalcohol which is a reduced form of carbohydrate, wherein the carbonyl group (aldehyde or ketone, reducing sugar) has been reduced to a primary or secondary hydroxyl group.

Non-limiting examples of polyols in some embodiments include erythritol, maltitol, mannitol, sorbitol, lactitol, xylitol, isomalt, propylene glycol, glycerol (glycerin), threitol, galactitol, palatinose, reduced isomalto-oligosaccharides, reduced xylo-oligosaccharides, reduced gentio-oligosaccharides, reduced maltose syrup, reduced glucose syrup, and sugar alcohols or any other carbohydrates capable of being reduced which do not adversely affect taste.

Suitable sweet taste improving amino acid additives include, but are not limited to, aspartic acid, arginine, glycine, glutamic acid, proline, threonine, theanine, cysteine, cystine, alanine, valine, tyrosine, leucine, arabinose, trans-4-hydroxyproline, isoleucine, asparagine, serine, lysine, histidine, omnithine, methionine, carnitine, aminobutyric acid (α-, β-, and/or δ-isomers), glutamine, hydroxyproline, taurine, norvaline, sarcosine, and their salt forms such as sodium or potassium salts or acid salts. The sweet taste improving amino acid additives also may be in the D or L-configuration and in the mono-, di-, or tri-form of the same or different amino acids. Additionally, the amino acids may be α-, β-, γ- and/or δ-isomers if appropriate. Combinations of the foregoing amino acids and their corresponding salts (e.g., sodium, potassium, calcium, magnesium salts or other alkali or alkaline earth metal salts thereof, or acid salts) also are suitable sweet taste improving additives in some embodiments. The amino acids may be natural or synthetic. The amino acids also may be modified. Modified amino acids refers to any amino acid wherein at least one atom has been added, removed, substituted, or combinations thereof (e.g., N-alkyl amino acid, N-acyl amino acid, or N-methyl amino acid). Non-limiting examples of modified amino acids include amino acid derivatives such as trimethyl glycine, N-methyl-glycine, and N-methyl-alanine. As used herein, modified amino acids encompass both modified and unmodified amino acids. As used herein, amino acids also encompass both peptides and polypeptides (e.g., dipeptides, tripeptides, tetrapeptides, and pentapeptides) such as glutathione and L-alanyl-L-glutamine. Suitable sweet taste improving polyamino acid additives include poly-L-aspartic acid, poly-L-lysine (e.g., poly-L-α-lysine or poly-L-ε-lysine), poly-L-ornithine (e.g., poly-L-□α-ornithine or poly-L-□ε-ornithine), poly-L-arginine, other polymeric forms of amino acids, and salt forms thereof (e.g., calcium, potassium, sodium, or magnesium salts such as L-glutamic acid mono sodium salt). The sweet taste improving poly-amino acid additives also may be in the D. or L-configuration. Additionally, the poly-amino acids may be α-, β-, γ-, δ-, and ε-isomers if appropriate. Combinations of the foregoing poly-amino acids and their corresponding salts (e.g., sodium, potassium, calcium, magnesium salts or other alkali or alkaline earth metal salts thereof or acid salts) also are suitable sweet taste improving additives in some embodiments. The poly-amino acids described herein also may comprise co-polymers of different amino acids. The poly-amino acids may be natural or synthetic. The poly-amino acids also may be modified, such that at least one atom has been added, removed, substituted, or combinations thereof (e.g., N-alkyl poly-amino acid or N-acyl poly-amino acid). As used herein, poly-amino acids encompass both modified and unmodified poly-amino acids. For example, modified poly-amino acids include, but are not limited to, poly-amino acids of various molecular weights (MW), such as poly-L-α-lysine with a MW of 1,500. MW of 6,000, MW of 25,200, MW of 63,000, MW of 83,000, or MW of 300,000.

Suitable sugar acid additives include, but are not limited to, aldonic, uronic, aldaric, alginic, gluconic, glucuronic, glucaric, galactaric, galacturonic, and salts thereof (e.g., sodium, potassium, calcium, magnesium salts or other physiologically acceptable salts), and combinations thereof.

Suitable nucleotide additives include, but are not limited to, inosine monophosphate (“IMP”), guanosine monophosphate (“GMP”), adenosine monophosphate (“AMP”), cytosine monophosphate (CMP), uracil monophosphate (UMP), inosine diphosphate, guanosine diphosphate, adenosine diphosphate, cytosine diphosphate, uracil diphosphate, inosine triphosphate, guanosine triphosphate, adenosine triphosphate, cytosine triphosphate, uracil triphosphate, alkali or alkaline earth metal salts thereof, and combinations thereof. The nucleotides described herein also may comprise nucleotide-related additives, such as nucleosides or nucleic acid bases (e.g., guanine, cytosine, adenine, thymine, uracil). In particular embodiments, the nucleotide is present in the sweetener composition in an amount from about 5 ppm to about 1,000 ppm.

Suitable organic acid additives include any compound which comprises a —COOH moiety, such as, for example, C2-C30 carboxylic acids, substituted hydroxyl C2-C30 carboxylic acids, benzoic acid, substituted benzoic acids (e.g., 2,4-dihydroxybenzoic acid), substituted cinnamic acids, hydroxyacids, substituted hydroxybenzoic acids, substituted cyclobexyl carboxylic acids, tannic acid, lactic acid, tartaric acid, citric acid, gluconic acid, glucoheptonic acids, adipic acid, hydroxycitric acid, malic acid, fruitaric acid (a blend of malic, fumaric, and tartaric acids), fumaric acid, maleic acid, succinic acid, chlorogenic acid, salicylic acid, creatine, caffeic acid, bile acids, acetic acid, ascorbic acid, alginic acid, erythorbic acid, polyglutamic acid, glucono delta lactone, and their alkali or alkaline earth metal salt derivatives thereof. In addition, the organic acid additives also may be in either the D- or L-configuration.

Suitable organic acid additive salts include, but are not limited to, sodium, calcium, potassium, and magnesium salts of all organic acids, such as salts of citric acid, malic acid, tartaric acid, fumaric acid, lactic acid (e.g., sodium lactate), alginic acid (e.g., sodium alginate), ascorbic acid (e.g., sodium ascorbate), benzoic acid (e.g., sodium benzoate or potassium benzoate), and adipic acid. The examples of the sweet taste improving organic acid additives described optionally may be substituted with at least one group chosen from hydrogen, alkyl, alkenyl, alkynyl, halo, haloalkyl, carboxyl, acyl, acyloxy, amino, amido, carboxyl derivatives, alkylamino, dialkylamino, arylamino, alkoxy, aryloxy, nitro, cyano, sulfo, thiol, imine, sulfonyl, sulfenyl, sulfinyl, sulfamyl, carboxalkoxy, carboxamido, phosphonyl, phosphinyl, phosphoryl, phosphino, thioester, thioether, anhydride, oximino, hydrazino, carbamyl, phosphor or phosphonato. In particular embodiments, the organic acid additive is present in the sweetener composition in an amount from about 10 ppm to about 5,000 ppm.

Suitable inorganic acid additives include, but are not limited to, phosphoric acid, phosphorous acid, polyphosphoric acid, hydrochloric acid, sulfuric acid, carbonic acid, sodium dihydrogen phosphate, and alkali or alkaline earth metal salts thereof (e.g., inositol hexaphosphate Mg/Ca).

Suitable bitter compound additives include, but are not limited to, caffeine, quinine, urea, bitter orange oil, naringin, quassia, and salts thereof.

Suitable flavorant and flavoring ingredient additives for include, but are not limited to, vanillin, vanilla extract, mango extract, cinnamon, Citrus, coconut, ginger, viridiflorol, almond, menthol (including menthol without mint), grape skin extract, and grape seed extract. “Flavorant” and “flavoring ingredient” are synonymous and can include natural or synthetic substances or combinations thereof. Flavorants also include any other substance which imparts flavor and may include natural or non-natural (synthetic) substances which are safe for human or animals when used in a generally accepted range. Non-limiting examples of proprietary flavorants include Döhler™ Natural Flavoring Sweetness Enhancer K14323 (Döhler™, Darmstadt, Germany), Symrise™ Natural Flavor Mask for Sweeteners 161453 and 164126 (Symrise™, Holzminden, Germany), Natural Advantage™ Bitterness Blockers 1, 2, 9 and 10 (Natural Advantage™, Freehold, N.J., U.S.A.), and Sucramask™ (Creative Research Management, Stockton, Calif., U.S.A.).

Suitable polymer additives include, but are not limited to, chitosan, pectin, pectie, pectinic, polyuronic, polygalacturonic acid, starch, food hydrocolloid or crude extracts thereof (e.g., gum acacia senegal (Fibergum™), gum acacia seyal, carageenan), poly-L-lysine (e.g., poly-L-α-lysine or poly-L-ε-lysine), poly-L-ornithine (e.g., poly-L-α-ornithine or poly-L-8-ornithine), polypropylene glycol, polyethylene glycol, poly (ethylene glycol methyl ether), polyarginine, polyaspartic acid, polyglutamic acid, polyethylene imine, alginic acid, sodium alginate, propylene glycol alginate, and sodium polyethyleneglycolalginate, sodium hexametaphosphate and its salts, and other cationic polymers and anionic polymers.

Other suitable polymer additives that also provide gelling and thickening properties include conventional low methoxyl (LMC) pectins. LMC pectin also acts as a food stabilizer. LMC apple pectin is a conventional low methoxyl pectin, extracted from apple pomace and standardized with sucrose. It is utilized for low-calorie jams and jellies since it relies on calcium instead of sugar to solidify. LMC pectin gets increasingly firmer as calcium is added until it hits a saturation point. At that time, the process reverses and it becomes less firm. In some embodiments, the products for oral administration described herein may comprise one or more food product selected from the group consisting of pie fillings and other sweet fillings, gelatins and puddings; yogurts; sauces, syrups and dressings; and sweet spreads. In one embodiment, the product for oral administration further comprises a low methoxyl pectin.

Suitable protein or protein hydrolysate additives include, but are not limited to, bovine serum albumin (BSA), whey protein (including fractions or concentrates thereof such as 90% instant whey protein isolate, 34% whey protein, 50% hydrolyzed whey protein, and 80% whey protein concentrate), soluble rice protein, soy protein, protein isolates, protein hydrolysates, reaction products of protein hydrolysates, glycoproteins, and/or proteoglycans containing amino acids (e.g., glycine, alanine, serine, threonine, asparagine, glutamine, arginine, valine, isoleucine, leucine, norvaline, methionine, proline, tyrosine, hydroxyproline, and the like), collagen (e.g., gelatin), partially hydrolyzed collagen (e.g., hydrolyzed fish collagen), and collagen hydrolysates (e.g., porcine collagen hydrolysate).

Suitable surfactant additives include, but are not limited to, polysorbates (e.g., polyoxyethylene sorbitan monoolcate (polysorbate 80), polysorbate 20, polysorbate 60), sodium dodecylbenzenesulfonate, dioctyl sulfosuccinate or dioctyl sulfosuccinate sodium, sodium dodecyl sulfate, cetylpyridinium chloride (hexadecylpyridinium chloride), hexadecyltrimethylammonium bromide, sodium cholate, carbamoyl, choline chloride, sodium glycocholate, sodium taurodeoxycholate, laurie arginate, sodium stearoyl lactylate, sodium taurocholate, lecitbins, sucrose oleate esters, sucrose stearate esters, sucrose palmitate esters, sucrose laurate esters, and other emulsifiers, and the like.

Suitable flavonoid additives are classified as flavonols, flavones, flavanones, flavan-3-ols, isoflavones, or anthocyanidins. Non-limiting examples of flavonoid additives include, but are not limited to, catechins (e.g., green tea extracts such as Polyphenon™ 60, Polyphenon™ 30, and Polyphenon™ 25 (Mitsui Norin Co., Ltd., Japan), polyphenols, rutins (e.g., enzyme modified rutin Sanmelin™ AO (San-fi Gen F.F.I., Inc., Osaka, Japan)), neohesperidin, naringin, neohesperidin dihydrochalcone, and the like.

Suitable alcohol additives include, but are not limited to, ethanol.

Suitable astringent compound additives include, but are not limited to, tannic acid, curopium chloride (EuCl3), gadolinium chloride (GdCl3), terbium chloride (TbCl₃), alum, tannic acid, and polyphenols (e.g., tea polyphenols). In particular embodiments, the astringent additive is present in an amount from about 10 ppm to about 5,000 ppm.

In particular embodiments, sweetener compositions or flavor modifying compositions comprise the Myd polypeptide (or variant thereof), optionally in combination with one or more steviol glycosides (e.g., Reb M, Reb A), a polyol selected from erythritol, maltitol, mannitol, xylitol, sorbitol, and combinations thereof; and optionally at least one additional sweetener and/or functional ingredient. In a particular embodiment, the polyol is erythritol. The steviol glycoside (e.g., Reb M, Reb A) can be provided as a pure compound or as part of a Stevia extract or steviol glycoside mixture, as described above. The steviol glycoside (e.g., Reb M, Reb A) can be present in an amount from about 5% to about 100% by weight on a dry basis in either a steviol glycoside mixture or a Stevia extract.

In particular embodiments, the sweetener compositions or flavor modifying compositions comprise the Myd polypeptide (or variant thereof), optionally in combination with one or more steviol glycosides (e.g., Reb M, Reb A); a carbohydrate sweetener selected from sucrose, fructose, glucose, maltose and combinations thereof; and optionally at least one additional sweetener and/or functional ingredient. The steviol glycoside (e.g., Reb M, Reb A) can be provided as a pure compound or as part of a Stevia extract or steviol glycoside mixture, as described above. The steviol glycoside (e.g., Reb M, Reb A) can be present in an amount from about 5% to about 100% by weight on a dry basis in either a steviol glycoside mixture or a Stevia extract.

In particular embodiments, the sweetener compositions or flavor modifying compositions comprise the Myd polypeptide (or variant thereof), optionally in combination with one or more steviol glycosides (e.g., Reb M, Reb A); an amino acid selected from glycine, alanine, proline and combinations thereof; and optionally at least one additional sweetener and/or functional ingredient. The steviol glycoside can be provided as a pure compound or as part of a Stevia extract or steviol glycoside mixture, as described above. The steviol glycoside (e.g., Reb M, Reb A) can be present in an amount from about 5% to about 100% by weight on a dry basis in either a steviol glycoside mixture or a Stevia extract.

In particular embodiments, sweetener compositions or flavor modifying compositions comprise the Myd polypeptide (or variant thereof)), optionally in combination with one or more steviol glycosides (e.g., Reb M, Reb A), a salt selected from sodium chloride, magnesium chloride, potassium chloride, calcium chloride and combinations thereof; and optionally at least one additional sweetener and/or functional ingredient. The steviol glycoside (e.g., Reb M, Reb A) can be provided as a pure compound or as part of a Stevia extract or steviol glycoside mixture, as described above.

### Functional Ingredients

The sweetener compositions or flavor modifying compositions disclosed herein can also contain one or more functional ingredients, which provide a real or perceived health benefit to the composition. Functional ingredients include, but are not limited to, saponins, antioxidants, dietary fiber sources, fatty acids, vitamins, glucosamine, minerals, preservatives, hydration agents, probiotics, prebiotics, weight management agents, osteoporosis management agents, phytoestrogens, long chain primary aliphatic saturated alcohols, phytosterols and combinations thereof.

Examples of suitable antioxidants for embodiments of this invention include, but are not limited to, vitamins, vitamin cofactors, minerals, hormones, carotenoids, carotenoid terpenoids, non-carotenoid terpenoids, flavonoids, flavonoid polyphenolics (e.g., bioflavonoids), flavonols, flavones, phenols, polyphenols, esters of phenols, esters of polyphenols, nonflavonoid phenolics, isothiocyanates, and combinations thereof. In some embodiments, the antioxidant is vitamin A, vitamin C, vitamin E, ubiquinone, mineral selenium, manganese, melatonin, α-carotene, β-carotene, lycopene, lutein, zeanthin, crypoxanthin, reservatol, eugenol, quercetin, catechin, gossypol, hesperetin, curcumin, ferulic acid, thymol, hydroxytyrosol, tumeric, thyme, olive oil, lipoic acid, glutathinone, gutamine, oxalic acid, tocopherol-derived compounds, butylated hydroxyanisole (BHA), butylated hydroxytoluene (BHT), ethylenediaminetetraacetic acid (EDTA), tert-butylhydroquinone, acetic acid, pectin, tocotrienol, tocopherol, coenzyme Q10, zeaxanthin, astaxanthin, canthaxantin, saponins, limonoids, kaempfedrol, myricetin, isorhamnetin, proanthocyanidins, quercetin, rutin, luteolin, apigenin, tangeritin, hesperetin, naringenin, erodictyol, flavan-3-ols (e.g., anthocyanidins), gallocatechins, epicatechin and its gallate forms, epigallocatechin and its gallate forms (ECGC) theaflavin and its gallate forms, thearubigins, isoflavone phytoestrogens, genistein, daidzein, glycitein, anythocyanins, cyaniding, delphinidin, malvidin, pelargonidin, peonidin, petunidin, ellagic acid, gallic acid, salicylic acid, rosmarinic acid, cinnamic acid and its derivatives (e.g., ferulic acid), chlorogenic acid, chicoric acid, gallotannins, ellagitannins, anthoxanthins, betacyanins and other plant pigments, silymarin, citric acid, lignan, antinutrients, bilirubin, uric acid, R-α-lipoic acid, N-acetylcysteine, emblicanin, and phytic acid, or combinations thereof. In alternate embodiments, the antioxidant is a synthetic antioxidant such as butylated hydroxytolune or butylated hydroxyanisole, for example. Other sources of suitable antioxidants for embodiments of this invention include, but are not limited to, fruits, vegetables, tea, cocoa, chocolate, spices, herbs, rice, organ meats from livestock, yeast, whole grains, or cereal grains.

Particular antioxidants belong to the class of phytonutrients called polyphenols (also known as “polyphenolics”), which are a group of chemical substances found in plants, characterized by the presence of more than one phenol group per molecule. Suitable polyphenols for embodiments of this invention, include catechins, proanthocyanidins, procyanidins, anthocyanins, quercerin, rutin, reservatrol, isoflavones, curcumin, punicalagin, ellagitannin, hesperidin, naringin, Citrus flavonoids, chlorogenic acid, other similar materials, and combinations thereof.

In particular embodiments, the antioxidant is a catechin such as, for example, epigallocatechin gallate (EGCG). Suitable sources of catechins for embodiments of this invention include, but are not limited to, green tea, white tea, black tea, oolong tea, chocolate, cocoa, red wine, grape seed, red grape skin, purple grape skin, red grape juice, purple grape juice, berries, pycnogenol, and red apple peel.

In some embodiments, the antioxidant is chosen from proanthocyanidins, procyanidins or combinations thereof. Suitable sources of proanthocyanidins and procyanidins for embodiments of this invention include, but are not limited to, red grapes, purple grapes, cocoa, chocolate, grape seeds, red wine, cacao beans, cranberry, apple peel, plum, blueberry, black currants, choke berry, green tea, sorghum, cinnamon, barley, red kidney bean, pinto bean, hops, almonds, hazelnuts, pecans, pistachio, pycnogenol, and colorful berries.

In particular embodiments, the antioxidant is an anthocyanin. Suitable sources of anthocyanins for embodiments of this invention include, but are not limited to, red berries, blueberries, bilberry, cranberry, raspberry, cherry, pomegranate, strawberry, elderberry, choke berry, red grape skin, purple grape skin, grape seed, red wine, black currant, red currant, cocoa, plum, apple peel, peach, red pear, red cabbage, red onion, red orange, and blackberries.

In some embodiments, the antioxidant is chosen from quercetin, rutin or combinations thereof. Suitable sources of quercetin and rutin for embodiments of this invention include, but are not limited to, red apples, onions, kale, bog whortleberry, lingonberrys, chokeberry, cranberry, blackberry, blueberry, strawberry, raspberry, black currant, green tea, black tea, plum, apricot, parsley, leek, broccoli, chili pepper, berry wine, and ginkgo.

In some embodiments, the antioxidant is resveratrol. Suitable sources of resveratrol for embodiments of this invention include, but are not limited to, red grapes, peanuts, cranberry, blueberry, bilberry, mulberry, Japanese Itadori tea, and red wine.

In particular embodiments, the antioxidant is an isoflavone. Suitable sources of isoflavones for embodiments of this invention include, but are not limited to, soy beans, soy products, legumes, alfalfa sprouts, chickpeas, peanuts, and red clover.

In some embodiments, the antioxidant is curcumin. Suitable sources of curcumin for embodiments of this invention include, but are not limited to, turmeric and mustard.

In particular embodiments, the antioxidant is chosen from punicalagin, ellagitannin or combinations thereof. Suitable sources of punicalagin and ellagitannin for embodiments of this invention include, but are not limited to, pomegranate, raspberry, strawberry, walnut, and oak-aged red wine.

In some embodiments, the antioxidant is a Citrus flavonoid, such as hesperidin or naringin. Suitable sources of Citrus flavonoids, such as hesperidin or naringin, for embodiments of this invention include, but are not limited to, oranges, grapefruits, and Citrus juices.

In particular embodiments, the antioxidant is chlorogenic acid. Suitable sources of chlorogenic acid for embodiments of this invention include, but are not limited to, green coffee, yerba mate, red wine, grape seed, red grape skin, purple grape skin, red grape juice, purple grape juice, apple juice, cranberry, pomegranate, blueberry, strawberry, sunflower, Echinacea, pycnogenol, and apple peel.

Suitable dietary fibers include, but are not limited to, non-starch polysaccharides, lignin, cellulose, methylcellulose, the hemicelluloses, β-glucans, pectins, gums, mucilage, waxes, inulins, oligosaccharides, fructooligosaccharides, cyclodextrins, chitins, and combinations thereof.

Food sources of dietary fiber include, but are not limited to, grains, legumes, fruits, and vegetables. Grains providing dietary fiber include, but are not limited to, oats, rye, barley, and wheat. Legumes providing fiber include, but are not limited to, peas and beans such as soybeans. Fruits and vegetables providing a source of fiber include, but are not limited to, apples, oranges, pears, bananas, berries, tomatoes, green beans, broccoli, cauliflower, carrots, potatoes, celery. Plant foods such as bran, nuts, and seeds (such as flax seeds) are also sources of dietary fiber, Parts of plants providing dietary fiber include, but are not limited to, the stems, roots, leaves, seeds, pulp, and skin.

Fatty acids include any straight chain or branched monocarboxylic acid and include saturated fatty acids, unsaturated fatty acids, long chain fatty acids, medium chain fatty acids, short chain fatty acids, fatty acid precursors (including omega-9 fatty acid precursors), and esterified fatty acids. In an embodiment, the fatty acid is a straight chain monocarboxylic acid. As used herein, “long chain polyunsaturated fatty acid” refers to any polyunsaturated carboxylic acid or organic acid with a long aliphatic tail. Suitable omega-3 fatty acids include, but are not limited to, linolenic acid, alpha-linolenic acid, eicosapentaenoic acid, docosahexaenoic acid, stearidonic acid, eicosatetraenoic acid and combinations thereof.

Suitable omega-6 fatty acids include, but are not limited to, linoleic acid, gamma-linolenic acid, dihommo-gamma-linolenic acid, arachidonic acid, cicosadienoic acid, docosadienoic acid, adrenic acid, docosapentaenoic acid and combinations thereof. Suitable esterified fatty acids for embodiments of the present invention include, but are not limited to, monoacylgycerols containing omega-3 and/or omega-6 fatty acids, diacylgycerols containing omega-3 and/or omega-6 fatty acids, or triacylgycerols containing omega-3 and/or omega-6 fatty acids and combinations thereof.

Suitable vitamins include, vitamin A, vitamin D, vitamin E, vitamin K, vitamin B1, vitamin B2, vitamin B3, vitamin B5, vitamin B6, vitamin B7, vitamin B9, vitamin B12, and vitamin C. Various other compounds have been classified as vitamins by some authorities. These compounds may be termed pseudo-vitamins and include, but are not limited to, compounds such as ubiquinone (coenzyme Q10), pangamic acid, dimethylglycine, taestrile, amygdaline, flavanoids, para-aminobenzoic acid, adenine, adenylic acid, and s-methylmethionine. As used herein, the term vitamin includes pseudo-vitamins.

Minerals are selected from bulk minerals, trace minerals or combinations thereof. Non-limiting examples of bulk minerals include calcium, chlorine, magnesium, phosphorous, potassium, sodium, and sulfur, Non-limiting examples of trace minerals include chromium, cobalt, copper, fluorine, iron, manganese, molybdenum, selenium, zinc, and iodine. Although iodine generally is classified as a trace mineral, it is required in larger quantities than other trace minerals and often is categorized as a bulk mineral.

In other particular embodiments of this invention, the mineral is a trace mineral, believed to be necessary for human nutrition, non-limiting examples of which include bismuth, boron, lithium, nickel, rubidium, silicon, strontium, tellurium, tin, titanium, tungsten, and vanadium.

Preservatives are selected from antimicrobials, antioxidants, antienzymatics or combinations thereof. Non-limiting examples of antimicrobials include sulfites, propionates, benzoates, sorbates, nitrates, nitrites, bacteriocins, salts, sugars, acetic acid, dimethyl dicarbonate (DMDC), ethanol, and ozone, Sulfites include, but are not limited to, sulfur dioxide, sodium bisulfite, and potassium hydrogen sulfite. Propionates include, but are not limited to, propionic acid, calcium propionate, and sodium propionate. Benzoates include, but are not limited to, sodium benzoate and benzoic acid. Sorbates include, but are not limited to, potassium sorbate, sodium sorbate, calcium sorbate, and sorbic acid. Nitrates and nitrites include, but are not limited to, sodium nitrate and sodium nitrite. In yet another particular embodiment, the at least one preservative is a bacteriocin, such as, for example, nisin. In another particular embodiment, the preservative is ethanol. In still another particular embodiment, the preservative is ozone. Antienzymatics suitable for use as preservatives in particular embodiments of the invention include ascorbic acid, citric acid, and metal chelating agents such as ethylenediaminetetraacetic acid (EDTA).

Hydration products can be electrolytes, non-limiting examples of which include sodium, potassium, calcium, magnesium, chloride, phosphate, bicarbonate, and combinations thereof. Suitable electrolytes for use in particular embodiments of this invention are also described in U.S. Pat. No. 5,681,569, the disclosure of which is expressly incorporated herein by reference. Non-limiting examples of salts for use in particular embodiments include chlorides, carbonates, sulfates, acetates, bicarbonates, citrates, phosphates, hydrogen phosphates, tartrates, sorbates, citrates, benzoates, or combinations thereof.

In particular embodiments of this invention, the hydration product is a carbohydrate to supplement energy stores burned by muscles. Suitable carbohydrates for use in particular embodiments of this invention are described in U.S. Pat. Nos. 4,312,856, 4,853,237, 5,681,569, and 6,989,171, the disclosures of which are expressly incorporated herein by reference. Non-limiting examples of suitable carbohydrates include monosaccharides, disaccharides, oligosaccharides, complex polysaccharides or combinations thereof. Non-limiting examples of suitable types of monosaccharides for use in particular embodiments include trioses, tetroses, pentoses, hexoses, heptoses, octoses, and nonoses. Non-limiting examples of specific types of suitable monosaccharides include glyceraldehyde, dihydroxyacetone, erythrose, threose, erythrulose, arabinose, lyxose, ribose, xylose, ribulose, xylulose, allose, altrose, galactose, glucose, gulose, idose, mannose, talose, fructose, psicose, sorbose, tagatose, mannoheptulose, sedoheltulose, octolose, and sialose. Non-limiting examples of suitable disaccharides include sucrose, lactose, and maltose. Non-limiting examples of suitable oligosaccharides include saccharose, maltotriose, and maltodextrin. In other particular embodiments, the carbohydrates are provided by a corn syrup, a beet sugar, a cane sugar, a juice, or a tea. In another particular embodiment, the hydration is a flavanol that provides cellular rehydration. Non-limiting examples of suitable flavanols for use in particular embodiments of this invention include catechin, epicatechin, gallocatechin, epigallocatechin, epicatechin gallate, epigallocatechin 3-gallate, theaflavin, theaflavin 3-gallate, theaflavin 3′-gallate, theaflavin 3,3′ gallate, thearubigin or combinations thereof. In a particular embodiment, the hydration product is a glycerol solution to enhance exercise endurance.

Probiotics comprise microorganisms that benefit health when consumed in an effective amount. Probiotics may include, without limitation, bacteria, yeasts, and fungi. Examples of probiotics include, but are not limited to, bacteria of the genus Lactobacilli, Bifidobacteria, Streptococci, or combinations thereof. In particular embodiments of the invention, the at least one probiotic is chosen from the genus Lactobacilli. Lactobacilli (i.e., bacteria of the genus Lactobacillus, hereinafter “L.”). Non-limiting examples of species of Lactobacilli found in the human intestinal tract include L. acidophilus, L. casei, L. fermentum, L. saliva roes, L. brevis, L. leichmannii, L. plantarum, L. cellobiosus, L. reuteri, L. rhamnosus, L. GG, L. bulgaricus, and L. thermophilus. According to other particular embodiments of this invention, the probiotic is chosen from the genus Bifidobacteria. Non-limiting species of Bifidobacteria found in the human gastrointestinal tract include B. angulatum, B. animalis, B. asteroides, B. bifidum, B. boum, B. breve, B. catenulatum, B. choerinum, B. coryneforme, B. cuniculi, B. dentium, B. gallicum, B. gallinarum, B indicum, B. longum, B. magnum, B. merycicum, B. minimum, B. pseudocatenulatum, B. pseudolongum, B. psychraerophilum, B. pullorum, B. ruminantium, B. saeculare, B. scardovii, B. simiae, B. subtile, B. thermacidophilum, B. thermophilum, B. urinalis, and B. sp. According to other particular embodiments of this invention, the probiotic is chosen from the genus Streptococcus. Streptococcus thermophilus is a gram-positive facultative anaerobe. Other non-limiting probiotic species of this bacteria include Streptococcus salivarus and Streptococcus cremoris.

Prebiotics are compositions that promote the growth of beneficial bacteria in the intestines. Prebiotics include, without limitation, mucopolysaccharides, oligosaccharides, polysaccharides, amino acids, vitamins, nutrient precursors, proteins and combinations thereof. According to a particular embodiment, the prebiotic is chosen from dietary fibers, including, without limitation, polysaccharides and oligosaccharides. Non-limiting examples of oligosaccharides that are categorized as prebiotics in accordance with particular embodiments of this invention include fructooligosaccharides, inulins, isomalto-oligosaccharides, lactilol, lactosucrose, lactulose, pyrodextrins, soy oligosaccharides, transgalacto-oligosaccharides, and xylo-oligosaccharides. According to other particular embodiments, the prebiotic is an amino acid.

As used herein, “a weight management agent” includes an appetite suppressant and/or a thermogenesis agent. As used herein, the phrases “appetite suppressant”, “appetite satiation compositions”, “satiety agents”, and “satiety ingredients” are synonymous. The phrase “appetite suppressant” describes macronutrients, herbal extracts, exogenous hormones, anorectics, anorexigenics, pharmaceutical drugs, and combinations thereof, that when delivered in an effective amount, suppress, inhibit, reduce, or otherwise curtail a person's appetite. The phrase “thermogenesis agent” describes macronutrients, herbal extracts, exogenous hormones, anorectics, anorexigenics, pharmaceutical drugs, and combinations thereof, that when delivered in an effective amount, activate or otherwise enhance a person's thermogenesis or metabolism.

Suitable weight management agents include macronutrients selected from the group consisting of proteins, carbohydrates, dietary fats, and combinations thereof. Carbohydrates generally comprise sugars, starches, cellulose and gums that the body converts into glucose for energy. Non-limiting examples of carbohydrates include polydextrose; inulin; monosaccharide-derived polyols such as erythritol, mannitol, xylitol, and sorbitol; disaccharide-derived alcohols such as isomalt, lactitol, and maltitol; and hydrogenated starch hydrolysates. Carbohydrates are described in more detail herein below. Dietary fats are lipids comprising combinations of saturated and unsaturated fatty acids. Polyunsaturated fatty acids have been shown to have a greater satiating power than mono-unsaturated fatty acids. Accordingly, the dietary fats embodied herein desirably comprise poly-unsaturated fatty acids, non-limiting examples of which include triacylglycerols.

In a particular embodiment, the weight management agent is a herbal extract. Non-limiting examples of plants whose extracts have appetite suppressant properties include plants of the genus Hoodia, Trichocaulon, Caralluma, Stapelia, Orbea, Asclepias, and Camelia. Other embodiments include extracts derived from Gymnema sylvestre, Kola Nut, Citrus aurantium, Yerba Mate, Griffonia simplicifolia, Guarana, myrrh, guggul Lipid, and black current seed oil. In a particular embodiment, the herbal extract is derived from a plant of the genus Hoodia, species of which include H. alstonii, H. currorii, H. dregei, H. flava, H. gordonii, H. jutatae, H. mossamedensis, H. officinalis, H. parviflorai, H. pedicellata, H. pifera, H. ruschil, and H. triebneri. Hoodia plants are stem succulents native to southern Africa.

In another particular embodiment, the herbal extract is derived from a plant of the genus Caralluma, species of which include C. indica, C. fimbriata, C. attenuate, C. tuberculata, C. edulis, C. adscendens, C. stalagmifera, C. umbellate, C. penicillata, C. russeliana, C. retrospicens, C. arabica, and C. lasiantha. Carralluma plants belong to the same Subfamily as Hoodia, Asclepiadaceae.

In another particular embodiment, the at least one herbal extract is derived from a plant of the genus Trichocaulon. Trichocaulon plants are succulents that generally are native to southern Africa, similar to Hoodia, and include the species T. piliferum and T. officinale. In another particular embodiment, the herbal extract is derived from a plant of the genus Stapelia or Orbea, species of which include S. gigantean and O. variegate, respectively. Both Stapelia and Orbea plants belong to the same Subfamily as Hoodia, Asclepiadaceae.

In another particular embodiment, the herbal extract is derived from a plant of the genus Asclepias. Asclepias plants also belong to the Asclepiadaceae family of plants. Non-limiting examples of Asclepias plants include A. incarnate, A. curassayica, A. syriaca, and A. tuberose. Not wishing to be bound by any theory, it is believed that the extracts comprise steroidal compounds, such as pregnane glycosides and pregnane aglycone, having appetite suppressant effects. In a particular embodiment, the weight management agent is an exogenous hormone having a weight management effect. Non-limiting examples of such hormones include CCK, peptide YY, ghrelin, bombesin and gastrin-releasing peptide (GRP), enterostatin, apolipoprotein A-IV, GLP-1, amylin, somastatin, and leptin.

In certain embodiments, the osteoporosis management agent is at least one calcium source, i.e. any compound containing calcium, including salt complexes, solubilized species, and other forms of calcium. Non-limiting examples of calcium sources include amino acid chelated calcium, calcium carbonate, calcium oxide, calcium hydroxide, calcium sulfate, calcium chloride, calcium phosphate, calcium hydrogen phosphate, calcium dihydrogen phosphate, calcium citrate, calcium malate, calcium citrate malate, calcium gluconate, calcium tartrate, calcium lactate, solubilized species thereof, and combinations thereof, According to a particular embodiment, the osteoporosis management agent is a magnesium source, i.e. any compound containing magnesium, including salt complexes, solubilized species, and other forms of magnesium. Non-limiting examples of magnesium sources include magnesium chloride, magnesium citrate, magnesium gluceptate, magnesium gluconate, magnesium lactate, magnesium hydroxide, magnesium picolate, magnesium sulfate, solubilized species thereof, and mixtures thereof. In another particular embodiment, the magnesium source comprises an amino acid chelated or creatine chelated magnesium. In other embodiments, the osteoporosis agent is chosen from vitamins D, C, K, their precursors and/or beta-carotene and combinations thereof. Numerous plants and plant extracts also have been identified as being effective in the prevention and treatment of osteoporosis. Not wishing to be bound by any theory, it is believed that the plants and plant extracts stimulates bone morphogenic proteins and/or inhibits bone resorption, thereby stimulating bone regeneration and strength. Non-limiting examples of suitable plants and plant extracts as osteoporosis management agents include species of the genus Taraxacum and Amelanchier, as disclosed in U.S. Patent Publication No. 2005/0106215, and species of the genus Lindera, Artemisia, Acorus, Carthamus, Carum, Cnidium, Curcuma, Cyperus, Juniperus, Prunus, Iris, Cichorium, Dodonaea, Epimedium, Erigonoum, Soya, Mentha, Ocimum, thymus, Tanacetum, Plantago, Spearmint, Bixa, Vitis, Rosemarinus, Rhus, and Anethum, as disclosed in U.S. Patent Publication No. 2005/0079232.

Examples of suitable phytoestrogens for embodiments of this invention include, but are not limited to, isoflavones, stilbenes, lignans, resorcyclic acid lactones, coumestans, coumestrol, equol, and combinations thereof. Isoflavones belong to the group of phytonutrients called polyphenols. In general, polyphenols (also known as “polyphenolics”), are a group of chemical substances found in plants, characterized by the presence of more than one phenol group per molecule. Suitable phytoestrogen isoflavones in accordance with embodiments of this invention include genistein, daidzein, glycitein, biochanin A, formononetin, their respective naturally occurring glycosides and glycoside conjugates, matairesinol, secoisolariciresinol, enterolactone, enterodiol, textured vegetable protein, and combinations thereof.

Long-chain primary aliphatic saturated alcohols are a diverse group of organic compounds. The term long-chain refers to the fact that the number of carbon atoms in these compounds is at least 8 carbons. Non-limiting examples of particular long-chain primary aliphatic saturated alcohols for use in particular embodiments of the invention include the 8 carbon atom 1-octanol, the 9 carbon 1-nonanol, the 10 carbon atom 1-decanol, the 12 carbon atom 1-dodecanol, the 14 carbon atom 1-tetradecanol, the 16 carbon atom 1-hexadecanol, the 18 carbon atom 1-octadecanol, the 20 carbon atom 1-eicosanol, the 22 carbon 1-docosanol, the 24 carbon 1-tetracosanol, the 26 carbon 1-hexacosanol, the 27 carbon 1-heptacosanol, the 28 carbon 1-octanosol, the 29 carbon 1-nonacosanol, the 30 carbon 1-triacontanol, the 32 carbon 1-dotriacontanol, and the 34 carbon 1-tetracontanol. In a particularly desirable embodiment of the invention, the long-chain primary aliphatic saturated alcohols are policosanol. Policosanol is the term for a mixture of long-chain primary aliphatic saturated alcohols composed primarily of 28 carbon 1-octanosol and 30 carbon 1-triacontanol, as well as other alcohols in lower concentrations such as 22 carbon 1-docosanol, 24 carbon 1-tetracosanol, 26 carbon 1-hexacosanol, 27 carbon 1-heptacosanol, 29 carbon 1-nonacosanol, 32 carbon 1-dotriacontanol, and 34 carbon 1-tetracontanol.

At least 44 naturally occurring phytosterols have been discovered, and generally are derived from plants, such as corn, soy, wheat, and wood oils; however, they also may be produced synthetically to form compositions identical to those in nature or having properties similar to those of naturally-occurring phytosterols. According to particular embodiments of this invention, non-limiting examples of phytosterols well known to those or ordinary skill in the art include 4-desmethylsterols (e.g., β-sitosterol, campesterol, stigmasterol, brassicasterol, 22-dehydrobrassicasterol, and Δ5-avenasterol), 4-monomethyl sterols, and 4,4-dimethyl sterols (triterpene alcohols) (e.g., cycloartenol, 24-methylenecycloartanol, and cyclobranol).

According to particular embodiments of this invention, non-limiting examples of phytostanols include β-sitostanol, campestanol, cycloartanol, and saturated forms of other triterpene alcohols.

Both phytosterols and phytostanols, as used herein, include the various isomers such as the α and β isomers (e.g., α-sitosterol and β-sitostanol, which comprise one of the most effective phytosterols and phytostanols, respectively, for lowering serum cholesterol in mammals). The phytosterols and phytostanols of the present invention also may be in their ester form. Non-limiting examples of suitable phytosterol and phytostanol esters include sitosterol acetate, sitosterol oleate, stigmasterol oleate, and their corresponding phytostanol esters. The phytosterols and phytostanols of the present invention also may include their derivatives.

Generally, the amount of functional ingredient in the composition varies widely depending on the particular composition and the desired functional ingredient. Those of ordinary skill in the art will readily ascertain the appropriate amount of functional ingredient for each composition.

### Consumables/Products for Oral Administration

Disclosed herein are consumables (e.g., food and/or beverages) comprising the sweetener compositions or flavor modifying compositions disclosed herein.

Myd (HTS) polypeptides (or variants thereof) or the Myd-containing sweetener compositions or flavor modifying compositions disclosed herein can be incorporated in any known edible material (referred to herein as a “consumable,” “consumable product”, a “sweetenable composition” or “flavor and/or taste modified composition”), such as, edible gel mixes and compositions, dental compositions, foodstuffs (confections, condiments, chewing gum, cereal compositions, baked goods, dairy products, and tabletop sweetener compositions) beverages and beverage products. “A consumable product” herein includes concentrates, mixes, powders and other solid materials which can be combined with other edible ingredients to produce a final consumable product (e.g., cake, cookie, bread or related mixes; or seasoning, salad dressing or gravy mixes or packets).

In one embodiment, the consumable is a beverage or beverage product. Put another way, disclosed herein is a beverage or beverage product comprising Myd polypeptides (or variants thereof) or the Myd-containing sweetener compositions or flavor modifying compositions disclosed herein.

As used herein a “beverage product” is a ready-to-drink beverage, a beverage concentrate, a beverage syrup, or a powdered beverage. Suitable ready-to-drink beverages include carbonated and non-carbonated beverages. Carbonated beverages include, but are not limited to, cola, ginger ale, soft drinks and root beer. Non-carbonated beverages include, but are not limited to fruit juice, fruit-flavored juice, vegetable juice, vegetable-flavored juice, sports drinks, energy drinks, plant protein beverage (including plant-based milks), near water drinks (e.g., water with natural or synthetic flavorants), tea type (e.g. black tea, green tea, red tea, oolong tea), coffee, cocoa drink, beverage containing milk components (e.g. milk beverages, coffee containing milk components, café au lait, milk tea, fruit milk beverages).

In particular embodiments, the beverage is a flavored black tea beverage, a zero calorie enhanced water beverage or an orange-flavored sparkling beverage.

Beverage concentrates and beverage syrups are prepared with an initial volume of liquid matrix (e.g. water) and the desired beverage ingredients, Full strength beverages are then prepared by adding further volumes of water. Powdered beverages are prepared by dry-mixing all of the beverage ingredients in the absence of a liquid matrix. Full strength beverages are then prepared by adding the full volume of water.

Beverages contain water as the liquid matrix. As used herein, a “liquid matrix” refers to the basic ingredient in which the ingredients—including the sweetener or sweetener compositions—are dissolved. Water of beverage quality, such as, for example deionized water, distilled water, reverse osmosis water, carbon-treated water, purified water, demineralized water and combinations thereof, can be used. Additional suitable liquid matrices include, but are not limited to phosphoric acid, phosphate buffer, citric acid, citrate buffer and carbon-treated water.

In one embodiment, a beverage or beverage product is disclosed that contains Myd polypeptides or a variant thereof. In a particular embodiment, the beverage contains a Myd variant that has the same or increased sweetness relative to a beverage containing the same amount of wild-type Myd.

In one embodiment, the concentration of Myd (or a variant thereof) in the beverage is at or above its sweetness recognition threshold concentration or flavor recognition threshold concentration. In a particular embodiment, the concentration of Myd is at least about 1%, at least about 5%, at least about 10%, at least about 15%, at least about 20%, at least about 25%, at least about 30%, about least about 35%, at least about 40%, about least about 45%, at least about 50% or more above its sweetness or flavor recognition threshold concentration.

In one embodiment, the beverage or beverage product contains Myd (or variant thereof) in an amount from about 1 ppm to about 50 ppm, such as, for example, from about 5 ppm to about 50 ppm, from about 5 ppm to about 40 ppm, from about 5 ppm to about 30 ppm, from about 5 ppm to about 20 ppm, from about 5 ppm to about 10 ppm, from about 10 ppm to about 50 ppm, from about 10 ppm to about 40 ppm, from about 10 ppm to about 30 ppm, from about 10 ppm to about 20 ppm, from about 20 ppm to about 50 ppm, from about 20 ppm to about 40 ppm, from about 20 ppm to about 30 ppm, from about 30 ppm to about 50 ppm, from about 30 ppm to about 40 ppm or from about 40 ppm to about 50 ppm.

In a more particular embodiment, the beverage or beverage product contains Myd (or variant thereof) in an amount from about 10 ppm to about 25 ppm.

In another embodiment, the beverage contains Myd (or variant thereof) in an amount from about 1 ppm to less than about 15 ppm. In one embodiment, the beverage contains Myd (or variant thereof) in an amount from about 1 ppm to about 5 ppm, about 5 ppm to about 10 ppm or about 10 ppm to about 15 ppm.

In another embodiment, the beverage contains Myd (or variant thereof) in an amount greater than about 15 ppm. In one embodiment, the beverage contains Myd (or variant thereof) in an amount between about 15 ppm and about 20 ppm, about 20 ppm and about 25 ppm, about 25 ppm and about 30 ppm, about 30 ppm and about 35 ppm, about 35 ppm and about 40 ppm, about 40 ppm and about 50 ppm, or about 50 ppm or greater.

In one embodiment, the beverage contains Myd (or variant thereof) in an amount of about 1 ppm, about 3 ppm, about 5 ppm, about 10 ppm, about 12 ppm, about 15 ppm, about 18 ppm, about 20 ppm, about 22 ppm, about 25 ppm, about 28 ppm, about 30 ppm, about 35 ppm, about 40 ppm, about 45 ppm, about 50 ppm, about 55 ppm, about 60 ppm, about 65 ppm, about 70 ppm or about 75 ppm or greater.

The beverage can further include at least one additional sweetener. Any of the sweeteners detailed herein can be used, including natural, non-natural, or synthetic sweeteners.

In a particular embodiment, the at least one addition sweetener is one or more steviol glycosides. In a particular embodiment, the steviol glycoside is Reb M. In another particular embodiment, the steviol glycoside is Reb A. In one embodiment, the beverage contains, in addition to Myd (or variant thereof) a steviol glycoside blend, e.g., Reb M and Reb A.

The amount of the steviol glycoside (e.g., Reb M, Reb A) may vary. In one embodiment, the amount of the steviol glycoside provides less than about 8 sucrose equivalence (SE), e.g., about 7.5 SE, about 7.0 SE, about 6.5 SE or about 6.0 SE or less. In another embodiment, the amount of steviol glycoside provides greater than about 8 sucrose equivalence (SE), e.g., about 8.5 SE, about 9.0 SE, about 9.5 SE, about 10 SE, about 10.5 SE, about 11 SE or about 12 SE or more.

In another embodiment, the amount of steviol glycoside (e.g., Reb M, Reb A) is less than about 450 ppm. In a particular embodiment, the amount of steviol glycoside is less than about 400 ppm, less than about 350 ppm, less than about 300 ppm, less than about 300 ppm, less than about 250 ppm, less than about 200 ppm, less than about 150 ppm or about 100 ppm or less.

In another embodiment, the amount of steviol glycoside (e.g., Reb M, Reb A) is between about 100 ppm and about 450 ppm, more particularly, between about 100 ppm and about 400 ppm, between about 100 ppm and about 350 ppm, between about 100 ppm and about 300 ppm, between about 100 ppm and about 250 ppm, between about 100 ppm and about 200 ppm, or between about 100 ppm and 250 ppm.

In a further embodiment, the amount of the steviol glycoside (e.g., Reb M, Reb A) is between about 100 ppm and about 200 ppm, more particularly, about 110 ppm, about 120 ppm, about 130 ppm, about 140 ppm, about 150 ppm, about 160 ppm, about 170 ppm, about 180 ppm, about 190 ppm or about 200 ppm.

In a further embodiment, the amount of the steviol glycoside (e.g., Reb M, Reb A) is between about 200 ppm and about 300 ppm, and more particularly, about 210 ppm, about 220 ppm, about 230 ppm, about 240 ppm, about 250 ppm, about 260 ppm, about 270 ppm, about 280 ppm, about 290 ppm, or about 300 ppm.

In another embodiment, the amount of the steviol glycoside (e.g., Reb M, Reb A) is between about 300 ppm and about 400 ppm, and more particularly, about 310 ppm, about 320 ppm, about 330 ppm, about 340 ppm, about 350 ppm, about 360 ppm, about 370 ppm, about 380 ppm, about 390 ppm or about 400 ppm.

In another embodiment, the amount of the steviol glycoside (e.g., Reb M, Reb A) is between about 400 ppm and about 500 ppm, and more particularly, about 410 ppm, about 420 ppm, about 430 ppm, about 440 ppm, about 450 ppm, about 460 ppm, about 470 ppm or about 480 ppm.

In a particular embodiment, the beverage comprises Myd (or variant thereof) in an amount between about 1 ppm and about 100 ppm and at least one steviol glycoside (e.g., Reb M, Reb A), wherein the amount of the at least one steviol glycoside is sufficient to provide less than about 8 SE, e.g., about 7.5 SE, about 7.0 SE, about 6.5 SE, about 6 SE, about 5.5 SE, about 5.0 SE, about 4.5 SE, about 4.0 SE, about 3.5 SE, about 3.0 SE, about 2.5 SE, about 2.0 SE, about 1.5 SE or about 1.0 SE.

In another particular embodiment, the beverage comprises beverage comprises Myd (or variant thereof) in an amount between about 1 ppm and about 100 ppm and at least one steviol glycoside (e.g., Reb M, Reb A), wherein the amount of the at least one steviol glycoside is about 450 ppm or less, e.g., about 425 ppm, about 400 ppm, about 375 ppm, about 350 ppm, about 325 ppm, about 300 ppm, about 275 ppm, about 250 ppm, about 225 ppm, about 200 ppm, about 175 ppm, about 150 ppm, about 125 ppm or about 100 ppm or less.

In a particular embodiment, the beverage comprises Myd (or variant thereof) in an amount between about 1 ppm and about 30 ppm, more particularly, about 1 ppm and about 15 ppm or about 15 ppm and about 30 ppm, and Reb M in an amount sufficient to provide less than about 8 SE, for example, e.g., about 7.5 SE, about 7.0 SE, about 6.5 SE, about 6 SE, about 5.5 SE, about 5.0 SE, about 4.5 SE, about 4.0 SE, about 3.5 SE, about 3.0 SE, about 2.5 SE, about 2.0 SE, about 1.5 SE or about 1.0 SE.

In another particular embodiment, the beverage comprises Myd (or variant thereof) in an amount between about 1 ppm and about 30 ppm, more particularly, between about 1 ppm and about 15 ppm or between about 15 ppm and about 30 ppm, and Reb M in an amount about 450 ppm or less, e.g., about 425 ppm, about 400 ppm, about 375 ppm, about 350 ppm, about 325 ppm, about 300 ppm, about 275 ppm, about 250 ppm, about 225 ppm, about 200 ppm, about 175 ppm, about 150 ppm, about 125 ppm or about 100 ppm or less.

In another particular embodiment, a beverage comprises Myd (or variant thereof) in an amount between about 1 ppm and about 30 ppm, more particularly, between about 1 ppm and about 15 ppm or between about 15 ppm and about 30 ppm, and Reb M in an amount about 450 ppm or less, e.g., about 425 ppm, about 400 ppm, about 375 ppm, about 350 ppm, about 325 ppm, about 300 ppm, about 275 ppm, about 250 ppm, about 225 ppm, about 200 ppm, about 175 ppm, about 150 ppm, about 125 ppm or about 100 ppm or less.

For example, a beverage comprises Myd (or variant thereof) in an amount from about 1 ppm to about 30 ppm and RebM80 in an amount from about 200 ppm to about 400 ppm. In a more particular embodiment, a beverage comprises Myd (or variant thereof) in an amount from about 20 ppm to about 30 ppm and RebM80 in an amount from about 300 to about 400 ppm. In a still more particular embodiment, a beverage comprises Myd (or variant thereof) in an amount of about 25 ppm and RebM80 in an amount of about 315 ppm.

A beverage of the present invention comprises Myd (or variant thereof) in an amount from about 1 ppm to about 50 ppm and RebM80 in an amount from about 200 ppm to about 400 ppm. In preferred embodiments, the beverage of the present invention tastes similar to a beverage sweetened with RebM or RebM80 only, wherein the beverage of the present invention and the RebM or RebM80-sweetened beverages have similar sucrose equivalence. In a more particular embodiment, a beverage having a citric acid matrix comprises Myd (or variant thereof) in an amount from about 20 ppm to about 30 ppm and RebM80 in an amount from about 300 to about 400 ppm. In a still more particular embodiment, a beverage having a citric acid matrix comprises Myd (or variant thereof) in an amount of about 25 ppm and RebM80 in an amount of about 315 ppm. In another more particular embodiment, a lemon-lime diet carbonated beverage comprises Myd (or variant thereof) in an amount from about 30 ppm to about 50 ppm and RebM80 in an amount from about 150 ppm to about 350 ppm. In a still more particular embodiment, a lemon-lime diet carbonated beverage comprises Myd (or variant thereof) in an amount of about 40 ppm and RebM80 in an amount of about 210 ppm.

In a particular embodiment, the beverage comprises Myd (or variant thereof) in an amount between about 10 ppm and about 25 ppm and Reb M in an amount sufficient to provide less than about 8 SE, e.g., about 7.5 SE, about 7.0 SE, about 6.5 SE, about 6 SE, about 5.5 SE, about 5.0 SE, about 4.5 SE, about 4.0 SE, about 3.5 SE, about 3.0 SE, about 2.5 SE, about 2.0 SE, about 1.5 SE or about 1.0 SE.

In another particular embodiment, the beverage comprises Myd (or variant thereof) in an amount between about 10 ppm and about 25 ppm, and Reb M in an amount about 450 ppm or less, e.g., about 425 ppm, about 400 ppm, about 375 ppm, about 350 ppm, about 325 ppm, about 300 ppm, about 275 ppm, about 250 ppm, about 225 ppm, about 200 ppm, about 175 ppm, about 150 ppm, about 125 ppm or about 100 ppm or less.

In another particular embodiment, the beverage comprises Myd (or variant thereof) in an amount between about 1 ppm and about 50 ppm and at least one additional sweetener (e.g., Reb M, Reb A, siamenoside I, mogroside IV), wherein the amount of the at least one additional sweetener is less than about 450 ppm, e.g., about 425 ppm, about 400 ppm, about 375 ppm, about 350 ppm, about 325 ppm, about 300 ppm, about 275 ppm, about 250 ppm, about 225 ppm, about 200 ppm, about 175 ppm, about 150 ppm, about 125 ppm or about 100 ppm or less.

In a particular embodiment, the beverage comprises Myd (or variant thereof) in an amount between about 1 ppm and about 100 ppm and Reb A in an amount sufficient to provide less than about 8 SE., e.g., about 7.5 SE, about 7.0 SE, about 6.5 SE, about 6 SE, about 5.5 SE, about 5.0 SE, about 4.5 SE, about 4.0 SE, about 3.5 SE, about 3.0 SE, about 2.5 SE, about 2.0 SE, about 1.5 SE or about 1.0 SE.

In a particular embodiment, the beverage comprises Myd (or variant thereof) in an amount between about 1 ppm and about 30 ppm, more particularly between about 1 ppm and about 15 ppm or between about 15 ppm and about 30 ppm, Reb A in an amount less than about 450 ppm, e.g., about 425 ppm, about 400 ppm, about 375 ppm, about 350 ppm, about 325 ppm, about 300 ppm, about 275 ppm, about 250 ppm, about 225 ppm, about 200 ppm, about 175 ppm, about 150 ppm, about 125 ppm or about 100 ppm or less.

In a particular embodiment, the beverage comprises Myd (or variant thereof) in an amount between about 10 ppm and about 25 ppm and Reb A in an amount sufficient to provide less than about 8 SE., e.g., about 7.5 SE, about 7.0 SE, about 6.5 SE, about 6 SE, about 5.5 SE, about 5.0 SE, about 4.5 SE, about 4.0 SE, about 3.5 SE, about 3.0 SE, about 2.5 SE, about 2.0 SE, about 1.5 SE or about 1.0 SE.

In a particular embodiment, the beverage comprises Myd (or variant thereof) in an amount between about 10 ppm and about 25 ppm, Reb A in an amount less than about 450 ppm, e.g., about 425 ppm, about 400 ppm, about 375 ppm, about 350 ppm, about 325 ppm, about 300 ppm, about 275 ppm, about 250 ppm, about 225 ppm, about 200 ppm, about 175 ppm, about 150 ppm, about 125 ppm or about 100 ppm or less.

The ppm ratio of Myd (or variant thereof) and the one or more steviol glycosides in the beverage may vary. In one embodiment, the ppm ratio of Myd (or variant thereof) and the one or more steviol glycosides is between about 1:2 and about 1:40, more particularly, between about 1:5 and about 1:35, even more particularly, between about 1:10 and about 1:25.

In a particular embodiment, the ppm ratio of Myd (or variant thereof) and the one or more steviol glycosides is about 1:30 or less, more particularly, about 1:25.

In one embodiment, the steviol glycoside is Reb M and the ppm ratio of Myd (or variant thereof) and Reb M is between about 1:10 and about 1:20, more particularly, about 1:10, about 1:12, about 1:14, about 1:16, about 1:18 or about 1:20.

In another embodiment, the steviol glycoside is Reb A and the ppm ratio of Myd (or variant thereof) and Reb A is about 1:40 or less, more particularly, between about 1:20 and about 1:30, even more particularly, about 1:20, about 1:22, about 1:24, about 1:26, about 1:28 or about 1:30.

In another embodiment, the steviol glycoside is Reb A and the ppm ratio of Myd (or variant thereof) and Reb A is about 1:70 or less, more particularly, less than about 1:60, less than about 1:50 or less than about 1:40. In a particular embodiment, the ppm ratio of Myd (or variant thereof) and Reb A is about 1:35.

In another embodiment, the steviol glycoside is Reb A and the ppm ratio of Myd (or variant thereof) and Reb A is about 1:15 or greater, more particularly, greater than about 1:17, about 1:18, about 1:19 or about 1:20.

In another embodiment, the steviol glycoside is Reb A and the ppm ratio of Myd (or variant thereof) and Reb A is about 1:10 or greater, more particularly, greater than about 1:12, about 1:14 or about 1:16.

In one embodiment, the beverage comprises Myd (or variant thereof) and Reb M, wherein Myd is present in an amount from about 15 ppm to about 30 ppm, more particularly, about 20 ppm to about 25 ppm, and Reb M (e.g., Reb M80) is present in an amount from about 300 ppm to about 350 ppm, more particularly, about 315 ppm to about 325 ppm (i.e. the Reb M provides about 8 SE). According to this embodiment, the ratio of Myd (or variant thereof) to Reb M is between about 1:10 and about 1:20, more particularly about 1:0 1:12, about 1:14, about 1:16, about 1:18 or about 1:20. In one embodiment, the amount of Reb M provides about 8 sucrose equivalence (SE).

In one embodiment, the beverage comprises Myd (or variant thereof) and Reb M, wherein Myd is present in an amount from about 1 ppm to about 20 ppm, more particularly about 1 ppm to about 15 ppm, and Reb M is present in an amount of about 110 ppm to about 140 ppm, more particularly about 115 ppm, and even more particularly, about 120 ppm. In a particular embodiment, the ppm ratio of Myd (or variant thereof) to Reb M is about 1:5 to about 1:10, more particularly about 1:7.

In one embodiment, the beverage comprises Myd (or variant thereof) and Reb M, wherein Myd is present in an amount from about 1 ppm to about 15 ppm, more particularly, about 1 ppm to about 5 ppm, even more particularly, about 3 ppm, and Reb M is present in an amount between about 450 ppm and about 500 ppm, more particularly, about 470 ppm to about 475 ppm, even more particularly, about 472 ppm. In a particular embodiment, Reb M is present in an amount that provides greater than about 9 sucrose equivalence (SE).

In one embodiment, the beverage comprises Myd (or variant thereof) and Reb A, wherein Myd is present in an amount from about 10 ppm to about 20 ppm, more particularly, about 15 ppm to about 20 ppm, and Reb A is present in an amount of about 375 ppm to about 425 ppm, more particularly, about 400 ppm. In one embodiment, the amount of Reb A provides about 7 SE. According to this embodiment, a ppm ratio of Myd (or variant thereof) to Reb A between about 1:20 ppm and about 1:30 ppm ratio of Myd (or variant thereof) to Reb A has at least one improved organoleptic property (e.g., taste) compared to a ppm ratio of Myd (or variant thereof) to Reb A, more particularly about 1:20 ppm, about 1:22 ppm, about 1:24 ppm, about 1:26 ppm, about 1:28 ppm or about 1:30 ppm.

In one embodiment, the beverage comprises Myd (or variant thereof) and Reb A, wherein Myd is present in an amount between about 5 ppm and about 15 ppm, more particular, about 10 ppm, and Reb A is present in an amount of between about 325 ppm and about 375 ppm, more particularly, about 350 ppm. In one embodiment, the amount of Reb A provides between about 6 and about 7 sucrose equivalence (SE). According to this embodiment, a ppm ratio of Myd (or variant thereof) to Reb A of about 1:30 has at least one improved organoleptic property compared to a ppm ratio of Myd (or variant thereof) to Reb A of about 1:70 or about 1:17.

In one embodiment, a beverage is provided that comprises Myd (or variant thereof) and Reb A, wherein the amount of Myd (or variant thereof) is between about 25 ppm and about 35 ppm, more particularly, about 30 ppm, and the amount of Reb A is between about 220 ppm and about 240 μm, more particularly, about 230 ppm. According to this embodiment, a ppm ratio of Myd (or variant thereof) to Reb A of greater than about 1:10 has at least one improved organoleptic property than a ppm ratio of Myd (or variant thereof) to Reb A of less than about 1:10 ppm. In a particular embodiment, a ppm ratio of Myd (or variant thereof) to Reb A of about 1:12, about 1:14, about 1:16 or about 1:18 has at least one improved organoleptic property than a ppm ratio of Myd (or variant thereof) of about 1:8, about 1:6, about 1:4, about 1:2 or about 1:1 ppm.

In one embodiment, a beverage is provided that comprises Myd (or variant thereof) and Reb A, wherein the amount of Myd (or variant thereof) is between about 50 ppm and about 70 ppm, more particularly, about 60 ppm, and the amount of Reb A is between about 100 ppm and about 130 ppm, more particularly, about 120 ppm. According to this embodiment, the ppm ratio of Myd (or variant thereof) to Reb A is about 1:2.

In one embodiment, a beverage comprises from about 300 ppm to about 350 ppm Reb A and from about 10 ppm to about 50 ppm Myd (or variant thereof). In preferred embodiments, the beverage comprising Reb A and Myd (or variant thereof) tastes better and/or has positive synergy compared to a Reb A-sweetened beverage, wherein the beverage comprising Reb A and Myd (or variant thereof) and beverage sweetened with Reb A have the same sucrose equivalence.

In one embodiment, a beverage comprises from about 30 ppm to about 50 ppm Myd (or variant thereof), from about 300 ppm to about 450 ppm siamenoside I, and optionally from about 50 ppm to about 100 ppm RebM80. In preferred embodiments, the beverage tastes similar or better than either a siamenoside I-sweetened control or a RebM80-sweetened control, wherein the beverage of the present invention and the siamenoside I- and/or RebM80-sweetened control beverages have the same sucrose equivalence. In a particular embodiment, a beverage comprises from about 40 ppm to about 50 ppm Myd (or variant thereof) and from about 350 ppm to about 450 ppm siamenoside I, with no RebM80. In a more particular embodiment, the beverage comprises from about 40 ppm to about 45 ppm Myd (or variant thereof) and from about 350 ppm to about 450 ppm siamenoside I, with no RebM80. In a still more particular embodiment, the beverage comprises about 45 ppm Myd (or variant thereof) and about 410 ppm siamenoside I, with no RebM80. In another embodiment, a beverage comprises from about 25 to about 30 ppm Myd (or variant thereof), from about 300 ppm to about 400 ppm siamenoside I and from about 50 ppm to about 100 ppm RebM80. In a more particular embodiment, a beverage comprises about 30 ppm Myd (or variant thereof), about 350 ppm siamenoside I and about 70 ppm RebM80.

In one embodiment, a beverage comprises from about 30 to about 50 ppm Myd (or variant thereof) and from about 7 Brix to about 10 Brix high fructose corn syrup (HFCS). In preferred embodiments, the beverage of the present invention tastes similar or better than a HFCS-sweetened control (10 Brix). In a more particular embodiment, the beverage comprises from about 35 ppm to about 45 ppm Myd (or variant thereof) and about 7 Brix to about 9 Brix HFCS, In a still more particular embodiment, the beverage comprises about 40 ppm Myd (or variant thereof) and about 8 Brix HFCS.

In one embodiment, a reduced calorie beverage comprises from about 5 ppm to about 20 ppm Myd (or variant thereof) and from about 100 ppm to about 200 ppm RebM. In preferred embodiments, the beverage of the present invention has a taste profile similar to a sucrose-sweetened control (10 Brix). In a particular embodiment, a reduced calorie still (non-carbonated) beverage comprises from about 10 ppm to about 15 ppm Myd (or variant thereof) and from about 100 ppm to about 150 ppm RebM. In a still more particular embodiment, a reduced calorie still beverage comprises from about 10 ppm to about 15 ppm Myd (or variant thereof) and about 115 ppm RebM. In another particular embodiment, a reduced calorie carbonated beverage comprises about 20 ppm Myd (or variant thereof) and from about 100 to about 150 ppm RebM.

In one embodiment, a beverage comprises from about 5 ppm to about 20 ppm Myd (or variant thereof) and from about 300 ppm to about 500 ppm A95. In a particular embodiment, the beverage comprises from about 15 ppm to about 20 ppm Myd (or variant thereof) and about 300 ppm to about 500 ppm A95. In a further embodiment, the beverage comprises about 15 ppm or about 20 ppm Myd (or variant thereof) and about 400 ppm A95.

In one embodiment, the temporal profile, flavor profile and/or taste profile of the beverage is improved relative to a beverage that either does not contain Myd (or variant thereof) or contains Myd (or variant thereof) in an amount less than from about 20 ppm, even more particularly, less than about 15 ppm. In a particular embodiment, the temporal profile, flavor profile and/or taste profile of the beverage is more sugar-like. In one embodiment, the beverage has reduced sweetness linger, reduced bitterness, reduced bitter aftertaste, reduced astringency, improved mouthfeel (e.g., greater fullness or body) or the like.

The beverages can further include additives including, but are not limited to, carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, flavorants and flavoring ingredients, astringent compounds, proteins or protein hydrolysates, surfactants, emulsifiers, flavonoids, alcohols, polymers and combinations thereof. Any suitable additive described herein can be used.

The beverage can further contain one or more functional ingredients, detailed above. Functional ingredients include, but are not limited to, vitamins, minerals, antioxidants, preservatives, glucosamine, polyphenols and combinations thereof. Any suitable functional ingredient described herein can be used.

It is contemplated that the pH of the sweetened composition, such as, for example, a beverage, does not materially or adversely affect the taste of the sweetener. A non-limiting example of the pH range of the sweetenable composition may be from about 1.8 to about 8. A further example includes a pH range from about 2 to about 5. In a particular embodiment, the pH of a beverage is from about 3 to about 3.25. In a particular embodiment, the pH of a beverage is from about 2.5 to about 3.0.

The temperature of a beverage comprising Myd (or variant thereof) may, for example, range from about 4° C. to about 100° C., such as, for example, from about 4° C. to about 25° C.

The beverage can be a high-calorie beverage that has about 120 calories per 8 oz serving.

The beverage can be a mid-calorie beverage that has about 80 calories per 8 oz serving.

The beverage can be a low-calorie beverage that has less than 40 calories per 8 oz serving.

The beverage can be a zero-calorie that has less than 5 calories per 8 oz. serving.

In one embodiment, the beverage or beverage product has a low glycemic index. The glycemic index is a value assigned to foods based on how slowly or how quickly those foods cause increases in blood glucose level. Low glycemic index diets (including but not limited to beverages) are intended to achieve a more beneficial effect on blood glucose control in people with diabetes mellitus and may also provide metabolic benefits for the general population.

In a particular embodiment, the beverage or beverage product has a glycemic index that is at least 10% lower than the glycemic index of a substantially similar product made using conventional sweeteners (e.g., sucrose-based sweeteners). Methods for testing the glycemic index of a beverage have been described, e.g., as provided in Wolever, et al. Nutrition Research 23:621-629, 2003. In a particular embodiment, the beverage of beverage product has a glycemic index (GI) of about 55 or less. In a particular embodiment, the beverage or beverage product is a nutritional beverage with a low glycemic index.

In one embodiment, a beverage is disclosed that comprises between about 1 and about 30 ppm and less than about 500 ppm of one or more steviol glycosides, wherein the liquid matrix of the beverage is selected from the group consisting of water, phosphoric acid, phosphate buffer, citric acid, citrate buffer, carbon-treated water and combinations thereof. The pH of the beverage can be from about 3 to about 3.5 or from about 2.5 to about 3. The beverage can further include additives, such as, for example, erythritol. The beverage can further include functional ingredients, such as, for example vitamins.

### Methods for Improving Temporal or Flavor Profile

A method is disclosed for imparting a more sugar-like temporal profile, flavor profile and/or taste profile to a consumable (e.g. a beverage), comprising adding Myd (HTS) (or variant thereof) or the Myd-containing sweetener compositions or flavor modifying compositions disclosed herein to the consumable. The consumable may be referred to as a sweetenable composition or a flavor modifiable composition.

Without being bound by any particular theory, it is believed that the HTS may bind to mucins in the oral cavity. Mucins are the principal organic constituents of mucus, the slimy visco-elastic material that coats all mucosal surfaces. Structurally, mucins are high-molecular weight epithelial glycoproteins with a high content of clustered oligosaccharides O-glycosidically linked to tandem repeat peptides rich in threonine, serine, and proline.

Within the mouth, muscins coat the hard and soft tissues. Several salivary mucins are known, including MUC5B, MUC7 (previously known as MG2), MUC19, MUC1, and MUC4. Based on macromolecular characteristics, they can be classified into high (>1000 kD) and low (200-300 kD) molecular weight forms. Salivary mucins are synthesized by the mucus acinar cells of the paired submandibular (SMG) and sublingual (SLG) glands, as well as minor salivary glands distributed throughout the palatal and buccal mucosa.

The method can further include the addition of other sweeteners, additives, functional ingredients and combinations thereof. Any sweetener, additive or functional ingredient detailed herein can be used.

In one embodiment, the method of imparting a more sugar-like temporal profile, flavor profile to a consumable (e.g., a beverage) comprises adding Myd (or variant thereof) or the Myd-containing sweetener composition or flavor modifying composition to the consumable, thereby imparting a more sugar-like temporal profile or flavor profile.

As used herein, the “sugar-like” characteristics include any characteristic similar to that of sucrose.

A sweetener is generally a compound or mixture of compounds which taste sweet, particularly to a human. It will be appreciated that the sweetness or other characteristic of a sweetener perceived from a given sweetener may vary by the individual tasting the sweetener. Natural sweeteners generally include sugars, particularly sucrose, fructose, galactose, glucose, lactose, maltose and mixtures thereof. Non-sugar sweeteners include polyols such as erythritol, isomalt, lactitol, maltitol, sorbitol and xylitol. Some sweeteners are lower in calories than sucrose and others are described as non-caloric. Artificial sweeteners include, among others, acesulfame potassium (Ace-K), Advantame, Aspartame, Neotame, Saccharin and Sucralose. There is also a group of plant-derived non caloric or low caloric sweeteners including allulose, Monk fruit, Stevia and Tagatose. In general, each sweetener or mixture of sweeteners will exhibit different levels of sweetness and different taste and flavor characteristics.

Characteristics inherent to sweeteners include maximal response, flavor profile (including taste), temporal profile, mouthfeel, dose response, taste/flavor interactions, and temperature effects. As used herein, “maximal response” refers to peak sweetness intensity. As used herein, “flavor profile” refers to the combination of aromatic and basic taste elicited by a substance. As used herein, “temporal profile” refers to how the flavor profile changes throughout the perception of a substance over time. As used herein, “mouthfeel” refers to sensations perceived in the mouth that are elicited by the chemical or physical properties of a substance. As used herein, “dose response” refers to the function of concentration of the substance versus the intensity of the measured flavor or taste, e.g., sweetness.

In some instances, the perception of a sweetener may be affected by adaptation. “Adaptation” refers to the decrease in perceived intensity of a substance over a prolonged period of exposure.

These characteristics are dimensions in which the taste of sucrose is different from the tastes of Myd (or variant thereof). Of these, however, the flavor profile and temporal profile are particularly important. In a single tasting of a sweet food or beverage, differences (1) in the attributes that constitute a sweetener's flavor profile and (2) in the rates of sweetness onset and dissipation, which constitute a sweetener's temporal profile, between those observed for sucrose and for Myd (or variant thereof) can be noted.

In one embodiment, the method disclosed herein improves one or more organoleptic property of the consumable compared to a consumable that does not contain the sweetener composition or flavor modifying composition disclosed herein. In certain embodiments, the improvement is measured by a sensory test. The sensory test may be a taste test, a blind test, or a combination thereof. A sensory test can use one or more various protocols. For example, a sensory test can be the “triangle method”, follow ISO requirements, or a combination thereof. The taste test can be the average of multiple trials.

A taste test may be a screening test, a professional taste test, or a market research test. A screening test may be performed by at least 1, 2, 3, 4, 5, 6, 7, 8, or 9 taste testers. A professional taste test may be performed by at least 10, 15, 20, 25, or 30 taste testers. A market research test may be performed by at least 31, 40, 50, 60, 70, 80, 90, 100, 150, 200, 300, 400, or 500 taste testers. In some cases, a taste tester can be a person with average taste perception, a professional taste tester, a person who has passed a tasting exam by correctly identifying foods or food components, or a person who can identify the relative amounts of a taste or flavor (e.g., correctly sequence varying amounts of sugar in water).

In one embodiment, whether or not a characteristic is more sugar-like is determined by an expert sensory panel who taste compositions comprising sugar and compositions comprising HTS (or variant thereof), optionally in combination with at least one steviol (e.g., Reb M, Reb A) or HFCS, both with and without additives and provide their impression as to the similarities of the characteristics of the sweetener compositions, both with and without additives, with those comprising sugar. A suitable procedure for determining whether a composition has a more sugar-like taste is described in embodiments described herein below.

In a particular embodiment, a panel of assessors is used to measure the reduction of sweetness linger. Briefly described, a panel of assessors (generally 8 to 12 individuals) is trained to evaluate sweetness perception and measure sweetness at several time points from when the sample is initially taken into the mouth until 3 minutes after it has been expectorated. Using statistical analysis, the results are compared between samples containing additives and samples that do not contain additives. A decrease in score for a time point measured after the sample has cleared the mouth indicates there has been a reduction in sweetness perception.

The panel of assessors may be trained using procedures well known to those of ordinary skill in the art. In a particular embodiment, the panel of assessors may be trained using the Spectrum™ Descriptive Analysis Method (Melgaard et al, Sensory Evaluation Techniques, 3rd edition, Chapter 11). Desirably, the focus of training should be the recognition of and the measure of the basic tastes; specifically, sweet. In order to ensure accuracy and reproducibility of results, each assessor should repeat the measure of the reduction of sweetness linger about three to about five times per sample, taking at least a five-minute break between each repetition and/or sample and rinsing well with water to clear the mouth.

Generally, the method of measuring sweetness comprises taking a 10 mL sample into the mouth, holding the sample in the mouth for 5 seconds and gently swirling the sample in the mouth, rating the sweetness intensity perceived at 5 seconds, expectorating the sample (without swallowing following expectorating the sample), rinsing with one mouthful of water (e.g., vigorously moving water in mouth as if with mouth wash) and expectorating the rinse water, rating the sweetness intensity perceived immediately upon expectorating the rinse water, waiting 45 seconds and, while waiting those 45 seconds, identifying the time of maximum perceived sweetness intensity and rating the sweetness intensity at that time (moving the mouth normally and swallowing as needed), rating the sweetness intensity after another 10 seconds, rating the sweetness intensity after another 60 seconds (cumulative 120 seconds after rinse), and rating the sweetness intensity after still another 60 seconds (cumulative 180 seconds after rinse). Between samples take a 5-minute break, rinsing well with water to clear the mouth.

In some embodiments, the temporal profile, flavor profile and/or taste profile is evaluated in vitro, e.g., in a suitable assay such an in vitro system based on a cellular model overexpressing sweet and/or bitter receptor. In a particular embodiment, the in vitro system comprises TAS1R2/TAS1R3 receptors.

In one embodiment, the sweetener composition or flavor and/or taste modifying composition disclosed herein is capable of modifying one or more properties of the consumable including but not limited to mouthfeel, bitterness, bitter aftertaste or sweetness linger.

Mouthfeel refers to the textural aspects of a food or beverage responsible for producing characteristic tactile sensations perceived at the lining of the mouth, including the tongue, gums and teeth. These may include, but are not limited to, astringency, viscosity, slipperiness and mouth-coating. Mouthfeel is a fundamental sensory attribute which, along with taste and smell, determines the overall flavor of a consumable.

In one embodiment, Myd (or variant thereof) or the Myd-containing sweetener composition and/or flavor modifying composition disclosed herein imparts an improved mouthfeel to a consumable (e.g., beverage) to which it is added relative to a conventional sweetener composition or flavor and/or taste modifying composition. In one embodiment, the “improved mouthfeel” can be determined by a taste panel consuming said beverage in comparison to the same beverage without the taste modifying composition or an active component thereof. In a particular embodiment, the mouthfeel is improved by about 5%, about 10%, about 15%, about 20% or about 25% or more. In a particular embodiment the improvement in mouthfeel is characterized as increased fullness, body or richness. In a particular embodiment, the improved mouthfeel is characterized as a more syrup-y feel.

In another embodiment, the sweetener composition or flavor modifying composition disclosed herein improves (reduces) bitterness in a consumable (e.g., a beverage) to which it is added, relative to a conventional sweetener composition or flavor and/or taste modifying composition. This “improved bitterness” or “reduced bitterness” can be determined by a taste panel consuming said consumable in comparison to the same consumable without the taste modifying composition or an active component thereof. In a particular embodiment, the bitterness is reduced by about 5%, about 10%, about 15%, about 20% or about 25% or more.

In another embodiment, the sweetener composition or flavor modifying composition disclosed herein reduces the bitter aftertaste in a consumable (e.g., a beverage) to which it is added, relative to a conventional sweetener composition or flavor and/or taste modifying composition. This “reduced bitter aftertaste” can be determined by a taste panel consuming said consumable in comparison to the same consumable without the taste modifying composition or an active component thereof. In a particular embodiment, the bitter aftertaste is reduced by about 5%, about 10%, about 15%, about 20% or about 25% or more.

In a particular embodiment, the sweetener composition or flavor modifying composition disclosed herein reduces the bitter taste of a consumable (e.g., a beverage) to which it is added where the reduction is measured by the Metachronic VAS Score Profile for Bitterness and more particularly, the reduction is at least about 0.5 points, about 1 point, about 1.5 points, about 2.0 points, about 2.5 points, about 3.0 points, about 3.5 points or about 4.0 points or more. In certain embodiments, the reduction is between about 0.5 and about 4.0 points, about 1.0 and about 3.5 points or about 1.5 and about 3.0 points.

In a further embodiment, the sweetener composition and/or flavor modifying composition disclosed herein improves (reduces) sweetness linger of a consumable (e.g., a beverage) to which it is added. In one embodiment, this “improved sweetness linger” can be examined by a taste panel consuming said beverage in comparison to the same beverage without the taste modifying composition or an active component thereof. In a particular embodiment, the sweetness linger is reduced by about 5%, about 10% about 15%, about 20% or about 25% or more.

In a further embodiment, a flavor modifying composition disclosed herein provides a reduction in sourness of a consumable (e.g., a beverage) to which it is added. In one embodiment, this “reduced sourness” can be examined by a taste panel consuming said consumable in comparison to the same consumable without the taste modifying composition or an active component thereof. In a particular embodiment, the sourness is reduced by about 5%, about 10% about 15%, about 20% or about 25% or more.

Additional details relating to HTS and HTS variants are found in the Table 31 which includes a list of amino acid and nucleic acid sequences.

The following examples further illustrate the invention but, of course, should not be construed as in any way limiting its scope.

### EXAMPLES

### Example 1

Fresh Mattirolomyces terfezioides truffles were obtained in situ using appropriate procedures and permissions in their natural range. Fresh samples (29 in total) were shipped to MycoTechnology, Inc. facilities and were gently washed in RO water, then frozen in liquid nitrogen and stored at −80° C. The average moisture content of the truffles was 83.6% plus or minus 4.6%.

Aqueous extraction of the truffles was performed as follows. Eight different samples of truffle were pestled in liquid nitrogen to grind into a powder, then 5:1 v/w truffle of 4° C. water was added and allowed to incubate at 30 minutes at 4° C. The extracted material was then subjected to low-speed brief centrifugation and the filtrate was tasted “neat.” The sweetness intensity was rated between 0 for no sweetness and 10 for extremely sweet. Of the samples, the sweetness was rated as follows:

TABLE 1
Sweetness of various truffle samples.

| | Sweetness | |
|---|---|---|
| sample | intensity | Notes |
| 1 | 5 | Sweet taste at end |
| 2 | 8 | Sweet taste is upfront and intensifies |
| | | at mid-end and lingers |
| 3 | 7 | Sweetness is upfront and intensifies |
| | | at mid-end and lingers. |
| 4 | 5 | Sweet upfront, low sweet linger |
| 5 | 4 | Sweetness less strong |
| 6 | 3 | Low sweetness |
| 7 | 6 | A mild and clean sweet taste |

Aqueous extract was stored at 4° C. at pH 7 and pH 2 in sodium phosphate buffer, and little to no change in sweetness was observed over an 8-day period.

### Example 2

Purification of sweet protein Myd from M. terfezoides. Fresh Mattirolomyces terfezioides truffles were obtained in situ using appropriate procedures and permissions in their natural range and stored at −80° C. A 16.3 g sample was removed from the freezer and was ground with a mortar and pestle (white ceramic) in liquid nitrogen. Grinding proceeded for 15 minutes to fully pulverize tissue and obtain a fine frozen powder. Powder was added to 50 mL Falcon tubes and 20 mL RO—H₂O was added, and vortexed to mix tissue, until no ice crystals were observed. A Rotor-Stator at a setting of 20 for 2×1 minutes at 4° C. was used to breakup fragment and make a homogenous solution (H1). The volume of slurry was adjusted to 53 mL with RO—H₂O and centrifuged at 7500×G for 30 minutes at 4° C. The supernatant (S1) from this step was collected into 2 mL Eppendorf tubes and centrifuged in a 5417 R and Eppendorf centrifuge at 20,000×G for 15 minutes at 4° C. The pellet (P1) was discarded. The sweet taste (via human sensory) was perceived in the supernatant. Supernatant from this step was collected and pooled. The supernatant was then filtered through a 0.45 micron syringe filter (Cellulose, VWR International), 25 mm, and called S1+0.22 μm Filtration. The filtrate was then washed with hexane (38 mL to 50 mL hexane 2 times, and the water phase was collected. S1FH is S1F+hexane wash. The hexane phase was saved and dried. The water phase was then precipitated with acetone (50 mL at −20° C. was added to 33 ml of fraction S1FH and set for 30 minutes at −20° C.). The sample was centrifuged at 3,000×g and the precipitate was collected. The precipitate was resuspended in 10 mM sodium phosphate pH 6, with the supernatant from this step called S2, and the precipitate called P2. The supernatant S2 was first applied to an AMICON centrifugal filter units with a molecular weight cutoff of 100 kD, obtaining a filtrate (flow-through) portion (called 100F) and a retentate (called 100R); followed by applying the 100F to a unit with a molecular weight cutoff of 30 kD, resulting in a filtrate (30F) and a retentate (30R). The sweet taste fraction flowed through the 100 kD column and appeared in 100F, and was retained on the 30 kD column (30R). A band was observed at approximately 13 kD on the SDS-PAGE of FIG. 1, shown by the arrow, and this band was cut out and subjected to N-terminal sequence analysis by Edman degradation using standard methods of detection e.g. liquid chromatography and mass spectrometry to identify the residues for each cycle.

### Example 3 (Identification of RNA)

### Sample Collection

Fresh Mattirolomyces terfezioides truffles were obtained in situ using appropriate procedures and permissions in their natural range. A wild isolate (BDP2_18) of Mattirolomyces terfezioides truffle (gleba) was sourced from a natural environment. BDP2_18 was the largest wild truffle scavenged and the truffle had a sweetness characteristic of “sweet upfront, more fungal and earthy, low sweet linger.”

### Sample Identification

The wild isolates of gleba were shipped frozen for GeneWiz® sequencing (Azenta US Inc.) Sequencing for Internal Transcribed Spacer (ITS) Sequencing. The genome loci sequenced are the ITS 1 & 2 region. The resulting Sanger sequencing reads were then aligned and trimmed of low-quality bases. Each sequence was then subjected to an individual Basic Local Alignment Search Tool (BLAST) (Altschul, Gish, Miller, Myers, & Lipman, 1990) search to verify identity. BLASTn search was employed using nucleotide collection (nr/nt) with unpublished samples sequences excluded. The entry with the highest percent identity to the wild isolate is Mattirolomyces terfezioides strain rib02.

### Sample Preparation

The wild isolate, BPD2_18, was superficially washed with sterile water and cut into cubes weighing ˜100 mg and flash frozen in liquid N₂ to be stored at −80° C. Shipment was carried out on dry ice for GeneWiz® (Azenta US Inc) sequencing.

### RNA Sequencing

The following samples were submitted through GeneWiz® sequening for Standard RNASeq going through Illumina HiSeq, 2×150 bp, single index, per lane with ˜350M raw paired-end reads per lane. This RNASeq study involved polyA+ selection for transcriptome profiling.

The GeneWiz® sequencing procedure extracted RNA using the RNeasy® Plus (Qiagen GmbH) Universal mini kit following manufacturer's instructions. RNA Library prep was done using the NEBNext® Ultra (New England Biolabs, Inc.) RNA Library Prep Kit for Illumina. The Illumina adapter sequences are outlined below.

(SEQ ID NO: 9)
5′-AATGATACGGCGACCACCGAGATCTACACTCTTTCCCTACACGACG
CTCTTCCGATCT-3′
(SEQ ID NO: 10)
5′-GATCGGAAGAGCACACGTCTGAACTCCAGTCAC[i7
barcode]ATCTCGTATGCCGTCTTCTGCTTG-3′

Fastq sequence files from the RNA-seq study on two strains of Mattirolomyces terfezioides (three replicates each) were trimmed and cleaned with HTStream to remove the following contaminants: PhiX (common contaminant from sequencing), rRNA reads, sequencing adapters, low quality and “N” bases, poly-a tracts, primers and reads <50 bp. Transcriptome assembler rnaSPAdes (Bushmanova E, Antipov D, Lapidus A, Prjibelski AD. rnaSPAdes: a de novo transcriptome assembler and its application to RNA-Seq data.

Gigascience. (2019) 8 (9): giz100. doi: 10.1093/gigascience/giz100) was then used for the de novo assembly of the transcriptome for each strain of M. terfezioides from the cleaned files. Bandage (a Bioinformatics Application for Navigating De novo Assembly Graphs Easily) (Ryan R. Wick, Mark B. Schultz, Justin Zobel, Kathryn E. Holt, Bandage: interactive visualization of de novo genome assemblies, Bioinformatics, Volume 31, Issue 20, 15 Oct. 2015, Pages 3350-3352) was used to visualize and search the assembly for the target peptide using a tBLASTn search (Gertz E M, Yu Y K, Agarwala R, Schäffer A A, Altschul S F. Composition-based statistics and translated nucleotide searches: improving the TBLASTN module of BLAST. BMC Biol. 2006; 4:41. Published 2006 Dec. 7. doi: 10.1186/1741-7007-4-41) that compares the protein query sequence against the assembled transcriptome sequence database dynamically translated in all six reading frames. Contigs that contained perfect matches to the query protein sequence were analyzed for open reading frames (ORFs) and eventually used to construct the full-length mRNA connected to the target peptide.

An RNA transcript was identified, and the DNA copy thereof has the sequence shown as SEQ ID NO:1, of which SEQ ID NO:2 is the predicted coding sequence based on the start and stop codons. A predicted protein, SEQ ID NO:3, is also provided, having 121 amino acids with an estimated size of 13.3 kD. Length: 122 aa, molecular weight: 13.381 kD, predicted isoelectric point: 8.64, and predicted charge at pH 7:1.01.

Blast analyses. Identity between the predicted protein SEQ ID NO:3, and other protein sequences in GENBANK was 31% or less. A hypothetical protein from Pisolithus tincturius was found (GenBank: KIN98154.1) with approximately 31% homology to SEQ ID NO:3; called hypothetical protein M404DRAFT_1005519 [Pisolithus tinctorius Marx 270]. It is hypothesized that this protein may also have sweet modulating activity. The complete cDNA copy of the Pisolithus tinctorius RNA transcript is identified in international patent application PCT/US2022/82443 (see also PCT/US2020/012955 and PCT/US2021/039176).

### Example 4 (Cloning and Heterologous Expression of Mycodulcein HTS in E. coli; Confirmation of Sweet Taste.)

Based on the nucleotide sequence identified as encoding SEQ ID NO:3, three expression vectors to express the nucleotide coding sequence SEQ ID NO:4 also containing sequence encoding a histidine tag were synthesized and cloned by Atum, Inc. (Newark, CA) into three different Atum vector backbones: pD454—SR (plasmid pMy_3000), pD454-MR (plasmid pMy_3001), and pD454-WR (plasmid pMy_3002), all of which are E. coli isopropyl beta-D-1-thiogalactopyranoside (IPTG)-inducible T7 promoter expression vectors with ampicillin-r, lacl, Lac01, Ori_pUC, and medium (M), strong(S) and weak (W) ribosome binding sites on the plasmid. E. coli BL21 DE3 (Studier et al. (1986) J. Mol. Biol. 189:113-130) (New England Biolabs) was transformed with pMy_3000, pMy_3001, pMy_3002 using manufacturer protocols. In short, previously frozen competent cells were thawed and mixed with 1 pg-100 ng of plasmid DNA and held on ice for 30 minutes. A heat shock of 42° C. for 45 seconds was applied to this mixture. Immediately thereafter, the mixture was placed into an ice bath for 10 minutes. 950 μL of pre-warmed LB media was added and then subjected to 1 hour of 225 rpm shaking at 37° C. Dilution of cells at 1:10 and 1:100 and plating 100 μL on the antibiotic plate was performed for each transformation reaction. An overnight growth at 37° C. allowed colonies to fully recover and become visible. To confirm successful transformation, csPCR (colony screen PCR) was performed to interrogate the cDNA region of the plasmid and expression was confirmed through lysate SDS-PAGE. Shake flask-scale induced expression was used to confirm heterologous expression in the E. coli host. This process yielded strains Z14CE, Z15CE, Z16CE containing the plasmids pMy_3000, pMy_3001, and pMy_3002 respectively. The three strains (Z14CE, Z15CE, Z16CE) were maintained on LB+ampicillin 100 μg/mL agar plates while the negative control (Eco_0001) was maintained on a LB agar plate. An overnight culture was grown for each strain in 50 mL of LB liquid media in un-baffled 250 mL culture shake flasks at 37° C., shaking at 150 rpm overnight with appropriate antibiotics. Each overnight culture was the following day seeded into 200 mL of TB liquid media in baffled 1000 mL culture shake flasks at 37° C., shaking at 200 rpm, and adjusted to 0.1 OD₆₀₀ with the appropriate antibiotic. Once the OD₆₀₀ reached 0.8, IPTG was added into the media to a final concentration of 0.66 mM. Shaking was continued at 37° C. for an additional 5 hrs. After expression, the cells were centrifuged at 4000 g for 20 min. The supernatant was discarded, and the cell pellet was suspended in ˜20 mL of wash buffer (10 mM Sodium Phosphate Buffer pH 7.0). Suspended cells were disrupted in a high-pressure homogenizer (C3 Emulsiflex, Avestin, Inc., Ottawa, ON, Canada) operated at a max of 2,000 bar. Disrupted cells were centrifuged at 13,000 g (30 min), the supernatant was collected, and the pellet was discarded. The supernatant containing solubilized protein was filtered through a 0.22 μm PES membrane unit (Millipore, Burlington, MA, USA). SDS PAGE was run and a 13.1 kD band (Coomassie stain) was observed to confirm expression.

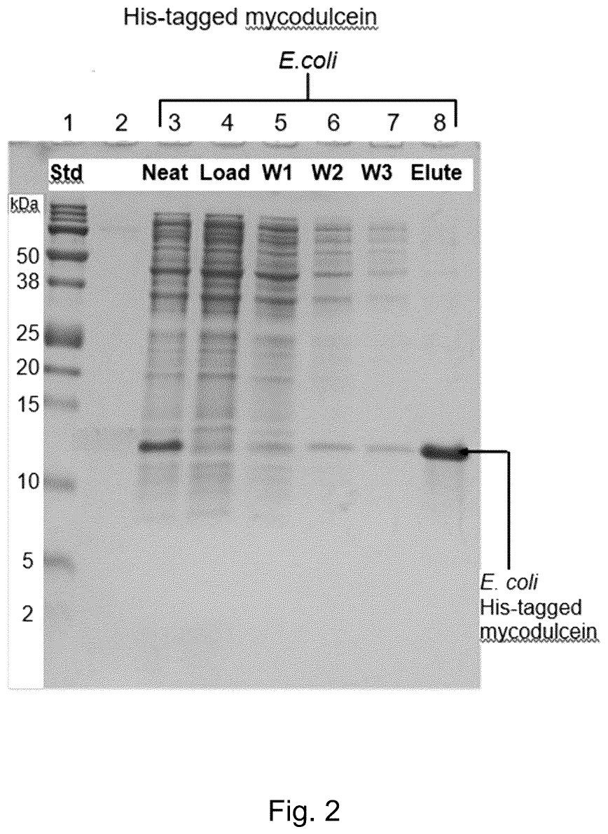

Thermo Scientific™ HisPur™ Ni-NTA resin was used to purify the his-tagged protein expressed from SEQ ID NO:4 using effective immobilized metal affinity chromatography (IMAC). The protein encoded by SEQ ID NO:4 was purified using nickel-charged nitrilotriacetic acid (NTA) chelate immobilized onto 6% crosslinked agarose resin. Lysate was loaded onto a prepared IMAC column, equilibrating with Binding Buffer: 20 mM sodium phosphate monobasic, 0.5M sodium chloride, 0.1M imidazole, pH 7.4, and eluted using Elution Buffer: 20 mM sodium phosphate monobasic, 0.5M sodium chloride, 0.5M imidazole. The column was washed three times with Binding Buffer followed by eluting his-tagged mycodulcein four times with elution buffer, followed by ultrafiltration of eluted fractions using a 30 kD MWCO filter followed by filtering filtrate through 10 kD filters at 4000×G for 15 minutes. The retentate was diluted and spun again for a wash step, which was repeated three times. Purification steps analysis by SDS-PAGE is shown in FIG. 2.

The purified fraction (shown in Lane 8 from FIG. 2, containing highly purified SEQ ID NO: 5) was tasted at 0.03 mg/ml by a trained sensory scientist (0.2 mL aliquot) and found to have a sweetness equivalent to 8° brix (approximately 8% sucrose solution), confirming that mycodulcein (HTS) isolated from M. terfezioides was responsible for the sweet taste activity observed in Examples 1 and 2. The sweet taste was very noticeably sweet, with a “clean” sweetness (sugar-like taste) with no other flavors, with a slightly delayed onset and a sweet aftertaste.

### Example 5 (Pilot-Scale Production of Mycodulcein (His-Tag))

E. coli strain Z14CE prepared in Example 4 containing nucleotide coding sequence SEQ ID NO: 4 was tested for its performance during fermentation in a lab-scale bioreactor. Bioreactor cultures were performed in 10.0 L Bioflo 320 round bottomed stirred fermentor (BioFlo/CelliGen 310, New Brunswick Scientific, Edison, NJ, USA). The fermenter was fitted with pH and dissolved oxygen sensors (Mettler Toledo, OH, USA). Temperature was controlled via a water-filled stainless-steel base. Agitation was provided by two mounted six-bladed Rushton turbines spaced 47 mm apart with the lowermost impeller positioned just above the bottom of the shaft. Aeration occurred through a perforated pipe sparger ring. Dissolved oxygen (DO) was controlled at 20% of air saturation by using a sequential cascade of agitation between 500 and 800 rpm and aeration between 5 and 8 L/min with air sparged at high-cell densities. The pH was controlled at 7.0 using 5.0 M ammonium hydroxide. Antifoam 204 (Sigma, St Louis, MO, USA) was added automatically to control the foaming. The latter was sensed using a conductivity probe mounted 10 cm above the culture level. The main fermentation medium contained (per liter) 24 g yeast extract, 12 g tryptone, 5.42 mL glycerol, 100 mL of phosphate buffer stock (0.17 M KH₂PO₄, 0.72 M K₂HPO₄) The medium was adjusted to pH 7.0 using 2 M HCl. When the original glucose supply was depleted (signaled by a rise in pH), a feed medium (per liter) consisting of 200 g glucose, 21.1 g (NH₄)₂SO₄ and 19.7 g MgSO₄ was pumped into the fermenter at an initial flow rate of 1.00 mL/min. Unless otherwise stated, the initial volume of the medium in the vessel was 4.0 L. Inoculum (200 mL) consisted of a culture that had been grown for 16 h in a 1 L baffled shake flask (37° C., 200 rpm) in LB started culture media. The fermenter temperature was 37° C. Fermentations were induced with 0.66 mM IPTG after optical density reached 10-20 and continued thereafter for 24 hours. The broth was subsequently centrifuged at 4000 g for 20 min after the 24-hour induction period. The supernatant was discarded, and pellet was suspended in 1 L of wash buffer (10 mM sodium phosphate buffer pH 7.0). Suspended cells were disrupted in a high-pressure homogenizer (C3 Emulsiflex, Avestin, Inc., Ottawa, ON, Canada) operated up to a max of 1,500 bar. Disrupted cells were centrifuged at 13,000 g (30 min), the supernatant was collected, and the pellet was discarded. The supernatant containing solubilized protein was filtered through a 0.22 μm PES membrane unit (Millipore, Burlington, MA, USA).

The purified supernatant prepared in this Example was tasted at 0.03 mg/mL by a trained sensory scientist (0.2 mL aliquot) and found to have a sweetness equivalent to 8 brix (approximately 8% sucrose solution). The sweet taste was very noticeably sweet, with a “clean” sweetness (sugar-like taste) with no other flavors, with a slightly delayed onset and a sweet aftertaste.

Supernatant was stored in aliquots at −20° C. and used for further experiments.

### Example 6 (Mycodulcein (HTS) from E. coli ELISA Quantification)

A direct ELISA was developed to quantify his-tagged mycodulcein (HTS) (SEQ ID NO: 5) with a horseradish peroxidase (HRP) conjugated antibody to the 6×His-Tag sequence on the carboxy terminus of SEQ ID NO:5. The ELISA enables the measurement of mycodulcein (HTS) concentration in complex lysates and purified protein. Recombinant 6×His-tagged E. coli mycodulcein has a molecular weight of 14.2 kD and a molar extinction coefficient of 27,960 M⁻¹ cm-1, calculated from the amino acid sequence by methods known in the art. Purity was assessed by SDS-PAGE as ≥98%. Mycodulcein (HTS) protein concentration was determined by absorbance at 280 nm using Beer-Lambert's Law, then a standard curve was generated using known concentrations of mycodulcein (ug/ml) for the ELISA.

ELISA procedure: Protein was bound to the walls of a high-protein binding 96-well plate in 50 mM carbonate buffer pH 9.4 coating buffer for 30 minutes at room temperature. The plates were washed 3 times with phosphate buffered saline (PBS) pH 7.4 with 0.02% Tween-20. Nonspecific binding sites on the microplate were blocked with 5% BSA in PBS pH 7.4 for 15 minutes at room temperature and washed three times with PBS 0.02% Tween-20. The primary antibody was diluted (1:1000) in blocking buffer and the microplates were incubated for 1 hour and washed 3 times in PBS 0.02% Tween-20. The colorimetric substrate, 3,3′,5,5′-tetramethylbenzidine (TMB), was used to develop the HRP reaction for 8 minutes and stopped with 2N sulfuric Acid.

### Example 7 (Quantitative Characterization of the Concentration-Dependence of Mycodulcein HTS)

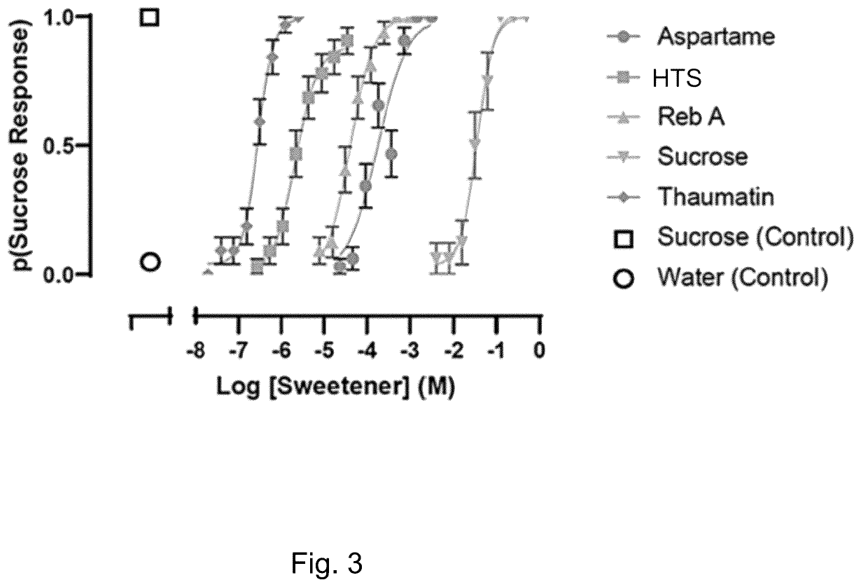

Opertech Bio (Philadelphia, PA) performed a quantitative characterization of the concentration-dependence of the taste properties of the purified his-tagged mycodulcein HTS (SEQ ID NO:141 with His-tag, SEQ ID NO:142) obtained from E. coli as described in Example 5 and purified as described in Example 4. The sweet-taste potency and relative efficacy of mycodulcein (HTS) was compared with sucrose and with other sweeteners thaumatin, rebaudioside A, and aspartame. Control standards were solutions of 200 mM sucrose, 100 mM NaCl, 0.5 mM quinine, and 10 mM citric acid. FIG. 3 shows the concentration-response functions for sweet taste for mycodulcein (HTS), aspartame, thaumatin, rebaudioside A. Data are plotted as the proportion (p) of responses occurring on the 200 mM sucrose-associated (“sweet”) target. Each data point in the curves for mycodulcein, aspartame, thaumatin, and rebaudioside A was calculated as the average across 32 replicates and averaged across 16 replicates for the sucrose curve; error bars are SEM. Points for water and sucrose controls were similarly calculated as the average across 128 and 64 replicates, respectively. Curves were fit by non-linear regression.

The concentrations that elicit half-maximal sweet taste response (EC50s, or potencies) were derived from the nonlinear regression. The EC50s (and 95% confidence intervals) for mycodulcein (MYC) also called HTS, sucrose (SUC), aspartame (ASP), rebaudioside A (REB), and thaumatin (THN) are given in Table 2. Also provided are the equivalencies to sucrose on a molar basis and on a weight basis.

TABLE 2

| | EC50 | | | Relative to | Relative to |
|---|---|---|---|---|---|
| | (molar | | EC50 | Sucrose | Sucrose |
| | units) | CI 95% | (mg/ml) | (mM) | (mg/ml) |
| MYC | 2 μM | 2-3 μM | 0.0286 | 18000 | 431 |
| THN | 0.3 μM | 0.2-0.3 μM | 0.0066 | 120000 | 1865 |
| REB | 42 μM | 33-57 μM | 0.0411 | 857 | 300 |
| ASP | 182 μM | 142-254 μM | 0.0532 | 198 | 231 |
| SUC | 36 mM | 27-45 mM | 12.3120 | 1 | 1 |

Evaluation of thaumatin, sucrose, and his-tagged mycodulcein (HTS) was carried out using a CATA (click all that apply) time intensity of the sweet sensation of named analytes in water at 0.045 mg/mL for proteins and 10% sucrose. Methodology: Time Intensity Technique; Data collection software: EyeQuestion, responses recorded every 2.57 seconds; Scaling Method: a 15-point sweet scale, e.g., score 5=5% sucrose, score 10=10% sucrose; Evaluation Protocol: small volume sip, tilt and spit test. There were 3-6 panelists and 2 replicates. All samples were blinded and presented with randomized 3-digit codes.

Training Strategies: An intense training on sweet scale over 6 weeks on 15-point sweet scale to confidently assign sweet values. Due to unique sweet behavioral patterns, it is necessary to train time intensity principles over 3 weeks. Samples were tasted with a stopwatch to note times and help reach a consensus. # of Panelists: 3-6, # of replications: 2. All samples were blinded and presented with randomized 3-digit codes. Statistical analysis: due to the small # of panelists, a statistical analysis cannot be performed.

The maximum intensity (Imax) of mycodulcein and thaumatin shows approximately 1 point higher on the 15 point scale at the amounts tested than sucrose. Thaumatin and mycodulcein have a flatter slope, indicating a longer peak time and more gradual/longer decline. Sucrose approaches threshold sweetness (Intensity <1) at 162 seconds, sooner than thaumatin and mycodulcein. When sucrose reaches threshold level, thaumatin and mycodulcein are recognizable at a low-moderate intensity. Mycodulcein and thaumatin appeared to be of similar potency in this experiment, which is on the order of 3000× sweeter than sucrose on a weight basis, or approximately 120,000× sweeter than sucrose on a molar basis. From these two experiments, mycodulcein is shown to be a high intensity sweet protein with a potency of between 400× sweeter than sucrose and 3000× sweeter than sucrose on a weight basis.

### Example 8 (Production and Testing of Variants of Mycodulcein (HTS) for Sweetness and Thermal Stability)

Method used to identify potential key residues in mycodulcein. Although sweet proteins have no primary sequence identity, the overall tertiary structures have a sweet finger motif (Tancredi, T., Pastore, A., Salvadori, S., Esposito, V. & Temussi, P. A. Interaction of sweet proteins with their receptor: A conformational study of peptides corresponding to loops of brazzein, monellin and thaumatin. European Journal of Biochemistry 271, (2004): 2231-2240.). Sweet proteins have antiparallel beta-sheets with an alpha-helix perpendicular to the beta-sheets. The sweet proteins thaumatin, monellin, brazzein, and lysozyme tertiary structures were analyzed using PyMOL 2.0 (The PyMOL Molecular Graphics System, Version 2.0 Schrödinger, LLC) and compared to a model of mycodulcein generated with Phyre2 (Kelley L A et al. The Phyre2 web portal for protein modeling, prediction, and analysis Nature Protocols 10, (2015): 845-858).

A number of single-site mutants were generated (International patent application PCT/US2022/82443) and are presented in Table 3.

Cloning: Eco_0001 aka E. coli BL21 DE3 (E. coli str. B F⁻ ompT gal dcm lon hsdS_{B}(r_{B}⁻ m_{B})λ(DE3 [lacI lacUV5-T7p07 indl sam7 nin5]) [malB⁺]_{K-12} (λ^{S})) (obtained from New England Biolabs #C2527I), was transformed with twenty-three plasmids (pMy_3018 to pMy_3040) using manufacturer protocols. In short, previously frozen competent cells were thawed and mixed with 1 pg-100 ng of plasmid DNA and held on ice for 30 minutes. A heat shock of 42° C. for 45 seconds was applied to this mixture. Immediately thereafter, the mixture was placed into an ice bath lasting for 10 minutes. 950 μL of pre-warmed LB media was added and then subjected to 1 hr of 225 rpm shaking at 37° C. Dilution of cells at 1:10 and 1:100 and plating 100 μL on the antibiotic plate was performed for each transformation reaction. An overnight growth at 37° C. allowed colonies to fully recover and become visible. This process yielded strains Z18CE to Z40CE containing the plasmids pMy_3018 to pMy_3040 sequentially. Shake flask-scale induced expression was used to confirm heterologous expression in the E. coli host. Post-transformational mutants were plated and maintained on LB+Ampicillin 100 μg/mL agar plates.

Plates were incubated for 16 hours at 37° C. Colony screen PCR was performed to confirm genotypes using colony screening primers designed to interrogate the flanking region along with the cDNA region of the plasmid. A successful transformation resulted in a DNA fragment of a certain size while a negative control and no template control result in no PCR band. Successful transformation was observed for all mutants.

Shake flask-scale induced expression was used to confirm heterologous expression in the E. coli host.

Strains were maintained on LB+ampicillin 100 μg/mL agar plates while the negative control (Eco_0001) was maintained on a LB agar plate. An overnight culture was grown for each strain in 50 mL of LB liquid media in un-baffled 250 mL culture shake flasks at 37° C. shaking at 150 rpm overnight with appropriate antibiotics. Each overnight culture was subsequently seeded into 200 mL of TB liquid media the following day into baffled 1000 mL culture shake flask at 37° C. shaking at 200 rpm and adjusted to 0.1 OD₆₀₀ with the appropriate antibiotic. Once the OD₆₀₀ reached 0.8, supplement of IPTG was added into the media to a final concentration of 0.66 mM. Continued the shaking at 37° C. for an additional 5 hrs. Afterwards, cells were collected by centrifugation at 5000 g at 4° C. for 5 min. E. coli cells were washed once with cold dH₂O and centrifuged again at 5000 g at 4° C. for 10 minutes. For confirmation of successful expression, cell lysate was created using liquid nitrogen and a mortar and pestle. The cell pellet was resuspended in 10 mL of cold dH₂O and the crude lysate was spun at 20,000 g at 4° C. for 5 minutes. Finally, the supernatant was aspirated and filtered through 0.2 μm PES filter and run on SDS-PAGE protein electrophoresis. Crude lysates were tasted in order to identify sweet-tasting mutants. Table 3 shows exemplary results of the testing.

TABLE 3
Results of sweetness testing on single point mutations
of mycodulcein (see international patent application
nos. PCT/US2021/039176 and PCT/US2022/82443)

| Content | Sweetness? | AA Substitution |
|---|---|---|
| Control-no mutation | Yes | N/A |
| (SEQ ID NO: 5) | | |
| Z38CE | Yes | D3E |
| Z39CE | Yes | K11R |
| Z40CE | No | R20K |
| Z41CE | Yes | K26R |
| Z42CE | No | E35D |
| Z43CE | No | K44R |
| Z44CE | No | D46E |
| Z45CE | Yes | K51R |
| Z46CE | No | D52E |
| Z47CE | Yes | R57K |
| Z48CE | Yes | R66K |
| Z49CE | Yes | D69E |
| Z50CE | No | R75K |
| Z51CE | Yes | D85E |
| Z52CE | Yes | E86D |
| Z53CE | Yes | E89D |
| Z54CE | No | D94E |
| Z55CE | Yes | D97E |
| Z56CE | Yes | K103R |
| Z57CE | Yes | R106K |
| Z58CE | Yes | R110K |
| Z59CE | Yes | E117D |
| Z60CE | Yes | K120R |

16 of the variants (Z38CE, Z39CE, Z41CE, Z45CE, Z47CE, Z48CE, Z49CE, Z51CE, Z52CE, Z53CE, Z55CE, Z56CE, Z57CE, Z58CE, Z59CE, Z60CE) all of which had sweet taste were additionally re-expressed using 200 mL of media. After expression, the cells were centrifuged at 4000 g for 20 min after the 24-hour induction period. The supernatant was discarded, and pellet was suspended in ˜20 mL of wash buffer (10 mM sodium phosphate buffer pH 7.0). Suspended cells were disrupted in a high-pressure homogenizer (C3 Emulsiflex, Avestin, Inc., Ottawa, ON, Canada) operated at a max of 2,000 bar. Disrupted cells were centrifuged at 13,000 g (30 min), the supernatant was collected, and the pellet was discarded. The supernatant containing solubilized protein was filtered through a 0.22 μm PES membrane unit (Millipore, Burlington, MA, USA). The material was subsequently isolated through IMAC Purification as described in Example 4.

The purified samples were tasted by a trained sensory scientist. Mycodulcein stocks were diluted to equal protein concentration as measured by ELISA. Subjects followed a sip and spit protocol approved by an Institutional Review Board. 0.2 ml of each purified mutant was placed on the tongue and intensity of sweetness perception, time of onset of sweet perception, and duration of sweetness perception were noted. Single site mutations (see Table 8) are assessed for sweetness as described above.

### REFERENCES

Korz, D. J., Rinas, U., Hellmuth, K., Sanders, E. A. & Deckwer, W.-D. Simple fed-batch technique for high cell density cultivation of Escherichia coli. Journal of Biotechnology 39, 59-65 (1995).
Norsyahida, A., Rahmah, N. & Ahmad, R. M. Y. Effects of feeding and induction strategy on the production of BmR1 antigen in recombinant E. coli. Letters in Applied Microbiology 49, 544-550 (2009).

### Example 9

Thermal Stability measurements are performed on protein samples from the IMAC Purification of HTS variants as described above in Example 8, normalized to 0.04 mg/mL, using the GloMelt™ Thermal Shift Protein Stability Kit (Biotium, Inc. Fremont, CA), using instructions provided by the manufacturer. In short, the individual reaction to measure thermal shift relies on mixing the following: mycodulcein or variant, 36 μg/ml in 25 mM sodium phosphate buffer pH 7.4 with reagents provided in the kit per manufacturer's instructions. For the thermal shift measurement, Bio-Rad CFX96 Touch system using BR Clear plates, scan-mode SYBER/FAM only, 25° C. for 30 seconds, melt curve 25° C. to 95° C., increment 0.5° C. for 10 sec plus plate read was used. The Tm is determined based on the midpoint determined for a curve fitted to the experimental data with a five-parameter equation using the techniques as described in Schulz, M. N., Landström, J. & Hubbard, R. E. MTSA-A Matlab program to fit thermal shift data. Analytical Biochemistry 433, 43-47 (2013). Results (Table 4) show the effect of amino acid changes on thermal stability.

TABLE 4
Exemplary results of thermal stability testing for mutants

| | Mutant STRAIN ID | |
|---|---|---|
| Amino Acid Subs | NumBER | Tm |
| D3E | Z38CE | 58.02135 |
| K11R | Z39CE | 58.9354 |
| K26R | Z41CE | 58.81657 |
| K51R | Z45CE | 60.66312 |
| R57K | Z47CE | 58.48098 |
| R66K | Z48CE | 58.99634 |
| D6E | Z49CE | 59.79641 |
| D85E | Z51CE | 59.99543 |
| E86D | Z52CE | 58.81274 |
| E89D | Z53CE | 57.96772 |
| D97E | Z55CE | 60.14034 |
| K103R | Z56CE | 59.29316 |
| R106K | Z57CE | 61.10271 |
| R110K | Z58CE | 57.92335 |
| K120 | Z60CE | 60.168 |

### Example 10 (Cloning and Heterologous Expression of Mycodulcein or Variants in Saccharomyces Cerevisiae; Confirmation of Sweet Taste)

Based on the nucleotide sequence identified as SEQ ID NO:2, two expression vectors to express his-tagged mycodulcein and (native) mycodulcein were synthesized and cloned by Atum, Inc. (Newark, CA) into Atum vector non-secretory backbone pD1234 containing URA3 marker, and strong constitutive promoter GPD (also described in International patent application nos. PCT/US2021/039176 and PCT/US2022/82443). The transformation procedure involves making electrocompetent cells and then introducing expression vectors through electroporation. In short, the electrocompetent cells are created by first growing the cells to between the early and mid-log phase with multiple washes to remove the salt from the growth medium. After mixing 1-5 μg of the expression vector, the sample is subjected to the following settings on a Gene Pulser® II Electroporator (BioRad) Charging Voltage: 1.5 kV, Capacitance: 25 μF, Resistance: 200 (2) 1 mL of prewarmed 30° C. YPD is added immediately, and the suspension is incubated for 1-2 h at 30° C. shaking at 200-250 rpm. Post-transformational mutants were plated and maintained on SC-ura agar plates. This process yielded strains Z19ES, Z20ES containing the plasmids pMy_4002 and pMy_4003 respectively. csPCR (colony screen PCR) was performed to interrogate the cDNA region of the plasmid. Thus, a successful transformation would result in the DNA fragment of 303 bp while a negative control and no template control would result in no PCR band and expression was confirmed. Analogous methods are used to express various mycodulcein variants and his-tagged variants.

The two strains (Z19ES, Z20ES) were maintained on SC-ura agar plates while the negative control was maintained on SC agar plates. An overnight culture was grown for each strain in 50 mL of SC-ura/SC liquid media in un-baffled 250 mL culture shake flask at 37° C. with shaking at 150 rpm overnight. Each overnight culture was inoculated into 200 mL of SC-ura/SC (O-RDL-R10_TB Media) liquid media in baffled 1000 mL culture shake flask at 30° C. shaking at 200 rpm and adjusted to 0.02 OD₆₀₀. The shaking was continued at 30° C. for an additional 48 hrs. Afterwards, cells were collected by centrifugation at 5000 g at 4° C. for 5 min. Afterwards, S. cerevisiae cells were washed with cold dH₂O and centrifuged again at 5000 g at 4° C. for 5 minutes. For confirmation of successful expression, cells were lysed using liquid nitrogen and a mortar and pestle. Cell pellets were resuspended in 10 mL of cold dH₂O and lysate was spun at 20,000 g at 4° C. for 5 minutes. Supernatant was filtered with a 0.2 μm PES filter. The filtrate (both strains) was confirmed to taste sweet by methods described in Example 3.

Thermo Scientific™ HisPur™ Ni-NTA resin was used to purify the his-tagged mycodulcein (or variant) protein from S. cerevisiae using effective immobilized metal affinity chromatography (IMAC). His-tagged protein was purified using nickel-charged nitrilotriacetic acid (NTA) chelate immobilized onto 6% crosslinked agarose resin. Lysate was loaded onto a prepared IMAC column, the column was washed three times with 0.02 M imidazole in PBS followed by eluting his-tagged mycodulcein four times with 0.3 M imidazole in PBS. Eluted fractions were subjected to ultrafiltration using a 50 kD MWCO filter followed by concentration and desalting with a 3 kD MWCO filter.

### Example 11 (Purification of Mycodulcein Expressed from Strain Z19ES (S. cerevisiae))

Three chromatographical techniques were assessed for effectiveness for purification of mycodulcein expressed in S. cerevisiae: cation exchange (CIEX), hydrophobic interaction (HIC), and size exclusion chromatography (SEC).

### Cation Exchange Assessment

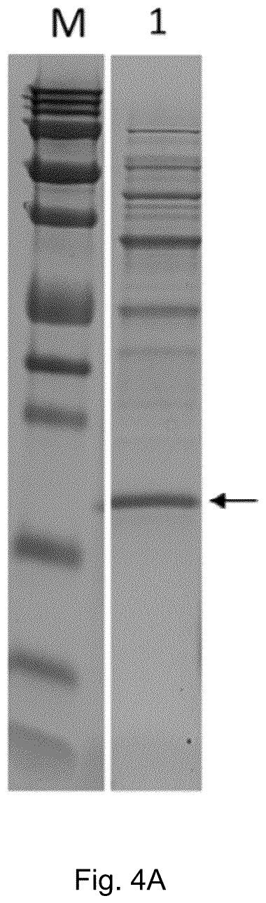

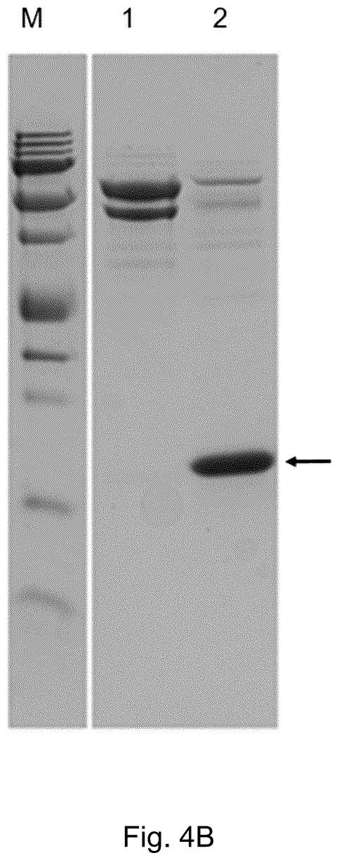

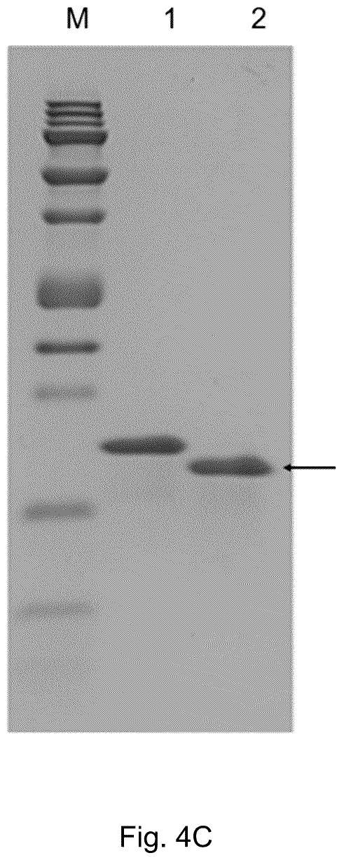

The isoelectric point for the native mycodulcein (HTS) was determined to be ˜9.5 by isoelectric focusing, suggesting a cation exchange column might be successful in purifying mycodulcein. The clarified cell lysate prepared as described in Example 10 was mixed with 2× starting buffer to obtain a cell lysate in 50 mM sodium phosphate, 1M ammonium sulfate at pH 7.0, and it was stored at 4° C. for future use. The purification procedure was performed on ÄKTA Explorer 100 system (GE Healthcare, Sweden), and the eluted proteins were monitored at 280 nm and 215 nm on a UV detector UV-900 (GE Healthcare, Sweden). A PreDictor plate (GE Healthcare, Sweden) prefilled with CIEX resins was used to screen binding conditions of native mycodulcein. The prefilled plate contains three main resins: Capto S (strong CIEX), Capto MMC (Weak CIEX), and SP Sepharose Fast Flow (strong CIEX). Lysate was dialyzed into 20 mM sodium phosphate dibasic, and different pH values ranging from 4 to 9 were screened and the optimal conditions were then scaled up using a HiScreen column. Equilibration was conducted using 25 mM sodium phosphate dibasic at pH 5 at flow rate of 3 mL/min. Binding proteins were eluted by an increasing sodium chloride gradient from 0 to 1 M using 25 mM sodium phosphate dibasic, 1M sodium chloride at pH 7. Different binding and eluting conditions were screened in which the weak cation exchanger Capto MMC showed the best binding capabilities at pH 5. However, due to low purity (25%) after this step, an alternative purification step was sought. FIG. 4A shows PAGE analysis of the eluted fraction from Capto MMC. M: protein marker; lane 1: eluted fraction, showing low purity after cation exchange. Arrow indicates mycodulcein band.

### HIC Assessment.

Cell lysate was also subjected to hydrophobic interaction chromatography (HIC) using a HiScreen CaptoButyl column (Cytiva Sweden AB, Upsala, Sweden); equilibration was carried out using 5 column volumes of 50 mM sodium phosphate, 1M ammonium sulfate at pH 7.0. Cell lysate was then loaded into the column at flow rate of 3 mL/min. Elution of bound proteins was performed by a decreasing ammonium sulfate gradient from 1 to 0 M using 50 mM sodium phosphate at pH 7.0. All obtained fractions were analyzed by SDS-PAGE.

FIG. 4B shows two eluted fractions collected during the gradient elution from the HiScreen Capto Butyl column analyzed on SDS-PAGE. Lane 1 shows eluted fraction 1 not containing mycodulcein and Lane 2 shows eluted mycodulcein. The purity of the eluted fraction was determined by GelAnalyzer to be ˜86%.

### SEC Assessment.

The eluted fraction containing native mycodulcein was then further purified using size exclusion chromatography (SEC) HiPrep 26/60 Sephacryl S-200 HR column (Cytiva Sweden AB, Upsala, Sweden), and eluted with buffer containing 50 mM sodium phosphate and 150 mM NaCl at pH 7.0. Fractions were collected and were then concentrated and desalted using 3 kD molecular weight cut-offs (MWCO) centrifugal filters (Millipore-Sigma, Germany) and then analyzed by SDS-PAGE.

Summary: although mycodulcein binds successfully to the weak cation exchanger Capto MMC, the relatively low purity of the eluted fraction made CIEX a less favorable capture/intermediate purification step. On the other hand, a higher purity fraction was obtained from the HIC, Capto Butyl column. As per the SDS-PAGE analysis, impurities seemed to have a relatively high molecular weight which made SEC a great candidate to obtain a high pure mycodulcein.

Purity after HIC/SEC is approximately 98% by GelAnalyzer of SDS-PAGE. FIG. 4C shows the eluted protein from the HIC column after chromatographing on HiPrep 26/60 Sephacryl S-200. Lane 1 shows purified his-tag mycodulcein and Lane 2 shows purified mycodulcein.

The purified native protein purified from S. cerevisiae was tasted at 0.03 mg/ml by a trained sensory scientist (0.2 mL aliquot) and found to have a sweetness equivalent to 8° brix (approximately 8% sucrose solution). The sweet taste was very noticeably sweet, with a “clean” sweetness (sugar-like taste) with no other flavors, with a slightly delayed onset and a sweet aftertaste. It has subsequently been determined that the mycodulcein expressed in S. cerevisiae is a mixture of two isoforms HTS-1 (SEQ ID NO:3) and HTS-2 (SEQ ID NO:141). The relative amounts of the HTS-1 to HTS-2 isoforms expressed are about 40% to 60% by weight. The two isoforms can be expressed in S. cerevisiae with or without His-tags or related protein tags.

### Example 12 (Exemplary Food Compositions)

His-tagged mycodulcein prepared as in Example 5 and purified as described herein was tested in a yogurt base. The yogurt base had the following recipe in Table 5.

TABLE 5
Yogurt Base Recipe

| Ingredient | % |
|---|---|
| Water | 81.1200% |
| Pea Protein | 10.6000% |
| Sunflower lecithin | 0.0600% |
| Coconut Oil (AAK) | 5.1000% |
| Ticaloid YG LP (TIC | 0.2000% |
| GUMS) | |
| Citri-Fi Citrus Fiber | 0.6000% |
| Canola Oil | 1.0000% |
| Cane Sugar | 1.3000% |
| YoFLEX YF-LO2 | 0.0200% |
| Cultures (CHR-Hansen) | |
| Total | 0.0000% |
| | 100.0000% |

The cane sugar is added as a carbon source for the yogurt cultures and is at least partially consumed by the cultures. Mycodulcein (HTS) was added to approximate the sweetness from 8° to 10° Brix of sucrose and the final concentration in the yogurt base is 0.05 mg/ml. Taste testing was performed by a trained sensory scientist and the yogurt was found to have a sweetness equivalent to 8° brix (approximately 8% sucrose solution). The sweet taste was very noticeably sweet, with a “clean” sweetness (sugar-like taste) with no other flavors, with a slightly delayed onset and a sweet aftertaste.

His-tagged mycodulcein (HTS-His-tagged) prepared as in Example 5 and purified as described herein was tested in whole milk; non-dairy pea-based milk (water, 93.75%, pea protein, 4.2%, canola oil, 1.7%, TIC Gum Blend Pro 181 AG (Acacia+Gellan) 0.3%, sunflower lecithin 0.05%); cold coffee; and water (control) at a final concentration of 0.04 mg/ml, predicted to provide a sweet level of between 8° to 10° Brix of sucrose. It was confirmed by taste testing that the sweet protein delivers a sweet level of between 8° to 10° Brix in all samples, and all samples had sweetness intensity, onset, and duration that was similar to the water control.

### Example 13 (Production and Testing of Various Mycodulcein (HTS) Variants for Sweetness and Thermal Stability)

Methods to identify potential key new mutants described herein able to be prepared in food compositions as described above. Specifically, single mutants as identified in Table 8 were generated. These mutants were generated on a background of mycodulcein, e.g., SEQ ID NO:3. Thus, all Table 8 sequences are, for example, mutants with a single amino acid change relative to SEQ ID NO:3 as indicated in Table 8. All mycodulcein variants in Table 8 can be prepared with a His-tag. All mycodulcein variants in Table 8 can be prepared with the methionine at position 1 absent.

Cloning: Eco_0001 aka E. coli BL21 DE3 (obtained from New England Biolabs #C2527I), was transformed with twenty plasmids (pMy_3083 to pMy_3103) using manufacturing protocols. The competent cells were thawed and mixed with 1 ng-100 ng of plasmid DNA and held on ice for 30 minutes. A heat shock of 42° C. for 45 seconds was applied to this mixture. Immediately after, the suspension was placed into an ice bath for 10 minutes. Roughly 950 μL of pre-warmed LB media was added and then placed in a 37° C. incubator for an hour, shaking at 225 rpm. For each transformation reaction, dilutions of 1:10 and 1:100 were made and 100 μL was plated on an antibiotic plate. These plates were allowed to grow overnight at 37° C. so colonies can recover and become visible. This transformation process yielded strains Z83CE-Z103CE, containing the plasmids pMy_3083-pMy_3103 sequentially. Shake flask-scale induced expression was used to confirm heterologous expression in the E. coli host. Post-transformational mutants were plated and maintained on LB+Ampicillin 100 μg/mL agar plates.

Plates were incubated for 16 hours at 37° C. Colony screen PCR was performed to confirm genotypes using colony screening primers designed to interrogate the flanking region along with the cDNA region of the plasmid. A successful transformation resulted in a DNA fragment of a certain size while a negative control and no template control result in no PCR band. Successful transformation was observed for all mutants.

Shake flask-scale induced expression was used to confirm heterologous expression in the E. coli host.

Strains were maintained on LB+ampicillin 100 μg/mL agar plates while the negative control was maintained on a LB plate. An overnight culture was grown for each strain in 50 mL of LB liquid media in a baffled 250 mL culture shake flask. These flasks were shaken at 37° C., at 150 rpm overnight with the appropriate antibiotics. Each overnight culture was subsequently seeded into 200 mL of TB liquid media after 16 hours of growth. These larger culture flasks were shaken at 37° C. at 200 rpm and adjusted to 0.1 OD₆₀₀ with the appropriate antibiotic. Once the OD₆₀₀ reached 0.8, a supplement of IPTG was added into the media to a final concentration of 0.66 mM. The flasks then were shaken for 5 more hours. After this time had elapsed, cells were collected by centrifugation at 4000 rpm for 5 min. E. coli cells were washed once with cold dH₂O and centrifuged again at 4000 rpm for 10 minutes. For confirmation of successful expression, cell lysate was created via sonication of the cell membrane. The cell lysate and cell debris were separated via centrifugation at 10,000 rpm for 5 minutes. Finally, the supernatant was aspirated and filtered through 0.2 μm PES filter and run on SDS-PAGE protein electrophoresis. Crude lysates were tasted to identify sweet-tasting mutants.

Table 6 shows results of exemplary testing. Thermal stability was tested in accordance with the methods of Example 9. Sensory testing was performed in accordance with Example 8.

TABLE 6
Exemplary results of sweetness testing on single
point mutations of mycodulcein

| Mutant | Sweetness? | AA Substitution* | Tm (° C.) |
|---|---|---|---|
| Control-No mutation | Yes | N/A | 59.4 |
| Z83CE | ND | S5Y | ND |
| Z84CE | ND | S6Y | ND |
| Z85CE | ND | F18Y | ND |
| Z86CE | ND | S25Y | ND |
| Z87CE | ND | V30A | ND |
| Z88CE | Yes | Q37K | 60.9 |
| Z89CE | Yes | Q37E | 57.7 |
| Z90CE | Yes | Q37N | 58.9 |
| Z91CE | ND | D46A | ND |
| Z92CE | ND | F48Y | ND |
| Z94CE | ND | V63A | ND |
| Z95CE | Yes | V74A | 59.5 |
| Z96CE | Yes | V76A | 62.8 |
| Z97CE | ND | F78Y | ND |
| Z98CE | Yes | V87A | 54.4 |
| Z99CE | Yes | V100A | 56.5 |
| Z100CE | Yes | Q102K | 61.23 |

*Substitution numbered with respect to SEQ ID NO: 3

Purified samples were tasted by a trained sensory scientist. Mycodulcein stocks were diluted to equal protein concentration as measured by ELISA. Subjects followed a sip and spit protocol approved by an Institutional Review Board. 0.2 ml of each purified mutant was placed on the tongue and intensity of sweetness perception, time of onset of sweet perception, and duration of sweetness perception were noted.

The exemplary results (Table 6) show that thermal stability is minimally affected by the amino acid changes in the mutants tested therein.

Additional single-site mutants for testing were generated by methods as described in this Example. Table 7 shows the mutants created. The mutations are identified relative to SEQ ID NO: 3. The first column in Table 7 shows the amino acid position relative to SEQ ID NO:3, the second column shows the amino acid at that position in SEQ ID NO:3 and the third column shows the mutations (either none, or in the format of original amino acid, position, and new amino acid, using single letter code). All single site mutations of Table 7 can be introduced into the HTS-2 isoform of SEQ ID NO:141. All single site mutations of Table 7 can also have methionine missing at position 1. All single site mutations of Table 7 can be generated with or without a His-tag or other protein tag.

TABLE 7
Summary of Single Site Mutations

| | | |
|---|---|---|
| 1 | M | None |
| 2 | P | None |
| 3 | D | D3A, D3F, D3H, D3I, D3K, D3L, D3N, D3Q, |
| | | D3R, D3V, D3W, D3Y |
| 4 | L | LAD, LAE, L4F, L4H, L4K, LAN, L4Q, L4R, L4W, L4Y |
| 5 | S | None |
| 6 | S | None |
| 7 | F | F7A, F7D, F7E, F7H, F7I, F7K, F7L, F7N, F7Q, |
| | | F7R, F7V, F7W, F7Y |
| 8 | I | I8D, 18E, I8F, I8H, I8I, I8K, I8N, I8Q, I8R, I8V, I8W, I8Y |
| 9 | T | None |
| 10 | I | I10D, I10E, I10F, I10H, I10K, 11OL, 11ON, |
| | | I10Q, I10R, I10V, I10W, I10Y |
| 11 | K | K11A, K11D, K11E, K11F, K11H, K11I, |
| | | K11L, K11N, K11Q, K11V, K11W, |
| | | K11Y |
| 12 | N | N12A, N12D, N12E, N12F, N12H, N12I, |
| | | N12K, N12L, N12Q, N12R, N12V, |
| | | N12W, N12Y |
| 13 | N | N13A, N13D, N13E, N13F, N13H, N13I, |
| | | N13K, N13L, N13Q, N13R, N13V, |
| | | N13W, N13Y |
| 14 | S | None |
| 15 | N | N15A, N15D, N15E, N15F, N15H, N15I, |
| | | N15K, N15L, N15Q, N15R, N15V, |
| | | N15W, N15Y |
| 16 | H | H16A, H16D, H16E, H16F, H16I, H16K, |
| | | H16L, H16N, H16Q, H16R, H16V, |
| | | H16W, H16Y |
| 17 | V | V17D, V17E, V17F, V17H, V17K, V17N, |
| | | V17Q, V17R, V17W, V17Y |
| 18 | F | F18A, F18D, F18E, F18H, F18I, F18K, F18L, |
| | | F18N, F18Q, F18R, F18V, F18W |
| 19 | T | None |
| 20 | R | R20A, R20D, R20E, R20F, R20H, R201, R20L, |
| | | R20N, R20Q, R20V, R20W, R20Y |
| 21 | T | None |
| 22 | A | A22D, A22E, A22F, A22H, A22K, A22N, |
| | | A22Q, A22R, A22W, A22Y |
| 23 | I | I23D, I23E, I23F, I23H, I23K, I23L, I23N, |
| | | I23Q, I23R, I23V, I23W, I23Y |
| 24 | Y | Y24A, Y24C, Y24D, Y24E, Y24F, Y24H, |
| | | Y24I, Y24K, Y24L, Y24N, Y24Q, |
| | | Y24R, Y24V, Y24W |
| 25 | S | None |
| 26 | K | K26A, K26D, K26E, K26F, K26H, K26I, |
| | | K26L, K26N, K26Q, K26R, K26V, |
| | | K26W, K26Y |
| 27 | Y | Y27A, Y27D, Y27E, Y27F, Y27H, Y27I, |
| | | Y27K, Y27L, Y27N, Y27Q, Y27R, |
| | | Y27V, Y27W |
| 28 | A | A28D, A28E, A28F, A28H, A28K, A28N, |
| | | A28Q, A28R, A28W, A28Y |
| 29 | A | A29D, A29E, A29F, A29H, A29K, A29N, |
| | | A29Q, A29R, A29W, A29Y |
| 30 | V | V30D, V30E, V30F, V30H, V30I, V30K, |
| | | V30L, V30N, V30Q, V30R, V30W, |
| | | V30Y |
| 31 | Q | Q31A, Q31D, Q31E, Q31F, Q31H, Q31I, |
| | | Q31K, Q31L, Q31N, Q31R, Q31V, |
| | | Q31W, Q31Y |
| 32 | W | W32A, W32D, W32E, W32F, W32H, W32I, |
| | | W32K, W32L, W32N, W32Q, |
| | | W32R, W32V, W32Y |
| 33 | S | S33T, S33M, S33C |
| 34 | P | P34A, P34D, P34E, P34F, P34H, P34I, P34K, |
| | | P34L, P34N, P34Q, P34R, P34V, |
| | | P34W, P34Y |
| 35 | E | E35A, E35F, E35H, E35I, E35K, E35L, E35N, |
| | | E35Q, E35R, E35V, E35W, E35Y |
| 36 | P | P36A, P36D, P36E, P36F, P36H, P36I, P36K, |
| | | P36L, P36N, P36Q, P36R, P36V, |
| | | P36W, P36Y |
| 37 | Q | Q37A, Q37D, Q37F, Q37H, Q37I, Q37L, Q37N, |
| | | Q37R, Q37V, Q37W, Q37Y |
| 38 | L | L38D, L38E, L38F, L38H, L38K, L38N, |
| | | L38Q, L38R, L38W, L38Y |
| 39 | S | S39T, S39M |
| 40 | I | I40D, I40E, I40F, I40H, 140K, I40L, I40N, |
| | | I40Q, I40R, I40V, I40W, I40Y |
| 41 | S | None |
| 42 | P | P42A, P42D, P42E, P42F, P42H, P42I, P42K, |
| | | P42L, P42N, P42Q, P42R, P42V, |
| | | P42W, P42Y |
| 43 | G | G43A, G43D, G43E, G43F, G43H, G43I, |
| | | G43K, G43L, G43N, G43Q, G43R, |
| | | G43V, G43W, G43Y |
| 44 | K | K44A, K44D, K44E, K44F, K44H, K44I, |
| | | K44L, K44N, K44Q, K44R, K44V, |
| | | K44W, K44Y |
| 45 | W | W45A, W45D, W45E, W45F, W45H, |
| | | W45I, W45K, W45L, W45N, W45Q, |
| | | W45R, W45V, W45Y |
| 46 | D | D46F, D46H, D46I, D46K, D46L, D46N, |
| | | D46Q, D46R, D46V, D46W, D46Y |
| 47 | L | L47D, L47E, L47F, L47H, L47K, L47N, |
| | | L47Q, L47R, L47W, L47Y |
| 48 | F | F48A, F48D, F48E, F48H, F481, F48K, F48L, |
| | | F48N, F48Q, F48R, F48V, F48W |
| 49 | I | I49D, I49E, I49F, I49H, I49K, I49N, I49Q, |
| | | I49R, I49W, I49Y, I49C |
| 50 | L | L50D, L50E, L50F, L50H, L50K, L50N, L50Q, |
| | | L50R, L50V, L50W, L50Y |
| 51 | K | K51A, K51D, K51E, K51F, K51H, K51I, K51L, |
| | | K51N, K51Q, K51R, K51V, |
| | | K51W, K51Y |
| 52 | D | D52F, D52H, D52I, D52K, D52L, D52N, |
| | | D52Q, D52R, D52V, D52W, D52Y |
| 53 | I | I53D, I53E, I53F, I53H, I53K, I53N, I53Q, |
| | | I53R, I53W, I53Y |
| 54 | L | L54D, L54E, L54F, L54H, L54K, L54N, |
| | | L54Q, L54R, L54W, L54Y |
| 55 | S | S55T, S55M |
| 56 | I | I56D, I56E, I56F, I56H, I56K, I56N, I56Q, |
| | | I56R, I56W, I56Y |
| 57 | R | R57A, R57D, R57E, R57F, R57H, R57I, R57K, |
| | | R57L, R57N, R57Q, R57V, |
| | | R57W, R57Y |
| 58 | G | G58A, G58D, G58E, G58F, G58H, G58I, |
| | | G58K, G58L, G58N, G58Q, G58R, |
| | | G58V, G58W, G58Y |
| 59 | T | None |
| 60 | S | None |
| 61 | G | G61A, G61D, G61E, G61F, G61H, G61I, G61K, |
| | | G61L, G61N, G61Q, G61R, |
| | | G61V, G61W, G61Y |
| 62 | Y | Y62A, Y62C, Y62D, Y62E, Y62F, Y62H, |
| | | Y621, Y62K, Y62L, Y62N, Y62Q, |
| | | Y62R, Y62V, Y62W |
| 63 | V | V63D, V63E, V63F, V63H, V63L, V63K, |
| | | V63N, V63Q, V63R, V63V, V63W, |
| | | V63Y |
| 64 | Q | Q64A, Q64D, Q64E, Q64F, Q64H, Q64I, |
| | | Q64K, Q64L, Q64N, Q64R, Q64V, |
| | | Q64W, Q64Y |
| 65 | Y | Y65A, Y65D, Y65E, Y65F, Y65H, Y65I, |
| | | Y65K, Y65L, Y65N, Y65Q, Y65R, |
| | | Y65V, Y65W |
| 66 | R | R66A, R66D, R66E, R66F, R66H, R66I, |
| | | R66K, R66L, R66N, R66Q, R66V, |
| | | R66W, R66Y |
| 67 | V | V67D, V67E, V67F, V67H, V67K, V67N, |
| | | V67Q, V67R, V67W, V67Y |
| 68 | G | G68A, G68D, G68E, G68F, G68H, G68I, |
| | | G68K, G68L, G68N, G68Q, G68R, |
| | | G68V, G68W, G68Y |
| 69 | D | D69A, D69E, D69F, D69H, D69I, D69K, D69L, |
| | | D69N, D69Q, D69R, D69V, |
| | | D69W, D69Y |
| 70 | G | G70A, G70D, G70E, G70F, G70H, G70I, G70K, |
| | | G70L, G70N, G70Q, G70R, |
| | | G70V, G70W, G70Y |
| 71 | P | P71A, P71D, P71E, P71F, P71H, P71I, P71K, |
| | | P71L, P71N, P71Q, P71R, P71V, |
| | | P71W, P71Y |
| 72 | G | G72A, G72D, G72E, G72F, G72H, G72I, |
| | | G72K, G72L, G72N, G72Q, G72R, |
| | | G72V, G72W, G72Y |
| 73 | W | W73A, W73D, W73E, W73F, W73H, W73I, |
| | | W73K, W73L, W73N, W73Q, |
| | | W73R, W73V, W73Y |
| 74 | V | V74D, V74E, V74F, V74H, V74I, V74K, V74L, |
| | | V74N, V74Q, V74R, V74W, |
| | | V74Y |
| 75 | R | R75A, R75D, R75E, R75F, R75H, R75I, R75K, |
| | | R75L, R75N, R75Q, R75V, |
| | | R75W, R75Y |
| 76 | V | V76D, V76E, V76F, V76H, V76K, V76I, |
| | | V76L, V76N, V76Q, V76R, V76W, |
| | | V76Y |
| 77 | T | None |
| 78 | F | F78A, F78D, F78E, F78H, F78I, F78K, F78L, |
| | | F78N, F78Q, F78R, F78V, F78W |
| 79 | S | None |
| 80 | S | None |
| 81 | L | L81D, L81E, L81F, L81H, L81I, L81K, L81N, |
| | | L81Q, L81R, L81V, L81W, L81Y |
| 82 | V | V82D, V82E, V82F, V82H, V82K, V82N, |
| | | V82Q, V82R, V82W, V82Y |
| 83 | G | G83A, G83E, G83F, G83H, G83I, G83K, G83L, |
| | | G83N, G83Q, G83R, G83V, |
| | | G83W, G83Y |
| 84 | A | A84D, A84F, A84H, A84K, A84N, A84Q, |
| | | A84R, A84W, A84Y |
| 85 | D | D85A, D85I, D85K, D85L, D85N, D85Q, D85R, |
| | | D85V, D85W, D85Y |
| 86 | E | E86A, E86D, E86F, E86H, E86I, E86K, E86L, |
| | | E86N, E86Q, E86R, E86V, E86W, |
| | | E86Y |
| 87 | V | V87D, V87E, V87F, V87H, V87I, V87K, V87L, |
| | | V87N, V87Q, V87R, V87W, |
| | | V87Y |
| 88 | A | A88D, A88E, A88F, A88H, A88I, A88K, A88L, |
| | | A88N, A88Q, A88R, A88V, |
| | | A88W, A88Y |
| 89 | E | E89A, E89D, E89F, E89H, E891, E89K, E89L, |
| | | E89N, E89Q, E89R, E89V, E89W, |
| | | E89Y |
| 90 | W | W90A, W90D, W90E, W90F, W90H, W90I, |
| | | W90K, W90L, W90N, W90Q, |
| | | W90R, W90V, W90Y |
| 91 | S | None |
| 92 | S | None |
| 93 | G | G93A, G93D, G93E, G93F, G93H, G93I, G93K, |
| | | G93L, G93N, G93Q, G93R, |
| | | G93V, G93W, G93Y |
| 94 | D | D94A, D94E, D94F, D94H, D94I, D94K, D94L, |
| | | D94N, D94Q, D94R, D94V, |
| | | D94W, D94Y |
| 95 | L | L95A, L95D, L95E, L95F, L95H, L95I, L95K, |
| | | L95N, L95Q, L95R, L95V, L95W, |
| | | L95Y |
| 96 | P | P96A, P96D, P96E, P96F, P96H, P96I, P96K, |
| | | P96L, P96N, P96Q, P96R, P96V, |
| | | P96W, P96Y |
| 97 | D | D97A, D97E, D97F, D97H, D97I, D97K, |
| | | D97L, D97N, D97Q, D97R, D97V, |
| | | D97W, D97Y |
| 98 | G | G98D, G98E, G98F, G98H, G98I, G98K, |
| | | G98L, G98N, G98Q, G98R, G98V, |
| | | G98W, G98Y |
| 99 | F | F99D, F99E, F99H, F99I, F99K, F99L, F99N, |
| | | F99Q, F99R, F99V, F99W, F99Y |
| 100 | V | V100D, V100E, V100F, V100H, V100I, V100K, |
| | | V100L, V100N, V100Q, V100R, |
| | | V100W, V100Y |
| 101 | L | L101A, L101D, L101E, L101F, L101H, L101I, |
| | | L101K, L101N, L101Q, L101R, |
| | | L101V, L101W, L101Y |
| 102 | Q | Q102A, Q102D, Q102F, Q102H, Q102I, Q102L, |
| | | Q102R, Q102V, Q102W, Q102Y |
| 103 | K | K103D, K103E, K103F, K103H, K103I, K103L, |
| | | K103N, K103Q, K103R, K103V, |
| | | K103W, K103Y |
| 104 | P | P104A, P104D, P104E, P104F, P104H, P104I, |
| | | P104K, P104L, P104N, P104Q, |
| | | P104R, P104V, P104W, P104Y |
| 105 | V | V105D, V105E, V105F, V105H, V105K, |
| | | V105N, V105Q, V105R, V105W, |
| | | V105Y |
| 106 | R | R106A, R106D, R106E, R106F, R106H, |
| | | R106I, R106K, R106L, R106N, R106Q, |
| | | R106V, R106W, R106Y |
| 107 | T | None |
| 108 | G | G108A, G108D, G108E, G108F, G108H, G108I, |
| | | G108K, G108L, G108N, G108Q, |
| | | G108R, G108V, G108W, G108Y |
| 109 | S | None |
| 110 | R | R110D, R110E, R110F, R110H, R110I, |
| | | R110K, R110L, R110N, R110Q, R110V, |
| | | R110W, R110Y |
| 111 | P | P111A, P111D, P111E, P111F, P111H, P111I, |
| | | P111K, P111L, P111N, P111Q, |
| | | P111R, P111V, P111W, P111Y |
| 112 | L | L112D, L112E, L112F, L112H, L112K, L112N, |
| | | L112Q, L112R, L112W, L112Y |
| 113 | Q | Q113A, Q113D, Q113E, Q113F, Q113H, Q113I, |
| | | Q113K, Q113L, Q113N, Q113R, |
| | | Q113V, Q113W, Q113Y |
| 114 | A | A114D, A114E, A114F, A114H, A114I, A114K, |
| | | A114L, A114N, A114Q, A114R, |
| | | A114V, A114W, A114Y |
| 115 | T | None |
| 116 | F | F116D, F116E, F116H, F116I, F116K, F116L, |
| | | F116N, F116Q, F116R, F116V, |
| | | F116W, F116Y |
| 117 | E | E117A, E117F, E117H, E117I, E117K, |
| | | E117L, E117N, E117Q, E117R, E117V, |
| | | E117W, E117Y |
| 118 | A | A118F, A118H, A118K, A118N, A118Q, |
| | | A118R, A118W, A118Y |
| 119 | T | None |
| 120 | K | K120F, K120H, K120I, K120L, K120N, K120Q, |
| | | K120R, K120V, K120W, K120Y |
| 121 | Q | Q121F, Q121H, Q121I, Q121K, Q121L, |
| | | Q121N, Q121R, Q121V, Q121W, |
| | | Q121Y. |

In variants of Table 7, variants in which the proline at any of positions 34, 36, 42, 71 or 111 (based on SEQ ID NO:3) is modified to any other amino acid are less preferred.

### Example 14: Generation and Characterization of HTS Variants with Increased Thermostability

This Example describes the design, expression, testing, and validation of Myd1 (wild-type HTS protein) variants that are more thermostable that the native (wild-type) protein for use, among others, in the food industry. Current standard pasteurization and sterilization techniques employed in food manufacturing can degrade native Myd1 structure due to its relative low melting temperature. The thermostable variants as described in this example provide sweet taste HTS protein that is useful in food and other comestibles that are subject to heating, such as in pasteurization.

This example relates to the design of focused amino acid substitutions for disulfide bridge formation. Disulfide bridges can increase stability of a protein.

A specific region of the HTS protein (amino acids acids ˜20-˜ 70) was chosen for these experiments. It was hypothesized that upon heating, this region would be the first to denature. Selected amino acids in this region were substituted with two cysteines, to determine if forming a disulfide bridge between the two cysteines would retain protein sweetness, as well as increase the melting temperature of the variant protein.

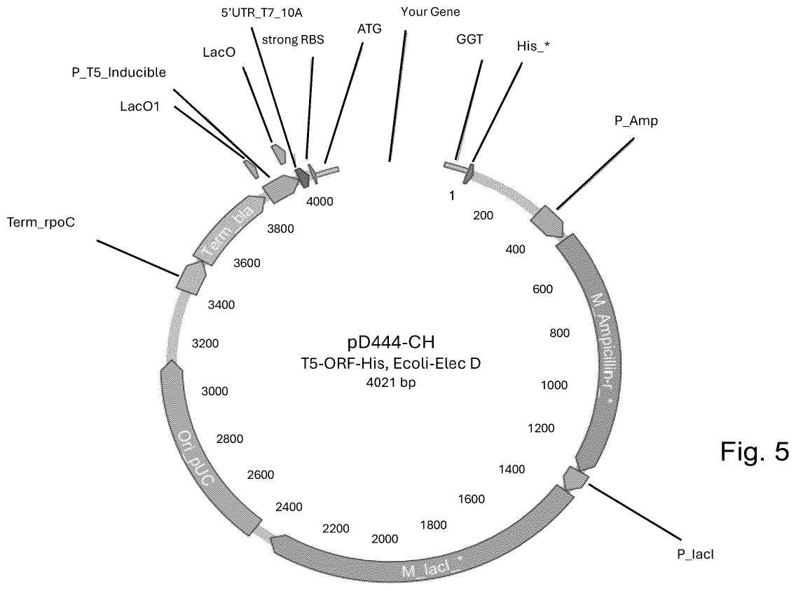

For one candidate double mutant (HTS S33C_I49C (SEQ ID NO: 143)), the E. coli optimized nucleotide sequence encoding the double mutant variant, provided as (SEQ ID NO: 144, with Met at position 1 with no stop codon), was inserted into the isopropyl beta-D-1-thiogalactopyranoside (IPTG)-inducible vector (pD444-CH, AU™, Newark, CA) (FIG. 5). The HTS variant translated from the pD44-CH vector contains two cysteins indicated and a C-terminal 6×His tag (encoded in the vector).

Pure circular plasmids (pD444-CH, AU™, Newark, CA) were transformed into chemically competent E. coli BL21-DE3 using heat shock transformation with transformant recovery on LB plates containing Ampicillin 100 μg/mL. Three colonies of successful transformants were grown overnight in LB_amp at 37° C., with shaking at 200 rpm. Glycerol stocks were made from overnight cultures of each of the three transformants.

A glycerol stock for each was then used to seed 50 mL of LB_amp and the cultures were grown overnight at 37° C., shaking at 200 rpm. The next morning, LB cultures were used to seed 200 mL of TB_amp in 2 L baffled flasks to bring the starting OD₆₀₀ values to 0.1. Cultures were then grown at 37° C., with shaking at 200 rpm. OD₆₀₀ values were monitored for about 1.5 hours when values reached OD₆₀₀ of 0.6-0.8. 100 mM IPTG was added to the flasks to bring the final concentration to 0.66 mM to induce protein expression. Flasks were grown for an additional 5 hours and harvested by spinning down at 4,000 g for 20 minutes. Pellets were washed with water and re-spun at 4,000 g for 20 minutes. Cell pellets were placed in the freezer for future lysis and protein purification.

Cell pellets for BL21-DE3 mutants expressing double mutant HTS_S33C_I49C were thawed on ice and resuspended in ˜25 mL HisTrap™ (Cytiva Sweden AB, Upsala, Sweden) working buffer (20 mM sodium phosphate, 0.5 M sodium chloride and 25 mM imidazole; (the imidazole concentration typically ranges from 20-40 mM and is adjusted for a given protein as is known in the art). Resuspended mutants were then passed through an Emulsiflex C3 high pressure homogenizer (Avestin, Ottawa, Ontario, Canada) and lysed at a pressure ≥18,000 psi. Cell lysate was collected on ice and then spun down at 4,000 g for 1 hour at 4° C. Supernatant was collected and passed through a 0.22 μm filter before loading on the FPLC (Fast Protein Liquid Chromatography) system.

Clarified lysate (20 mL) was loaded onto a 5 mL HisTrap™ FF column using an FPLC (ÄKTA FPLC™, Cytiva Sweden AB, Upsala, Sweden). The column was washed with 5 column volumes of working buffer and then protein was eluted using a gradient elution and protein eluted at ˜45% elution buffer (250 mM imidazole). Resulting protein samples were desalted on 3 kD Molecular weight Cut Off (MWCO) filters and buffer exchanged with 5 mM sodium phosphate. Resulting protein samples were run on an 16.5% Tris Tricine gel, using a native HTS sample expressed from S. cerevisiae (˜1 mg/mL) as a control. The purified double mutant variant also tasted sweet. Upon this observation, it was decided to move forward with testing to see if melting temperature was increased.

A GLOMELT™ thermostability assay (Biotrend Chemikalien GmbH, Köln, DE) was carried out to determine Tm of the purified variant. This assay uses a quantitative (q) PCR thermocycler to read fluorescence as temperature increases. This assay determines unfolding and denaturization of a protein as a function of temperature as measured by change in fluorescence of a fluorescent dye. The dye does not bond to the native protein, but bonds to the protein as it unfolds and fully denatures.

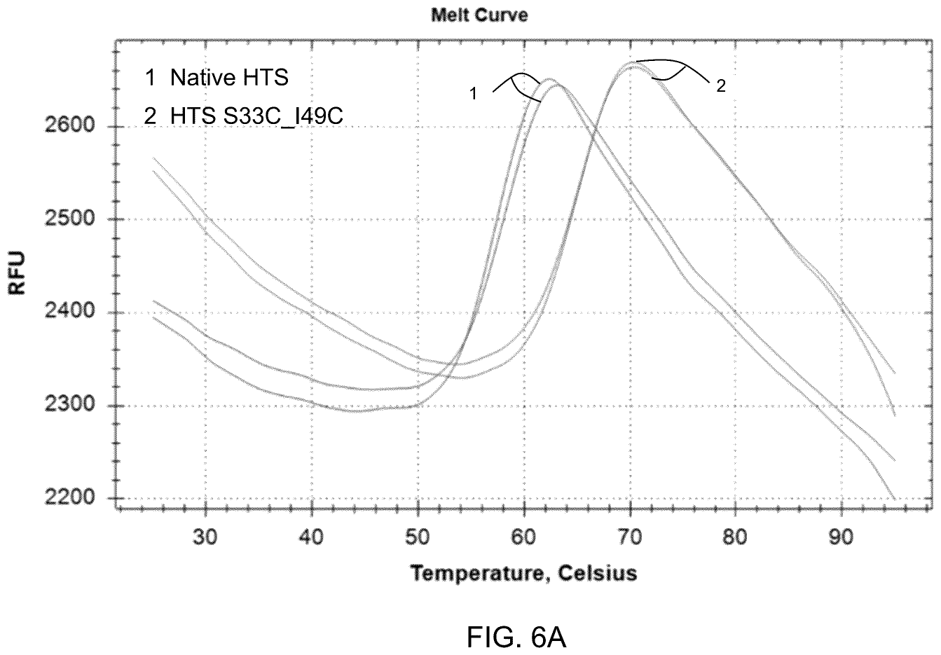

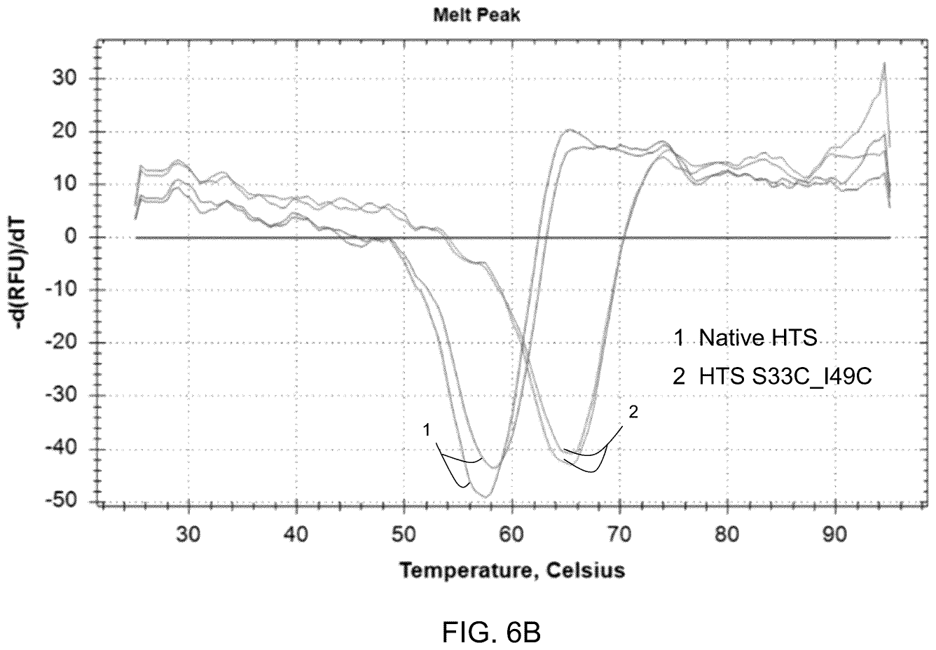

Using a CFX96™ (Bio-Rad, Hercules, CA) PCR thermocycler system, the parameters for the Glomelt™ assay (FIGS. 6A and 6B) are as follows:
25° C.: 30 seconds;
25° C.: 10 seconds;
Increase 0.5° C. every 10 seconds: Read RFU in SYBR (channel every 10 seconds).

Protein was observed to be pure enough for the assay requirements based on SDS-PAGE (at least 80% pure). Initial assays were run using unknown concentrations of the variant protein as a first pass. It was assumed that protein concentrations met the assay requirements of 0.5-5.0 ug/uL. CFX Maestro™ (Bio-Rad, Hercules, CA) software was used to analyze the raw data and generate melt curves.

The results of the thermostability assay are shown in FIGS. 6A and 6B,

The increase in Tm seen with the HTS_S33C_I49C variant compared to native HTS shows that it is a protein that can survive the harsh conditions employed in food manufacturing to ensure food safety.

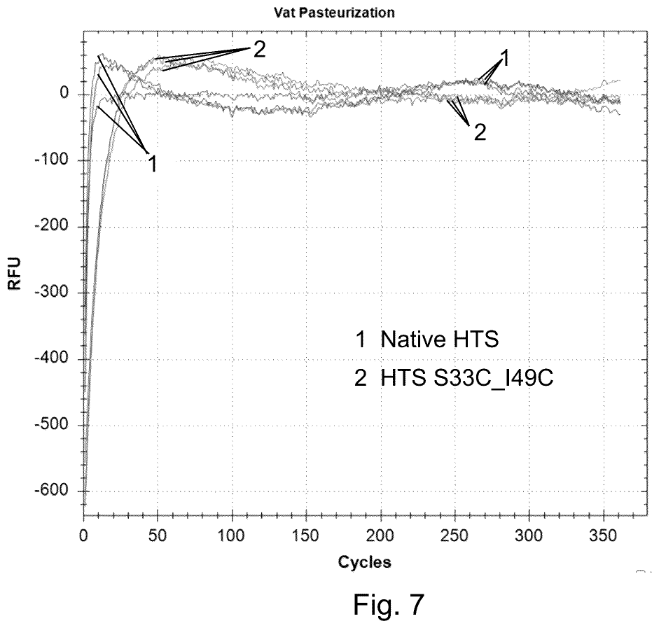

Since a shift in Tm was seen with the double mutant a more functional assay was designed to better mimic the conditions of a low temperature, long time pasteurization technique commonly used in the dairy industry (vat pasteurization). This assay employed the same GloMelt™ assay kit, but instead of ramping the temperature up over time, the sample is brought to a low pasteurization temperature (63° C.) and is held for an hour. If max fluorescence is seen before 30 minutes, then it is known the protein will not survive this type of manufacturing process (vat pasteurization). However, if there is a shift in max fluorescence over time vs. the native protein, then the variant is indeed more thermostable under vat pasteurization conditions and is a protein that can be added in food using this practice. The results of this assay are shown in FIG. 7 for Native HTS protein (1) and the double mutant (2).

Thermocycler parameters for FIG. 7:
Bring sample temp to 63° C.;
63° for 60 min° C.: read RFU every 10 seconds.

As illustrated in FIG. 7, the shift in peak RFU of 6.22 minutes showed that the double mutant protein survives that much longer under these pasteurization conditions than the native HTS protein.

A similar thermostable HTS variant is prepared by introducing two cysteines into the polypeptide of SEQ ID NO:3 at amino acid positions 24 and 62 (double mutant Y24C_Y62C SEQ ID NO:145). Analogous thermostable variants of SEQ ID NO:141 are the variants of SEQ ID NO: 146 and SEQ ID NO:147 which are optionally generated with a His-tag or other protein tag.

### Example 15 (Honey Truffle Sweetener (HTS) Protein Variants Generated by Site Mutations)

Amino acid mutations able to generate HTS protein variants are summarized in Table 8. The mutations of Table 8 are identified relative to the amino acid sequence of SEQ ID NO:3, with one column indicating the amino acid substitution of the mature protein (i.e., with no methionine at amino acid position 1) and a second column indicating the amino acid substitution of the protein where methionine is still present at amino acid position 1. The presence or absence of the methionine at position 1 of the HTS protein does not affect sweet taste of the variant protein. A polypeptide having an amino acid sequence of a variant listed in Table 8 will have the amino acid sequence of SEQ ID NO:3, but with one or more site mutations identified in Table 8.

TABLE 8
Summary of single site mutations producing protein variants

| | | Amino Acid |
|---|---|---|
| | Amino Acid | Substitution |
| | Substitution | (Met present |
| No. | (no Met at position 1) | at position 1) |
| 1 | AA113A to F | A114F |
| 2 | AA113A to L | A114L |
| 3 | AA113A to W | A114W |
| 4 | AA113A to Y | A114Y |
| 5 | AA117A to F | A118F |
| 6 | AA87A to Q | A88Q |
| 7 | AA2D to L | D3L |
| 8 | AA45D to F | D46F |
| 9 | AA45D to I | D46I |
| 10 | AA45D to K | D46K |
| 11 | AA45D to L | D46L |
| 12 | AA45D to Q | D46Q |
| 13 | AA45D to R | D46R |
| 14 | AA45D to V | D46V |
| 15 | AA45D to W | D46W |
| 16 | AA45D to Y | D46Y |
| 17 | AA51D to F | D52F |
| 18 | AA51D to H | D52H |
| 19 | AA51D to I | D52I |
| 20 | AA51D to K | D52K |
| 21 | AA51D to L | D52L |
| 22 | AA51D to R | D52R |
| 23 | AA51D to V | D52V |
| 24 | AA51D to W | D52W |
| 25 | AA51D to Y | D52Y |
| 26 | AA34E to H | E35H |
| 27 | AA34E to N | E35N |
| 28 | AA34E to W | E35W |
| 29 | AA85E to H | E86H |
| 30 | AA85E to I | E86I |
| 31 | AA115F to L | F116L |
| 32 | AA115F to W | F116W |
| 33 | AA115F to Y | F116Y |
| 34 | AA17F to A | F18A |
| 35 | AA17F to D | F18D |
| 36 | AA17F to E | F18E |
| 37 | AA17F to H | F18H |
| 38 | AA17F to K | F18K |
| 39 | AA17F to N | F18N |
| 40 | AA17F to Q | F18Q |
| 41 | AA17F to R | F18R |
| 42 | AA47F to A | F48A |
| 43 | AA47F to D | F48D |
| 44 | AA47F to E | F48E |
| 45 | AA47F to H | F48H |
| 46 | AA47F to I | F48I |
| 47 | AA47F to K | F48K |
| 48 | AA47F to L | F48L |
| 49 | AA47F to N | F48N |
| 50 | AA47F to Q | F48Q |
| 51 | AA47F to R | F48R |
| 52 | AA47F to V | F48V |
| 53 | AA57G to K | G58K |
| 54 | AA77F to V | F78V |
| 55 | AA6F to D | F7D |
| 56 | AA98F to E | F99E |
| 57 | AA98F to H | F99H |
| 58 | AA98F to N | F99N |
| 59 | AA98F to Q | F99Q |
| 60 | AA98F to W | F99W |
| 61 | AA98F to Y | F99Y |
| 62 | AA107G to E | G108E |
| 63 | AA107G to D | G108D |
| 64 | AA107G to L | G108L |
| 65 | AA107G to Q | G108Q |
| 66 | AA107G to F | G108F |
| 67 | AA107G to W | G108W |
| 68 | AA107G to Y | G108Y |
| 69 | AA42G to I | G43I |
| 70 | AA42G to K | G43K |
| 71 | AA42G to L | G43L |
| 72 | AA42G to R | G43R |
| 73 | AA42G to V | G43V |
| 74 | AA42G to Y | G43Y |
| 75 | AA57G to A | G58A |
| 76 | AA57G to D | G58D |
| 77 | AA57G to E | G58E |
| 78 | AA57G to F | G58F |
| 79 | AA57G to H | G58H |
| 80 | AA57G to I | G58I |
| 81 | AA57G to L | G58L |
| 82 | AA57G to N | G58N |
| 83 | AA57G to Q | G58Q |
| 84 | AA57G to R | G58R |
| 85 | AA57G to V | G58V |
| 86 | AA57G to W | G58W |
| 87 | AA57G to Y | G58Y |
| 88 | AA60G to A | G61A |
| 89 | AA60G to D | G61D |
| 90 | AA60G to E | G61E |
| 91 | AA60G to F | G61F |
| 92 | AA60G to H | G61H |
| 93 | AA60G to I | G61I |
| 94 | AA60G to K | G61K |
| 95 | AA60G to L | G61L |
| 96 | AA60G to N | G61N |
| 97 | AA60G to Q | G61Q |
| 98 | AA60G to R | G61R |
| 99 | AA60G to V | G61V |
| 100 | AA60G to W | G61W |
| 101 | AA60G to Y | G61Y |
| 102 | AA71G to D | G72D |
| 103 | AA71G to E | G72E |
| 104 | AA9I to D | I10D |
| 105 | AA9I to W | I10W |
| 106 | AA9I to Y | I10Y |
| 107 | AA22I to D | I23D |
| 108 | AA22I to E | I23E |
| 109 | AA22I to N | I23N |
| 110 | AA39I to D | I40D |
| 111 | AA39I to E | I40E |
| 112 | AA39I to F | I40F |
| 113 | AA39I to K | I40K |
| 114 | AA39I to N | I40N |
| 115 | AA39I to Q | I40Q |
| 116 | AA39I to R | I40R |
| 117 | AA39I to W | I40W |
| 118 | AA39I to Y | I40Y |
| 119 | AA48I to D | I49D |
| 120 | AA48I to F | I49F |
| 121 | AA48I to H | I49H |
| 122 | AA48I to N | I49N |
| 123 | AA48I to W | I49W |
| 124 | AA48I to Y | I49Y |
| 125 | AA7I to D | I8D |
| 126 | AA7I to H | I8H |
| 127 | AA7I to K | I8K |
| 128 | AA7I to N | I8N |
| 129 | AA7I to Q | I8Q |
| 130 | AA7I to W | I8W |
| 131 | AA7I to Y | I8Y |
| 132 | AA10K to D | K11D |
| 133 | AA10K to E | K11E |
| 134 | AA50K to D | K51D |
| 135 | AA50K to F | K51F |
| 136 | AA100L to A | L101A |
| 137 | AA100L to F | L101F |
| 138 | AA100L to K | L101K |
| 139 | AA100L to Q | L101Q |
| 140 | AA100L to R | L101R |
| 141 | AA100L to Y | L101Y |
| 142 | AA111L to H | L112H |
| 143 | AA111L to N | L112N |
| 144 | AA111L to Q | L112Q |
| 145 | AA111L to Y | L112Y |
| 146 | AA37L to D | L38D |
| 147 | AA37L to N | L38N |
| 148 | AA3L to E | L4E |
| 149 | AA49L to D | L50D |
| 150 | AA49L to E | L50E |
| 151 | AA49L to F | L50F |
| 152 | AA49L to H | L50H |
| 153 | AA49L to K | L50K |
| 154 | AA49L to N | L50N |
| 155 | AA49L to R | L50R |
| 156 | AA49L to V | L50V |
| 157 | AA49L to W | L50W |
| 158 | AA49L to Y | L50Y |
| 159 | AA11N to A | N12A |
| 160 | AA11N to D | N12D |
| 161 | AA11N to E | N12E |
| 162 | AA11N to F | N12F |
| 163 | AA11N to H | N12H |
| 164 | AA11N to I | N12I |
| 165 | AA11N to K | N12K |
| 166 | AA11N to L | N12L |
| 167 | AA11N to Q | N12Q |
| 168 | AA11N to R | N12R |
| 169 | AA11N to W | N12W |
| 170 | AA11N to Y | N12Y |
| 171 | AA12N to I | N13I |
| 172 | AA12N to V | N13V |
| 173 | AA12N to W | N13W |
| 174 | AA103P to E | P104E |
| 175 | AA103P to I | P104I |
| 176 | AA33P to V | P34V |
| 177 | AA33P to W | P34W |
| 178 | AA33P to Y | P34Y |
| 179 | AA35P to A | P36A |
| 180 | AA35P to D | P36D |
| 181 | AA35P to E | P36E |
| 182 | AA35P to F | P36F |
| 183 | AA35P to H | P36H |
| 184 | AA35P to I | P36I |
| 185 | AA35P to K | P36K |
| 186 | AA35P to L | P36L |
| 187 | AA35P to N | P36N |
| 188 | AA35P to Q | P36Q |
| 189 | AA35P to R | P36R |
| 190 | AA35P to V | P36V |
| 191 | AA35P to W | P36W |
| 192 | AA35P to Y | P36Y |
| 193 | AA41P to D | P42D |
| 194 | AA41P to F | P42F |
| 195 | AA41P to H | P42H |
| 196 | AA41P to L | P42L |
| 197 | AA41P to N | P42N |
| 198 | AA41P to V | P42V |
| 199 | AA41P to W | P42W |
| 200 | AA41P to Y | P42Y |
| 201 | AA95P to K | P96K |
| 202 | AA95P to N | P96N |
| 203 | AA112Q to D | Q113D |
| 204 | AA36Q to F | Q37F |
| 205 | AA36Q to W | Q37W |
| 206 | AA36Q to Y | Q37Y |
| 207 | AA105R to A | R106A |
| 208 | AA105R to D | R106D |
| 209 | AA105R to E | R106E |
| 210 | AA105R to N | R106N |
| 211 | AA105R to W | R106W |
| 212 | AA109R to D | R110D |
| 213 | AA109R to E | R110E |
| 214 | AA109R to L | R110L |
| 215 | AA109R to V | R110V |
| 216 | AA109R to W | R110W |
| 217 | AA109R to I | R110I |
| 218 | AA19R to D | R20D |
| 219 | AA19R to E | R20E |
| 220 | AA19R to F | R20F |
| 221 | AA19R to H | R20H |
| 222 | AA19R to N | R20N |
| 223 | AA19R to W | R20W |
| 224 | AA19R to Y | R20Y |
| 225 | AA56R to F | R57F |
| 226 | AA56R to L | R57L |
| 227 | AA91S to T | S92T |
| 228 | AA104V to D | V105D |
| 229 | AA62V to D | V63D |
| 230 | AA62V to E | V63E |
| 231 | AA62V to F | V63F |
| 232 | AA62V to H | V63H |
| 233 | AA62V to K | V63K |
| 234 | AA62V to L | V63L |
| 235 | AA62V to N | V63N |
| 236 | AA62V to Q | V63Q |
| 237 | AA62V to R | V63R |
| 238 | AA62V to W | V63W |
| 239 | AA62V to Y | V63Y |
| 240 | AA72W to A | W73A |
| 241 | AA72W to E | W73E |
| 242 | AA72W to I | W73I |
| 243 | AA72W to K | W73K |
| 244 | AA72W to N | W73N |
| 245 | AA72W to Q | W73Q |
| 246 | AA72W to R | W73R |
| 247 | AA23Y to D | Y24D |
| 248 | AA23Y to L | Y24L |
| 249 | AA61Y to A | Y62A |
| 250 | AA61Y to D | Y62D |
| 251 | AA61Y to E | Y62E |
| 252 | AA61Y to K | Y62K |
| 253 | AA61Y to L | Y62L |
| 254 | AA61Y to N | Y62N |
| 255 | AA61Y to R | Y62R |
| 256 | AA64Y to D | Y65D |
| 257 | AA63Q to W | Q64W |
| 258 | AA64Y to A | Y65A |
| 259 | AA64Y to E | Y65E |
| 260 | AA64Y to F | Y65F |
| 261 | AA64Y to H | Y65H |
| 262 | AA64Y to I | Y65I |
| 263 | AA64Y to K | Y65K |
| 264 | AA64Y to L | Y65L |
| 265 | AA64Y to N | Y65N |
| 266 | AA64Y to Q | Y65Q |
| 267 | AA64Y to R | Y65R |
| 268 | AA64Y to V | Y65V |
| 269 | AA65R to F | R66F |
| 270 | AA65R to Y | R66Y |
| 271 | AA66V to D | V67D |
| 272 | AA66V to E | V67E |
| 273 | AA66V to F | V67F |
| 274 | AA66V to H | V67H |
| 275 | AA66V to K | V67K |
| 276 | AA66V to N | V67N |
| 277 | AA66V to Q | V67Q |
| 278 | AA66V to R | V67R |
| 279 | AA66V to W | V67W |
| 280 | AA66V to Y | V67Y |
| 281 | AA67G to E | G68E |
| 282 | AA67G to F | G68F |
| 283 | AA67G to H | G68H |
| 284 | AA67G to I | G68I |
| 285 | AA67G to K | G68K |
| 286 | AA67G to L | G68L |
| 287 | AA67G to Q | G68Q |
| 288 | AA67G to R | G68R |
| 289 | AA67G to V | G68V |
| 290 | AA67G to W | G68W |
| 291 | AA67G to Y | G68Y |
| 292 | AA69G to A | G70A |
| 293 | AA69G to D | G70D |
| 294 | AA69G to F | G70F |
| 295 | AA69G to H | G70H |
| 296 | AA69G to I | G70I |
| 297 | AA69G to K | G70K |
| 298 | AA69G to L | G70L |
| 299 | AA69G to N | G70N |
| 300 | AA69G to Q | G70Q |
| 301 | AA69G to R | G70R |
| 302 | AA69G to V | G70V |
| 303 | AA69G to W | G70W |
| 304 | AA69G to Y | G70Y |
| 305 | AA70P to W | P71W |
| 306 | AA71G to F | G72F |
| 307 | AA71G to I | G72I |
| 308 | AA71G to K | G72K |
| 309 | AA71G to L | G72L |
| 310 | AA71G to Q | G72Q |
| 311 | AA71G to R | G72R |
| 312 | AA71G to V | G72V |
| 313 | AA72W to D | W73D |
| 314 | AA72W to L | W73L |
| 315 | AA72W to V | W73V |
| 316 | AA73V to D | V74D |
| 317 | AA73V to E | V74E |
| 318 | AA73V to H | V74H |
| 319 | AA73V to K | V74K |
| 320 | AA73V to N | V74N |
| 321 | AA73V to Q | V74Q |
| 322 | AA73V to R | V74R |
| 323 | AA73V to W | V74W |
| 324 | AA73V to Y | V74Y |
| 325 | AA74R to D | R75D |
| 326 | AA74R to F | R75F |
| 327 | AA74R to N | R75N |
| 328 | AA74R to W | R75W |
| 329 | AA74R to Y | R75Y |
| 330 | AA75V to D | V76D |
| 331 | AA75V to E | V76E |
| 332 | AA75V to H | V76H |
| 333 | AA75V to K | V76K |
| 334 | AA75V to N | V76N |
| 335 | AA75V to Q | V76Q |
| 336 | AA75V to R | V76R |
| 337 | AA75V to W | V76W |
| 338 | AA75V to Y | V76Y |
| 339 | AA77F to A | F78A |
| 340 | AA77F to D | F78D |
| 341 | AA77F to E | F78E |
| 342 | AA77F to H | F78H |
| 343 | AA77F to I | F78I |
| 344 | AA77F to K | F78K |
| 345 | AA77F to L | F78L |
| 346 | AA77F to N | F78N |
| 347 | AA77F to Q | F78Q |
| 348 | AA77F to R | F78R |
| 349 | AA80L to Y | L81Y |
| 350 | AA81V to W | V82W |
| 351 | AA82G to W | G83W |
| 352 | AA84D to W | D85W |
| 353 | AA85E to F | E86F |
| 354 | AA85E to K | E86K |
| 355 | AA85E to L | E86L |
| 356 | AA85E to N | E86N |
| 357 | AA85E to Q | E86Q |
| 358 | AA85E to R | E86R |
| 359 | AA85E to V | E86V |
| 360 | AA85E to W | E86W |
| 361 | AA85E to Y | E86Y |
| 362 | AA86V to N | V87N |
| 363 | AA86V to W | V87W |
| 364 | AA87A to D | A88D |
| 365 | AA87A to E | A88E |
| 366 | AA87A to H | A88H |
| 367 | AA87A to I | A88I |
| 368 | AA87A to K | A88K |
| 369 | AA87A to R | A88R |
| 370 | AA88E to W | E89W |
| 371 | AA89W to A | W90A |
| 372 | AA89W to D | W90D |
| 373 | AA89W to E | W90E |
| 374 | AA89W to H | W90H |
| 375 | AA89W to K | W90K |
| 376 | AA89W to N | W90N |
| 377 | AA89W to Q | W90Q |
| 378 | AA89W to R | W90R |
| 379 | AA92G to F | G93F |
| 380 | AA92G to I | G93I |
| 381 | AA92G to V | G93V |
| 382 | AA93D to W | D94W |
| 383 | AA94L to D | L95D |
| 384 | AA94L to F | L95F |
| 385 | AA94L to H | L95H |
| 386 | AA94L to K | L95K |
| 387 | AA94L to N | L95N |
| 388 | AA94L to R | L95R |
| 389 | AA94L to W | L95W |
| 390 | AA94L to Y | L95Y |
| 391 | AA95P to A | P96A |
| 392 | AA95P to D | P96D |
| 393 | AA95P to E | P96E |
| 394 | AA95P to F | P96F |
| 395 | AA95P to H | P96H |
| 396 | AA95P to I | P96I |
| 397 | AA95P to L | P96L |
| 398 | AA95P to Q | P96Q |
| 399 | AA95P to R | P96R |
| 400 | AA95P to V | P96V |
| 401 | AA95P to W | P96W |
| 402 | AA95P to Y | P96Y |
| 403 | AA97G to I | G98I |
| 404 | AA97G to L | G98L |
| 405 | AA97G to V | G98V |
| 406 | AA97G to W | G98W |
| 407 | AA98F to D | F99D |
| 408 | AA98F to K | F99K |
| 409 | AA98F to R | F99R |
| 410 | AA100L to D | L101D |
| 411 | AA100L to E | L101E |
| 412 | AA100L to H | L101H |
| 413 | AA100L to N | L101N |
| 414 | AA100L to W | L101W |
| 415 | AA103P to D | P104D |
| 416 | AA103P to F | P104F |
| 417 | AA103P to H | P104H |
| 418 | AA103P to K | P104K |
| 419 | AA103P to L | P104L |
| 420 | AA103P to N | P104N |
| 421 | AA103P to Q | P104Q |
| 422 | AA103P to R | P104R |
| 423 | AA103P to W | P104W |
| 424 | AA103P to Y | P104Y |
| 425 | AA107G to I | G108I |
| 426 | AA107G to V | G108V |
| 427 | AA110P to A | P111A |
| 428 | AA110P to D | P111D |
| 429 | AA110P to E | P111E |
| 430 | AA110P to F | P111F |
| 431 | AA110P to H | P111H |
| 432 | AA110P to I | P111I |
| 433 | AA110P to K | P111K |
| 434 | AA110P to L | P111L |
| 435 | AA110P to N | P111N |
| 436 | AA110P to Q | P111Q |
| 437 | AA110P to R | P111R |
| 438 | AA110P to V | P111V |
| 439 | AA110P to W | P111W |
| 440 | AA110P to Y | P111Y |
| 441 | AA111L to D | L112D |
| 442 | AA111L to E | L112E |
| 443 | AA111L to K | L112K |
| 444 | AA111L to R | L112R |
| 445 | AA111L to W | L112W |
| 446 | AA113A to D | A114D |
| 447 | AA113A to E | A114E |
| 448 | AA113A to H | A114H |
| 449 | AA113A to K | A114K |
| 450 | AA113A to N | A114N |
| 451 | AA113A to Q | A114Q |
| 452 | AA113A to R | A114R |
| 453 | AA115F to D | F116D |
| 454 | AA115F to E | F116E |
| 455 | AA115F to H | F116H |
| 456 | AA115F to I | F116I |
| 457 | AA115F to K | F116K |
| 458 | AA115F to N | F116N |
| 459 | AA115F to Q | F116Q |
| 160 | AA115F to R | F116R |
| 461 | AA115F to V | F116V |
| 462 | AA117A to H | A118H |
| 463 | AA117A to K | A118K |
| 464 | AA117A to N | A118N |
| 465 | AA117A to Q | A118Q |
| 466 | AA117A to R | A118R |
| 467 | AA117A to W | A118W |
| 468 | AA117A to Y | A118Y |
| 469 | AA6F to I | F7I |
| 470 | AA98F to I | F99I |
| 471 | AA7I to R | I8R |
| 472 | AA17F to L | F18L |
| 473 | AA98F to V | F99V |
| 474 | AA6F to H | F7H |
| 475 | AA98F to L | F99L |
| 476 | AA105R to I | R106I |
| 477 | AA6F to V | F7V |
| 478 | AA6F to Y | F7Y |
| 479 | AA46L to F | L47F |
| 480 | AA101Q to L | Q102L |
| 481 | AA3L to H | L4H |
| 482 | AA119K to Q | K120Q |
| 483 | AA55I to D | I56D |
| 484 | AA80L to E | L81E |
| 485 | AA97G to D | G98D |
| 486 | AA6F to K | F7K |
| 487 | AA52I to K | I53K |
| 488 | AA119K to F | K120F |
| 489 | AA6F to Q | F7Q |
| 490 | AA46L to R | L47R |
| 491 | AA53L to D | L54D |
| 492 | AA6F to R | F7R |
| 493 | AA112Q to R | Q113R |
| 494 | AA61Y to W | Y62W |
| 495 | AA3L to F | L4F |
| 496 | AA46L to H | L47H |
| 497 | AA46L to Y | L47Y |
| 498 | AA36Q to A | Q37A |
| 499 | AA119K to V | K120V |
| 500 | AA50K to R | K51R |
| 501 | AA83A to Q | A84Q |
| 502 | AA46L to Q | L47Q |
| 503 | AA56R to H | R57H |
| 504 | AA46L to K | L47K |
| 505 | AA9I to V | I10V |
| 506 | AA44W to Q | W45Q |
| 507 | AA119K to I | K120I |
| 508 | AA53L to E | L54E |
| 509 | AA55I to N | I56N |
| 510 | AA13S to T | S14T |
| 511 | AA52I to N | I53N |
| 512 | AA105R to K | R106K |
| 513 | AA44W to E | W45E |
| 514 | AA119K to Y | K120Y |
| 515 | AA71G to W | G72W |
| 516 | AA87A to Y | A88Y |
| 517 | AA55I to Q | I56Q |
| 518 | AA53L to H | L54H |
| 519 | AA9I to R | I10R |
| 520 | AA105R to V | R106V |
| 521 | AA14N to D | N15D |
| 522 | AA119K to W | L120W |
| 523 | AA83A to R | A84R |
| 524 | AA10K to I | K11I |
| 525 | AA54S to M | S55M |
| 526 | AA113A to V | A114V |
| 527 | AA55I to E | I56E |
| 528 | AA112Q to K | Q113K |
| 529 | AA48I to R | I49R |
| 530 | AA52I to R | I53R |
| 531 | AA120Q to L | Q121L |
| 532 | AA83A to H | A84H |
| 533 | AA52I to Q | I53Q |
| 534 | AA96D to Y | D97Y |
| 535 | AA119K to H | K120H |
| 536 | AA53L to K | L54K |
| 537 | AA53L to N | L54N |
| 538 | AA43K to Q | K44Q |
| 539 | AA55I to Y | I56Y |
| 540 | AA56R to Y | R57Y |
| 541 | AA38S to T | S39T |
| 542 | AA120Q to I | Q121I |
| 543 | AA101Q to I | Q102I |
| 544 | AA52I to E | I53E |
| 545 | AA10K to V | K11V |
| 546 | AA120Q to V | Q121V |
| 547 | AA116E to I | E117I |
| 548 | AA43K to H | K44H |
| 549 | AA119K to N | K120N |
| 550 | AA53L to Y | L54Y |
| 551 | AA116E to V | E117V |
| 552 | AA23Y to W | Y24W |
| 553 | AA55I to H | I56H |
| 554 | AA101Q to R | Q102R |
| 555 | AA14N to A | N15A |
| 556 | AA120Q to R | Q121R |
| 557 | AA21A to Q | A22Q |
| 558 | AA68D to N | D69N |
| 559 | AA81V to N | V82N |
| 560 | AA55I to K | I56K |
| 561 | AA97G to N | G98N |
| 562 | AA120Q to K | Q121K |
| 563 | AA56R to N | R57N |
| 564 | AA56R to Q | R57Q |
| 565 | AA61Y to F | Y62F |
| 566 | AA101Q to V | Q102V |
| 567 | AA120Q to W | Q121W |
| 568 | AA83A to K | A84K |
| 569 | AA44W to A | W45A |
| 570 | AA36Q to R | Q37R |
| 571 | AA9I to E | I10E |
| 572 | AA53L to Q | L54Q |
| 573 | AA99V to F | V100F |
| 574 | AA50K to I | K51I |
| 575 | AA82G to E | G83E |
| 576 | AA6F to L | F7L |
| 577 | AA73V to F | V74F |
| 578 | AA71G to Y | G72Y |
| 579 | AA44W to H | W45H |
| 580 | AA14N to E | N15E |
| 581 | AA55I to W | I56W |
| 582 | AA44W to K | W45K |
| 583 | AA73V to I | V74I |
| 584 | AA82G to Y | G83Y |
| 585 | AA22I to Y | I23Y |
| 586 | AA6F to E | F7E |
| 587 | AA83A to F | A84F |
| 588 | AA96D to E | D97E |
| 589 | AA53L to F | L54F |
| 590 | AA119K to L | K120L |
| 591 | AA9I to Q | I10Q |
| 592 | AA96D to A | D97A |
| 593 | AA46L to W | L47W |
| 594 | AA63Q to E | Q64E |
| 595 | AA93D to F | D94F |
| 596 | AA55I to R | I56R |
| 597 | AA14N to L | N15L |
| 598 | AA81V to E | V82E |
| 599 | AA3L to W | L4W |
| 600 | AA68D to A | D69A |
| 601 | AA43K to R | K44R |
| 602 | AA68D to E | D69E |
| 603 | AA83A to Y | A84Y |
| 604 | AA44W to V | W45V |
| 605 | AA9I to K | I10K |
| 606 | AA82G to N | G83N |
| 607 | AA43K to N | K44N |
| 608 | AA99V to Y | V100Y |
| 609 | AA68D to Q | D69Q |
| 610 | AA55I to F | I56F |
| 611 | AA53L to R | L54R |
| 612 | AA16V to K | V17K |
| 613 | AA61Y to H | Y62H |
| 614 | AA44W to R | W45R |
| 615 | AA119K to R | K120R |
| 616 | AA22I to L | I23L |
| 617 | AA43K to E | K44E |
| 618 | AA21A to E | A22E |
| 619 | AA96D to R | D97R |
| 620 | AA50K to H | K51H |
| 621 | AA92G to A | G93A |
| 622 | AA93D to N | D94N |
| 623 | AA99V to I | V100I |
| 624 | AA96D to K | D97K |
| 625 | AA93D to H | D94H |
| 626 | AA52I to D | I53D |
| 627 | AA15H to F | H16F |
| 628 | AA54S to T | S55T |
| 629 | AA87A to F | A88F |
| 630 | AA96D to Q | D97Q |
| 631 | AA120Q to N | Q121N |
| 632 | AA9I to N | I10N |
| 633 | AA65R to Q | R66Q |
| 634 | AA81V to K | V82K |
| 635 | AA68D to K | D69K |
| 636 | AA81V to Y | V82Y |
| 637 | AA93D to Y | D94Y |
| 638 | AA14N to Y | N15Y |
| 639 | AA104V to K | V105K |
| 640 | AA81V to F | V82F |
| 641 | AA88E to Y | E89Y |
| 642 | AA92G to Q | G93Q |
| 643 | AA120Q to F | Q121F |
| 644 | AA81V to Q | V82Q |
| 645 | AA82G to H | G83H |
| 646 | AA81V to H | V82H |
| 647 | AA14N to F | N15F |
| 648 | AA68D to H | D69H |
| 649 | AA21A to H | A22H |
| 650 | AA68D to L | D69L |
| 651 | AA81V to D | V82D |
| 652 | AA92G to E | G93E |
| 653 | AA1P to H | P2H |
| 654 | AA96D to W | D97W |
| 655 | AA23Y to F | Y24F |
| 656 | AA48I to K | I49K |
| 657 | AA16V to H | V17H |
| 658 | AA65R to K | R66K |
| 659 | AA68D to R | D69R |
| 660 | AA21A to K | A22K |
| 661 | AA96D to L | D97L |
| 662 | AA22I to V | I23V |
| 663 | AA96D to V | D97V |
| 664 | AA80L to V | L81V |
| 665 | AA56R to K | R57K |
| 666 | AA56R to V | R57V |
| 667 | AA52I to H | I53H |
| 668 | AA85E to D | E86D |
| 669 | AA14N to Q | N15Q |
| 670 | AA105R to L | R106L |
| 671 | AA120Q to H | Q121H |
| 672 | AA101Q to H | Q102H |
| 673 | AA14N to R | N15R |
| 674 | AA120Q to Y | Q121Y |
| 675 | AA82G to F | G83F |
| 676 | AA74R to Q | R75Q |
| 677 | AA93D to Q | D94Q |
| 678 | AA17F to I | F18I |
| 679 | AA39I to L | I40L |
| 680 | AA21A to R | A22R |
| 681 | AA93D to E | D94E |
| 682 | AA16V to D | V17D |
| 683 | AA96D to N | D97N |
| 684 | AA83A to D | A84D |
| 685 | AA38S to M | S39M |
| 686 | AA68D to V | D69V |
| 687 | AA46L to N | L47N |
| 688 | AA102K to R | K103R |
| 689 | AA53L to W | L54W |
| 690 | AA37L to E | L38E |
| 691 | AA86V to I | V87I |
| 692 | AA44W to Y | W45Y |
| 693 | AA71G to A | G72A |
| 694 | AA44W to I | W45I |
| 695 | AA63Q to D | Q64D |
| 696 | AA96D to H | D97H |
| 697 | AA93D to A | D94A |
| 698 | AA112Q to H | Q113H |
| 699 | AA43K to Y | K44Y |
| 700 | AA14N to I | N15I |
| 701 | AA68D to Y | D69Y |
| 702 | AA39I to H | I40H |
| 703 | AA68D to I | D69I |
| 704 | AA44W to F | W45F |
| 705 | AA37L to K | L38K |
| 706 | AA96D to I | D97I |
| 707 | AA56R to W | R57W |
| 708 | AA96D to F | D97F |
| 709 | AA25K to A | K26A |
| 710 | AA90S to T | S91T |
| 711 | AA68D to F | D69F |
| 712 | AA99V to L | V100L |
| 713 | AA22I to K | I23K |
| 714 | AA68D to W | D69W |
| 715 | AA102K to Q | K103Q |
| 716 | AA92G to D | G93D |
| 717 | AA92G to K | G93K |
| 718 | AA16V to Y | V17Y |
| 719 | AA12N to H | N13H |
| 720 | AA15H to Y | H16Y |
| 721 | AA15H to L | H16L |
| 722 | AA21A to F | A22F |
| 723 | AA74R to K | R75K |
| 724 | AA6F to N | F7N |
| 725 | AA97G to Q | G98Q |
| 726 | AA81V to R | V82R |
| 727 | AA43K to A | K44A |
| 728 | AA97G to E | G98E |
| 729 | AA74R to A | R75A |
| 730 | AA84D to A | D85A |
| 731 | AA14N to V | N15V |
| 732 | AA92G to R | G93R |
| 733 | AA65R to N | R66N |
| 734 | AA16V to R | V17R |
| 735 | AA76T to S | T77S |
| 736 | AA75V to I | V76I |
| 737 | AA83A to W | A84W |
| 738 | AA14N to W | N15W |
| 739 | AA92G to H | G93H |
| 740 | AA69G to E | G70E |
| 741 | AA15H to I | H16I |
| 742 | AA56R to E | R57E |
| 743 | AA50K to V | K51V |
| 744 | AA16V to W | V17W |
| 745 | AA34E to A | E35A |
| 746 | AA23Y to H | Y24H |
| 747 | AA99V to K | V100K |
| 748 | AA21A to Y | A22Y |
| 749 | AA102K to E | K103E |
| 750 | AA84D to Q | D85Q |
| 751 | AA93D to L | D94L |
| 752 | AA22I to F | I23F |
| 753 | AA104V to R | V105R |
| 754 | AA92G to Y | G93Y |
| 755 | AA21A to N | A22N |
| 756 | AA84D to N | D85N |
| 757 | AA67G to D | G68D |
| 758 | AA45D to N | D46N |
| 759 | AA15H to E | H16E |
| 760 | AA97G to H | G98H |
| 761 | AA43K to D | K44D |
| 762 | AA86V to K | V87K |
| 763 | AA65R to A | R66A |
| 764 | AA92G to N | G93N |
| 765 | AA112Q to A | Q113A |
| 766 | AA44W to L | W45L |
| 767 | AA14N to H | N15H |
| 768 | AA16V to F | V17F |
| 769 | AA97G to Y | G98Y |
| 770 | AA82G to A | G83A |
| 771 | AA97G to R | G98R |
| 772 | AA15H to K | H16K |
| 773 | AA6F to A | F7A |
| 774 | AA97G to F | G98F |
| 775 | AA16V to Q | V17Q |
| 776 | AA94L to I | L95I |
| 777 | AA37L to H | L38H |
| 778 | AA101Q to A | Q102A |
| 779 | AA99V to R | V100R |
| 780 | AA80L to I | L81I |
| 781 | AA40S to T | S41T |
| 782 | AA83A to N | A84N |
| 783 | AA44W to N | W45N |
| 784 | AA21A to D | A22D |
| 785 | AA86V to Y | V87Y |
| 786 | AA44W to D | W45D |
| 787 | AA63Q to A | Q64A |
| 788 | AA3L to R | L4R |
| 789 | AA48I to Q | I49Q |
| 790 | AA86V to Q | V87Q |
| 791 | AA7I to F | I8F |
| 792 | AA70P to A | P71A |
| 793 | AA15H to W | H16W |
| 794 | AA93D to K | D94K |
| 795 | AA9I to L | I10L |
| 796 | AA109R to K | R110K |
| 797 | AA21A to W | A22W |
| 798 | AA43K to F | K44F |
| 799 | AA43K to V | K44V |
| 800 | AA70P to R | P71R |
| 801 | AA37L to Q | L38Q |
| 802 | AA99V to H | V100H |
| 803 | AA71G to H | G72H |
| 804 | AA15H to R | H16R |
| 805 | AA41P to I | P42I |
| 806 | AA99V to Q | V100Q |
| 807 | AA93D to R | D94R |
| 808 | AA10K to Y | K11Y |
| 809 | AA94L to V | L95V |
| 810 | AA80L to Q | L81Q |
| 811 | AA63Q to H | Q64H |
| 812 | AA73V to L | V74L |
| 813 | AA70P to N | P71N |
| 814 | AA56R to I | R57I |
| 815 | AA70P to K | P71K |
| 816 | AA15H to N | H16N |
| 817 | AA87A to V | A88V |
| 818 | AA65R to D | R66D |
| 819 | AA101Q to Y | Q102Y |
| 820 | AA67G to A | G68A |
| 821 | AA102K to N | K103N |
| 822 | AA46L to E | L47E |
| 823 | AA3L to N | L4N |
| 824 | AA18T to S | T19S |
| 825 | AA43K to W | K44W |
| 826 | AA63Q to V | Q64V |
| 827 | AA43K to L | K44L |
| 828 | AA63Q to I | Q64I |
| 829 | AA102K to D | K103D |
| 830 | AA3L to Q | L4Q |
| 831 | AA80L to T | L81T |
| 832 | AA56R to A | R57A |
| 833 | AA56R to D | R57D |
| 834 | AA84D to K | D85K |
| 835 | AA86V to L | V87L |
| 836 | AA93D to V | D94V |
| 837 | AA70P to Q | P71Q |
| 838 | AA99V to W | V100W |
| 839 | AA52I to Y | 153Y |
| 840 | AA65R to V | R66V |
| 841 | AA16V to N | V17N |
| 842 | AA14N to K | N15K |
| 843 | AA50K to L | K51L |
| 844 | AA74R to V | R75V |
| 845 | AA7I to E | I8E |
| 846 | AA87A to L | A88L |
| 847 | AA15H to Q | H16Q |
| 848 | AA3L to Y | L4Y |
| 849 | AA65R to E | R66E |
| 850 | AA74R to L | R75L |
| 851 | AA11N to V | N12V |
| 852 | AA74R to I | R75I |
| 853 | AA6F to W | F7W |
| 854 | AA86V to E | V87E |
| 855 | AA88E to F | E89F |
| 856 | AA97G to K | G98K |
| 857 | AA70P to H | P71H |
| 858 | AA61Y to Q | Y62Q |
| 859 | AA61Y to I | Y62I |
| 860 | AA112Q to I | Q113I |
| 861 | AA52I to F | I53F |
| 862 | AA105R to Q | R106Q |
| 863 | AA102K to H | K103H |
| 864 | AA84D to V | D85V |
| 865 | AA112Q to V | Q113V |
| 866 | AA42G to H | G43H |
| 867 | AA82G to Q | G83Q |
| 868 | AA63Q to K | Q64K |
| 869 | AA34E to Y | E35Y |
| 870 | AA88E to A | E89A |
| 871 | AA70P to D | P71D |
| 872 | AA70P to Y | P71Y |
| 873 | AA92G to W | G93W |
| 874 | AA86V to H | V87H |
| 875 | AA37L to W | L38W |
| 876 | AA70P to F | P71F |
| 877 | AA104V to Q | V105Q |
| 878 | AA16V to E | V17E |
| 879 | AA12N to Y | N13Y |
| 880 | AA37L to F | L38F |
| 881 | AA104V to Y | V105Y |
| 882 | AA88E to Q | E89Q |
| 883 | AA86V to F | V87F |
| 884 | AA93D to I | D94I |
| 885 | AA2D to H | D3H |
| 886 | AA82G to V | G83V |
| 887 | AA105R to F | R106F |
| 888 | AA63Q to N | Q64N |
| 889 | AA24S to T | S25T |
| 890 | AA42G to N | G43N |
| 891 | AA70P to V | P71V |
| 892 | AA12N to F | N13F |
| 893 | AA2D to N | D3N |
| 894 | AA63Q to R | Q64R |
| 895 | AA86V to R | V87R |
| 896 | AA42G to A | G43A |
| 897 | AA75V to L | V76L |
| 898 | AA36Q to L | Q37L |
| 899 | AA92G to L | G93L |
| 900 | AA65R to L | R66L |
| 901 | AA99V to E | V100E |
| 902 | AA41P to Q | P42Q |
| 903 | AA84D to R | D85R |
| 904 | AA70P to I | P71I |
| 905 | AA37L to R | L38R |
| 906 | AA15H to V | H16V |
| 907 | AA12N to R | N13R |
| 908 | AA42G to Q | G43Q |
| 909 | AA22I to H | I23H |
| 910 | AA101Q to F | Q102F |
| 911 | AA37L to Y | L38Y |
| 912 | AA41P to A | P42A |
| 913 | AA42G to F | G43F |
| 914 | AA70P to E | P71E |
| 915 | AA15H to A | H16A |
| 916 | AA50K to Q | K51Q |
| 917 | AA3L to K | L4K |
| 918 | AA99V to N | V100N |
| 919 | AA34E to K | E35K |
| 920 | AA102K to V | K103V |
| 921 | AA7I to V | I8V |
| 922 | AA70P to L | P71L |
| 923 | AA39I to V | I40V |
| 924 | AA22I to R | I23R |
| 925 | AA82G to I | G83I |
| 926 | AA5S to T | S6T |
| 927 | AA82G to L | G83L |
| 928 | AA94L to E | L95E |
| 929 | AA10K to F | K11F |
| 930 | AA19R to L | R20L |
| 931 | AA10K to H | K11H |
| 932 | AA65R to H | R66H |
| 933 | AA103P to V | P104V |
| 934 | AA50K to Y | K51Y |
| 935 | AA23Y to V | Y24V |
| 936 | AA17F to V | F18V |
| 937 | AA12N to Q | N13Q |
| 938 | AA63Q to L | Q64L |
| 939 | AA34E to L | E35L |
| 940 | AA94L to A | L95A |
| 941 | AA2D to Q | D3Q |
| 942 | AA9I to F | I10F |
| 943 | AA109R to Q | R110Q |
| 944 | AA12N to K | N13K |
| 945 | AA36Q to V | Q37V |
| 946 | AA88E to D | E89D |
| 947 | AA36Q to N | Q37N |
| 948 | AA9I to H | I10H |
| 949 | AA88E to H | E89H |
| 950 | AA77F to W | F78W |
| 951 | AA42G to E | G43E |
| 952 | AA17F to W | F18W |
| 953 | AA10K to L | K11L |
| 954 | AA65R to W | R66W |
| 955 | AA34E to Q | E35Q |
| 956 | AA61Y to V | Y62V |
| 957 | AA15H to D | H16D |
| 958 | AA84D to L | D85L |
| 959 | AA2D to V | D3V |
| 960 | AA104V to F | V105F |
| 961 | AA67G to N | G68N |
| 962 | AA80L to H | L81H |
| 963 | AA51D to N | D52N |
| 964 | AA104V to E | V105E |
| 965 | AA2D to Y | D3Y |
| 966 | AA102K to Y | K103Y |
| 967 | AA112Q to Y | Q113Y |
| 968 | AA111L to F | L112F |
| 969 | AA112Q to E | Q113E |
| 970 | AA84D to I | D85I |
| 971 | AA43K to I | K44I |
| 972 | AA52I to W | 153W |
| 973 | AA102K to I | K103I |
| 974 | AA116E to L | E117L |
| 975 | AA88E to K | E89K |
| 976 | AA65R to I | R66I |
| 977 | AA10K to A | K11A |
| 978 | AA80L to W | L81W |
| 979 | AA34E to F | E35F |
| 980 | AA50K to W | K51W |
| 981 | AA116E to Q | E117Q |
| 982 | AA51D to Q | D52Q |
| 983 | AA102K to L | K103L |
| 984 | AA45D to H | D46H |
| 985 | AA36Q to I | Q37I |
| 986 | AA10K to W | K11W |
| 987 | AA10K to Q | K11Q |
| 988 | AA42G to W | G43W |
| 989 | AA80L to D | L81D |
| 990 | AA82G to K | G83K |
| 991 | AA72W to F | W73F |
| 992 | AA46L to D | L47D |
| 993 | AA74R to H | R75H |
| 994 | AA48I to E | I49E |
| 995 | AA2D to A | D3A |
| 996 | AA10K to N | K11N |
| 997 | AA79S to T | S80T |
| 998 | AA82G to R | G83R |
| 999 | AA100L to I | L101I |
| 1000 | AA23Y to A | Y24A |
| 1001 | AA74R to E | R75E |
| 1002 | AA2D to K | D3K |
| 1003 | AA4S to T | S5T |
| 1004 | AA20T to S | T21S |
| 1005 | AA34E to R | E35R |
| 1006 | AA104V to H | V105H |
| 1007 | AA104V to N | V105N |
| 1008 | AA88E to V | E89V |
| 1009 | AA64Y to W | Y65W |
| 1010 | AA88E to L | E89L |
| 1011 | AA112Q to N | Q113N |
| 1012 | AA80L to R | L81R |
| 1013 | AA19R to V | R20V |
| 1014 | AA2D to W | D3W |
| 1015 | AA58T to S | T59S |
| 1016 | AA87A to W | A88W |
| 1017 | AA104V to W | V105W |
| 1018 | AA72W to Y | W73Y |
| 1019 | AA2D to R | D3R |
| 1020 | AA12N to A | N13A |
| 1021 | AA41P to E | P42E |
| 1022 | AA100L to V | L101V |
| 1023 | AA2D to I | D3I |
| 1024 | AA75V to F | V76F |
| 1025 | AA102K to W | K103W |
| 1026 | AA2D to F | D3F |
| 1027 | AA88E to N | E89N |
| 1028 | AA105R to Y | R106Y |
| 1029 | AA116E to A | E117A |
| 1030 | AA34E to V | E35V |
| 1031 | AA59S to T | S60T |
| 1032 | AA22I to Q | I23Q |
| 1033 | AA105R to H | R106H |
| 1034 | AA50K to A | K51A |
| 1035 | AA99V to D | V100D |
| 1036 | AA12N to E | N13E |
| 1037 | AA86V to D | V87D |
| 1038 | AA47F to W | F48W |
| 1039 | AA116E to H | E117H |
| 1040 | AA88E to I | E89I |
| 1041 | AA112Q to W | Q113W |
| 1042 | AA89W to V | W90V |
| 1043 | AA12N to L | N13L |
| 1044 | AA34E to I | E35I |
| 1045 | AA109R to H | R110H |
| 1046 | AA84D to Y | D85Y |
| 1047 | AA3L to D | L4D |
| 1048 | AA101Q to W | Q102W |
| 1049 | AA109R to N | R110N |
| 1050 | AA41P to R | P42R |
| 1051 | AA88E to R | E89R |
| 1052 | AA116E to N | E117N |
| 1053 | AA50K to N | K51N |
| 1054 | AA89W to I | W90I |
| 1055 | AA113A to I | A114I |
| 1056 | AA112Q to F | Q113F |
| 1057 | AA102K to F | K103F |
| 1058 | AA116E to Y | E117Y |
| 1059 | AA71G to N | G72N |
| 1060 | AA94L to Q | L95Q |
| 1061 | AA42P to D | P42D |
| 1062 | AA23Y to Q | Y24Q |
| 1063 | AA107G to R | G108R |
| 1064 | AA107G to H | G108H |
| 1065 | AA12N to D | N13D |
| 1066 | AA87A to N | A88N |
| 1067 | AA23Y to E | Y24E |
| 1068 | AA36Q to H | Q37H |
| 1069 | AA116E to K | E117K |
| 1070 | AA49L to Q | L50Q |
| 1071 | AA23Y to K | Y24K |
| 1072 | AA80L to K | L81K |
| 1073 | AA116E to R | E117R |
| 1074 | AA116E to W | E117W |
| 1075 | AA23Y to R | Y24R |
| 1076 | AA36Q to D | Q37D |
| 1077 | AA107G to A | G108A |
| 1078 | AA116E to F | E117F |
| 1079 | AA63Q to F | Q64F |
| 1080 | AA23Y to I | Y24I |
| 1081 | AA41P to K | P42K |
| 1082 | AA80L to F | L81F |
| 1083 | AA112Q to L | Q113L |
| 1084 | AA63Q to Y | Q64Y |
| 1085 | AA50K to E | K51E |
| 1086 | AA80L to N | L81N |
| 1087 | AA103P to A | P104A |
| 1088 | AA89W to Y | W90Y |
| 1089 | AA89W to L | W90L |
| 1090 | AA19R to I | R20I |
| 1091 | AA19R to Q | R20Q |
| 1092 | AA107G to N | G108N |
| 1093 | AA23Y to N | Y24N |
| 1094 | AA8T to S | T9S |
| 1095 | AA22I to W | I23W |
| 1096 | AA72W to H | W73H |
| 1097 | AA89W to F | W90F |
| 1098 | AA107G to K | G108K |
| 1099 | AA19R to A | R20A |
| 1100 | AA109R to Y | R110Y |
| 1101 | AA101Q to D | Q102D |
| 1102 | AA85E to A | E86A |
| 1103 | AA109R to F | R110F |
| 1104 | AA48I to C | I49C |
| 1105 | AA32S to C | S33C |
| 1106 | AAY23 to C | Y24C |
| 1107 | AAY61 to C | Y62C |

All single site mutations of Table 8 can be introduced into the HTS-2 isoform of SEQ ID NO: 141. All single site mutations of Table 8 can also have methionine missing at position 1. All single site mutations of Table 8 can be generated with or without a His-tag or other protein tag.

### Example 16: Additional HTS Variants Exhibit Sweet Taste in Tasting Assays

Table 9 includes additional HTS single site mutational variants which exhibit sweet taste. Variants are identified (column 2) with respect to the variation from SEQ ID NO:3 as well as based on amino acid numbering when the methionine at position 1 is absent. The presence or absence of methionine does not affect sweet taste of variants. The concentration at which sweet taste was detected in shown in column 3, each sample volume was 100 μL, thus between 40-60 μg of protein was tested. Entries where the concentration is labeled with “*” indicate that not all taste testers indicated that the sample was sweet.

TABLE 9
Variants Exhibiting Sweet Taste

| | Amino Acid | Amino Acid Substitution | |
|---|---|---|---|
| Mutant | Substitution | (Met present at position 1) | Sweetness and |
| No. as in | (no Met at | Position with respect to | Concentration, |
| Table 8 | position 1) | SEQ ID NO: 3 | mg/mL |
| 1002 | AA2D to K | D3K | 0.60 |
| 893 | AA2D to N | D3N | 0.65 |
| 941 | AA2D to Q | D3Q | 0.62 |
| 495 | AA3L to F | L4F | 0.65 |
| 848 | AA3L to Y | L4Y | 0.62 |
| 469 | AA6F to I | F6I | 0.42 |
| 921 | AA7I to V | I8V | 0.84 |
| 795 | AA9I to L | I10L | 0.64 |
| 931 | AA10K to H | K11H | 0.77 |
| 937 | AA12N to Q | N13Q | 0.47 |
| 669 | AA14N to Q | N14Q | 0.75 |
| 804 | AA15H to R | H16R | 0.33 |
| 768 | AA16V to F | V17F | 0.53 |
| 472 | AA17F to L | F18L | 0.57 |
| 722 | AA21A to F | A22F | 0.48 |
| 616 | AA22I to L | I23L | 0.69 |
| 552 | AA23Y to W | Y24W | 0.68 |
| 27 | AA34E to N | E35N | 0.85 |
| 947 | AA36Q to N | Q37N | 0.73 |
| 880 | AA37L to F | L38F | 0.71 |
| 541 | AA38S to T | S39T | 0.61 |
| 679 | AA39I to L | I40L | 0.76 |
| 896 | AA42G to A | G43A | 0.23* |
| 601 | AA43K to R | K44R | 0.42 |
| 692 | AA44W to Y | W45Y | 0.47 |
| 479 | AA46L to F | L47F | 0.51 |
| 46 | AA47F to I | F48I | 0.61* |
| 134 | AA50K to D | K51D | 0.62 |
| 500 | AA50K to R | K51R | 0.33 |
| 18 | AA51D to H | D52H | 0.48 |
| 20 | AA51D to K | D52K | 0.40 |
| 487 | AA52I to K | I53K | 0.45 |
| 839 | AA52I to Y | I53Y | 0.48 |
| 628 | AA54S to T | S55T | 0.38 |
| 610 | AA55I to F | I56F | 0.46 |
| 494 | AA61Y to W | Y62W | 0.25 |
| 888 | AA63Q to N | Q64N | 0.25* |
| 1009 | AA64Y to W | Y65W | 0.44 |
| 658 | AA65R to K | R66K | 0.47 |
| 273 | AA66V to F | V67F | 0.23* |
| 820 | AA67G to A | G68A | 0.40* |
| 602 | AA68D to E | D69E | 0.43 |
| 693 | AA71G to A | G72A | 0.40 |
| 245 | AA72W to Q | W73Q | 0.35* |
| 583 | AA73V to I | V74I | 0.28 |
| 723 | AA74R to K | R75K | 0.41 |
| 664 | AA80L to V | L81V | 0.40 |
| 651 | AA81V to D | V82D | 0.40* |
| 598 | AA81V to E | V82E | 0.40* |
| 640 | AA81V to F | V82F | 0.40 |
| 770 | AA82G to A | G83A | 0.40* |
| 645 | AA82G to H | G83H | 0.40 |
| 587 | AA83A to F | A84F | 0.40 |
| 756 | AA84D to N | D85N | 0.40 |
| 835 | AA86V to L | V87L | 0.40 |
| 946 | AA88E to D | E89D | 0.40 |
| 621 | AA92G to A | G93A | 0.35 |
| 681 | AA93D to E | D94E | 0.53 |
| 809 | AA94L to V | L95V | 0.41 |
| 588 | AA96D to E | D97E | 0.26 |
| 404 | AA97G to L | G97L | 0.40 |
| 470 | AA98F to I | F99I | 0.40 |
| 712 | AA99V to L | V100L | 0.40 |
| 1022 | AA100L to V | L101V | 0.40* |
| 688 | AA102K to R | K103R | 0.40 |
| 933 | AA103P to V | P104V | 0.40 |
| 960 | AA104V to F | V105F | 0.40 |
| 512 | AA105R to K | R106K | 0.40 |
| 1077 | AA107G to A | G108A | 0.40 |
| 213 | AA109R to E | R110E | 0.31 |
| 796 | AA109R to K | R110K | 0.27 |
| 968 | AA111L to F | L112F | 0.33* |
| 1011 | AA112Q to N | Q113N | 0.43 |
| 615 | AA119K to R | K120R | 0.40 |
| 631 | AA120Q to N | Q121N | 0.40* |

All single site mutations of Table 9 can be introduced into the HTS-2 isoform of SEQ ID NO:141. All single site mutations of Table 9 can also have methionine missing at position 1. All single site mutations of Table 9 can be generated with or without a His-tag or other protein tag.

### Example 17: Additional HTS Variants Exhibiting Sweet Taste

Table 10 includes additional HTS single site mutational variants which exhibit sweet taste. Variants are identified (column 2) with respect to the variation from SEQ ID NO:3 as well as based on amino acid numbering when the methionine at position 1 is absent. The presence or absence of methionine does not affect sweet taste of variants.

For sweet tasting variants the following are preferred where amino acid numbering is based on SEQ ID NO:3:
Positions 20, 66 and 75 should maintain a positively charged side-chain;
Substitutions of aspartate or glutamate are not preferred at positions 18-21, 58-67, or 73-81;
Aspartates or glutamates should not be added in positions 1-4, 26-29, 53-57, 82-86, 109-111;
Substitutions of D85 and E86 are allowed, but substitution of D for E and E for D are not preferred;
Substitutions of non-polar side chains (F, G, I, P, T, V, W, Y) in 5-13, 18-21, 24-25, 30-34, 39-40, 45-52, 58-67, 73-81, 87-93 or 112-120 may not be for alpha-helix favoring residues (A, K, L, M) except that I can be substituted for L;
Prolines at positions 2, 36, 42, 71 and 96 should be maintained;
Prolines at positions 34 and 104 should only be substituted to amino acid that favor beta sheet formation (e.g., F, G, I, T, V, W, Y); and/or
Substitutions of aliphatic, aromatic, or neutral base residues (e.g., A, F, G, I, L, N, Q, W, V, Y) in positions 5-13, 18-21, 24-25, 30-34, 39-40, 45-52, 58-67, 73-81, 87-93 or 112-120 should not have a change in side chain volume greater than 50 Å3.

TABLE 10
Additional Sweet Taste Variants

| | | Amino Acid | | |
|---|---|---|---|---|
| Mutant | Variant ID | Substitution | Amino Acid | |
| No. | Without | (Met present | in SEQ | Variant |
| (Table 8) | Met01Table 10: | at position 1) | ID NO: 3 | Amino Acid |
| 7 | D2L | 3 | D | L |
| 8 | D45F | 46 | D | F |
| 9 | D45I | 46 | D | I |
| 10 | D45K | 46 | D | K |
| 11 | D45L | 46 | D | L |
| 12 | D45Q | 46 | D | Q |
| 13 | D45R | 46 | D | R |
| 14 | D45V | 46 | D | V |
| 15 | D45W | 46 | D | W |
| 16 | D45Y | 46 | D | Y |
| 17 | D51F | 52 | D | F |
| 18 | D51H | 52 | D | H |
| 19 | D51I | 52 | D | I |
| 20 | D51K | 52 | D | K |
| 21 | D51L | 52 | D | L |
| 22 | D51R | 52 | D | R |
| 23 | D51V | 52 | D | V |
| 24 | D51W | 52 | D | W |
| 25 | D51Y | 52 | D | Y |
| 26 | E34H | 35 | E | H |
| 27 | E34N | 35 | E | N |
| 28 | E34W | 35 | E | W |
| 29 | E85H | 86 | E | H |
| 30 | E85I | 86 | E | I |
| 32 | F115W | 116 | F | W |
| 33 | F115Y | 116 | F | Y |
| 40 | F17Q | 18 | F | Q |
| 46 | F47I | 48 | F | I |
| 50 | F47Q | 48 | F | Q |
| 56 | F98E | 99 | F | E |
| 57 | F98H | 99 | F | H |
| 58 | F98N | 99 | F | N |
| 59 | F98Q | 99 | F | Q |
| 60 | F98W | 99 | F | W |
| 61 | F98Y | 99 | F | Y |
| 62 | G107E | 108 | G | E |
| 63 | G107D | 108 | G | D |
| 64 | G107L | 108 | G | L |
| 65 | G107Q | 108 | G | Q |
| 66 | G107F | 108 | G | F |
| 67 | G107W | 108 | G | W |
| 68 | G107Y | 108 | G | Y |
| 69 | G42I | 43 | G | I |
| 70 | G42K | 43 | G | K |
| 71 | G42L | 43 | G | L |
| 72 | G42R | 43 | G | R |
| 73 | G42V | 43 | G | V |
| 74 | G42Y | 43 | G | Y |
| 102 | G71D | 72 | G | D |
| 103 | G71E | 72 | G | E |
| 106 | I9Y | 10 | I | Y |
| 107 | I22D | 23 | I | D |
| 108 | I22E | 23 | I | E |
| 109 | I22N | 23 | I | N |
| 112 | I39F | 40 | I | F |
| 114 | I39N | 40 | I | N |
| 115 | I39Q | 40 | I | Q |
| 118 | I39Y | 40 | I | Y |
| 119 | I48D | 49 | I | D |
| 120 | I48F | 49 | I | F |
| 122 | I48N | 49 | I | N |
| 124 | I48Y | 49 | I | Y |
| 128 | I7N | 8 | I | N |
| 129 | I7Q | 8 | I | Q |
| 131 | I7Y | 8 | I | Y |
| 132 | K10D | 11 | K | D |
| 133 | K10E | 11 | K | E |
| 134 | K50D | 51 | K | D |
| 135 | K50F | 51 | K | F |
| 136 | L100A | 101 | L | A |
| 137 | L100F | 101 | L | F |
| 138 | L100K | 101 | L | K |
| 139 | L100Q | 101 | L | Q |
| 140 | L100R | 101 | L | R |
| 141 | L100Y | 101 | L | Y |
| 143 | L111N | 112 | L | N |
| 144 | L111Q | 112 | L | Q |
| 145 | L111Y | 112 | L | Y |
| 146 | L37D | 38 | L | D |
| 147 | L37N | 38 | L | N |
| 151 | L49F | 50 | L | F |
| 154 | L49N | 50 | L | N |
| 156 | L49V | 50 | L | V |
| 158 | L49Y | 50 | L | Y |
| 159 | N11A | 12 | N | A |
| 167 | N11Q | 12 | N | Q |
| 172 | N12V | 13 | N | V |
| 175 | P103I | 104 | P | I |
| 176 | P33V | 34 | P | V |
| 177 | P33W | 34 | P | W |
| 178 | P33Y | 34 | P | Y |
| 204 | Q36F | 37 | Q | F |
| 205 | Q36W | 37 | Q | W |
| 206 | Q36Y | 37 | Q | Y |
| 207 | R105A | 106 | R | A |
| 208 | R105D | 106 | R | D |
| 209 | R105E | 106 | R | E |
| 210 | R105N | 106 | R | N |
| 211 | R105W | 106 | R | W |
| 214 | R109L | 110 | R | L |
| 215 | R109V | 110 | R | V |
| 216 | R109W | 110 | R | W |
| 217 | R109I | 110 | R | I |
| 221 | R19H | 20 | R | H |
| 225 | R56F | 57 | R | F |
| 226 | R56L | 57 | R | L |
| 227 | S91T | 92 | S | T |
| 228 | V104D | 105 | V | D |
| 235 | V62N | 63 | V | N |
| 236 | V62Q | 63 | V | Q |
| 260 | Y64F | 65 | Y | F |
| 266 | Y64Q | 65 | Y | Q |
| 276 | V66N | 67 | V | N |
| 277 | V66Q | 67 | V | Q |
| 281 | G67E | 68 | G | E |
| 282 | G67F | 68 | G | F |
| 283 | G67H | 68 | G | H |
| 284 | G67I | 68 | G | I |
| 285 | G67K | 68 | G | K |
| 286 | G67L | 68 | G | L |
| 287 | G67Q | 68 | G | Q |
| 288 | G67R | 68 | G | R |
| 289 | G67V | 68 | G | V |
| 290 | G67W | 68 | G | W |
| 291 | G67Y | 68 | G | Y |
| 292 | G69A | 70 | G | A |
| 293 | G69D | 70 | G | D |
| 294 | G69F | 70 | G | F |
| 295 | G69H | 70 | G | H |
| 296 | G69I | 70 | G | I |
| 297 | G69K | 70 | G | K |
| 298 | G69L | 70 | G | L |
| 299 | G69N | 70 | G | N |
| 300 | G69Q | 70 | G | Q |
| 301 | G69R | 70 | G | R |
| 302 | G69V | 70 | G | V |
| 303 | G69W | 70 | G | W |
| 304 | G69Y | 70 | G | Y |
| 306 | G71F | 72 | G | F |
| 307 | G71I | 72 | G | I |
| 308 | G71K | 72 | G | K |
| 309 | G71L | 72 | G | L |
| 310 | G71Q | 72 | G | Q |
| 31l | G71R | 72 | G | R |
| 312 | G71V | 72 | G | V |
| 320 | V73N | 74 | V | N |
| 321 | V73Q | 74 | V | Q |
| 334 | V75N | 76 | V | N |
| 335 | V75Q | 76 | V | Q |
| 343 | F77I | 78 | F | I |
| 347 | F77Q | 78 | F | Q |
| 349 | L80Y | 81 | L | Y |
| 350 | V81W | 82 | V | W |
| 351 | G82W | 83 | G | W |
| 352 | D84W | 85 | D | W |
| 353 | E85F | 86 | E | F |
| 354 | E85K | 86 | E | K |
| 355 | E85L | 86 | E | L |
| 356 | E85N | 86 | E | N |
| 357 | E85Q | 86 | E | Q |
| 358 | E85R | 86 | E | R |
| 359 | E85V | 86 | E | V |
| 360 | E85W | 86 | E | W |
| 361 | E85Y | 86 | E | Y |
| 362 | V86N | 87 | V | N |
| 370 | E88W | 89 | E | W |
| 382 | D93W | 94 | D | W |
| 383 | L94D | 95 | L | D |
| 384 | L94F | 95 | L | F |
| 385 | L94H | 95 | L | H |
| 386 | L94K | 95 | L | K |
| 387 | L94N | 95 | L | N |
| 388 | L94R | 95 | L | R |
| 389 | L94W | 95 | L | W |
| 390 | L94Y | 95 | L | Y |
| 403 | G97I | 98 | G | I |
| 404 | G97L | 98 | G | L |
| 405 | G97V | 98 | G | V |
| 406 | G97W | 98 | G | W |
| 407 | F98D | 99 | F | D |
| 408 | F98K | 99 | F | K |
| 409 | F98R | 99 | F | R |
| 410 | L100D | 101 | L | D |
| 411 | L100E | 101 | L | E |
| 412 | L100H | 101 | L | H |
| 413 | L100N | 101 | L | N |
| 414 | L100W | 101 | L | W |
| 416 | P103F | 104 | P | F |
| 423 | P103W | 104 | P | W |
| 424 | P103Y | 104 | P | Y |
| 425 | G107I | 108 | G | I |
| 426 | G107V | 108 | G | V |
| 427 | P110A | 111 | P | A |
| 430 | P110F | 111 | P | F |
| 431 | P110H | 111 | P | H |
| 432 | P110I | 111 | P | I |
| 433 | P110K | 111 | P | K |
| 434 | P110L | 111 | P | L |
| 435 | P110N | 111 | P | N |
| 436 | P110Q | 111 | P | Q |
| 437 | P110R | 111 | P | R |
| 438 | P110V | 111 | P | V |
| 439 | P110W | 111 | P | W |
| 440 | P110Y | 111 | P | Y |
| 450 | A113N | 114 | A | N |
| 456 | F115I | 116 | F | I |
| 459 | F115Q | 116 | F | Q |
| 464 | A117N | 118 | A | N |
| 469 | F6I | 7 | F | I |
| 470 | F98I | 99 | F | I |
| 473 | F98V | 99 | F | V |
| 475 | F98L | 99 | F | L |
| 476 | R105I | 106 | R | I |
| 478 | F6Y | 7 | F | Y |
| 479 | L46F | 47 | L | F |
| 480 | Q101L | 102 | Q | L |
| 481 | L3H | 4 | L | H |
| 482 | K119Q | 120 | K | Q |
| 487 | I52K | 53 | I | K |
| 488 | K119F | 120 | K | F |
| 489 | F6Q | 7 | F | Q |
| 494 | Y61W | 62 | Y | W |
| 495 | L3F | 4 | L | F |
| 497 | L46Y | 47 | L | Y |
| 498 | Q36A | 37 | Q | A |
| 499 | K119V | 120 | K | V |
| 500 | K50R | 51 | K | R |
| 501 | A83Q | 84 | A | Q |
| 502 | L46Q | 47 | L | Q |
| 503 | R56H | 57 | R | H |
| 505 | I9V | 10 | I | V |
| 509 | I55N | 56 | I | N |
| 510 | S13T | 14 | S | T |
| 511 | I52N | 53 | I | N |
| 512 | R105K | 106 | R | K |
| 514 | K119Y | 120 | K | Y |
| 515 | G71W | 72 | G | W |
| 517 | I55Q | 56 | I | Q |
| 518 | L53H | 54 | L | H |
| 520 | R105V | 106 | R | V |
| 521 | N14D | 15 | N | D |
| 523 | A83R | 84 | A | R |
| 524 | K10I | 11 | K | I |
| 525 | S54M | 55 | S | M |
| 526 | A113V | 114 | A | V |
| 530 | I52R | 53 | I | R |
| 531 | Q120L | 121 | Q | L |
| 532 | A83H | 84 | A | H |
| 533 | I52Q | 53 | I | Q |
| 534 | D96Y | 97 | D | Y |
| 535 | K119H | 120 | K | H |
| 536 | L53K | 54 | L | K |
| 537 | L53N | 54 | L | N |
| 538 | K43Q | 44 | K | Q |
| 539 | I55Y | 56 | I | Y |
| 540 | R56Y | 57 | R | Y |
| 541 | S38T | 39 | S | T |
| 542 | Q120I | 121 | Q | I |
| 543 | Q101I | 102 | Q | I |
| 545 | K10V | 11 | K | V |
| 546 | Q120V | 121 | Q | V |
| 547 | E116I | 117 | E | I |
| 548 | K43H | 44 | K | H |
| 549 | K119N | 120 | K | N |
| 550 | L53Y | 54 | L | Y |
| 551 | E116V | 117 | E | V |
| 552 | Y23W | 24 | Y | W |
| 553 | I55H | 56 | I | H |
| 554 | Q101R | 102 | Q | R |
| 555 | N14A | 15 | N | A |
| 556 | Q120R | 121 | Q | R |
| 557 | A21Q | 22 | A | Q |
| 558 | D68N | 69 | D | N |
| 559 | V81N | 82 | V | N |
| 560 | I55K | 56 | I | K |
| 561 | G97N | 98 | G | N |
| 562 | Q120K | 121 | Q | K |
| 563 | R56N | 57 | R | N |
| 564 | R56Q | 57 | R | Q |
| 565 | Y61F | 62 | Y | F |
| 566 | Q101V | 102 | Q | V |
| 567 | Q120W | 121 | Q | W |
| 568 | A83K | 84 | A | K |
| 570 | Q36R | 37 | Q | R |
| 572 | L53Q | 54 | L | Q |
| 573 | V99F | 100 | V | F |
| 574 | K50I | 51 | K | I |
| 578 | G71Y | 72 | G | Y |
| 580 | N14E | 15 | N | E |
| 581 | I55W | 56 | I | W |
| 583 | V73I | 74 | V | I |
| 584 | G82Y | 83 | G | Y |
| 585 | I22Y | 23 | I | Y |
| 587 | A83F | 84 | A | F |
| 588 | D96E | 97 | D | E |
| 589 | L53F | 54 | L | F |
| 590 | K119L | 120 | K | L |
| 591 | I9Q | 10 | I | Q |
| 592 | D96A | 97 | D | A |
| 595 | D93F | 94 | D | F |
| 596 | I55R | 56 | I | R |
| 597 | N14L | 15 | N | L |
| 599 | L3W | 4 | L | W |
| 600 | D68A | 69 | D | A |
| 601 | K43R | 44 | K | R |
| 602 | D68E | 69 | D | E |
| 603 | A83Y | 84 | A | Y |
| 606 | G82N | 83 | G | N |
| 607 | K43N | 44 | K | N |
| 608 | V99Y | 100 | V | Y |
| 609 | D68Q | 69 | D | Q |
| 610 | I55F | 56 | I | F |
| 611 | L53R | 54 | L | R |
| 612 | V16K | 17 | V | K |
| 615 | K119R | 120 | K | R |
| 616 | I22L | 23 | I | L |
| 617 | K43E | 44 | K | E |
| 618 | A21E | 22 | A | E |
| 619 | D96R | 97 | D | R |
| 620 | K50H | 51 | K | H |
| 622 | D93N | 94 | D | N |
| 623 | V99I | 100 | V | I |
| 624 | D96K | 97 | D | K |
| 625 | D93H | 94 | D | H |
| 627 | H15F | 16 | H | F |
| 628 | S54T | 55 | S | T |
| 630 | D96Q | 97 | D | Q |
| 631 | Q120N | 121 | Q | N |
| 632 | I9N | 10 | I | N |
| 634 | V81K | 82 | V | K |
| 635 | D68K | 69 | D | K |
| 636 | V81Y | 82 | V | Y |
| 637 | D93Y | 94 | D | Y |
| 638 | N14Y | 15 | N | Y |
| 639 | V104K | 105 | V | K |
| 640 | V81F | 82 | V | F |
| 641 | E88Y | 89 | E | Y |
| 643 | Q120F | 121 | Q | F |
| 644 | V81Q | 82 | V | Q |
| 645 | G82H | 83 | G | H |
| 646 | V81H | 82 | V | H |
| 647 | N14F | 15 | N | F |
| 648 | D68H | 69 | D | H |
| 649 | A21H | 22 | A | H |
| 650 | D68L | 69 | D | L |
| 654 | D96W | 97 | D | W |
| 655 | Y23F | 24 | Y | F |
| 657 | V16H | 17 | V | H |
| 658 | R65K | 66 | R | K |
| 659 | D68R | 69 | D | R |
| 660 | A21K | 22 | A | K |
| 661 | D96L | 97 | D | L |
| 662 | I22V | 23 | I | V |
| 663 | D96V | 97 | D | V |
| 664 | L80V | 81 | L | V |
| 665 | R56K | 57 | R | K |
| 666 | R56V | 57 | R | V |
| 667 | I52H | 53 | I | H |
| 669 | N14Q | 15 | N | Q |
| 670 | R105L | 106 | R | L |
| 671 | Q120H | 121 | Q | H |
| 672 | Q101H | 102 | Q | H |
| 673 | N14R | 15 | N | R |
| 674 | Q120Y | 121 | Q | Y |
| 675 | G82F | 83 | G | F |
| 677 | D93Q | 94 | D | Q |
| 678 | F17I | 18 | F | I |
| 679 | I39L | 40 | I | L |
| 680 | A21R | 22 | A | R |
| 681 | D93E | 94 | D | E |
| 682 | V16D | 17 | V | D |
| 683 | D96N | 97 | D | N |
| 685 | S38M | 39 | S | M |
| 686 | D68V | 69 | D | V |
| 687 | L46N | 47 | L | N |
| 688 | K102R | 103 | K | R |
| 689 | L53W | 54 | L | W |
| 690 | L37E | 38 | L | E |
| 691 | V86I | 87 | V | I |
| 692 | W44Y | 45 | W | Y |
| 693 | G71A | 72 | G | A |
| 696 | D96H | 97 | D | H |
| 697 | D93A | 94 | D | A |
| 699 | K43Y | 44 | K | Y |
| 700 | N14I | 15 | N | I |
| 701 | D68Y | 69 | D | Y |
| 703 | D68I | 69 | D | I |
| 704 | W44F | 45 | W | F |
| 705 | L37K | 38 | L | K |
| 706 | D96I | 97 | D | I |
| 707 | R56W | 57 | R | W |
| 708 | D96F | 97 | D | F |
| 709 | K25A | 26 | K | A |
| 710 | S90T | 91 | S | T |
| 711 | D68F | 69 | D | F |
| 712 | V99L | 100 | V | L |
| 713 | I22K | 23 | I | K |
| 714 | D68W | 69 | D | W |
| 715 | K102Q | 103 | K | Q |
| 718 | V16Y | 17 | V | Y |
| 720 | H15Y | 16 | H | Y |
| 721 | H15L | 16 | H | L |
| 722 | A21F | 22 | A | F |
| 723 | R74K | 75 | R | K |
| 725 | G97Q | 98 | G | Q |
| 726 | V81R | 82 | V | R |
| 727 | K43A | 44 | K | A |
| 728 | G97E | 98 | G | E |
| 730 | D84A | 85 | D | A |
| 731 | N14V | 15 | N | V |
| 734 | V16R | 17 | V | R |
| 735 | T76S | 77 | T | S |
| 736 | V75I | 76 | V | I |
| 737 | A83W | 84 | A | W |
| 738 | N14W | 15 | N | W |
| 740 | G69E | 70 | G | E |
| 741 | H15I | 16 | H | I |
| 743 | K50V | 51 | K | V |
| 744 | V16W | 17 | V | W |
| 745 | E34A | 35 | E | A |
| 747 | V99K | 100 | V | K |
| 748 | A21Y | 22 | A | Y |
| 749 | K102E | 103 | K | E |
| 750 | D84Q | 85 | D | Q |
| 751 | D93L | 94 | D | L |
| 752 | I22F | 23 | I | F |
| 753 | V104R | 105 | V | R |
| 755 | A21N | 22 | A | N |
| 756 | D84N | 85 | D | N |
| 757 | G67D | 68 | G | D |
| 758 | D45N | 46 | D | N |
| 759 | H15E | 16 | H | E |
| 760 | G97H | 98 | G | H |
| 761 | K43D | 44 | K | D |
| 767 | N14H | 15 | N | H |
| 768 | V16F | 17 | V | F |
| 769 | G97Y | 98 | G | Y |
| 770 | G82A | 83 | G | A |
| 771 | G97R | 98 | G | R |
| 772 | H15K | 16 | H | K |
| 774 | G97F | 98 | G | F |
| 775 | V16Q | 17 | V | Q |
| 776 | L94I | 95 | L | I |
| 777 | L37H | 38 | L | H |
| 778 | Q101A | 102 | Q | A |
| 779 | V99R | 100 | V | R |
| 780 | L80I | 81 | L | I |
| 781 | S40T | 41 | S | T |
| 782 | A83N | 84 | A | N |
| 784 | A21D | 22 | A | D |
| 788 | L3R | 4 | L | R |
| 789 | I48Q | 49 | I | Q |
| 790 | V86Q | 87 | V | Q |
| 791 | I7F | 8 | I | F |
| 793 | H15W | 16 | H | W |
| 794 | D93K | 94 | D | K |
| 795 | I9L | 10 | I | L |
| 796 | R109K | 110 | R | K |
| 797 | A21W | 22 | A | W |
| 798 | K43F | 44 | K | F |
| 799 | K43V | 44 | K | V |
| 801 | L37Q | 38 | L | Q |
| 802 | V99H | 100 | V | H |
| 803 | G71H | 72 | G | H |
| 804 | H15R | 16 | H | R |
| 806 | V99Q | 100 | V | Q |
| 807 | D93R | 94 | D | R |
| 808 | K10Y | 11 | K | Y |
| 809 | L94V | 95 | L | V |
| 810 | L80Q | 81 | L | Q |
| 814 | R56I | 57 | R | I |
| 816 | H15N | 16 | H | N |
| 817 | A87V | 88 | A | V |
| 819 | Q101Y | 102 | Q | Y |
| 820 | G67A | 68 | G | A |
| 821 | K102N | 103 | K | N |
| 823 | L3N | 4 | L | N |
| 824 | T18S | 19 | T | S |
| 825 | K43W | 44 | K | W |
| 826 | Q63V | 64 | Q | V |
| 827 | K43L | 44 | K | L |
| 829 | K102D | 103 | K | D |
| 830 | L3Q | 4 | L | Q |
| 832 | R56A | 57 | R | A |
| 834 | D84K | 85 | D | K |
| 836 | D93V | 94 | D | V |
| 838 | V99W | 100 | V | W |
| 839 | I52Y | 53 | I | Y |
| 841 | V16N | 17 | V | N |
| 842 | N14K | 15 | N | K |
| 843 | K50L | 51 | K | L |
| 847 | H15Q | 16 | H | Q |
| 848 | L3Y | 4 | L | Y |
| 851 | N11V | 12 | N | V |
| 853 | F6W | 7 | F | W |
| 855 | E88F | 89 | E | F |
| 856 | G97K | 98 | G | K |
| 858 | Y61Q | 62 | Y | Q |
| 861 | I52F | 53 | I | F |
| 862 | R105Q | 106 | R | Q |
| 863 | K102H | 103 | K | H |
| 864 | D84V | 85 | D | V |
| 865 | Q112V | 113 | Q | V |
| 866 | G42H | 43 | G | H |
| 867 | G82Q | 83 | G | Q |
| 869 | E34Y | 35 | E | Y |
| 870 | E88A | 89 | E | A |
| 875 | L37W | 38 | L | W |
| 877 | V104Q | 105 | V | Q |
| 878 | V16E | 17 | V | E |
| 880 | L37F | 38 | L | F |
| 881 | V104Y | 105 | V | Y |
| 882 | E88Q | 89 | E | Q |
| 884 | D93I | 94 | D | I |
| 885 | D2H | 3 | D | H |
| 886 | G82V | 83 | G | V |
| 887 | R105F | 106 | R | F |
| 888 | Q63N | 64 | Q | N |
| 889 | S24T | 25 | S | T |
| 890 | G42N | 43 | G | N |
| 893 | D2N | 3 | D | N |
| 896 | G42A | 43 | G | A |
| 898 | Q36L | 37 | Q | L |
| 901 | V99E | 100 | V | E |
| 903 | D84R | 85 | D | R |
| 905 | L37R | 38 | L | R |
| 906 | H15V | 16 | H | V |
| 908 | G42Q | 43 | G | Q |
| 909 | I22H | 23 | I | H |
| 910 | Q101F | 102 | Q | F |
| 911 | L37Y | 38 | L | Y |
| 913 | G42F | 43 | G | F |
| 915 | H15A | 16 | H | A |
| 916 | K50Q | 51 | K | Q |
| 917 | L3K | 4 | L | K |
| 918 | V99N | 100 | V | N |
| 919 | E34K | 35 | E | K |
| 920 | K102V | 103 | K | V |
| 921 | I7V | 8 | I | V |
| 923 | I39V | 40 | I | V |
| 924 | I22R | 23 | I | R |
| 925 | G82I | 83 | G | I |
| 926 | S5T | 6 | S | T |
| 927 | G82L | 83 | G | L |
| 928 | L94E | 95 | L | E |
| 929 | K10F | 11 | K | F |
| 931 | K10H | 11 | K | H |
| 932 | R65H | 66 | R | H |
| 933 | P103V | 104 | P | V |
| 934 | K50Y | 51 | K | Y |
| 937 | N12Q | 13 | N | Q |
| 939 | E34L | 35 | E | L |
| 940 | L94A | 95 | L | A |
| 941 | D2Q | 3 | D | Q |
| 942 | I9F | 10 | I | F |
| 943 | R109Q | 110 | R | Q |
| 945 | Q36V | 37 | Q | V |
| 946 | E88D | 89 | E | D |
| 947 | Q36N | 37 | Q | N |
| 949 | E88H | 89 | E | H |
| 950 | F77W | 78 | F | W |
| 951 | G42E | 43 | G | E |
| 952 | F17W | 18 | F | W |
| 953 | K10L | 11 | K | L |
| 955 | E34Q | 35 | E | Q |
| 957 | H15D | 16 | H | D |
| 958 | D84L | 85 | D | L |
| 959 | D2V | 3 | D | V |
| 960 | V104F | 105 | V | F |
| 961 | G67N | 68 | G | N |
| 963 | D51N | 52 | D | N |
| 964 | V104E | 105 | V | E |
| 965 | D2Y | 3 | D | Y |
| 966 | K102Y | 103 | K | Y |
| 967 | Q112Y | 113 | Q | Y |
| 968 | L111F | 112 | L | F |
| 970 | D84I | 85 | D | I |
| 971 | K43I | 44 | K | I |
| 972 | I52W | 53 | I | W |
| 973 | K102I | 103 | K | I |
| 974 | E116L | 117 | E | L |
| 975 | E88K | 89 | E | K |
| 977 | K10A | 11 | K | A |
| 979 | E34F | 35 | E | F |
| 980 | K50W | 51 | K | W |
| 981 | E116Q | 117 | E | Q |
| 982 | D51Q | 52 | D | Q |
| 983 | K102L | 103 | K | L |
| 984 | D45H | 46 | D | H |
| 985 | Q36I | 37 | Q | I |
| 986 | K10W | 11 | K | W |
| 987 | K10Q | 11 | K | Q |
| 988 | G42W | 43 | G | W |
| 990 | G82K | 83 | G | K |
| 991 | W72F | 73 | W | F |
| 993 | R74H | 75 | R | H |
| 995 | D2A | 3 | D | A |
| 996 | K10N | 11 | K | N |
| 997 | S79T | 80 | S | T |
| 998 | G82R | 83 | G | R |
| 999 | L100I | 101 | L | I |
| 1002 | D2K | 3 | D | K |
| 1003 | S4T | 5 | S | T |
| 1004 | T20S | 21 | T | S |
| 1005 | E34R | 35 | E | R |
| 1006 | V104H | 105 | V | H |
| 1007 | V104N | 105 | V | N |
| 1008 | E88V | 89 | E | V |
| 1009 | Y64W | 65 | Y | W |
| 1010 | E88L | 89 | E | L |
| 1011 | Q112N | 113 | Q | N |
| 1014 | D2W | 3 | D | W |
| 1015 | T58S | 59 | T | S |
| 1017 | V104W | 105 | V | W |
| 1018 | W72Y | 73 | W | Y |
| 1019 | D2R | 3 | D | R |
| 1020 | N12A | 13 | N | A |
| 1022 | L100V | 101 | L | V |
| 1023 | D2I | 3 | D | I |
| 1025 | K102W | 103 | K | W |
| 1026 | D2F | 3 | D | F |
| 1027 | E88N | 89 | E | N |
| 1028 | R105Y | 106 | R | Y |
| 1029 | E116A | 117 | E | A |
| 1030 | E34V | 35 | E | V |
| 1031 | S59T | 60 | S | T |
| 1032 | I22Q | 23 | I | Q |
| 1033 | R105H | 106 | R | H |
| 1034 | K50A | 51 | K | A |
| 1035 | V99D | 100 | V | D |
| 1038 | F47W | 48 | F | W |
| 1039 | E116H | 117 | E | H |
| 1040 | E88I | 89 | E | I |
| 1044 | E34I | 35 | E | I |
| 1045 | R109H | 110 | R | H |
| 1046 | D84Y | 85 | D | Y |
| 1048 | Q101W | 102 | Q | W |
| 1049 | R109N | 110 | R | N |
| 1051 | E88R | 89 | E | R |
| 1052 | E116N | 117 | E | N |
| 1053 | K50N | 51 | K | N |
| 1056 | Q112F | 113 | Q | F |
| 1057 | K102F | 103 | K | F |
| 1058 | E116Y | 117 | E | Y |
| 1059 | G71N | 72 | G | N |
| 1060 | L94Q | 95 | L | Q |
| 1062 | Y23Q | 24 | Y | Q |
| 1063 | G107R | 108 | G | R |
| 1064 | G107H | 108 | G | H |
| 1066 | A87N | 88 | A | N |
| 1068 | Q36H | 37 | Q | H |
| 1069 | E116K | 117 | E | K |
| 1070 | L49Q | 50 | L | Q |
| 1073 | E116R | 117 | E | R |
| 1074 | E116W | 117 | E | W |
| 1076 | Q36D | 37 | Q | D |
| 1077 | G107A | 108 | G | A |
| 1078 | E116F | 117 | E | F |
| 1079 | Q63F | 64 | Q | F |
| 1082 | L80F | 81 | L | F |
| 1084 | Q63Y | 64 | Q | Y |
| 1085 | K50E | 51 | K | E |
| 1086 | L80N | 81 | L | N |
| 1088 | W89Y | 90 | W | Y |
| 1092 | G107N | 108 | G | N |
| 1094 | T8S | 9 | T | S |
| 1095 | I22W | 23 | I | W |
| 1097 | W89F | 90 | W | F |
| 1098 | G107K | 108 | G | K |
| 1100 | R109Y | 110 | R | Y |
| 1101 | Q101D | 102 | Q | D |
| 1102 | E85A | 86 | E | A |
| 1103 | R109F | 110 | R | F |

All single site mutations of Table 10 can be introduced into the HTS-2 isoform of SEQ ID NO: 141. All single site mutations of Table 10 can also have methionine missing at position 1. All single site mutations of Table 10 can be generated with or without a His-tag or other protein tag.

### Example 18: HTS and Sucrose in a Citric Acid Solution (Sweetener Composition)

Sucrose is the standard for sweetness intensity perception in the food and beverage industry. The absolute reference scale for sweetness intensity is based on sucrose dissolved in water (% sucrose (w/v) in water) and demonstrates a linear relationship between concentration (sucrose w/v) and response (sweetness intensity). Other carbohydrate and polyol sweeteners exhibit a linear potency curve similar to sucrose at the following multipliers, Erythritol (0.7×), and Allulose (0.7×) (DuBois G E, Walters D E, Schiffman S S, Warwick Z S, Booth B J, et al. (1991) Concentration-response relationships of sweeteners: a systematic study. In Sweeteners: Discovery, Molecular Design and Chemoreception, ed. DE Walters, FT Orthoefer, GE DuBois, pp. 261-276. Washington, DC: Am. Chem. Soc). High potency (HP) sweeteners exhibit hyperbolic curves. With regard to the following Examples 18-38, sucrose equivalent (SE) values of non-sucrose sweeteners were calculated from Dubois et al. (1991) and cross referenced with other studies (Wee, M., Tan, V., & Forde, C. (2018). A Comparison of Psychophysical Dose-Response Behaviour across 16 Sweeteners. Nutrients, 10 (11), 1632).

The relationship between sweetness intensity and concentration for HP sweeteners has been demonstrated to be well modeled by the law of mass action, a hyperbolic function of the form R═RmC/(Kd+C), where R is the response in units of sucrose equivalence (SE) on a percentage (w/v) basis, Rm is the maximal response in SE units, Kd is the apparent sweetener/receptor dissociation L constant, and C is the sweetener concentration in mg/L (DuBois G E, Walters D E, Schiffman S S, Warwick Z S, Booth B J, et al. 1991. Concentration-response relationships of sweeteners: a systematic study. In Sweeteners: Discovery, Molecular Design and Chemoreception, ed. DE Walters, FT Orthoefer, GE DuBois, pp. 261-276. Washington, DC: Am. Chem. Soc).

An HTS sweetness concentration response curve was developed. These values were used to determine approximate sucrose equivalents for the following blending examples (Examples 18-37).

For Examples 18-38, HTS generated by expression in Pichia pastoris was utilized which has been determined to be a mixture of HTS-1 and HTS-2 (with no His-tags) of relative amounts of 10% HTS-1 to 90% HTS-2 by weight.

HTS ((0.0 mg/mL, 0.007 mg/mL, 0.015 mg/mL and 0.035 mg/mL) and sucrose (8% w/w, 6% w/w, 4% w/w, and 2% w/w (respectively)) were added to a citric acid solution. The sweetener mixture was compared to a control with only sucrose added to the matrix. The sweetener mix was expected to be as sweet as or sweeter than the sucrose control for this matrix. Of total perceived sweetness intensity in each of the experimental samples in Table 11, 75% comes from sucrose and 25% comes from HTS, 50% comes from sucrose and 50% comes from HTS, 25% comes from sucrose and 75% comes from HTS, respectively.

TABLE 11
HTS and Sucrose Mixtures in a Citric Acid Solution Compared to Controls

| | Sucrose w/w | | | |
|---|---|---|---|---|
| Sample | Concentration | HTS Amount | | Observations |
| Unsweetened Control | NA | NA | | Sour, astringent, harsh. |
| Sucrose Control | 8% | 0.0 | mg/mL | Reminiscent of sweet dessert and ice |
| | | | | wines, limoncello, fruit candy, apple |
| | | | | juice. |
| 75 Sucrose/25 HTS : | 6% | 0.007 | mg/mL | Less acidic, slightly enhanced |
| 75% total perceived | | | | sweetness, better balance, fruitier. |
| sweetness from Sucrose | | | | |
| and 25% perceived | | | | |
| sweetness from HTS | | | | |
| 50 Sucrose/50 HTS: | 4% | 0.015 | mg/mL | Increased acidity up front, sweet |
| 50% total perceived | | | | aftertaste. |
| sweetness from Sucrose | | | | |
| and 50% perceived | | | | |
| sweetness from HTS | | | | |
| 25 Sucrose/75 HTS: | 2% | 0.035 | mg/mL | Intensely sour up front, slow build to |
| 25% total perceived | | | | max sweetness intensity, minimal |
| sweetness from Sucrose | | | | sweet aftertaste instead mostly sour. |
| and 75% perceived | | | | |
| sweetness from HTS | | | | |

### Example 19: HTS and Sucrose in a Lemon/Lime Soda (Sweetener Composition)

HTS ((0.0 mg/mL, 0.008 mg/mL, 0.023 mg/mL and 0.064 mg/mL) and sucrose (10% w/w, 7.5% w/w, 5% w/w, and 2.5% w/w (respectively)) were added to a lemon/lime soda. The sweetener mixture was compared to a control with only sucrose added to the matrix. The sweetener mix was expected to be as sweet as or sweeter than the sucrose control for this matrix. Of total perceived sweetness intensity in each of the experimental samples in Table 12, 75% comes from sucrose and 25% comes from HTS, 50% comes from sucrose and 50% comes from HTS, 25% comes from sucrose and 75% comes from HTS, respectively.

TABLE 12
HTS and Sucrose Mixtures in a Lemon/Lime Soda Compared to Controls

| | Sucrose w/w | | | |
|---|---|---|---|---|
| Sample | Concentration | HTS Amount | | Observations |
| Unsweetened | NA | NA | | Sour, bitter, astringent, harsh, citrus. |
| Control | | | | |
| Sucrose Control | 10% | 0.0 | mg/mL | Good, sweet to sour balance and pleasant |
| | | | | citrus flavor. Reminiscent of commercial |
| | | | | lemon sides carbonated soft drinks. |
| 75 Sucrose /25 HTS | 7.5% | 0.008 | mg/mL | Very similar to sucrose control, slightly |
| | | | | lower sweet intensity up front but reaches |
| | | | | same max sweetness intensity. Pleasant, |
| | | | | sweet lemon lime aftertaste. |
| Sucrose 50/50 HTS | 5% | 0.023 | mg/mL | Sour up front, sweetness intensity builds |
| | | | | slowly and is present in aftertaste. Lacking |
| | | | | mouthfeel and body. |
| Sucrose 25/75 HTS | 2.5% | 0.064 | mg/mL | Harsh acidity up front, sweetness intensity |
| | | | | builds around 10 seconds peaking near 30. |
| | | | | Strong sweet aftertaste. |

### Example 20: HTS and Sucrose in an Almond Milk (Sweetener Composition)

HTS ((0.0 mg/mL, 0.005 mg/mL, 0.007 mg/mL and 0.010 mg/mL) and sucrose (4% w/w, 3.0% w/w, 2% w/w, and 1.0% w/w (respectively)) were added to an unsweetened almond milk. The sweetener mixture was compared to a control with only sucrose added to the matrix. The sweetener mix was expected to be as sweet as or sweeter than the control for this matrix. Of total perceived sweetness intensity in each of the experimental samples in Table 13, 75% comes from sucrose and 25% comes from HTS, 50% comes from sucrose and 50% comes from HTS, 25% comes from sucrose and 75% comes from HTS, respectively.

TABLE 13
HTS and Sucrose Mixtures in an Almond Milk Compared to Controls

| | Sucrose w/w | | | |
|---|---|---|---|---|
| Sample | Concentration | HTS Amount | | Observations |
| Unsweetened | NA | NA | | Nutty, creamy, low sweetness. |
| Control | | | | |
| Sucrose | 4% | 0.0 | mg/mL | Pleasantly sweetened almond milk, creamy |
| Control | | | | mouthfeel. |
| Sucrose | 3.0% | 0.005 | mg/mL | Similar max sweetness intensity, slight |
| 75/25 HTS | | | | sweet aftertaste, full creamy mouthfeel. |
| Sucrose | 2% | 0.007 | mg/mL | Lower initial sweetness intensity, but higher |
| 50/50 HTS | | | | max sweetness intensity overall. Saltiness |
| | | | | up front and sweet aftertaste. |
| Sucrose | 1.0% | 0.010 | mg/mL | Increased almond flavor intensity. Lower |
| 25/75 HTS | | | | max sweetness intensity but strong sweet |
| | | | | aftertaste. |

### Example 21: HTS and Sucrose in a Chocolate Spread (Sweetener Composition)

HTS ((0.0 mg/mL, 0.008 mg/mL, 0.023 mg/mL and 0.064 mg/mL) and sucrose (45% w/w, 33.8% w/w, 23% w/w, and 11.3% w/w (respectively)) were added to a chocolate spread. The sweetener mixture was compared to a control with only sucrose added to the matrix. The sweetener mix was expected to be as sweet as or sweeter than the control for this matrix. Of total perceived sweetness intensity in each of the experimental samples in Table 14, 75% comes from sucrose and 25% comes from HTS, 50% comes from sucrose and 50% comes from HTS, 25% comes from sucrose and 75% comes from HTS, respectively.

TABLE 14
HTS and Sucrose Mixtures in a Chocolate Spread Compared to Controls

| | Sucrose w/w | | | |
|---|---|---|---|---|
| Sample | Concentration | HTS Amount | | Observations |
| Unsweetened | NA | NA | | Intense cocoa and bitter, salty, astringent. |
| Control | | | | |
| Sucrose | 45% | 0.0 | mg/mL | Strong cocoa- sweet, bitter, salty, and |
| Control | | | | roasted brown notes. Reminiscent of ice |
| | | | | cream chocolate coating. |
| Sucrose | 33.8% | 0.008 | mg/mL | Lower max sweetness intensity, higher salt |
| 75/25 HTS | | | | intensity. Reminiscent of cacao nibs. |
| Sucrose | 23% | 0.023 | mg/mL | Similar max sweetness intensity to sucrose |
| 50/50 HTS | | | | control. Pleasant nutty and cocoa flavors |
| | | | | perceived. Some sweet and bitter cocoa |
| | | | | aftertaste |
| Sucrose | 11.3% | 0.064 | mg/mL | Bitter cocoa and salt notes. Low max |
| 25/75 HTS | | | | sweetness intensity but some sweet |
| | | | | aftertaste. |

### Example 22: HTS and Erythritol in a Citric Acid Solution (Sweetener Composition)

HTS ((0.0 mg/mL, 0.007 mg/mL, 0.015 mg/mL and 0.035 mg/mL) and erythritol (11.43% w/w, 8.57% w/w, 5.71% w/w, and 2.86% w/w (respectively)) were added to a citric acid solution. The sweetener mixture was compared to a control with only erythritol added to the matrix. The sweetener mix was expected to be as sweet as or sweeter than the control for this matrix. Of total perceived sweetness intensity in each of the experimental samples in Table 15, 75% comes from erythritol and 25% comes from HTS, 50% comes from erythritol and 50% comes from HTS, 25% comes from erythritol and 75% comes from HTS, respectively.

TABLE 15
HTS and Erythritol Mixtures in a Citric Acid Solution Compared to Controls

| | Erythritol w/w | | | |
|---|---|---|---|---|
| Sample | Concentration | HTS Amount | | Observations |
| Unsweetened | NA | NA | | Sour, astringent, harsh. |
| Control | | | | |
| Erythritol | 11.43% | 0 | mg/mL | Reduced sour intensity compared to sucrose |
| Control | | | | examples, mouth cooling effect, metallic |
| | | | | aftertaste. |
| Erythritol | 8.57% | 0.007 | mg/mL | Increased initial sour intensity compared to |
| 75/25 HTS | | | | control, increased astringency and chalky |
| | | | | mouthfeel. |
| Erythritol | 5.71% | 0.015 | mg/mL | Increased initial sour intensity compared |
| 50/50 HTS | | | | to control. Mouth puckering, minimal |
| | | | | sweetness linger or aftertaste. |
| Erythritol | 2.86% | 0.035 | mg/mL | Increased initial sour intensity compared |
| 25/75 HTS | | | | to control, astringency, sweet aftertaste. |

### Example 23: HTS and Erythritol in a Lemon/Lime Soda (Sweetener Composition)

HTS ((0.0 mg/mL, 0.008 mg/mL, 0.023 mg/mL and 0.064 mg/mL) and erythritol (13.30% w/w, 9.31% w/w, 6.90% w/w, and 3.10% w/w (respectively)) were added to a lemon/lime soda. The sweetener mixture was compared to a control with only erythritol added to the matrix. The sweetener mix was expected to be as sweet as or sweeter than the control for this matrix. Of total perceived sweetness intensity in each of the experimental samples in Table 16, 75% comes from erythritol and 25% comes from HTS, 50% comes from erythritol and 50% comes from HTS, 25% comes from erythritol and 75% comes from HTS, respectively.

TABLE 16
HTS and Erythritol Mixtures in a Lemon/Lime Soda Compared to Controls

| Samples | Erythritol w/w | | | |
|---|---|---|---|---|
| Name | Concentration | HTS Amount | | Observations |
| Unsweetened | NA | NA | | Sour, bitter, astringent, harsh, citrus. |
| Control | | | | |
| Erythritol | 13.30% | 0.0 | mg/mL | Reduced citrus flavor intensity. Strong mouth |
| Control | | | | cooling and metallic aftertaste. |
| Erythritol | 9.31% | 0.008 | mg/mL | Increased citrus flavor intensity compared to |
| 75/25 HTS | | | | control. Similar max sweetness intensity. More |
| | | | | intense and extended citrus aftertaste compared to |
| | | | | control. |
| Erythritol | 6.90% | 0.023 | mg/mL | Reduced max sweetness intensity compared to |
| 50/50 HTS | | | | control. Increased citrus flavor intensity compared |
| | | | | to control. Reminiscent of tonic water, lemon |
| | | | | lime candy. |
| Erythritol | 3.10% | 0.064 | mg/mL | Reduced initial and max sweetness intensity |
| 25/75 HTS | | | | compared to control. Strong sweet aftertaste. |
| | | | | Reminiscent of flavored sparkling water. |

### Example 24: HTS and Erythritol in an Almond Milk (Sweetener Composition)

HTS ((0.0 mg/mL, 0.005 mg/mL, 0.007 mg/mL and 0.010 mg/mL) and erythritol (5.71% w/w, 4.29% w/w, 2.86% w/w, and 1.43% w/w (respectively)) were added to an unsweetened almond milk. The sweetener mixture was compared to a control with only erythritol added to the matrix. The sweetener mix was expected to be as sweet as or sweeter than the control for this matrix. Of total perceived sweetness intensity in each of the experimental samples in Table 17, 75% comes from erythritol and 25% comes from HTS, 50% comes from erythritol and 50% comes from HTS, 25% comes from erythritol and 75% comes from HTS, respectively.

TABLE 17
HTS and Erythritol Mixtures in an Almond Milk Compared to Controls

| Samples | Erythritol w/w | HTS | | |
|---|---|---|---|---|
| Name | Concentration | Amount | | Observations |
| Unsweetened | NA | NA | | Nutty, creamy, low sweetness. |
| Control | | | | |
| Erythritol | 5.71% | 0.0 | mg/mL | Reduced salt and almond intensity compared to |
| Control | | | | unsweetened control. Mouth cooling and metallic |
| | | | | aftertaste. |
| Erythritol | 4.29% | 0.005 | mg/mL | Increased initial sweetness intensity. |
| 75/25 HTS | | | | Increased almond intensity. Reduced mouth |
| | | | | cooling and metallic aftertaste. Sweet and nutty |
| | | | | aftertaste. |
| Erythritol | 2.86% | 0.007 | mg/mL | Increased almond intensity. Lower max |
| 50/50 HTS | | | | sweetness intensity but strong sweet aftertaste. |
| | | | | Reminiscent of true dairy rounded sweetness |
| | | | | profile. |
| Erythritol | 1.43% | 0.010 | mg/mL | Similar max sweetness intensity to control. |
| 25/75 HTS | | | | Reduced mouth cooling and metallic |
| | | | | intensity. Strong sweet aftertaste. |

### Example 25: HTS and Erythritol in a Chocolate Spread (Sweetener Composition)

HTS ((0.0 mg/mL, 0.008 mg/mL, 0.023 mg/mL and 0.064 mg/mL) and erythritol (45% w/w, 33.8% w/w, 23% w/w, and 11.3% w/w (respectively)) were added to a chocolate spread. The sweetener mixture was compared to a control with only erythritol added to the matrix. The sweetener mix was expected to be as sweet as or sweeter than the control for this matrix. Of total perceived sweetness intensity in each of the experimental samples in Table 18, 75% comes from erythritol and 25% comes from HTS, 50% comes from erythritol and 50% comes from HTS, 25% comes from erythritol and 75% comes from HTS, respectively.

TABLE 18
HTS and Erythritol Mixtures in a Chocolate Spread Compared to Controls

| Samples | Erythritol w/w | HTS | | |
|---|---|---|---|---|
| Name | Concentration | Amount | | Observations |
| Unsweetened | NA | NA | | Intense cocoa and bitter, salty, astringent. |
| Control | | | | |
| Erythritol | 45% | 0.0 | mg/mL | Brief initial sweetness, mouth cooling, cocoa |
| Control | | | | associated bitter. |
| Erythritol | 33.8% | 0.008 | mg/mL | Increased nutty and salt intensity, reduced max |
| 75/25 HTS | | | | sweetness intensity and mouth cooling. |
| Erythritol | 23% | 0.023 | mg/mL | Increased salt and cocoa intensity. Lower max |
| 50/50 HTS | | | | sweetness intensity compared to control. |
| | | | | Increased salty and cocoa aftertaste. |
| Erythritol | 11.3% | 0.064 | mg/mL | Reduced max sweetness intensity. |
| 25/75 HTS | | | | |

### Example 26: HTS and Sucralose in a Citric Acid Solution (Sweetener Composition)

HTS ((0.0 mg/mL, 0.007 mg/mL, 0.015 mg/mL and 0.035 mg/mL) and sucralose (0.0170% w/w, 0.0100% w/w, 0.0053% w/w, and 0.0023% w/w (respectively)) were added to a citric acid solution. The sweetener mixture was compared to a control with only sucralose added to the matrix. The sweetener mix was expected to be as sweet as or sweeter than the control for this matrix. Of total perceived sweetness intensity in each of the experimental samples in Table 19, 75% comes from sucralose and 25% comes from HTS, 50% comes from sucralose and 50% comes from HTS, 25% comes from sucralose and 75% comes from HTS, respectively.

TABLE 19
HTS and Sucralose Mixtures in a Citric Acid Solution Compared to Controls

| Samples | Sucralose w/w | HTS | | |
|---|---|---|---|---|
| Name | Concentration | Amount | | Observations |
| Unsweetened | NA | NA | | Sour, astringent, harsh. |
| Control | | | | |
| Sucralose | 0.0170% | 0.0 | mg/mL | Intense initial sweetness that overpowers acidity. |
| Control | | | | Citrus and fruit flavors. Strong sweet aftertaste. |
| Sucralose | 0.0100% | 0.007 | mg/mL | Similar max sweet intensity as control. Reduced |
| 75/25 HTS | | | | astringency compared to control. Sour aftertaste. |
| Sucralose | 0.0053% | 0.015 | mg/mL | Increased sour intensity compared to control. |
| 50/50 HTS | | | | Reduced max sweet intensity compared to control. |
| Sucralose | 0.0023% | 0.035 | mg/mL | Reduced max sweet intensity compared to control. |
| 25/75 HTS | | | | Strong sweet aftertaste. |

### Example 27: HTS and Sucralose in a Lemon/Lime Soda (Sweetener Composition)

HTS ((0.0 mg/mL, 0.008 mg/mL, 0.023 mg/mL and 0.064 mg/mL) and sucralose (0.0387% w/w, 0.0247% w/w, 0.0096% w/w, and 0.0030% w/w (respectively)) were added to a lemon/lime soda. The sweetener mixture was compared to a control with only sucralose added to the matrix. The sweetener mix was expected to be as sweet as or sweeter than the control for this matrix. Of total perceived sweetness intensity in each of the experimental samples in Table 20, 75% comes from sucralose and 25% comes from HTS, 50% comes from sucralose and 50% comes from HTS, 25% comes from sucralose and 75% comes from HTS, respectively.

TABLE 20
HTS and Sucralose Mixtures in a Lemon/Lime Soda Compared to Controls

| Samples | Sucralose w/w | HTS | | |
|---|---|---|---|---|
| Name | Concentration | Amount | | Observations |
| Unsweetened | NA | NA | | Sour, bitter, astringent, harsh, citrus. |
| Control | | | | |
| Sucralose | 0.0387% | 0.0 | mg/mL | Intense initial sourness. Strong sweet aftertaste. |
| Control | | | | |
| Sucralose | 0.0247% | 0.008 | mg/mL | Increased citrus flavor intensity compared to |
| 75/25 HTS | | | | control. Enhanced sweetness profile, similar to |
| | | | | sucrose control. |
| Sucralose | 0.0096% | 0.023 | mg/mL | Intense initial sourness. Reduced max sweetness |
| 50/50 HTS | | | | intensity compared to control. Strong sweet |
| | | | | aftertaste. |
| Sucralose | 0.0030% | 0.064 | mg/mL | Intense initial sourness. Reduced max sweetness |
| 25/75 HTS | | | | intensity compared to control. Strong sweet |
| | | | | aftertaste. |

### Example 28: HTS and Sucralose in an Almond Milk (Sweetener Composition)

HTS ((0.0 mg/mL, 0.005 mg/mL, 0.007 mg/mL and 0.010 mg/mL) and sucralose (0.0053% w/w, 0.0037% w/w, 0.0023% w/w, and 0.0011% w/w (respectively)) were added to an unsweetened almond milk. The sweetener mixture was compared to a control with only sucralose added to the matrix. The sweetener mix was expected to be as sweet as or sweeter than the control for this matrix. Of total perceived sweetness intensity in each of the experimental samples in Table 21, 75% comes from sucralose and 25% comes from HTS, 50% comes from sucralose and 50% comes from HTS, 25% comes from sucralose and 75% comes from HTS, respectively.

TABLE 21
HTS and Sucralose Mixtures in an Almond Milk Compared to Controls

| Sample | Sucralose w/w | HTS | | |
|---|---|---|---|---|
| Name | Concentration | Amount | | Observations |
| Unsweetened | NA | NA | | Nutty, creamy, low sweetness. |
| Control | | | | |
| Sucralose | 0.0053% | 0.0 | mg/mL | Pleasantly sweetened almond milk. Little |
| Control | | | | mouthfeel or body. Sweet aftertaste. |
| Sucralose | 0.0037% | 0.005 | mg/mL | Increased mouthfeel and body compared to |
| 75/25 HTS | | | | control. |
| | | | | Enhanced sweetness profile similar to sucrose |
| | | | | control. Sweet and nutty aftertaste. |
| Sucralose | 0.0023% | 0.007 | mg/mL | Increase almond flavor intensity. Lower max |
| 50/50 HTS | | | | sweetness intensity but strong sweet aftertaste. |
| Sucralose | 0.0011% | 0.010 | mg/mL | Increased almond flavor intensity. Lower max |
| 25/75 HTS | | | | sweetness intensity but strong sweet aftertaste. |

### Example 29: HTS and Sucralose in a Chocolate Spread (Sweetener Composition)

HTS ((0.0 mg/mL, 0.008 mg/mL, 0.023 mg/mL and 0.064 mg/mL) and sucralose (0.0387% w/w, 0.0247% w/w, 0.0096% w/w, and 0.0030% w/w (respectively)) were added to a chocolate spread. The sweetener mixture was compared to a control with only sucralose added to the matrix. The sweetener mix was expected to be as sweet as or sweeter than the control for this matrix. Of total perceived sweetness intensity in each of the experimental samples in Table 22, 75% comes from sucralose and 25% comes from HTS, 50% comes from sucralose and 50% comes from HTS, 25% comes from sucralose and 75% comes from HTS, respectively.

TABLE 22
HTS and Sucralose Mixtures in a Chocolate Spread Compared to Controls

| Samples | Sucralose w/w | HTS | | |
|---|---|---|---|---|
| Name | Concentration | Amount | | Observations |
| Unsweetened | NA | NA | | Intense cocoa and bitter, salty, astringent |
| Control | | | | |
| Sucralose | 0.0387% | 0.0 | mg/mL | Reduced cocoa flavor intensity compared to sucrose |
| Control | | | | control, sweet aftertaste. |
| Sucralose | 0.0247% | 0.008 | mg/mL | Increased cocoa flavor intensity. Sweet aftertaste. |
| 75/25 HTS | | | | |
| Sucralose | 0.0096% | 0.023 | mg/mL | Increased cocoa flavor intensity. Sweet aftertaste. |
| 50/50 HTS | | | | |
| Sucralose | 0.0030% | 0.064 | mg/mL | Increased cocoa flavor intensity. Sweet aftertaste. |
| 25/75 HTS | | | | |

### Example 30: HTS and Stevia (Stevia Extract) in a Citric Acid Solution (Sweetener Composition)

HTS ((0.0 mg/mL, 0.007 mg/mL, 0.015 mg/mL and 0.035 mg/mL) and stevia (0.0500% w/w, 0.0190% w/w, 0.0085% w/w, and 0.0032% w/w (respectively)) were added to a citric acid solution. The sweetener mixture was compared to a control with only stevia added to the matrix. The sweetener mix was expected to be as sweet as or sweeter than the control for this matrix. Of total perceived sweetness intensity in each of the experimental samples in Table 23, 75% comes from stevia and 25% comes from HTS, 50% comes from stevia and 50% comes from HTS, 25% comes from stevia and 75% comes from HTS, respectively.

TABLE 23
HTS and Stevia Mixtures in a Citric Acid Solution Compared to Controls

| | Stevia (RA95) | | | |
|---|---|---|---|---|
| Samples | w/w | HTS | | |
| Name | Concentration | Amount | | Observations |
| Unsweetened | NA | NA | | Sour, astringent, harsh. |
| Control | | | | |
| Stevia RA95 | 0.0500% | 0.0 | mg/mL | Similar max sweetness intensity to sucrose control. |
| Control | | | | Bitter and metallic notes. Bitter and metallic |
| | | | | aftertaste. |
| Stevia RA95 | 0.0190% | 0.007 | mg/mL | Increased citrus and fruit flavor intensity compared |
| 75/25 HTS | | | | to control. Reduced maximum sweetness intensity |
| | | | | and sourness intensity compared to control. Reduced |
| | | | | bitter and metallic intensity and aftertaste. |
| Stevia RA95 | 0.0085% | 0.015 | mg/mL | Initial sourness. Reduced bitter and metallic |
| 50/50 HTS | | | | intensity and aftertaste. Reduced maximum |
| | | | | sweetness intensity compared to control. |
| Stevia RA95 | 0.0032% | 0.035 | mg/mL | Reduced max sweet intensity compared to control. |
| 25/75 HTS | | | | Strong sweet aftertaste. |

### Example 31: HTS and Stevia in a Lemon/Lime Soda (Sweetener Composition)

HTS ((0.0 mg/mL, 0.008 mg/mL, 0.023 mg/mL and 0.064 mg/mL) and stevia (0.0750% w/w, 0.0400% w/w, 0.0130% w/w, and 0.0042% w/w (respectively)) were added to a lemon/lime soda. The sweetener mixture was compared to a control with only stevia added to the matrix. The sweetener mix was expected to be as sweet as or sweeter than the control for this matrix. Of total perceived sweetness intensity in each of the experimental samples in Table 24, 75% comes from stevia and 25% comes from HTS, 50% comes from stevia and 50% comes from HTS, 25% comes from stevia and 75% comes from HTS, respectively.

TABLE 24
HTS and Stevia Mixtures in a Lemon/Lime Soda Compared to Controls

| | Stevia (RA95) | | | |
|---|---|---|---|---|
| Samples | w/w | HTS | | |
| Name | Concentration | Amount | | Observations |
| Unsweetened | NA | NA | | Sour, bitter, astringent, harsh, citrus. |
| Control | | | | |
| Stevia RA95 | 0.0750% | 0.0 | mg/mL | Intense initial sweetness. Intense bitter and metallic. |
| Control | | | | Strong sweet aftertaste. Reduced citrus flavor |
| | | | | intensity compared to control. |
| Stevia RA95 | 0.0400% | 0.008 | mg/mL | Increased citrus flavor intensity compared to control. |
| 75/25 HTS | | | | Increased initial sourness. Reduced maximum |
| | | | | sweetness intensity compared to control. |
| Stevia RA95 | 0.0130% | 0.023 | mg/mL | Increased initial sourness. Reduced maximum |
| 50/50 HTS | | | | sweetness intensity compared to control. Sweet |
| | | | | aftertaste. |
| Stevia RA95 | 0.0042% | 0.064 | mg/mL | Increased initial sourness. Reduced maximum |
| 25/75 HTS | | | | sweetness intensity compared to control. Sweet |
| | | | | aftertaste. |

### Example 32: HTS and Stevia in an Almond Milk (Sweetener Composition)

HTS ((0.0 mg/mL, 0.005 mg/mL, 0.007 mg/mL and 0.010 mg/mL) and stevia (0.0085% w/w, 0.0055% w/w, 0.0032% w/w, and 0.0015% w/w (respectively)) were added to an unsweetened almond milk. The sweetener mixture was compared to a control with only stevia added to the matrix. The sweetener mix was expected to be as sweet as or sweeter than the control for this matrix. Of total perceived sweetness intensity in each of the experimental samples in Table 25, 75% comes from stevia and 25% comes from HTS, 50% comes from stevia and 50% comes from HTS, 25% comes from stevia and 75% comes from HTS, respectively.

TABLE 25
HTS and Stevia Mixtures in an Almond Milk Compared to Controls

| | Stevia (RA95) | | | |
|---|---|---|---|---|
| Sample | w/w | HTS | | |
| Name | Concentration | Amount | | Observations |
| Unsweetened | NA | NA | | Nutty, creamy, low sweetness. |
| Control | | | | |
| Stevia RA95 | 0.0085% | 0.0 | mg/mL | Similar max sweetness intensity compared to |
| Control | | | | sucrose control. Bitter and metallic notes. |
| | | | | Increased grain flavor intensity. Sweet |
| | | | | aftertaste. |
| Stevia RA95 | 0.0055% | 0.005 | mg/mL | Reduced bitter, grain, and metallic notes. |
| 75/25 HTS | | | | Similar max sweetness intensity to control. |
| Stevia RA95 | 0.0032% | 0.007 | mg/mL | Increased almond flavor intensity. Similar |
| 50/50 HTS | | | | max sweetness intensity to control. Increased |
| | | | | sweet aftertaste compared to control. |
| Stevia RA95 | 0.0015% | 0.010 | mg/mL | Increased almond flavor intensity. Similar |
| 25/75 HTS | | | | max sweetness intensity to control. Increased |
| | | | | sweet aftertaste compared to control. |

### Example 33: HTS and Stevia in a Chocolate Spread (Sweetener Composition)

HTS ((0.0 mg/mL, 0.008 mg/mL, 0.023 mg/mL and 0.064 mg/mL) and stevia (0.0750% w/w, 0.0400% w/w, 0.0130% w/w, and 0.0042% w/w (respectively)) were added to a chocolate spread. The sweetener mixture was compared to a control with only stevia added to the matrix. The sweetener mix was expected to be as sweet as or sweeter than the control for this matrix. Of total perceived sweetness intensity in each of the experimental samples in Table 26, 75% comes from stevia and 25% comes from HTS, 50% comes from stevia and 50% comes from HTS, 25% comes from stevia and 75% comes from HTS, respectively.

TABLE 26
HTS and Stevia Mixtures in a Chocolate Spread Compared to Controls

| | Stevia RA95 | | | |
|---|---|---|---|---|
| | w/w | HTS | | |
| Sample | Concentration | Amount | | Observations |
| Unsweetened | NA | NA | | Intense cocoa and bitter, salty, astringent. |
| Control | | | | |
| Stevia RA95 | 0.0750% | 0.0 | mg/mL | Initial cocoa notes up front then strong sweetness |
| Control | | | | similar to sucrose control. |
| Stevia RA95 | 0.0400% | 0.008 | mg/mL | Increased initial sweetness compared to control. |
| 75/25 HTS | | | | Increased initial cocoa and salt flavor intensity |
| | | | | compared to control. Increased sweet aftertaste |
| | | | | compared to control. |
| Stevia RA95 | 0.0130% | 0.023 | mg/mL | Increased initial cocoa and salt flavor intensity |
| 50/50 HTS | | | | compared to control. Reduced max sweetness |
| | | | | intensity compared to control. |
| Stevia RA95 | 0.0042% | 0.064 | mg/mL | Reduced max sweetness intensity compared to |
| 25/75 HTS | | | | control. Strong sweet and salty aftertaste. |

### Example 34: HTS and Sucrose (Flavor Modifying Composition)

HTS (0.004 mg/mL) and sucrose (10% w/w concentration) were added to a mock beverage matrix (citric acid, a flavor and carbonation). The mock beverage had a pH of about 3 and 0.25% w/w lemon/lime flavor. HTS and sucrose were added to hit a target of 10% w/w sucrose equivalent sweetness. The HTS/sucrose mixture was compared to an unsweetened control, an unsweetened control+0.004 mg/mL HTS, and a sucrose sweetened control (10% w/w concentration of sucrose). See Table 27. The HTS/sucrose mixture increased the perception of the flavor as compared to the controls.

TABLE 27
HTS and Sucrose Mixtures Compared to Controls

| | Sucrose w/w | HTS | | |
|---|---|---|---|---|
| Sample | Concentration | Amount | | Observations |
| Unsweetened | NA | 0 | | Reduced citrus flavor intensity; sour, |
| Control | | | | astringent, and harsh. |
| Unsweetened | NA | 0.004 | mg/mL | More body, mellowed mouthfeel, more citrus |
| Control + HTS | | | | |
| Sucrose | 10% | 0 | | Good sweetness to acid ratio; quick onset of |
| | | | | sweet followed by sour; pleasant citrus flavor |
| Sucrose + HTS | 10% | 0.004 | mg/mL | Extended sweetness intensity, more body. |

Enhanced lime note from citrus blend

### Example 35: HTS and Erythritol (Flavor Modifying Composition)

HTS (0.004 mg/mL) and erythritol (13.3% w/w concentration) were added to a mock beverage matrix (citric acid, a flavor, and carbonation). The mock beverage had a pH of about 3 and 0.25% w/w lemon/lime flavor. HTS and erythritol were added to hit a target of 10% w/w sucrose equivalent sweetness. The HTS/erythritol mixture was compared to an unsweetened control, an unsweetened control+0.004 mg/mL HTS, and an erythritol sweetened control (13.3% w/w concentration). See Table 28. The HTS/erythritol mixture increased the perception of the flavor as compared to the controls.

TABLE 28
HTS and Erythritol Mixtures in Beverage Samples Compared to Controls

| | Erythritol w/w | HTS | | |
|---|---|---|---|---|
| Sample | Concentration | Amount | | Observations |
| Unsweetened | NA | 0 | | Reduced citrus flavor intensity; sour, |
| Control | | | | astringent, and harsh. |
| Unsweetened | NA | 0.004 | mg/mL | More body, mellowed mouthfeel, more citrus |
| Control + HTS | | | | |
| Erythritol | 13.30% | 0 | | More sour, bitter, mineral, mouth drying, |
| | | | | less sweetness |
| Erythritol + HTS | 13.30% | 0.004 | mg/mL | More citrus, less bitter, less astringency, more |

sweet than erythritol alone, better body

### Example 36: HTS and Sucralose (Flavor Modifying Composition)

HTS (0.004 mg/mL) and sucralose (0.0387% w/w concentration) were added to a mock beverage matrix (citric acid, a flavor, and carbonation). The mock beverage had a pH of about 3 and 0.25% w/w lemon/lime flavor. HTS and sucralose were added to hit a target of 10% w/w sucrose equivalent sweetness. The HTS/sucralose mixture was compared to an unsweetened control, an unsweetened control+0.004 mg/mL HTS, and a sucralose sweetened control (0.0387% w/w concentration). See Table 29. The HTS/sucralose mixture increased the perception of the flavor as compared to the two controls.

TABLE 29
HTS and Sucralose Mixtures in Beverage Samples Compared to Controls

| | Sucralose w/w | HTS | | |
|---|---|---|---|---|
| Sample | Concentration | Amount | | Observations |
| Unsweetened | NA | 0 | | Reduced citrus flavor intensity; sour, |
| Control | | | | astringent, and harsh. |
| Unsweetened | NA | 0.004 | mg/mL | More body, mellowed mouthfeel, more citrus |
| Control + HTS | | | | |
| Sucralose | 0.0387% | 0 | | Sweet linger, little flavor carry over |
| Sucralose + HTS | 0.0387% | 0.004 | mg/mL | Enhanced sweetness intensity, more body, |

lemon and lime extension, strong sweet
linger but bright and fruity. Reduced artificial
sweetness.

### Example 37: HTS and Stevia (Stevia Extract) (Flavor Modifying Composition)

HTS (0.004 mg/mL) and stevia (0.0395% w/w concentration) were added to a mock beverage matrix (citric acid, a flavor, and carbonation). The mock beverage had a pH of about 3 and 0.25% w/w lemon/lime flavor. HTS and stevia were added to hit a target of 10% w/w sucrose equivalent sweetness. The HTS/stevia mixture was compared to an unsweetened control, an unsweetened control+0.004 mg/mL HTS, and a stevia sweetened control (0.0395% w/w concentration). See Table 30. The HTS/stevia mixture increased the perception of the flavor as compared to the two controls.

TABLE 30
HTS and Stevia Mixtures in Beverage Samples Compared to Controls

| | Stevia w/w | HTS | | |
|---|---|---|---|---|
| Sample | Concentration | Amount | | Observations |
| Unsweetened | NA | 0 | | Reduced citrus flavor intensity; sour, |
| Control | | | | astringent, and harsh. |
| Unsweetened | NA | 0.004 | mg/mL | More body, mellowed mouthfeel, more citrus |
| Control + HTS | | | | |
| Stevia | 0.0395% | 0 | | Stevia gives sweetness with bitter and |
| | | | | metallic off notes; no body, medicinal, |
| | | | | astringent, lemon lime flavor |
| Stevia + HTS | 0.0395% | 0.004 | mg/mL | Helped to reduce Stevia off note intensity, juicier |

fresher citrus character, and longer linger

### Example 38: HTS and Allulose in a Lemon/Lime Soda (Sweetener Composition)

HTS ((0.0 mg/mL and 0.023 mg/mL) and allulose (14.3% w/w and 7.14% w/w (respectively)) were added to a lemon/lime soda. The sweetener mixture was compared to a control with only allulose added to the matrix. The sweetener mix was expected to be as sweet as or sweeter than the allulose control for this matrix. Of total perceived sweetness intensity in the experimental sample in Table 31, 50% comes from allulose and 50% comes from HTS.

TABLE 31
HTS and Allulose Mixture in a Lemon/Lime Soda Compared to Controls

| | Allulose w/w | HTS | | |
|---|---|---|---|---|
| Sample | Concentration | Amount | | Observations |
| Unsweetened | NA | NA | | Sour, bitter, astringent, harsh, citrus. |
| Control | | | | |
| Allulose | 14.3% | 0.0 | mg/mL | Muted citrus flavor and sweetness intensity. |
| Control | | | | Metallic and astringent. |
| Allulose | 7.14% | 0.023 | mg/mL | Enhanced citrus flavor intensity and max |
| 50/50 HTS | | | | sweetness intensity. |

### INCORPORATION BY REFERENCE

The contents of all references (including literature references, issued patents, published patent applications, co-pending patent applications, and GenBank numbers) cited throughout this application are hereby expressly incorporated herein in their entireties by reference. Unless otherwise defined, all technical and scientific terms used herein are accorded the meaning commonly known to one with ordinary skill in the art.

International patent application PCT/US2020/012955, filed Jan. 9, 2020, published as WO2020/146650 on Jul. 16, 2020 is incorporated by reference herein in its entirety at least to provide additional description, which is not inconsistent with the description herein, of sweetening composition comprising mycelia of an ascomycete or an aqueous extract thereof or comprising an aqueous extract of a fruiting body of an ascomycete, as well as uses of such composition to provide improve flavor to a product for oral administration.

International patent application PCT/US2021/039176, filed Jun. 25, 2021, published as WO2021/263158 on Dec. 30, 2021 is incorporated by reference herein in its entirety at least to provide additional description, which is not inconsistent with the description herein, of fungal sweet-taste modifying proteins and cDNA encoding such proteins. This patent application can also provide description of methods for isolating such cDNA and for isolating and expressing such proteins as well as description of sweetening compositions and methods to provide improved flavor to products for oral administration.

International patent application PCT/US2022/82443, filed Dec. 27, 2022, published as WO2023/129938, on Jul. 6, 2023, is also incorporated by reference herein in its entirety at least to provide additional description, which is not inconsistent with the description herein, of sweet protein mutants, including proteins, genes, cDNA encoding said proteins, and compositions thereof. The sequence listing of this published patent application is incorporated by reference herein in its entirety.

U.S. provisional application 63/444,185, filed Feb. 8, 2023 is incorporated by reference herein in its entirety. If the disclosure of the '185 provisional application is inconsistent with that of the present application, the present application controls.

U.S. provisional application 63/524,794, filed Jul. 3, 2023 is incorporated by reference herein in its entirety. If the disclosure of the '794 provisional application is inconsistent with that of the present application, the present application controls.

U.S. provisional application 63/541,591, filed Sep. 29, 2023 is incorporated by reference herein in its entirety. If the disclosure of the '591 provisional application is inconsistent with that of the present application, the present application controls.

### EQUIVALENTS

Those skilled in the art will recognize, or be able to ascertain using no more than routine experimentation, many equivalents of the specific embodiments of the invention described herein. Such equivalents are intended to be encompassed by the following claims.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “comprises”, “having,” “including,” and “containing” are to be construed as open-ended terms (i.e., meaning “including, but not limited to,”) unless otherwise noted. The terms “consisting of” or “consists of” are construed as a close-ended term (i.e., excluding components or steps other than those listed). The terms “consisting essentially of” or “consists essentially of” allow for the inclusion of components or steps that are not essential to the function or activity of the product or method and do not materially affect the function or activity. Any recitation herein of the term “comprising”, particularly in a description of components of a composition, polynucleotide or polypeptide, is understood to encompass those compositions and methods “consisting essentially of and consisting of the recited components or elements.

When a group of materials, compositions, components or compounds is disclosed herein, it is understood that all individual members of those groups and all subgroups thereof are disclosed separately. Every formulation or combination of components described or exemplified herein can be used to practice the invention, unless otherwise stated. Recitation of ranges of values herein are merely intended to serve as a shorthand method of referring individually to each separate value falling within the range, unless otherwise indicated herein, and each separate value is incorporated into the specification as if it were individually recited herein. Additionally, the end points in a given range are to be included within the range. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All publications referred to herein are incorporated herein to the extent not inconsistent herewith. Some references provided herein are incorporated by reference to provide details of additional uses of the invention. All patents and publications mentioned in the specification are indicative of the levels of skill of those skilled in the art to which the invention pertains. References cited herein are incorporated by reference herein in their entirety to indicate the state of the art as of their filing date and it is intended that this information can be employed herein, if needed, to exclude specific embodiments that are in the prior art.

TABLE 31
TABLE OF SEQUENCES:

| Sequence Number (ID): 1 | |
|---|---|
| Length: 1072 | |
| Molecule Type: DNA | |
| > organism, Mattirolomyces terfezioides | |
| Residues: | |
| caagtcttta agtgttgatc gagcagtaga aatattaatc gcccaattaa ttgctcttac | 60 |
| tctaggaaat gtagtactca agttggccct ctaagtccct gattcagtgt cactttttct | 120 |
| cctttgacag taggctagat tctagctctt gtgtccgttt cttaaaatcc tgatatggca | 180 |
| ccttagegtc agagcagaat acctgttctc atacaccttg gctatgatcc gatatttaaa | 240 |
| tagagcaatc acccttctgg atatcttccc catagctccc attgacctca aacattttct | 300 |
| aatcccctat accagccata ttacttgtct aagcacttca cattcttcct cacaatacga | 360 |
| aacctcctaa aatgcctgat ctctcctcct tcattacgat taagaacaac tctaaccacg | 420 |
| tcttcactcg gacggcaatt tattctaagt atgccgctgt gcagtggagt cccgagccgc | 480 |
| aactctcaat ctctcccggc aaatgggatt tgtttatttt aaaggacatc ttgtctatcc | 540 |
| gtgggacttc gggatatgta caatatcggg ttggggatgg tcctggatgg gttagggtca | 600 |
| ccttttcttc tctagtcggg gccgatgaag tggcagagtg gagctcaggt gacctacctg | 660 |
| atggctttgt tctccaaaaa ccagttcgca ctgggtccag gcctttgcag gcaacgttcg | 720 |
| aggctacaaa acagtaaaga tcgatgatgc ctaatatgcc tccataacac tgacccgcgt | 780 |
| gcacatggcc gcatgaatga taagggggat atcgatgatg atgggattag ttattggaat | 840 |
| tttcacaatg gacgtcggct tggatttaca ataatcgttt catttgtatt caaatattcc | 900 |
| tatttccttg ggtttttgta tttatctcct tcatcacgcc ttctgaggcc gtgggaagat | 960 |
| gaatatgtaa tcaaaagaag ttaggatatg catcatgtac agaaagtgga ccgcaacccc | 1020 |
| ttcagcgaaa tgttataaag atgatatcta agacgccaaa gcacattctc ag | 1072 |
| Sequence Number (ID): 2 | |
| Length: 366 | |
| Molecule Type: DNA | |
| > organism, Mattirolomyces terfezioides | |
| Residues: | |
| atgcctgatc tctcctcctt cattacgatt aagaacaact ctaaccacgt cttcactcgg | 60 |
| acggcaattt attctaagta tgccgctgtg cagtggagtc cegagccgca actctcaatc | 120 |
| tctcccggca aatgggattt gtttatttta aaggacatct tgtctatccg tgggacttcg | 180 |
| ggatatgtac aatatcgggt tggggatggt cctggatggg ttagggtcac cttttcttct | 240 |
| ctagtcgggg ccgatgaagt ggcagagtgg agctcaggtg acctacctga tggctttgtt | 300 |
| ctccaaaaac cagttcgcac tgggtccagg cctttgcagg caacgttcga ggctacaaaa | 360 |
| cagtaa | 366 |
| Sequence Number (ID): 3 | |
| Length: 121 | |
| Molecule Type: AA | |
| Features Location/Qualifiers: | |
| > organism, Mattirolomyces terfezioides | |
| Residues: | |
| MPDLSSFITI KNNSNHVFTR TAIYSKYAAV QWSPEPQLSI SPGKWDLFIL KDILSIRGTS | 60 |
| GYVQYRVGDG PGWVRVTFSS LVGADEVAEW SSGDLPDGFV LQKPVRTGSR | 121 |
| PLQATFEATK Q | |
| Sequence Number (ID): 4 | |
| Length: 384 | |
| Molecule Type: DNA | |
| Features Location/Qualifiers: | |
| > note, Modified coding sequence for E. coli expression for protein from | |
| Mattirolomyces terfezioides with His-Tag | |
| Residues: | |
| atgccggatt tgagctcgtt cattactatt aagaataact ctaaccatgt ttttacgcgt | 60 |
| accgcaatct attccaaata tgccgcagtc cagtggagcc cggaaccgca gctgagcatt | 120 |
| agcccaggta aatgggacct gttcatcctg aaagacatcc tgagcatccg cggtacgtcc | 180 |
| ggctacgtgc agtaccgtgt cggcgacggt ccgggctggg tgcgtgttac ctttagcage | 240 |
| ctggtgggtg cggacgaagt tgctgagtgg agcagcggtg atctgccgga tggctttgtt | 300 |
| ctgcaaaagc ctgtccgcac cggtagccgt ccgctgcaag cgacgttcga ggcgaccaag | 360 |
| caacatcacc atcaccacca ctaa | 384 |
| Sequence Number (ID): 5 | |
| Length: 127 | |
| Molecule Type: AA | |
| Features Location/Qualifiers: | |
| > note, Synthetic polypeptide with His-tag | |
| Residues: | |
| MPDLSSFITI KNNSNHVFTR TAIYSKYAAV QWSPEPQLSI SPGKWDLFIL | 60 |
| KDILSIRGTS | |
| GYVQYRVGDG PGWVRVTFSS LVGADEVAEW SSGDLPDGFV LQKPVRTGSR | 120 |
| PLQATFEATK | |
| QHHHHHH | 127 |
| Sequence Number (ID): 6 | |
| Length: 366 | |
| Molecule Type: DNA | |
| Features Location/Qualifiers: | |
| > note, Modified coding sequence for Saccharomyces cerevisiae | |
| expression for protein from Mattirolomyces terfezioides to which His-Tag coding region is added | |
| Residues: | |
| atgcctgatt tatcaagttt tatcaccatt aaaaataact ctaaccatgt ttttactaga | 60 |
| acagccatct actcaaagta cgcagccgtc caatggtccc cagaacctca gttgtctata | 120 |
| tctccaggta aatgggatct tttcatctta aaagatattc taagtattag aggcacttct | 180 |
| ggttacgtac agtatcgtgt tggtgatgga cctggttggg ttagagtaac attcagctca | 240 |
| ttggttgggg ctgacgaagt ggctgagtgg tcctcaggtg acctcccaga tggcttcgtg | 300 |
| ctgcaaaagc cagtcagaac tggatctaga ccattgcaag cgacattcga agcaacaaag | 360 |
| caatga | 366 |
| Sequence Number (ID): 7 | |
| Length: 5 | |
| Molecule Type: AA | |
| Features Location/Qualifiers: | |
| > note, Amino Acid Linker | |
| > note, May be repeated n times, where n is any integer | |
| Residues: | |
| GGGGS | 5 |
| Sequence Number (ID): 8 | |
| Length: 5 | |
| Molecule Type: AA | |
| Features Location/Qualifiers: | |
| > note, Amino Acid Linker | |
| > note, May be repeated n times, where n is any integer | |
| Residues: | |
| EAAAK | 5 |
| Sequence Number (ID): 9 | |
| Length: 58 | |
| Molecule Type: DNA | |
| Features Location/Qualifiers: | |
| > note, Synthetic Adapter Sequence | |
| Residues: | |
| aatgatacgg cgaccaccga gatctacact ctttccctac acgacgctct tccgatct | 58 |
| Sequence Number (ID): 10 | |
| Length: 58 | |
| Molecule Type: DNA | |
| Features Location/Qualifiers: | |
| > note, Synthetic Adapter Sequence | |
| > note, insertion of i7 barcode | |
| Residues: | |
| gatcggaaga gcacacgtct gaactccagt cacnatcteg tatgccgtct tctgcttg | 58 |
| SEQ ID NO: 11-SEQ 1D NO: 140 see: WO2023/129938 published Jul. 6, 2023 | |
| Sequence Number (ID): 141 | |
| Length: 121 | |
| Molecule Type: AA | |
| Features Location/Qualifiers: | |
| > organism, Mattirolomyces terfezioides (mature protein HTS-2 isoform, | |
| SEQ ID NO: 3 with methionine missing at position 1) | |
| Residues: | |
| PDLSSFITI KNNSNHVFTR TAIYSKYAAV QWSPEPQLSI SPGKWDLFIL KDILSIRGTS | 60 |
| GYVQYRVGDG PGWVRVTFSS LVGADEVAEW SSGDLPDGFV LQKPVRTGSR | 120 |
| PLQATFEATK Q | |
| AMINO ACID SEQUENCE (SEQ ID NO: 142) HTS-2 isoform with His-tag | |
| PDLSSFITIK NNSNHVFTRT AIYSKYAAVQ WSPEPQLSIS PGKWDLFILK DILSIRGTSG | 60 |
| YVQYRVGDGP GWVRVTFSSL VGADEVAEWS SGDLPDGFVL QKPVRTGSRPL | 110 |
| QATFEATKQH HHHHH | 205 |
| AMINO ACID SEQUENCE: (SEQ ID NO: 143) Artificial (Double Mutant S33C_I49C) | |
| MPDLSSFITI KNNSNHVFTR TAIYSKYAAV QWCPEPQLSI SPGKWDLFCL KDILSIRGTS | 121 aa + optional His6 tag |
| GYVQYRVGDGPGWVRVTFSS LVGADEVAEW SSGDLPDGFV LQKPVRTGSR PLQATFEATK | |
| QHHHHHH | |
| REVERSE TRANSLATION of HTS variant of SEQ ID NO: 143-DNA Sequence (SEQ ID NO: 144) | |
| atgccggatc tgagcagctt tattaccatt aaaaacaaca gcaaccatgt gtttacccgc | 60 |
| accgcgattt atagcaaata tgcggcggtg cagtggtgcc cggaaccgca gctgagcatt | 120 |
| agcccgggca aatgggatct gttttgcctg aaagatattc tgagcattcg cggcaccagc | 180 |
| ggctatgtgc agtatcgcgt gggcgatggc ccgggctggg tgcgcgtgac ctttagcagc | 240 |
| ctggtgggcg cggatgaagt ggcggaatgg agcagcggcg atctgccgga tggctttgtg | 300 |
| ctgcagaaac cggtgcgcac cggcagccgc ccgctgcagg cgacctttga agcgaccaaa | 360 |
| cag | 363 (no taa and no His-tag) |
| AMINO ACID SEQUENCE; (SEQ ID NO: 145)(Double mutant Y24C_Y62C) | |
| MPDLSSFITI KNNSNHVFTR TAICSKYAAV QWSPEPQLSI SPGKWDLFIL KDILSIRGTS | |
| GCVQYRVGDG PGWVRVTFSS LVGADEVAEW SSGDLPDGFV LQKPVRTGSR PLQATFEATK | |
| Q (121 AA) | |
| Amino Acid SEequence (SEQ ID NO: 146)(Double Mutant S33C_149C with methionine at position 1 | |
| missing) | |
| PDLSSFITIK NNSNHVFTRT AIYSKYAAVQ WCPEPQLSIS PGKWDLFCLK DILSIRGTSG | |
| YVQYRVGDGP GWVRVTFSSL VGADEVAEWS SGDLPDGFVL QKPVRTGSRP LQATFEATKQ | |
| (120 AA) | |
| Amino Acid SEequence (SEQ ID NO: 147)(Double Mutant Y24C_Y62C with methionine at position 1 | |
| missing) | |
| PDLSSFITIK NNSNHVFTRT AICSKYAAVQ WSPEPQLSIS PGKWDLFILK DILSIRGTSG | |
| CVQYRVGDGP GWVRVTFSSL VGADEVAEWS SGDLPDGFVL QKPVRTGSRP LQATFEATKQ | |
| (120 AA) | |
| E. COLI CODON OPTIMIZED Sequence (SEQ ID NO: 148) Artificial | |
| atgccggacc tgtcttcctt tatcactatc aaaaataact ctaaccatgt tttcacccgc | 60 |
| acggcgatct acagcaaata cgccgcggtg cagtggtgtc cggaaccgca gctgtctatc | 120 |
| agcccgggca aatgggacct gttctgtctg aaagatatcc tgagcatccg tggtacctct | 180 |
| ggttatgttc agtaccgtgt cggcgatggt ccgggttggg ttcgcgttac cttcagctct | 240 |
| ctggttggtg cggacgaagt tgcggaatgg tcctctggtg acctgccaga tggtttcgtg | 300 |
| ctgcagaaac cggttcgtac tggctctcgt ccgctccagg ccactttcga agctaccaaa | 360 |
| caa | 363 (no taa and no His Tag) |
| EXEMPLARY SEQUENCE ENCODING (HIS)6 (SEQ ID NO: 149) | |
| caacatcaccatcaccaccac | |

The His-Tag encoding sequence (or one that is optimized for expression in a selected host) is inserted after the last amino acid codon in polynucleotide sequence and before stop codon taa.

1. An isolated polynucleotide encoding a polypeptide selected from the group consisting of:
(a) a polypeptide comprising the amino acid sequence of SEQ ID NO:141; and
(b) a polypeptide comprising an amino acid sequence having at least 95% sequence identity to the amino acid sequence of SEQ ID NO: 141; wherein the polypeptide has sweet-taste modulation activity wherein the isolated polynucleotide is operably linked to a heterologous regulatory element.

2. The isolated polynucleotide of claim 1, wherein the polynucleotide sequence further encodes a protein tag or label.

3. The isolated polynucleotide of claim 2, wherein the protein tag is a histidine tag.

4. The isolated polynucleotide of claim 3, wherein the polypeptide comprises the amino acid sequence of SEQ ID NO:142.

5. The isolated polynucleotide of claim 1, wherein the polypeptide comprises the amino acid sequence of SEQ ID NO:141.

6. The isolated polynucleotide of claim 1, wherein the polypeptide is that of the amino acid sequence of SEQ ID NO:141.

7. The isolated polynucleotide of claim 1, wherein the polypeptide is that of the amino acid sequence of SEQ ID NO:142.

8. The isolated polynucleotide of claim 1, wherein the polypeptide comprises an amino acid sequence having 1 or 2 amino acid deletions, insertions or substitutions from the amino acid sequence of SEQ ID NO:141, wherein the polypeptide has sweet-taste modulation activity.

9. The isolated polynucleotide of claim 1, wherein the polypeptide comprises an amino acid sequence having 1 or 2 amino acid deletions, insertions or substitutions from the amino acid sequence of SEQ ID NO:142, wherein the polypeptide has sweet-taste modulation activity.

10. An expression cassette comprising the isolated polynucleotide of claim 1.

11. A vector comprising the isolated polynucleotide of claim 1.

12. A host cell transformed with the vector of claim 11.

13. A method of producing a protein having sweet-taste modulation activity, comprising culturing the host cell of claim 12 in a medium under conditions that result in producing the protein having sweet-taste modulation activity.

14. The method of claim 13, wherein the host cell is transformed with a vector comprising an isolated polynucleotide which encodes a polypeptide comprising the amino acid sequence of SEQ ID NO:141.

15. The method of claim 13, wherein the host cell is transformed with a vector comprising an isolated polynucleotide which encodes a polypeptide comprising the amino acid sequence of SEQ ID NO:142.

16. An isolated polypeptide comprising an amino acid sequence having at least 95% sequence identity to the amino acid sequence of SEQ ID NO:141; wherein the polypeptide has sweet-taste modulation activity and wherein the polypeptide is a non-naturally occurring polypeptide.

17. The isolated polypeptide of claim 16, wherein the polypeptide comprises an amino acid sequence having 1 or 2 amino acid deletions, insertions or substitutions from the amino acid sequence of SEQ ID NO:141.

18. The isolated polynucleotide of claim 16, wherein the polypeptide comprises an amino acid sequence having 1 or 2 amino acid deletions, insertions or substitutions from the amino acid sequence of SEQ ID NO:142, wherein the polypeptide has sweet-taste modulation activity.

19. The isolated polypeptide of claim 16, which is a polypeptide comprising the amino acid sequence of SEQ ID NO:142.

20. A composition, comprising a combination of:
(a) a product for oral administration, and
(b) a sweetening composition comprising an isolated protein produced by the method of claim 13,
wherein the product for oral administration is not Mattirolomyces terfezioides truffle, and
wherein the combination has enhanced sweet taste compared to the product for oral administration.

21. The composition of claim 20, wherein the product for oral administration is a food, a beverage, a dietary supplement composition, or a pharmaceutical composition.

22. A composition, comprising a combination of:
(a) a product for oral administration, and
(b) a sweetening composition comprising:
(i) an isolated polypeptide comprising the amino acid sequence of SEQ ID NO: 141 or SEQ ID NO:142; or
(ii) an isolated polypeptide comprising an amino acid sequence having at least 95% sequence identity to the amino acid sequence of SEQ ID NO:141; wherein the polypeptide has sweet-taste modulation activity,
wherein the product for oral administration is not Mattirolomyces terfezioides truffle, and
wherein the combination has enhanced sweet taste compared to the product for oral administration.

23. The composition of claim 22, wherein the sweetening composition comprises an isolated polypeptide comprising the amino acid sequence of SEQ ID NO:141 or SEQ ID NO: 142.

24. The composition of claim 22, wherein the product for oral administration is a food, a beverage, a dietary supplement composition, or a pharmaceutical composition.

25. A method for modulating the taste of a product for oral administration, comprising:
combining a product for oral administration with an effective amount of a sweetening composition comprising an isolated protein produced by the method of claim 13,
wherein the product for oral administration is not Mattirolomyces terfezioides truffle, and
wherein the combination has enhanced sweet taste compared to the product for oral administration.

26. The method of claim 25, the sweetening composition comprising an isolated protein which comprises the amino acid sequence of SEQ ID NO:141 or SEQ ID No:142.

27. The method of claim 25, wherein the product for oral administration is a food, a beverage, a dietary supplement composition, or a pharmaceutical composition.

28. A method for modulating the taste of a product for oral administration, comprising:
combining a product for oral administration with an effective amount of a sweetening composition comprising:
(i) an isolated polypeptide comprising the amino acid sequence of SEQ ID NO: 141 or SEQ ID NO:142; or
(ii) an isolated polypeptide comprising an amino acid sequence having at least 95% sequence identity to the amino acid sequence of SEQ ID NO:141; wherein the polypeptide has sweet-taste modulation activity;
wherein the product for oral administration is not Mattirolomyces terfezioides truffle, and
wherein the combination has enhanced sweet taste compared to the product for oral administration.

29. The method of claim 28, wherein the product for oral administration is a food, a beverage, a dietary supplement composition, or a pharmaceutical composition.

30. A method for purifying a protein having sweet-taste modulation activity comprising:
(a) producing the protein by the method of claim 13, and
(b) purifying the protein via hydrophobic interaction chromatography (HIC) followed by size exclusion chromatography (SEC).
